(19) United States
(12) Patent Application Publication
(10) Pub. No.: US 2024/0216410 A1
(43) Pub. Date: Jul. 4, 2024

(54) METHODS AND COMPOSITIONS FOR TREATING CANCER THERAPY-INDUCED NEUTROPENIC FEVER AND/OR GVHD

(71) Applicant: BOARD OF REGENTS, THE UNIVERSITY OF TEXAS SYSTEM, Austin, TX (US)

(72) Inventors: Robert R. JENQ, Houston, TX (US); Mohamed A. JAMAL, Houston, TX (US); Jennifer L. KARMOUCH, Houston, TX (US); Eiko HAYASE, Houston, TX (US)

(73) Assignee: BOARD OF REGENTS, THE UNIVERSITY OF TEXAS SYSTEM, Austin, TX (US)

(21) Appl. No.: 18/283,408

(22) PCT Filed: Mar. 24, 2022
(86) PCT No.: PCT/US2022/021660

§ 371 Date: Sep. 21, 2023

Related U.S. Application Data
(60) Provisional application No. 63/165,639, filed on Mar. 24, 2021.
(60) Provisional application No. 63/273,051, filed on Oct. 28, 2021.

(51) Int. Cl. A61K 31/7052; A61K 9/00; A61K 31/12; A61K 31/19; A61K 31/4045; A61K 31/407; A61K 31/546; A61K 31/7004; A61K 31/714; A61K 45/06; A61P 1/04

(52) U.S. Cl. CPC A61K 31/7052; A61K 9/0053; A61K 31/12; A61K 31/19; A61K 31/4045; A61K 31/407; A61K 31/546; A61K 31/7004; A61K 31/714; A61K 45/06; A61P 1/04

(57) ABSTRACT

The current disclosure provides methods and compositions related to treating or preventing neutropenic fever (e.g., cancer therapy-induced neutropenic fever) and/or GVHD (e.g., HCT-related and/or neutropenic fever therapy-induced GVHD). In specific cases, it may be determined whether or not the subject is likely to develop or is at risk of developing neutropenic fever and/or GVHD, such as cancer therapy-induced neutropenic fever and/or HCT-related and/or neutropenic fever therapy-induced GVHD, based on the subject's gut microbiome. A subject may be provided a composition comprising one or more agents targeting growth or expansion the one or more genera of mucus-degrading gut bacteria, one or more mucus-degrading enzyme inhibitors, one or more mediators of organic acid metabolite levels, and/or one or more carbohydrate substrates metabolized by the one or more genera of mucus-degrading gut bacteria based on analysis of the gut microbiome of the subject receiving a HCT therapy and/or a neutropenic fever therapy following administration of a cancer therapy, in specific embodiments.

### CROSS-REFERENCE TO RELATED APPLICATIONS

This application claims priority to U.S. Provisional Application Ser. No. 63/165,639, filed Mar. 24, 2021, and U.S. Provisional Application Ser. No. 63/273,051, filed Oct. 28, 2021, which are incorporated by reference herein in their entirety.

### STATEMENT REGARDING FEDERALLY SPONSORED RESEARCH OR DEVELOPMENT

This invention was made with government support under R01HL124112 awarded by the National Institutes of Health and RR160089 awarded by the Cancer Prevention and Research Institute of Texas. The government has certain rights in the invention.

### TECHNICAL FIELD

Embodiments of the disclosure concern at least the fields of cell biology, molecular biology, microbiology, and medicine.

### BACKGROUND

One of the most common and potentially serious complications of cancer therapy is neutropenia and subsequent infectious complications, with an estimated mortality of nearly 10% (1) as well as 100,000 hospitalizations and over $2.7 billion in hospitalization costs annually in the United States (2). At particularly high risk are patients undergoing chemotherapy for hematological malignancies including acute leukemias and high-grade lymphomas or receiving hematopoietic cell transplantation (HCT) after myeloablative conditioning (3).

Additionally, patients undergoing HCT, including allogeneic hematopoietic stem cell transplantation (allo-HSCT), are at risk for graft-versus-host disease (GVHD). The gastrointestinal tract is a primary target of allogeneic donor T-cells in allo-HSCT. The intestinal microbiota interacts with the host immune system and is an important modulator of GVHD. Broad-spectrum antibiotics such as carbapenems are often used in allo-HSCT patients to treat infections but have been found to increase the risk for intestinal GVHD.

Recognized is a need for methods and compositions to treat, prevent, or predict the development of neutropenic fever and/or GVHD, including cancer therapy-induced neutropenic fever and/or GVHD (e.g., HCT-related GVHD and/or neutropenic fever therapy-induced GVHD).

### SUMMARY

The current disclosure fulfills these needs in the art by providing methods and compositions for treating, preventing, or predicting the development of neutropenic fever and/or GVHD, including cancer therapy-induced neutropenic fever and/or HCT-related GVHD and/or neutropenic fever therapy-induced GVHD. Accordingly, aspects of the present disclosure provide methods and compositions useful for preventing or reducing the severity of, and/or delaying the onset of, neutropenic fever and/or GVHD, including cancer therapy-induced neutropenic fever and/or HCT-related GVHD and/or neutropenic fever therapy-induced GVHD. The present disclosure is also directed to systems, methods, and compositions related at least to determining or predicting a therapy outcome for a chemotherapy and/or determining or predicting development of neutropenic fever and/or GVHD, including cancer therapy-induced neutropenic fever and/or HCT-related GVHD and/or neutropenic fever therapy-induced GVHD, in a subject receiving a cancer therapy, an infusion of hematopoietic donor cells (HCT therapy), and/or a neutropenic fever therapy.

In particular embodiments, the gut microbiome composition correlates with the risk of developing neutropenic fever and/or GVHD. The present disclosure relates to compositions and methods for predicting development of neutropenic fever and/or GVHD in a subject, including by analyzing the intestinal microbiome of the subject. The disclosure concerns compositions and methods for determining a therapy outcome for a subject in need of chemotherapy, including by analyzing the intestinal microbiome of the subject. The disclosure concerns compositions and methods for determining a therapy outcome for a subject in need of HCT therapy, including by analyzing the intestinal microbiome of the subject. The disclosure concerns compositions and methods for determining a therapy outcome for a subject in need of neutropenic fever therapy, including by analyzing the intestinal microbiome of the subject. The present disclosure further provides therapeutic compositions and methods for treating a subject having neutropenic fever and/or GVHD, including to prevent or reduce the risk of development of neutropenic fever and/or GVHD. In some cases, analysis of the gut microbiome of a subject in need of chemotherapy, HCT therapy, and/or neutropenic fever therapy provides information that prevents or reduces the risk of development of neutropenic fever and/or GVHD in the patient. Such intervention may include, for example, one or more bacterial growth-suppressing agent compositions, mucus-degrading enzyme inhibitor compositions, compositions comprising mediators of organic acid metabolite levels in the gut, and/or compositions comprising one or more carbohydrate substrates metabolized by mucus-degrading gut bacteria.

Embodiments of the disclosure include methods for preventing neutropenic fever and/or GVHD, methods for preventing cancer therapy-induced neutropenic fever and/or HCT-related GVHD and/or neutropenic fever therapy-induced GVHD, methods for reducing the severity of neutropenic fever and/or GVHD, methods for reducing the severity and/or delaying the onset of cancer therapy-induced neutropenic fever and/or HCT-related GVHD and/or neutropenic fever therapy-induced GVHD, methods for treating neutropenic fever and/or GVHD, methods for treating cancer therapy-induced neutropenic fever and/or HCT-related GVHD and/or neutropenic fever therapy-induced GVHD, methods for determining a risk of developing neutropenic fever and/or GVHD, methods for determining a risk of developing cancer therapy-induced neutropenic fever and/or HCT-related GVHD and/or neutropenic fever therapy-induced GVHD, bacterial growth-suppressing agent compositions, mucus-degrading enzyme inhibitor compositions, compositions comprising mediators of organic acid metabolite levels in the gut, and/or compositions comprising one or more carbohydrate substrates metabolized by mucus-degrading gut bacteria.

Methods of the present disclosure can include at least 1, 2, 3, 4, 5, or more of the following steps: administering one or more bacterial growth-suppressing agents to a subject, administering one or more mucus-degrading enzyme inhibitors to a subject, administering one or more compositions comprising mediators of organic acid metabolite levels in the gut to a subject, administering one or more compositions comprising one or more carbohydrate substrates metabolized by gut bacteria, determining a subject to have a higher risk of developing neutropenic fever and/or GVHD, determining that neutropenic fever and/or GVHD poses a greater risk to the health or life of the subject, determining a subject to have increased abundance of mucus-degrading bacteria in the gut microbiome, determining a subject to have increased functional activity and/or expression levels of one or more mucus-degrading enzymes secreted by one or more genera of mucus-degrading bacteria in the gut microbiome, determining a subject to have increased levels of one or more organic acid metabolites produced following metabolism of mucin-derived carbohydrates by one or more genera of mucus-degrading bacteria in the gut microbiome, determining a subject to have decreased levels of one or more carbohydrate substrates metabolized by one or more genera of mucus-degrading bacteria in the gut microbiome, diagnosing a subject with neutropenia, identifying a risk of development of neutropenic fever in a subject, diagnosing a subject with neutropenic fever, diagnosing a subject with GVHD, predicting a therapy outcome for a subject in need of cancer therapy, predicting a therapy outcome for a subject in need of HCT therapy, predicting a therapy outcome for a subject in need of neutropenic fever therapy, obtaining a biological sample from a subject, obtaining a fecal sample from a subject, and comparing a fecal sample from a healthy subject with a fecal sample from a subject having neutropenia, neutropenic fever, and/or GVHD. It is contemplated that any one or more of these steps may be excluded from certain embodiments of the disclosure.

Compositions of the present disclosure can include at least 1, 2, 3, or more of the following components: a bacterial growth-suppressing agent composition, an antibiotic, an antibacterial protein or peptide, azithromycin, bucine, methyl-β-D-galactopyranoside, resacetophenone, serotonin, ruminal metabolites, malic acid, 3-indole acetic acid, hydrocinnamic acid, methylmalonic acid, gluconic acid, galacturonic acid, bis-hydroxy methyl propionic acid, a mucus-degrading enzyme inhibitor, a mediator of organic acid metabolite levels, a carbohydrate substrate metabolized by gut bacteria, propionate, acetate, butyrate, isovalerate, valerate, vitamins, vitamin B12, a probiotic, a prebiotic, carbapenems, meropenem, cefepime, monosaccharides, polysaccharides, mannose, glucose, xylose, and a pharmaceutical excipient. It is contemplated that any one or more of these components may be excluded from certain embodiments of the disclosure.

Disclosed herein, in some aspects, is a method of preventing or reducing the severity of cancer therapy-induced neutropenic fever, the method comprising prophylactically administering to a subject receiving a cancer therapy a therapeutically effective amount of a composition comprising one or more of the following: a) one or more agents targeting growth or expansion of one or more genera of mucus-degrading bacteria in the gut microbiome of the subject; b) one or more mucus-degrading enzyme inhibitors to inhibit mucus degradation by one or more genera of mucus-degrading bacteria in the gut microbiome of the subject; and/or c) one or more mediators of organic acid metabolite levels produced following metabolism of mucin-derived carbohydrates by one or more genera of mucus-degrading bacteria in the gut microbiome of the subject; wherein the one or more genera of mucus-degrading bacteria comprise Akkermansia or Bacteroides.

In some embodiments, the subject is at a higher risk than an average person in the general population receiving the cancer therapy of developing cancer therapy-induced neutropenic fever. In some embodiments, the cancer therapy-induced neutropenic fever poses a greater risk to the health or life of the subject than such a condition would pose to an average person in the general population receiving the cancer therapy.

In some embodiments, the subject was determined to have an increased abundance of mucus-degrading bacteria in the gut microbiome compared to a control or reference sample. In some embodiments, the increased abundance of mucus-degrading bacteria in the gut microbiome was determined from a fecal sample from the subject.

In some embodiments, the subject was determined to have an increase in functional activity and/or expression levels of one or more mucus-degrading enzymes secreted by one or more genera of mucus-degrading bacteria in the gut microbiome compared to a control or reference sample. In some embodiments, the increase in functional activity and/or expression levels of one or more mucus-degrading enzymes secreted by one or more genera of mucus-degrading bacteria in the gut microbiome was determined from a fecal sample from the subject.

In some embodiments, the subject was determined to have a decrease in the levels of one or more organic acid metabolites produced following metabolism of mucin-derived carbohydrates by one or more genera of mucus-degrading bacteria in the gut microbiome compared to a control or reference sample. In some embodiments, the decrease in the levels of one or more organic acid metabolites produced following metabolism of mucin-derived carbohydrates by one or more genera of mucus-degrading bacteria in the gut microbiome was determined from a fecal sample from the subject.

Disclosed herein, in some aspects, is a method of treating cancer therapy-induced neutropenic fever in a subject receiving a cancer therapy and having an increased abundance of the one or more genera of mucus-degrading bacteria in the gut microbiome of the subject compared to a control or reference sample, the method comprising administering to the subject a therapeutically effective amount of a composition comprising one or more of the following: a) one or more agents targeting growth or expansion of one or more genera of mucus-degrading bacteria in the gut microbiome of the subject; b) one or more mucus-degrading enzyme inhibitors to inhibit mucus degradation by one or more genera of mucus-degrading bacteria in the gut microbiome of the subject; and/or c) one or more mediators of organic acid metabolite levels produced following metabolism of mucin-derived carbohydrates by one or more genera of mucus-degrading bacteria in the gut microbiome of the subject; wherein the one or more genera of mucus-degrading bacteria comprise Akkermansia or Bacteroides.

Disclosed herein, in some aspects, is a method of treating cancer therapy-induced neutropenic fever in a subject receiving a cancer therapy and having increased functional activity or expression levels of one or more mucus-degrading enzymes secreted by one or more genera of mucus-degrading bacteria in the gut microbiome of the subject compared to a control or reference sample, the method comprising administering to the subject a therapeutically effective amount of a composition comprising one or more of the following: a) one or more agents targeting growth or expansion of one or more genera of mucus-degrading bacteria in the gut microbiome of the subject; b) one or more mucus-degrading enzyme inhibitors to inhibit mucus degradation by one or more genera of mucus-degrading bacteria in the gut microbiome of the subject; and/or c) one or more mediators of organic acid metabolite levels produced following metabolism of mucin-derived carbohydrates by one or more genera of mucus-degrading bacteria in the gut microbiome of the subject; wherein the one or more genera of mucus-degrading bacteria comprise Akkermansia or Bacteroides.

Disclosed herein, in some aspects, is a method of treating cancer therapy-induced neutropenic fever in a subject receiving a cancer therapy and having decreased levels of one or more organic acid metabolites produced following metabolism of mucin-derived carbohydrates by one or more genera of mucus-degrading bacteria in the gut microbiome of the subject compared to a control or reference sample, the method comprising administering to the subject a therapeutically effective amount of a composition comprising one or more of the following: a) one or more agents targeting growth or expansion of one or more genera of mucus-degrading bacteria in the gut microbiome of the subject; b) one or more mucus-degrading enzyme inhibitors to inhibit mucus degradation by one or more genera of mucus-degrading bacteria in the gut microbiome of the subject; and/or c) one or more mediators of organic acid metabolite levels produced following metabolism of mucin-derived carbohydrates by one or more genera of mucus-degrading bacteria in the gut microbiome of the subject; wherein the one or more genera of mucus-degrading bacteria comprise Akkermansia or Bacteroides.

In some embodiments, the subject does not exhibit symptoms of cancer therapy-induced neutropenic fever when the composition is administered. In some embodiments, the subject has been diagnosed with neutropenia. In some embodiments, the composition is administered after the subject has been diagnosed with neutropenia. In some embodiments, the composition is administered to the subject every day until the subject is no longer neutropenic.

In some embodiments, the subject is neutropenic due to the cancer therapy received by the subject. In some embodiments, the cancer therapy received by the subject comprises one or more chemotherapies, radiotherapies, and/or immunotherapies. In some embodiments, the one or more chemotherapies comprise alkylating agents, marrow-suppressive agents, reduced intensity conditioning, myeloablative conditioning, non-myeloablative conditioning, or immunosuppressive drugs. In some embodiments, the one or more radiotherapies comprise external radiotherapy, internal radiotherapy, radioimmunotherapy, or intraoperative radiation therapy (IORT). In some embodiments, the one or more immunotherapies comprise checkpoint inhibitors, inhibitors of co-stimulatory molecules, dendritic cell therapy, CAR-T cell therapy, cytokine therapy, or adoptive T cell therapy.

In some embodiments, the method further comprises administering to the subject a therapeutically effective amount of a composition comprising one or more broad-spectrum antibiotics to treat, prevent, or reduce the severity of cancer therapy-induced neutropenic fever in the subject. In some embodiments, the one or more broad-spectrum antibiotics comprise one or more cefepime and/or carbapenems. In some embodiments, the carbapenems comprise meropenem, imipenem/cilastatin, panipenem/betamipron, biapenem, ertapenem, and/or doripenem. In some embodiments, administration of the one or more broad-spectrum antibiotics increases the risk of graft-versus-host disease (GVHD) to the subject compared to a subject to whom the one or more broad-spectrum antibiotics are not administered. In some embodiments, the GVHD poses a greater risk to the health or life of the subject than such a condition would pose to an average person in the general population receiving the cancer therapy and/or the one or more broad-spectrum antibiotics. In some embodiments, the subject has GVHD due to the one or more broad-spectrum antibiotics received by the subject to treat, prevent, and/or reduce the severity of cancer therapy-induced neutropenic fever in the subject.

In some embodiments, the subject was determined to have decreased levels of one or more carbohydrate substrates metabolized by the one or more genera of mucus-degrading bacteria in the gut microbiome of the subject, an increased abundance of the one or more genera of mucus-degrading bacteria in the gut microbiome of the subject, and/or a decreased abundance of one or more commensal bacteria in the gut microbiome of the subject compared to a control or reference sample. In some embodiments, the decreased levels of one or more carbohydrate substrates metabolized by the one or more genera of mucus-degrading bacteria in the gut microbiome of the subject, increased abundance of the one or more genera of mucus-degrading bacteria in the gut microbiome of the subject, and/or decreased abundance of one or more commensal bacteria in the gut microbiome of the subject was determined from a fecal sample from the subject. In some embodiments, the control or reference sample is a sample from a healthy subject or a subject to whom the one or more broad-spectrum antibiotics are not administered.

In some embodiments, the methods further comprise administering to the subject a therapeutically effective amount of a composition comprising one or more carbohydrate substrates metabolized by the one or more genera of mucus-degrading bacteria in the gut microbiome to treat, prevent, and/or reduce the severity of GVHD. In some embodiments, the subject does not exhibit symptoms of GVHD when the composition is administered. In some embodiments, the subject has been diagnosed with GVHD. In some embodiments, the composition is administered after the subject has been diagnosed with GVHD. In some embodiments, the composition is administered to the subject every day until the subject no longer exhibits symptoms of GVHD and/or is determined to be cured of GVHD. In some embodiments, e composition comprising one or more carbohydrate substrates metabolized by the one or more genera of mucus-degrading bacteria in the gut microbiome is orally administered. In some embodiments, the composition is encapsulated.

Disclosed herein, in some aspects, is a method of predicting development of cancer therapy-induced neutropenic fever in a subject receiving a cancer therapy, the method comprising measuring an abundance of one or more genera of mucus-degrading bacteria in the gut microbiome of the subject, wherein: a) the subject is likely to develop cancer therapy-induced neutropenic fever or is at risk of developing cancer therapy-induced neutropenic fever when the abundance of the one or more genera of mucus-degrading bacteria in the gut microbiome of the subject is increased compared to a control or reference sample; and/or b) the subject is not at risk or is at reduced risk of developing cancer therapy-induced neutropenic fever when the abundance of the one or more genera of mucus-degrading bacteria in the gut microbiome of the subject are similar to or decreased compared to a control or reference sample; and wherein the one or more genera of mucus-degrading bacteria comprise Akkermansia or Bacteroides.

In some embodiments, the subject is likely to develop cancer therapy-induced neutropenic fever or is at risk of developing cancer therapy-induced neutropenic fever when the abundance of the one or more genera of mucus-degrading bacteria in the gut microbiome of the subject comprises more than 0.5%, more than 0.6%, more than 0.7%, more than 0.8%, more than 0.9%, more than 1.0%, more than 1.1%, more than 1.2%, more than 1.3%, more than 1.4%, or more than 1.5% of the total gut microbiome bacterial population compared to a control or reference sample. In some embodiments, when the abundance of the one or more genera of mucus-degrading bacteria in the gut microbiome of the subject is increased, the subject is provided a therapeutically effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

Disclosed herein, in some aspects, is a method of predicting a therapy outcome for a subject in need of cancer therapy, the method comprising measuring an abundance of one or more genera of mucus-degrading bacteria in the gut microbiome of the subject, wherein when the abundance of the one or more genera of mucus-degrading bacteria in the gut microbiome of the subject is increased compared to control or reference sample, the subject has an increased likelihood of developing cancer therapy-induced neutropenic fever, and wherein the one or more genera of mucus-degrading bacteria comprise Akkermansia or Bacteroides.

In some embodiments, the subject has an increased likelihood of developing cancer therapy-induced neutropenic fever when the abundance of the one or more genera of mucus-degrading bacteria in the gut microbiome of the subject comprises more than 0.5%, more than 0.6%, more than 0.7%, more than 0.8%, more than 0.9%, more than 1.0%, more than 1.1%, more than 1.2%, more than 1.3%, more than 1.4%, or more than 1.5% of the total gut microbiome bacterial population compared to a control or reference sample. In some embodiments, when the abundance of the one or more genera of mucus-degrading bacteria in the gut microbiome of the subject is increased, the subject is provided a therapeutically effective amount of one or more agents targeting growth or expansion of one or more genera of mucus-degrading bacteria in the gut microbiome of the subject.

Disclosed herein, in some aspects, is a method of predicting development of cancer therapy-induced neutropenic fever in a subject receiving a cancer therapy, the method comprising measuring functional activity and/or expression levels of one or more mucus-degrading enzymes secreted by one or more genera of mucus-degrading bacteria in the gut microbiome of the subject, wherein: a) the subject is likely to develop cancer therapy-induced neutropenic fever or is at risk of developing cancer therapy-induced neutropenic fever when the functional activity or expression levels of one or more mucus-degrading enzymes secreted by one or more genera of mucus-degrading bacteria in the gut microbiome of the subject are increased compared to a control or reference sample; and/or b) the subject is not at risk or is at reduced risk of developing cancer therapy-induced neutropenic fever when the functional activity or expression levels of one or more mucus-degrading enzymes secreted by one or more genera of mucus-degrading bacteria in the gut microbiome of the subject are similar to or decreased compared to a control or reference sample; and wherein the one or more genera of mucus-degrading bacteria comprise Akkermansia or Bacteroides.

In some embodiments, the subject is likely to develop cancer therapy-induced neutropenic fever or is at risk of developing cancer therapy-induced neutropenic fever when the functional activity or expression levels of one or more mucus-degrading enzymes secreted by one or more genera of mucus-degrading bacteria in the gut microbiome of the subject are increased greater than 1-fold to greater than 100000-fold compared to a control or reference sample. In some embodiments, when the functional activity or expression levels of one or more mucus-degrading enzymes secreted by one or more genera of mucus-degrading bacteria are increased, the subject is provided an effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

Disclosed herein, in some aspects, is a method of predicting a therapy outcome for a subject in need of cancer therapy, the method comprising measuring functional activity or expression levels of one or more mucus-degrading enzymes secreted by one or more genera of mucus-degrading bacteria in the gut microbiome of the subject, wherein when the functional activity or expression levels of one or more mucus-degrading enzymes secreted by one or more genera of mucus-degrading bacteria are increased compared to a control or reference sample, the subject has an increased likelihood of developing cancer therapy-induced neutropenic fever, and wherein the one or more genera of mucus-degrading bacteria comprise Akkermansia or Bacteroides.

In some embodiments, the subject has an increased likelihood of developing cancer therapy-induced neutropenic fever when the functional activity or expression levels of one or more mucus-degrading enzymes secreted by one or more genera of mucus-degrading bacteria in the gut microbiome of the subject are increased 1-fold to 100000-fold compared to a control or reference sample. In some embodiments, when the functional activity or expression levels of one or more mucus-degrading enzymes secreted by one or more genera of mucus-degrading bacteria are increased, the subject is provided an effective amount of one or more mucus-degrading enzyme inhibitors to inhibit mucus degradation by one or more genera of mucus-degrading bacteria in the gut microbiome of the subject.

In some embodiments of the methods disclosed herein, the one or more mucus-degrading enzymes comprise proteases, sulfatases, mucinases, or glycoside hydrolases. In some embodiments, the glycoside hydrolases comprise neuraminidases/sialidases, fucosidases, N-acetylglucosaminidases, galactosidases, N-acetylglucosaminidases, or N-acetylgalactosaminidases.

Disclosed herein, in some aspects, is a method of predicting development of cancer therapy-induced neutropenic fever in a subject receiving a cancer therapy, the method comprising measuring levels of one or more organic acid metabolites produced following metabolism of mucin-derived carbohydrates by one or more genera of mucus-degrading bacteria in the gut microbiome of the subject, wherein: a) the subject is likely to develop cancer therapy-induced neutropenic fever or is at risk of developing cancer therapy-induced neutropenic fever when the levels of one or more organic acid metabolites produced following metabolism of mucin-derived carbohydrates by one or more genera of mucus-degrading bacteria in the gut microbiome of the subject are decreased compared to a control or reference sample; and/or b) the subject is not at risk or is at reduced risk of developing cancer therapy-induced neutropenic fever when the levels of one or more organic acid metabolites produced following metabolism of mucin-derived carbohydrates by one or more genera of mucus-degrading bacteria in the gut microbiome of the subject are similar to or increased compared to a control or reference sample; and wherein the one or more genera of mucus-degrading bacteria comprise Akkermansia or Bacteroides.

In some embodiments, the subject is likely to develop cancer therapy-induced neutropenic fever or is at risk of developing cancer therapy-induced neutropenic fever when the levels of one or more organic acid metabolites produced following metabolism of mucin-derived carbohydrates by one or more genera of mucus-degrading bacteria in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample. In some embodiments, when the levels of one or more organic acid metabolites produced following metabolism of mucin-derived carbohydrates by one or more genera of mucus-degrading bacteria are decreased, the subject is provided an effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

Disclosed herein, in some aspects, is a method of predicting a therapy outcome for a subject in need of cancer therapy, the method comprising measuring levels of one or more organic acid metabolites produced following metabolism of mucin-derived carbohydrates by one or more genera of mucus-degrading bacteria in the gut microbiome of the subject, wherein when the levels of one or more organic acid metabolites produced following metabolism of mucin-derived carbohydrates by one or more genera of mucus-degrading bacteria are decreased compared to a control or reference sample, the subject has an increased likelihood of developing cancer therapy-induced neutropenic fever, and wherein the one or more genera of mucus-degrading bacteria comprise Akkermansia or Bacteroides.

In some embodiments, the subject has an increased likelihood of developing cancer therapy-induced neutropenic fever when the levels of one or more organic acid metabolites produced following metabolism of mucin-derived carbohydrates by one or more genera of mucus-degrading bacteria in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample. In some embodiments, when the levels of one or more organic acid metabolites produced following metabolism of mucin-derived carbohydrates by one or more genera of mucus-degrading bacteria are decreased, the subject is provided an effective amount of one or more mediators of organic acid metabolite levels produced following metabolism of mucin-derived carbohydrates by one or more genera of mucus-degrading bacteria in the gut microbiome of the subject.

In some embodiments of the methods disclosed herein, the organic acid metabolites comprise propionate, acetate, butyrate, isovalerate, or valerate.

Disclosed herein, in some aspects, is a method of predicting development of cancer therapy-induced neutropenic fever in a subject receiving a cancer therapy, the method comprising measuring levels of one or more ruminal metabolites in the gut microbiome of the subject, wherein: the subject is likely to develop cancer therapy-induced neutropenic fever or is at risk of developing cancer therapy-induced neutropenic fever when the levels of one or more ruminal metabolites that target the growth or expansion of one or more genera of mucus-degrading bacteria in the gut microbiome of the subject are decreased compared to a control or reference sample; and/or the subject is not at risk or is at reduced risk of developing cancer therapy-induced neutropenic fever when the levels of one or more ruminal metabolites that target the growth or expansion of one or more genera of mucus-degrading bacteria in the gut microbiome of the subject are similar to or increased compared to a control or reference sample; and wherein the one or more genera of mucus-degrading bacteria comprise Akkermansia or Bacteroides.

In some embodiments, the subject is likely to develop cancer therapy-induced neutropenic fever or is at risk of developing cancer therapy-induced neutropenic fever when the levels of one or more ruminal metabolites in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample. In some embodiments, when the levels of one or more ruminal metabolites that target the growth or expansion of one or more genera of mucus-degrading bacteria in the gut microbiome of the subject are decreased, the subject is provided an effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

Disclosed herein, in some aspects, is a method of predicting a therapy outcome for a subject in need of cancer therapy, the method comprising measuring levels of one or more ruminal metabolites that target the growth or expansion of one or more genera of mucus-degrading bacteria in the gut microbiome of the subject, wherein when the levels of one or more ruminal metabolites that target the growth or expansion of one or more genera of mucus-degrading bacteria in the gut microbiome of the subject are decreased compared to a control or reference sample, the subject has an increased likelihood of developing cancer therapy-induced neutropenic fever, and wherein the one or more genera of mucus-degrading bacteria comprise Akkermansia or Bacteroides.

In some embodiments, the subject has an increased likelihood of developing cancer therapy-induced neutropenic fever when the levels of one or more ruminal metabolites that target the growth or expansion of one or more genera of mucus-degrading bacteria in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample.

In some embodiments, when the levels of one or more ruminal metabolites that target the growth or expansion of one or more genera of mucus-degrading bacteria in the gut microbiome of the subject are decreased, the subject is provided an effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

In some embodiments of the methods disclosed herein, the one or more ruminal metabolites comprise malic acid, 3-indole acetic acid, hydrocinnamic acid, methylmalonic acid, gluconic acid, galacturonic acid, or bis-hydroxy methyl propionic acid.

In some embodiments, the subject has been diagnosed with neutropenia. In some embodiments, the subject is neutropenic due to the cancer therapy received by the subject. In some embodiments, the cancer therapy received by the subject comprise one or more chemotherapies, radiotherapies, and/or immunotherapies. In some embodiments, the one or more chemotherapies comprise alkylating agents, marrow-suppressive agents, reduced intensity conditioning, myeloablative conditioning, non-myeloablative conditioning, or immunosuppressive drugs. In some embodiments, the one or more radiotherapies comprise external radiotherapy, internal radiotherapy, radioimmunotherapy, or intraoperative radiation therapy (IORT). In some embodiments, the one or more immunotherapies comprise checkpoint inhibitors, inhibitors of co-stimulatory molecules, dendritic cell therapy, CAR-T cell therapy, cytokine therapy, or adoptive T cell therapy.

In some embodiments, measuring the abundance of one or more genera of mucus-degrading bacteria, the functional activity and/or expression levels of one or more mucus-degrading enzymes secreted by one or more genera of mucus-degrading bacteria, the levels of one or more organic acid metabolites produced following metabolism of mucin-derived carbohydrates by one or more genera of mucus-degrading bacteria, and/or the levels of one or more ruminal metabolites that target the growth or expansion of one or more genera of mucus-degrading bacteria in the gut microbiome of the subject occurs after the subject has been diagnosed with neutropenia.

Disclosed herein, in some aspects, is a method of preventing or reducing the severity of graft-versus-host disease (GVHD), the method comprising prophylactically administering to a subject receiving a hematopoietic cell transplantation (HCT) therapy and/or a neutropenic fever therapy after administration of a cancer therapy a therapeutically effective amount of a composition comprising: one or more agents targeting growth or expansion of one or more genera of mucus-degrading bacteria in the gut microbiome of the subject; and/or one or more carbohydrate substrates metabolized by the one or more genera of mucus-degrading bacteria in the gut microbiome of the subject; wherein the one or more genera of mucus-degrading bacteria comprise Bacteroides, Akkermansia, Ruminococcus, and Bifidobacterium.

In some embodiments, the subject is at a higher risk than an average person in the general population receiving the HCT therapy and/or the neutropenic fever therapy of developing GVHD. In some embodiments, the GVHD poses a greater risk to the health or life of the subject than such a condition would pose to an average person in the general population receiving the HCT therapy and/or the neutropenic fever therapy.

In some embodiments, the subject was determined to have an increased abundance of mucus-degrading bacteria in the gut microbiome compared to a control or reference sample. In some embodiments, the increased abundance of mucus-degrading bacteria in the gut microbiome was determined from a fecal sample from the subject.

In some embodiments, the subject was determined to have a decreased abundance of one or more commensal bacteria in the gut microbiome compared to a control or reference sample. In some embodiments, the one or more commensal bacteria in the gut microbiome comprise Clostridia. In some embodiments, the decreased abundance of commensal bacteria in the gut microbiome was determined from a fecal sample from the subject.

In some embodiments, the subject was determined to have decreased levels of one or more carbohydrate substrates metabolized by the one or more genera of mucus-degrading bacteria in the gut microbiome compared to a control or reference sample. In some embodiments, the decreased levels of one or more carbohydrate substrates metabolized by the one or more genera of mucus-degrading bacteria in the gut microbiome was determined from a fecal sample from the subject.

Disclosed herein, in some aspects, is a method of treating GVHD in a subject receiving a HCT therapy and/or a neutropenic fever therapy after administration of a cancer therapy and having an increased abundance of one or more genera of mucus-degrading bacteria in the gut microbiome of the subject compared to a control or reference sample, the method comprising administering to the subject a therapeutically effective amount of a composition comprising one or more of the following: one or more agents targeting growth or expansion of the one or more genera of mucus-degrading bacteria in the gut microbiome of the subject; and/or one or more carbohydrate substrates metabolized by the one or more genera of mucus-degrading bacteria in the gut microbiome of the subject; wherein the one or more genera of mucus-degrading bacteria comprise Bacteroides, Akkermansia, Ruminococcus, and Bifidobacterium.

Disclosed herein, in some aspects, is a method of treating GVHD in a subject receiving a HCT therapy and/or a neutropenic fever therapy after administration of a cancer therapy and having and having decreased levels of levels of one or more carbohydrate substrates metabolized by one or more genera of mucus-degrading bacteria in the gut microbiome of the subject, the method comprising administering to the subject a therapeutically effective amount of a composition comprising one or more of the following: one or more agents targeting growth or expansion of one or more genera of mucus-degrading bacteria in the gut microbiome of the subject; and/or one or more carbohydrate substrates metabolized by the one or more genera of mucus-degrading bacteria in the gut microbiome of the subject; wherein the one or more genera of mucus-degrading bacteria comprise Bacteroides, Akkermansia, Ruminococcus, and Bifidobacterium.

Disclosed herein, in some aspects, is a method of treating GVHD in a subject receiving a HCT therapy and/or a neutropenic fever therapy after administration of a cancer therapy and having a decreased abundance of one or more commensal bacteria in the gut microbiome of the subject compared to a control or reference sample, the method comprising administering to the subject a therapeutically effective amount of a composition comprising one or more of the following: one or more agents targeting growth or expansion of one or more genera of mucus-degrading bacteria in the gut microbiome of the subject; and/or one or more carbohydrate substrates metabolized by the one or more genera of mucus-degrading bacteria in the gut microbiome of the subject; wherein the one or more genera of mucus-degrading bacteria comprise Bacteroides, Akkermansia, Ruminococcus, and Bifidobacterium.

Disclosed herein, in some aspects, is a method of predicting development of GVHD in a subject receiving a HCT therapy and/or a neutropenic fever therapy after administration of a cancer therapy, the method comprising measuring an abundance of one or more genera of mucus-degrading bacteria in the gut microbiome of the subject, wherein: the subject is likely to develop GVHD or is at risk of developing GVHD when the abundance of the one or more genera of mucus-degrading bacteria in the gut microbiome of the subject is increased compared to a control or reference sample; and/or the subject is not at risk or is at reduced risk of developing GVHD when the abundance of the one or more genera of mucus-degrading bacteria in the gut microbiome of the subject is similar to or decreased compared to a control or reference sample; and wherein the one or more genera of mucus-degrading bacteria comprise Bacteroides, Akkermansia, Ruminococcus, and Bifidobacterium.

In some embodiments, the subject is likely to develop GVHD or is at risk of developing GVHD when the abundance of the one or more genera of mucus-degrading bacteria in the gut microbiome of the subject comprises more than 5%, more than 6%, more than 7%, more than 8%, more than 9%, more than 10%, more than 11%, more than 12%, more than 13%, more than 14%, or more than 15% of the total gut microbiome bacterial population compared to a control or reference sample. In some embodiments, when the abundance of the one or more genera of mucus-degrading bacteria in the gut microbiome of the subject is increased, the subject is provided a therapeutically effective amount of a composition comprising one or more agents targeting growth or expansion of one or more genera of mucus-degrading bacteria in the gut microbiome of the subject and/or one or more carbohydrate substrates metabolized by the one or more genera of mucus-degrading bacteria in the gut microbiome of the subject.

Disclosed herein, in some aspects, is a method of predicting development of GVHD in a subject receiving a HCT therapy and/or a neutropenic fever therapy after administration of a cancer therapy, the method comprising measuring the levels of one or more carbohydrate substrates metabolized by the one or more genera of mucus-degrading bacteria in the gut microbiome of the subject, wherein: the subject is likely to develop GVHD or is at risk of developing GVHD when the levels of one or more carbohydrate substrates metabolized by the one or more genera of mucus-degrading bacteria in the gut microbiome of the subject are decreased compared to a control or reference sample; and/or the subject is not at risk or is at reduced risk of developing GVHD when the levels of one or more carbohydrate substrates metabolized by the one or more genera of mucus-degrading bacteria in the gut microbiome of the subject are similar to or increased compared to a control or reference sample; and wherein the one or more genera of mucus-degrading bacteria comprise Bacteroides, Akkermansia, Ruminococcus, and Bifidobacterium.

In some embodiments, when the levels of one or more carbohydrate substrates metabolized by the one or more genera of mucus-degrading bacteria in the gut microbiome of the subject are decreased, the subject is provided a therapeutically effective amount of a composition comprising one or more agents targeting growth or expansion of one or more genera of mucus-degrading bacteria in the gut microbiome of the subject and/or one or more carbohydrate substrates metabolized by the one or more genera of mucus-degrading bacteria in the gut microbiome of the subject.

Disclosed herein, in some aspects, is a method of predicting development of GVHD in a subject receiving a HCT therapy and/or a neutropenic fever therapy after administration of a cancer therapy, the method comprising measuring the abundance of one or more commensal bacteria in the gut microbiome of the subject, wherein: the subject is likely to develop GVHD or is at risk of developing GVHD when the abundance of one or more commensal bacteria in the gut microbiome of the subject is decreased compared to a control or reference sample; and/or the subject is not at risk or is at reduced risk of developing GVHD when the abundance of one or more commensal bacteria in the gut microbiome of the subject is similar to or increased compared to a control or reference sample.

In some embodiments, the subject is likely to develop GVHD or is at risk of developing GVHD when the abundance of one or more commensal bacteria in the gut microbiome of the subject is decreased to less than 0.5%, less than 1%, less than 2%, less than 3%, less than 4%, less than 5%, less than 6%, less than 7%, less than 8%, less than 9%, or less than 10% of the total gut microbiome bacterial population compared to a control or reference sample. In some embodiments, when the abundance of one or more commensal bacteria in the gut microbiome of the subject is decreased, the subject is provided a therapeutically effective amount of a composition comprising one or more agents targeting growth or expansion of one or more genera of mucus-degrading bacteria in the gut microbiome of the subject and/or one or more carbohydrate substrates metabolized by the one or more genera of mucus-degrading bacteria in the gut microbiome of the subject.

Disclosed herein, in some aspects, is a method of predicting development of GVHD in a subject receiving a HCT therapy and/or a neutropenic fever therapy after administration of a cancer therapy, the method comprising measuring the levels of one or more ruminal metabolites that target the growth or expansion of one or more genera of mucus-degrading bacteria in the gut microbiome of the subject, wherein: the subject is likely to develop GVHD or is at risk of developing GVHD when the levels of the one or more ruminal metabolites are increased compared to a control or reference sample; and/or the subject is not at risk or is at reduced risk of developing GVHD when the levels of the one or more ruminal metabolites are similar to or decreased compared to a control or reference sample; and wherein the one or more genera of mucus-degrading bacteria comprise Bacteroides, Akkermansia, Ruminococcus, and Bifidobacterium.

In some embodiments, the subject is likely to develop GVHD or is at risk of developing GVHD when the levels of the one or more ruminal metabolites that target the growth or expansion of one or more genera of mucus-degrading bacteria in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample. In some embodiments, when the levels of the one or more ruminal metabolites that target the growth or expansion of one or more genera of mucus-degrading bacteria in the gut microbiome of the subject are decreased, the subject is provided a therapeutically effective amount of a composition comprising one or more agents targeting growth or expansion of one or more genera of mucus-degrading bacteria in the gut microbiome of the subject and/or one or more carbohydrate substrates metabolized by the one or more genera of mucus-degrading bacteria in the gut microbiome of the subject.

Disclosed herein, in some aspects, is a method of predicting development of GVHD in a subject receiving a HCT therapy and/or a neutropenic fever therapy after administration of a cancer therapy, the method comprising measuring the levels of the one or more ruminal metabolites that target the growth or expansion the levels of one or more genera of mucus-degrading bacteria in the gut microbiome of the subject, wherein: the subject is likely to develop GVHD or is at risk of developing GVHD when the levels of the ruminal metabolites are decreased compared to a control or reference sample; and/or the subject is not at risk or is at reduced risk of developing GVHD when the levels of the ruminal metabolites are similar to or increased compared to a control or reference sample.

In some embodiments, the subject is likely to develop GVHD or is at risk of developing GVHD when the levels of the one or more ruminal metabolites that target the growth or expansion of one or more genera of mucus-degrading bacteria in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample. In some embodiments, when levels of the one or more ruminal metabolites that target the growth or expansion of one or more genera of mucus-degrading bacteria in the gut microbiome of the subject are decreased, the subject is provided a therapeutically effective amount of a composition comprising one or more agents targeting growth or expansion of one or more genera of mucus-degrading bacteria in the gut microbiome of the subject and/or one or more carbohydrate substrates metabolized by the one or more genera of mucus-degrading bacteria in the gut microbiome of the subject.

In some embodiments, the control or reference sample is a sample from a healthy subject. In some embodiments, the control or reference sample is a sample from a subject to whom the HCT therapy and/or neutropenic fever therapy is not administered.

In some embodiments, the subject does not exhibit symptoms of GVHD when the composition is administered. In some embodiments, the subject has been diagnosed with GVHD. In some embodiments, the composition is administered after the subject has been diagnosed with GVHD. In some embodiments, the composition is administered to the subject every day until the subject no longer exhibits symptoms of GVHD and/or is determined to be cured of GVHD.

In some embodiments, the subject is diagnosed with GVHD due to the HCT therapy and/or the neutropenic fever therapy received by the subject. In some embodiments, the cancer therapy administered to the subject comprises one or more chemotherapies, radiotherapies, and/or immunotherapies. In some embodiments, the one or more chemotherapies comprise alkylating agents, marrow-suppressive agents, reduced intensity conditioning, myeloablative conditioning, non-myeloablative conditioning, or immunosuppressive drugs. In some embodiments, the one or more radiotherapies comprise external radiotherapy, internal radiotherapy, radioimmunotherapy, or intraoperative radiation therapy (IORT). In some embodiments, molecules, dendritic cell therapy, CAR-T cell therapy, cytokine therapy, or adoptive T cell therapy. In some embodiments, the neutropenic fever therapy comprises one or more broad-spectrum antibiotics. In some embodiments, the one or more broad-spectrum antibiotics comprise cefepime and/or carbapenems. In some embodiments, the carbapenems comprise meropenem, imipenem/cilastatin, panipenem/betamipron, biapenem, ertapenem, and/or doripenem. In some embodiments, the HCT therapy comprises autologous, allogeneic, and/or syngeneic HCT therapy. In some embodiments, the HCT therapy comprises allogeneic HCT therapy.

In some embodiments, measuring the abundance of one or more genera of mucus-degrading bacteria, the levels of one or more carbohydrate substrates metabolized by the one or more genera of mucus-degrading bacteria, the abundance of one or more commensal bacteria in the gut microbiome of the subject, and/or the levels of one or more ruminal metabolites that target the growth or expansion of one or more genera of mucus-degrading bacteria in the gut microbiome of the subject occurs after the subject has been diagnosed with GVHD.

In some of the methods disclosed herein, the one or more agents targeting growth or expansion of one or more genera of mucus-degrading bacteria in the gut microbiome of the subject comprise antibiotics or antimicrobial proteins or peptides. In some embodiments, the antibiotics comprise azithromycin. In some of the methods disclosed herein, the one or more agents targeting growth or expansion of one or more genera of mucus-degrading bacteria in the gut microbiome of the subject comprise bucine, methyl-β-D-galactopyranoside, resacetophenone, or serotonin. In some embodiments, the one or more agents targeting growth or expansion of one or more genera of mucus-degrading bacteria in the gut microbiome of the subject comprise one or more ruminal metabolites. In some embodiments, the one or more ruminal metabolites comprise malic acid, 3-indole acetic acid, hydrocinnamic acid, methylmalonic acid, gluconic acid, galacturonic acid, or bis-hydroxy methyl propionic acid. In some embodiments, the one or more mucus-degrading enzyme inhibitors comprise inhibitors of proteases, sulfatases, mucinases, or glycoside hydrolases. In some embodiments, the glycoside hydrolases comprise neuraminidases/sialidases, fucosidases, N-acetylglucosaminidases, galactosidases, N-acetylglucosaminidases, or N-acetylgalactosaminidases. In some embodiments, the neuramidase/sialidase inhibitors comprise siastatin B, zanamivir, peramivir, oseltamivir, or laninamivir. In some embodiments, the one or more mediators of organic acid metabolite levels comprise one or more vitamins, probiotics, prebiotics, or direct or indirect delivery of organic acid metabolites. In some embodiments, the one or more vitamins comprise vitamin B12. In some embodiments, the one or more organic acid metabolites comprise propionate, acetate, butyrate, isovalerate, or valerate. In some embodiments, the one or more carbohydrate substrates metabolized by the one or more genera of mucus-degrading bacteria in the gut microbiome comprise arabinose, fructose, fucose, galactose, galacturonic acid, glucuronic acid, glucosamine, glucose, mannose, N-acetylglucosamine, N-acetylgalactosamine, rhamnose, ribose, xylose, pullulan, glycogen, amylopectin, inulin, levan, heparin, hyaluronan, chondroitin sulfate, polygalacturonate, rhamnogalacturonan, pectic galactan, arabinogalactan, arabinan, xylan, arabinoxylan, galactomannan, glucomannan, xyloglucan, β-glucan, cellobiose, laminarin, lichenin, dextran, and/or α-mannan. In some embodiments, the one or more carbohydrate substrates metabolized by the one or more genera of mucus-degrading bacteria in the gut microbiome comprise mannose, glucose, and/or xylose. In some embodiments, the one or more carbohydrate substrates metabolized by the one or more genera of mucus-degrading bacteria in the gut microbiome comprise xylose.

In some embodiments of the methods disclosed herein, the composition is administered multiple times per day. In some embodiments, the composition is administered 2, 3, 4, 5, or 6 times per day. In some embodiments of the methods disclosed herein, the compositions are orally administered. In some embodiments, the compositions are encapsulated.

In some embodiments of the methods disclosed herein, the subject has been diagnosed with cancer. In some embodiments, the cancer comprises a solid tumor or is a hematological malignancy. In some embodiments, the subject is in need of a transplant therapy. In some embodiments, the subject has a leukemia, myeloma, or lymphoma and is in need of a hematopoietic stem cell transplant therapy.

In some embodiments of the methods disclosed herein, the identity or abundance of the one or more genera of mucus-degrading bacteria in the gut microbiome is determined by shotgun sequencing of the genome of the one or more genera of mucus-degrading bacteria. In some embodiments, the identity or abundance of the one or more genera of mucus-degrading bacteria in the gut microbiome is determined by directed sequencing of the genome of the one or more genera of mucus-degrading bacteria. In some embodiments, the directed sequencing is of 16S rRNA of the one or more genera of mucus-degrading bacteria.

In some embodiments of the methods disclosed herein, the control or reference sample is a sample from a healthy subject. In some embodiments, the control or reference sample is a sample from a subject who is diagnosed with neutropenia but who does not become febrile or develop neutropenic fever. In some embodiments, the control or reference sample is a sample from a subject who is diagnosed with neutropenia after administration of the cancer therapy but who does not become febrile or develop neutropenic fever. In some embodiments, the control or reference sample is a sample from a subject who is diagnosed with neutropenia who becomes febrile or develops neutropenic fever. In some embodiments, the control or reference sample is a sample from a subject who is diagnosed with neutropenia after administration of the cancer therapy who becomes febrile or develops neutropenic fever.

As used herein, the term “cancer therapy-induced neutropenic fever” refers to neutropenic fever induced by one or more cytotoxic cancer therapies, including but not limited to chemotherapy, such as alkylating agents and other marrow-suppressive agents commonly used in the treatment of cancer patients; radiotherapy; and immunotherapy. Thus, in some embodiments, the cancer therapy-induced neutropenic fever is chemotherapy-induced neutropenic fever. In some embodiments, the chemotherapy-induced neutropenic fever is alkylating agent-induced neutropenic fever. In some embodiments, the chemotherapy-induced neutropenic fever is marrow-suppressive agent-induced neutropenic fever. In some embodiments, the cancer therapy-induced neutropenic fever is radiotherapy-induced neutropenic fever. In some embodiments, the cancer therapy-induced neutropenic fever is immunotherapy-induced neutropenic fever.

As used herein, the term “HCT-related GVHD” refers to GVHD induced as a result of one or more cytotoxic cancer therapies followed by infusion of allogeneic donor hematopoietic cells. The allogeneic donor hematopoietic cells, in some embodiments, are allogeneic hematopoietic stem cells (HSCT), and thus, in some embodiments, the HCT-related GVHD is HSCT-related GVHD. The one or more cytotoxic cancer therapies may include but are not limited to chemotherapy, such as alkylating agents and other marrow-suppressive agents commonly used in the treatment of cancer patients; radiotherapy; and immunotherapy. Side effects of the one or more cytotoxic cancer therapies can include but are not limited to neutropenia, which may or may not be associated with neutropenic fever caused at least in part by microbial infection, and therefore, in at least in some embodiments, the GVHD is neutropenic fever therapy-induced GVHD. As used herein, the term “neutropenic fever therapy-induced GVHD” refers to GVHD induced as a result of a therapy administered to treat, prevent, or reduce the severity of neutropenic fever caused at least in part by the one or more cytotoxic cancer therapies. In some embodiments, neutropenic fever may be treated with broad-spectrum antibiotics, including cefepime and/or carbapenems comprising, for example, meropenem, imipenem/cilastatin, panipenem/betamipron, biapenem, ertapenem, and doripenem. Thus, in some embodiments, the neutropenic fever therapy-induced GVHD is carbapenem-induced GVHD. In some embodiments, the neutropenic fever therapy-induced GVHD is meropenem-induced GVHD. In some embodiments, the neutropenic fever therapy-induced GVHD is cefepime-induced GVHD.

The term “detecting” is used broadly herein to include appropriate means of determining the presence or absence of an entity of interest or any form of measurement of an entity of interest in a sample. Thus, “detecting” may include determining, measuring, assessing, or assaying the presence or absence, level, amount, and/or location of an entity of interest. Quantitative and qualitative determinations, measurements or assessments are included, including semi-quantitative. Such determinations, measurements or assessments may be relative, for example when an entity of interest is being detected relative to a control, reference, or absolute. As such, the term “quantifying” when used in the context of quantifying an entity of interest can refer to absolute or to relative quantification. Absolute quantification may be accomplished by correlating a detected level of an entity of interest to known control standards (e.g., through generation of a standard curve). Alternatively, relative quantification can be accomplished by comparison of detected levels or amounts between two or more different entities of interest to provide a relative quantification of each of the two or more different entities of interest, i.e., relative to each other.

Those of ordinary skill in the art, reading the present specification, will appreciate that a step of “measuring” or “determining” can utilize or be accomplished through use of any of a variety of techniques available to those skilled in the art, including for example specific techniques explicitly referred to herein. In some embodiments, measuring or determining involves manipulation of a physical sample. In some embodiments, measuring or determining involves consideration and/or manipulation of data or information, for example utilizing a computer or other processing unit adapted to perform a relevant analysis. In some embodiments, measuring or determining involves receiving relevant information and/or materials from a source. In some embodiments, measuring or determining involves comparing one or more features of a sample or entity to a comparable reference.

As used herein, the terms “improved,” “increased.” “reduced,” “decreased,” or grammatically comparable comparative terms, indicate values that are relative to a comparable reference measurement. For example, in some embodiments, an assessed value achieved with an treatment of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an treatment of interest), or in a different, comparable subject (e.g., in a comparable subject or system that differs from the subject or system of interest in presence of one or more indicators of a particular disease, disorder or condition of interest, or in prior exposure to a condition or treatment, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, the term “reference,” “standard,” “control,” or grammatically comparable comparative terms, describe a value relative to which a comparison is performed. For example, in some embodiments, a treatment, animal, individual, population, sample, or value of interest is compared with a reference, standard, or control treatment, animal, individual, population, sample, or value. In some embodiments, a reference, standard, or control is tested and/or determined substantially simultaneously with the testing or determination of interest. Typically, as would be understood by those skilled in the art, a reference, standard, or control is determined or characterized under comparable conditions or circumstances to those under assessment. Those skilled in the art will appreciate when sufficient similarities are present to justify reliance on and/or comparison to a particular possible reference or control.

As used herein, the term “therapeutically effective amount” is synonymous with “effective amount,” “therapeutically effective dose.” and/or “effective dose,” and refers to an amount of an agent sufficient to produce a desired result or exert a desired influence on the particular condition being treated. In some embodiments, a therapeutically effective amount is an amount sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. For instance, in some embodiments, the effective amount refers to the amount of a composition comprising one or more agents targeting growth or expansion of one or more genera of mucus-degrading bacteria in the gut microbiome; one or more mucus-degrading enzyme inhibitors to inhibit mucus degradation by one or more genera of mucus-degrading bacteria in the gut microbiome; one or more mediators of organic acid metabolite levels produced following metabolism of mucin-derived carbohydrates by one or more genera of mucus-degrading bacteria in the gut microbiome; and/or one or more carbohydrate substrates metabolized by one or more genera of mucus-degrading bacteria in the gut microbiome of the subject, that can treat or prevent neutropenic fever and/or GVHD in a subject. The effective amount may vary depending on the organism or individual treated. The appropriate effective amount to be administered for a particular application of the disclosed methods can be determined by those skilled in the art, using the guidance provided herein.

As used herein, the terms “treatment,” “treat,” or “treating” refers to intervention in an attempt to alter the natural course of the subject being treated, and may be performed either for prophylaxis or during the course of pathology of a disease or condition. Treatment may serve to accomplish one or more of various desired outcomes, including, for example, preventing occurrence or recurrence of disease, alleviation or reduction in severity of symptoms, and diminishment of any direct or indirect pathological consequences of the disease, preventing disease spread, lowering the rate of disease progression, amelioration or palliation of the disease state, and remission or improved prognosis.

In some cases, the subject receiving a cancer therapy was determined to have one or more of: (1) an increased abundance of one or more genera of mucus-degrading bacteria in the gut microbiome compared to a control or reference sample; (2) a decreased abundance of one or more commensal bacteria in the gut microbiome compared to a control or reference sample; (3) an increase in functional activity and/or expression levels of one or more mucus-degrading enzymes secreted by one or more genera of mucus-degrading bacteria in the gut microbiome compared to a control or reference sample; (4) decreased levels of one or more organic acid metabolites produced following metabolism of mucin-derived carbohydrates by one or more genera of mucus-degrading bacteria in the gut microbiome compared to a control or reference sample; and/or (5) a decrease in levels of one or more carbohydrate substrates metabolized by one or more genera of mucus-degrading bacteria in the gut microbiome compared to a control or reference sample. In some cases, when a subject receiving a cancer-therapy was determined to have any one or more of (1)-(5), the subject is administered any one or more of: (1) an effective amount of one or more bacterial growth-suppressing agents that would reduce levels of one or more microbes that were determined to be excessive in the gut microbiome of a subject; (2) an effective amount of one or more one or more mucus-degrading enzyme inhibitors that would inhibit mucus degradation by enzymes produced by one or more genera of mucus-degrading bacteria in the gut microbiome that were determined to be excessive in the gut microbiome of a subject; (3) an effective amount of one or more mediators of organic acid metabolite levels produced following metabolism of mucin-derived carbohydrates by one or more genera of mucus-degrading bacteria in the gut microbiome that serve as a feedback mechanism to suppress excessive utilization of mucin glycans, which would otherwise be metabolized by one or more genera of mucus-degrading bacteria in the gut microbiome; and/or (4) an effective amount of one or more one or more carbohydrate substrates for metabolism by one or more genera of mucus-degrading bacteria in the gut microbiome.

Throughout this application, the term “about” is used to indicate that a value includes the inherent variation of error for the measurement or quantitation method.

The use of the word “a” or “an” when used in conjunction with the term “comprising” may mean “one.” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

Use of the one or more compositions may be employed based on any of the methods described herein. Other embodiments are discussed throughout this application. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Example section are understood to be embodiments that are applicable to all aspects of the technology described herein.

It is contemplated that any embodiment discussed in this specification can be implemented with respect to any method or composition of the disclosure, and vice versa. Furthermore, compositions of the disclosure can be used to achieve methods of the disclosure.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1F. Intestinal microbiome parameters at neutropenia onset and subsequent fever were evaluated in a cohort of patients undergoing HCT. Stool samples were collected at onset of neutropenia (+/−2 days), and fever outcome was determined by inpatient monitoring every 4 hours in the subsequent 4 days after collection. FIG. 1A. Following 16S rRNA gene sequencing, Principal Coordinates Analysis (PCoA) was performed on weighted UniFrac distances. Statistical significance was determined by Permutational Multivariate Analysis Of Variance (PERMANOVA) testing. FIG. 1B. Volcano plot of bacterial taxa that were differentially abundant in FIG. 1A. Taxa above the green line have a p value less than 0.05; p values were adjusted for multiple comparisons. FIG. 1C. Relative abundances of bacteria at the genus level in samples from FIG. 1A are indicated in stacked bar graphs. FIG. 1D. Relative abundances of bacteria of the indicated taxa are depicted for samples from FIG. 1A; p values were adjusted for multiple comparisons. FIG. 1E. Mucin glycan consumption by frozen aliquots of stool samples in FIG. 1A was assayed. Fecal bacteria were cultivated in liquid media supplemented with porcine gastric mucin as the predominant source of carbon, followed by quantification of remaining mucin glycans after 48 hours. Samples were stratified by median sum abundance of Akkermansia and Bacteroides. FIG. 1F. In the subset of patients who later developed neutropenic fever, relative abundances of bacteria from the indicated taxa in stool samples collected at onset of neutropenia were compared to results of a baseline stool sample collected earlier in the hospitalization, using the Wilcoxon signed-rank test.

FIGS. 2A-2K. Evaluation of intestinal microbiome parameters was performed in adult C57BL/6 female mice 6 days following total body radiotherapy (9 Gy RT, panels A-E) or 6 days following melphalan therapy (20 mg/kg, panels G-K). FIG. 2A. After 9 Gy RT, PCoA was performed on weighted UniFrac distances; combined results of 3 experiments. FIG. 2B. Volcano plot of bacterial taxa that were differentially abundant in FIG. 2A; p values were adjusted for multiple comparisons. FIG. 2C. Heat map of scaled relative bacterial abundances of the indicated taxa are depicted for samples from FIG. 2A. FIG. 2D. Relative abundances of bacteria at the genus level in samples from FIG. 2A are indicated in stacked bar graphs. FIG. 2E. Bacteria from frozen stool samples collected from mice in FIG. 2A were evaluated for mucin glycan consumption; combined results of 2 experiments. FIG. 2F. Thickness of the dense inner colonic mucus layer was evaluated histologically in mice in FIG. 2A. Representative images are provided with combined results of 3 experiments. FIG. 2G. After melphalan therapy. PCA was performed on weighted UniFrac distances; combined results of 3 experiments. FIG. 2H. Volcano plot of bacterial taxa that were differentially abundant in FIG. 2G; p values were adjusted for multiple comparisons. FIG. 2I. Heat map of scaled relative bacterial abundances of the indicated taxa are depicted for samples from FIG. 2G. FIG. 2J. Relative abundances of bacteria at the genus level in samples from FIG. 2G are indicated in stacked bar graphs. FIG. 2K. Thickness of the dense inner colonic mucus layer was evaluated histologically in mice in FIG. 2G. Representative images are provided with combined results of 2 experiments.

FIGS. 3A-3I. FIG. 3A. After 9 Gy RT, mice were individually housed in metabolic cages and monitored daily for food consumption, water consumption, and weight. FIG. 3B. Intestinal microbiome parameters were evaluated in normal mice after undergoing dietary restriction (2 g/mouse/day) for one week. PCoA was performed on weighted UniFrac distances; combined results of 3 experiments. FIG. 3C. Volcano plot of bacterial taxa that were differentially abundant in FIG. 3B; p values were adjusted for multiple comparisons. FIG. 3D. Heat map of scaled relative bacterial abundances of the indicated taxa are depicted for samples from FIG. 3B. FIG. 3E. Relative abundances of bacteria at the genus level in samples from FIG. 3A are indicated in stacked bar graphs. FIG. 3F. Bacteria from frozen stool samples collected from mice in FIG. 3B were evaluated for mucin glycan consumption; combined results of 2 experiments. FIG. 3G. Thickness of the dense inner colonic mucus layer was evaluated histologically in mice in FIG. 3B. Representative images are provided with combined results of 3 experiments. FIG. 3H. Mice underwent dietary restriction as in FIG. 3B, with the addition of narrow-spectrum antibiotics administered in the drinking water starting 5 days prior to onset of restriction. Relative abundances of bacteria at the genus level in samples are indicated in stacked bar graphs; combined results of 2 experiments. FIG. 3I. Thickness of the dense inner colonic mucus layer was evaluated histologically in mice in FIG. 3H. Representative images are provided with combined results of 2 experiments.

FIGS. 4A-4H. FIG. 4A. In mice that underwent one week of dietary restriction, cecal luminal contents were assessed for caloric content by bomb calorimetry; combined results of 2 experiments. FIG. 4B. Colonic luminal contents were assessed for pH in mice following one week of dietary restriction; combined results of 3 experiments. FIG. 4C. Metabolites from samples in FIG. 4B were quantified using ion chromatography-mass spectrometry (IC-MS); combined results of 2 experiments. FIG. 4D. A murine isolate of A. muciniphila (MDA-JAX AM001) was cultivated under anaerobic conditions of varying pH in 4 replicates, and growth and mucin glycan consumption were quantified after 48 hours of culture; results of one of two experiments with similar results. FIG. 4E. A. muciniphila (MDA-JAX AM001) was cultivated under varying pH and varying concentrations of sodium acetate, sodium propionate, and sodium butyrate in 4 replicates, and mucin glycan consumption was quantified after 48 hours of culture; results of one of two experiments with similar results. FIG. 4F. Normal mice received one week of dietary restriction, as well as supplementation with sodium acetate or sodium propionate in the drinking water, acidified to pH3. Relative abundances of Akkermansia was quantified by 16S rRNA gene sequencing; combined results of 3 experiments. FIG. 4G. Thickness of the dense inner colonic mucus layer was evaluated histologically in mice in FIG. 4F. Representative images are provided with combined results of 3 experiments. FIG. 4H. Transcriptomic profiling identifies A. muciniphila genes similarly regulated by diet in vivo and propionate in vitro. RNA sequencing was performed on A. muciniphila (MDA-JAX AM001) cultivated at pH 6.5 with varying concentrations of sodium propionate as in FIG. 4E in 3 replicates, and on fecal pellets from mice following one week of dietary restriction (n=5). Sequences aligning with the genome of A. muciniphila (MDA-JAX AM001) were quantified, and the scaled abundances of the subset of genes similarly regulated by diet and propionate are depicted in the heat map, along with annotations obtained using both the CAZy and NCBI RefSeq Protein databases.

FIGS. 5A-5D. In the setting of 9 Gy RT, mice were treated with azithromycin or sodium propionate. FIG. 5A. Thickness of the dense inner colonic mucus layer was evaluated histologically. Representative images are provided with combined results of 2 experiments. FIG. 5B. Ocular temperatures were monitored daily. Representative images 6 days after RT are provided with combined results of 2 experiments. FIG. 5C. On day 6 after RT, mice were harvested and colonic tissues was processed to quantify levels of cytokines. Combined results of 3 experiments. FIG. 5D. Relative abundances of Akkermansia on day 6 after RT was quantified by 16S rRNA gene sequencing. Combined results of 3 experiments.

FIGS. 6A-6C. FIG. 6A. Workflow schematic of bacterial mucin glycan consumption assay. FIG. 6B. Results of mucin glycan quantification following 48-hour culture of indicated bacterial isolates. FIG. 6C. In the subset of patients who did not develop neutropenic fever, relative bacterial abundances of the indicated taxa in stool samples collected at onset of neutropenia were compared to results of a baseline stool sample collected earlier in the hospitalization, using the Wilcoxon signed-rank test.

FIG. 7. Schematic of histological quantification of the dense inner colonic mucus layer histologically, following PAS staining. Eight equally radially spaced sites are identified for mucus layer thickness quantification which are then averaged for each sample.

FIGS. 8A-8B. Radiation does not directly lead to a selective advantage for Akkermansia or Bacteroides. FIG. 8A. Fecal samples from normal mice were exposed to 9 Gy RT and then cultivated on Columbia blood agar plates in anaerobic conditions. Bacterial composition was determined by swabbing the plates and performing 16S rRNA gene sequencing. FIG. 8B. Fecal samples from normal mice were exposed to 9 Gy RT as in FIG. 8A, and then administered by gavage to mice following antibiotic decontamination with ampicillin, metronidazole and vancomycin in the drinking water. One week after fecal transplantation, stool pellets were collected and the bacterial composition was evaluated by 16S rRNA gene sequencing.

FIGS. 9A-9B. Dietary restriction has no clear impact on colonic mucus producing cells. FIG. 9A. Mice were subjected to dietary restriction for one week, and then colonic tissues were harvested and examined histologically. Goblet cell numbers were quantified, as well as goblet cell surface area. FIG. 9B. Gene expression of muc2 in colonic tissues was quantified in mice following one week of dietary restriction.

FIGS. 10A-10E. FIG. 10A. Raw values (without normalization) of metabolite quantification from sample depicted in FIG. 4C. FIG. 10B. A. muciniphila growth in samples depicted in Figure FIG. 4E, quantified by optical density (OD) 600 mm. FIG. 10C. Mice underwent dietary restriction and treatment with supplemental sodium acetate and sodium propionate adjusted to the indicated pH levels for one week, followed by quantification of the pH of colonic luminal contents. FIG. 10D. Mice were treated with dietary restriction and supplemental sodium acetate and sodium propionate adjusted to the indicated pH levels for one week, and fecal bacterial composition was evaluated by 16S rRNA gene sequencing; combined results of 2 experiments. FIG. 10E. Mice were treated as in FIG. 10D and the colonic mucus thickness was quantified histologically; combined results of 2 experiments.

FIGS. 11A-11C. FIG. 11A. Circularized genome of A. muciniphila (MDA-JAX AM001). G- and C-dominant 12 regions depict results of 10,000 bp moving averages. FIG. 11B. Changes in A. muciniphila gene expression were quantified in the settings of dietary restriction and in vitro exposure to propionate. Effect size statistics were quantified by the Mann-Whitney and Kruskal-Wallis methods, respectively, followed by using Spearman's rank-order correlation test (p<0.0001). FIG. 11C. Changes in gene expression from a genomic perspective are depicted, along with 10,000 bp moving averages.

FIG. 12A-12B. Relative abundance of short-chain fatty acids, propionate (FIG. 12A) and succinate (FIG. 12B), from Parabacteroides distasonis (PD)-grown cell-free culture supernatant (CFCS) as quantified using ion chromatography-mass spectrometry (IC-MS). PD was grown in modified chopped meat broth (MCMB) with/without tapioca (Tap) for three days anaerobically.

FIG. 13A-13B. Effect of oral administration of Parabacteroides distasonis (PD) and tapioca starch together with vitamin B12 on Akkermansia expansion and colonic mucus thickness. FIG. 13A. 16S sequencing data. FIG. 13B. Colonic mucus layer quantification.

FIGS. 14A-14H. Meropenem increased the incidence of intestinal GVHD in allo-HSCT patients and mice. FIG. 14A. Incidence of intestinal GVHD in 295 patients with AML or MDS transplanted with allo-HSCT following conditioning with fludarabine and busulfan with tacrolimus and methotrexate as GVHD prophylaxis from 2011 to 2016. Patients were stratified by antibiotic treatment with neither cefepime nor meropenem, cefepime alone, meropenem alone and both cefepime and meropenem from days −10 to 30 relative to allo-HSCT infusion date. FIG. 14B. Experimental schema of murine GVHD model using meropenem treatment. Lethally irradiated B6D2F1 mice were transplanted with bone marrow (BM) cells and splenocytes from B6 (allogeneic arm) or B6D2F1 (syngeneic arm) donors on day 0. Mice were treated with meropenem from days 3 to 15. TBI, total body irradiation. FIG. 14C. Overall survival of mice after HSCT (Syngeneic, n=10, Allogeneic, n=15, Allogeneic+meropenem, n=20). Data are combined from three independent experiments. FIG. 14D. H&E staining of histological sections of small intestine, colon, and liver collected on day 18 after allo-HSCT. Bar, 100 μM. FIG. 14E. GVHD histology scores of small intestine, colon, and liver. These target organs were harvested from mice on day 18, and GVHD histology scores were quantified by a blinded pathologist. Data are combined from two independent experiments and are shown as means±SE. (F) Experimental schema of murine GVHD model using meropenem treatment and decontamination therapy. Allo-HSCT and meropenem treatment were performed as in FIG. 14B. Mice were decontaminated with piperacillin/tazobactam plus nystatin from days 5 to 15. FIG. 14G. Bacterial density of stool in normal mice and allogeneic mice treated or untreated with meropenem with or without decontamination therapy collected on days 7, 14 and 21 after allo-HSCT. Data are shown from one representative experiment. FIG. 14H. Overall survival of mice after allo-HSCT treated as depicted in FIG. 14F (Allogeneic, n=10, Allogeneic+meropenem, n=20, Allogeneic+meropenem+cocktail, n=15). Data are combined from three independent experiments.

FIGS. 15A-15L. The effects of meropenem treatment on the composition of the intestinal microbiome in both patients and mice is characterized loss of Clostridia and expansion of Bacteroides. FIGS. 15A-15C. Composition of fecal samples from allo-HSCT patients (n=44) collected pre-HSCT and on day 14. FIG. 15A. Stacked bar graphs of bacterial genera composition of fecal samples collected pre-HSCT (top) and on day 14 (bottom). FIG. 15B. Volcano plot of differentially abundant bacterial genera comparing pre-HSCT and day 14 samples collected from meropenem-treated patients, analyzed by the paired-Wilcoxon test and adjusted for false discovery. FIG. 15C. Volcano plot of differentially abundant bacterial genera when comparing pre-HSCT and day 14 samples collected from meropenem-untreated patients. FIG. 15D. Paired-Wilcoxon test of the genus Bacteroides between at pre-HSCT and on day 14 in meropenem-treated patients. FIG. 15E. Paired-Wilcoxon test of the genus Bacteroides between at pre-HSCT and on day 14 in meropenem-untreated patients. FIG. 15F. Experimental schema of murine GVHD model using meropenem treatment. Allo-HSCT and meropenem treatment were performed as in FIG. 14. The composition of the intestinal microbiome of fecal samples collected on day 21 was evaluated. FIG. 15G. Bacterial density on days 7, 14, and 21 after transplant was measured using qPCR of 16S rRNA. FIG. 15H. α-diversity, measured by the inverse Simpson index, was quantified in fecal samples collected on day 21. FIG. 15I. PCoA of fecal samples collected on day 21. FIG. 15J. Stacked bar graphs of bacterial genera composition of fecal samples collected on day 21. FIG. 15K. Volcano plot of differentially abundant bacterial genera comparing fecal samples collected on day 21. FIG. 15L. Relative abundance of Bacteroides in fecal samples collected on day 21. FIGS. 15G-15L. Data are combined from three independent experiments.

FIGS. 16A-16E. Murine BT associated with meropenem-induced colonic GVHD. FIG. 16A. Relative abundance of distinguishable Bacteroides sequence variants on day 21 after allo-HSCT. FIG. 16B. Longitudinal relative abundance of BT on day 0, 7, 14 and 21. FIG. 16C. Meropenem-treated mice (FIG. 14C) were stratified by median abundance of BT into a high abundant BT arm (relative abundance >0.2, n=9) and low abundant BT arm (relative abundance <0.2, n=8). FIG. 16D. Experimental schema of murine GVHD model using decontamination therapy followed by oral introduction of BT. Mice were decontaminated as in FIG. 14F followed by oral gavage of 20 million CFU of BT (MDA-JAX BT001) daily for 3 days. FIG. 16E. Overall survival after allo-HSCT (Allogeneic+meropenem+cocktail, n=18. Allogeneic+meropenem+cocktail+BT, n=22). FIGS. 16A-16C, 16E. Data are combined from three independent experiments.

FIGS. 17A-17G. Meropenem-induced compromise of the colonic mucus layer in mice with GVHD. FIG. 17A. PAS staining of histological colon sections collected on day 18. Bar. 100 μM. FIG. 17B. Mucus thickness on day 18. Combined data from two independent experiments are shown as means±SE. FIG. 17C. Immunofluorescent staining of colon sections for MUC2 (green) with universal bacterial 16S rRNA gene in situ hybridization probe EUB338 (red) counterstained with DAPI. Bar. 100 μM. Arrowheads indicate infiltrated bacteria into inner mucus layer and colon. Areas in the white squares are magnified and shown to the below of the original images. The area inside dotted lines indicate the inner dense colonic mucus layer. FIG. 17D. Numbers of bacterial CFUs cultivated from MLNs on day 18. Combined data from three independent experiments shown as means±SE (Syngeneic, n=5. Allogeneic, n=15. Allogeneic+meropenem, n=15). FIG. 17E. Identified translocated bacteria in MLNs by MALDI Biotyper. Number indicates number of identified bacterial colonies. FIG. 17F. Immunohistochemistry staining of CD11b in histological colon sections. Bar, 100 μM. FIG. 17G. Numbers of dendritic cells and neutrophils in the colon were determined by flow cytometric analysis. Combined data from three independent experiments shown as means±SE. (Allogeneic, n=10, Allogencic+meropenem, n=10).

FIGS. 18A-18I. Mucolytic activity of BT is suppressed by ambient xylose. FIG. 18A. Heatmap of the relative expression levels of PULs in BT RNA transcripts sequenced from stool collected from allogeneic mice treated or untreated with meropenem on day 18. Left column provides the PUL identification numbers from the Polysaccharide-Utilization Loci DataBase. Right column provides enzymatic functional annotations. FIG. 18B. Relative abundances of monosaccharides following acid hydrolysis of supernatants from stool collected from normal mice and allo-HSCT mice treated or untreated with meropenem on day 18 measured by IC-MS. FIG. 18C. Experimental schema of in vitro bacterial culture assay of BT in media with porcine gastric mucin-containing medium with or without monosaccharides. FIG. 18D. Relative concentrations of porcine gastric mucin in medium following culture with BT (MDA-JAX BT001). BT was first introduced to porcine gastric mucin-containing medium. At 15 hours of culture, monosaccharides were added into the culture broth and incubated for an additional 3 hours. Levels of mucin glycans in the culture supernatant were determined using a colorimetric assay. Mucin concentrations were normalized to levels of mucin glycans from non-sugar-exposed BT conditions in two independent experiments. FIG. 18E. mRNA was extracted from BT pellets cultured with or without monosaccharides and subjected to qPCR analysis of GH2, GH29 and GH33 BT loci. Relative expressions of mRNA were shown by the comparative ΔCt method. FIG. 18F. Experimental schema of murine GVHD model using meropenem treatment and administration of xylose. FIG. 18G. Relative abundance of BT on day 20. Combined data from two independent experiments are shown. FIG. 18H. PAS staining of histological colon sections collected on day 20. Bar, 100 μM. FIG. 18I. Mucus thickness on day 20. Combined data from two independent experiments are shown as means±SE.

FIGS. 19A-19B. Meropenem concentrations and bacterial densities in normal SPF mice with meropenem treatment. FIG. 19A. Meropenem concentrations in the cecal contents of mice were measured 4, 8, 24, 48 and 96 hours after subcutaneously injection of meropenem using LC-MS. FIG. 19B. Bacterial densities of mouse stool samples collected 7 days after administering with meropenem by drinking water. Bacterial densities were measured by qPCR of 16S rRNA.

FIGS. 20A-20B. The relative abundances of Clostridia and SCFAs were significantly decreased by meropenem treatment. FIG. 20A. Relative abundance of the class Clostridia on days 0, 7, 14, and 21. FIG. 20B. Relative abundances of SCFAs in stool samples from normal mice, allo-HSCT mice and meropenem-treated allo-HSCT mice on day 18 measured by IC-MS.

FIGS. 21A-21D. The intestinal microbiome in allo-HSCT patients treated or untreated with meropenem at pre-HSCT and on day 14. Additional analyses of intestinal microbiome profiling results of allo-HSCT patient samples presented in FIGS. 15A-15E. FIG. 21A. α-diversity shown using the inverse Simpson index at pre-HSCT and on day 14. FIG. 21B. PCoA between meropenem-untreated and treated patients at pre-HSCT (left) and on day 14 (right). FIG. 21C. Volcano plot on day 14 by 16S rRNA sequencing. FIG. 21D. Paired-Wilcoxon test of bacteria between at pre-HSCT and on day 14 in meropenem-treated patients (top row, shown in red) or meropenem-untreated patients (bottom row, shown in blue).

FIGS. 22A-22B. Both mouse-derived and human-derived BT show mucolytic activity against porcine gastric mucin. FIG. 22A. Bacterial culture with or without porcine gastric mucin. OD600 nm was measured after 48 hours of anaerobic culture. FIG. 22B. Levels of porcine gastric mucin in the culture supernatant was determined by using a PAS-based colorimetric assay. Mouse-derived Enterococcus faecalis were used as non-mucolytic bacterial control.

FIGS. 23A-23B. Whole genome sequence of mouse-derived BT isolate (MDA-JAX BT001) and monosaccharide utilization. FIG. 23A. Circular plot of open reading frames (ORFs) derived from the complete genome (MDA-JAX BT001). Blue and green bars represent ORFs on the plus strand and the minus strand, respectively. Inner purple-olive ring depicts degree of GC skewing. FIG. 23B. Bacterial culture with or without the indicated monosaccharide. Monosaccharides were added at 0 hr. OD600 nm was measured every 2 hours up to 30 hours.

FIGS. 24A-24D. Identification of A. muciniphila growth inhibitors. FIG. 24A. Absorbance at OD600 nm indicative of effect of the SCFAs, isovalerate and valerate, on growth of Akkermansia muciniphila (MDA-JAX AM001) in BYEM10+ mucin medium. FIG. 24B. Absorbance at OD600 nm indicative of effect of natural compounds identified by a high-throughput screen on growth of MDA-JAX AM001 in BYEM10+ mucin medium. FIG. 24C. Absorbance at OD600 nm indicative of effect of ruminal metabolites on growth of MDA-JAX AM001 in BYEM10+ mucin medium. FIG. 24D. Absorbance at OD600 nm indicative of effect of further ruminal metabolites on growth of MDA-JAX AM001 in BYEM10+ mucin medium.

### DETAILED DESCRIPTION

The degree and duration of neutropenia has long been identified as a critical clinical parameter predicting infection (4). More recently, the role of the intestinal microbiome in neutropenia-related infections has been increasingly appreciated with the majority of documented bacterial infections arising from the gastrointestinal tract (5, 6). Most patients, however, will not have an infectious etiology identified, and it is not well-understood why only some 50% of patients with profound neutropenia become febrile. In this disclosure, it is shown that the gut microbiome composition of a subject correlates with development of neutropenic fever in subjects receiving a cancer therapy.

Additionally, the intestinal microbiota is known to interact with the host immune system (19). Allogeneic hematopoietic stem cell transplantation (allo-HSCT) is one setting where this is particularly true. Patients undergoing allo-HSCT are at risk for graft-versus-host disease (GVHD), a life-threatening inflammatory process where allogeneic donor T-cells recognize the recipient as foreign, and the composition of the intestinal microbiome is an important modulator of GVHD (20). In the lower intestinal tract, commensal microbes are dependent on diet- and host-derived metabolic substrates (21). In turn, they participate in the digestive process and can modulate both local and systemic immunity. Broad-spectrum antibiotics are often used in this patient population to treat infections that occur in the setting of conditioning-mediated neutropenia and mucosal injury. They have been found, however, to increase the risk for intestinal GVHD. The gastrointestinal tract has been identified as a primary target of allogeneic donor T-cells in allo-HSCT, and intestinal GVHD often serves to amplify systemic inflammation (22). Indeed, intestinal microbiome injury following allo-HSCT is consistently and reproducibly associated with GVHD-related mortality and reduced overall survival (20, 23). In this disclosure, it is shown that antibiotic treatment aggravated GVHD primarily in the colon, and that the gut microbiome composition of a subject receiving an antibiotic correlates with development of GVHD in subjects receiving a cancer therapy and/or a neutropenic fever therapy. Antibiotic-treated mice developed a thinned colonic mucus layer and increased colonic epithelial damage, myeloid cell infiltration and bacterial translocation into mesenteric lymph nodes (MLNs), as well as increased expression of mucolytic enzymes and altered levels of carbohydrates in the colonic lumen. Surprisingly, supplementation of antibiotic-treated mice with carbohydrates metabolized by mucus-degrading bacteria in the gut resulted in a significantly thicker mucus layer, which can reduce the severity of or prevent GVHD.

The present disclosure relates to methods and compositions for the treatment of neutropenic fever and/or GVHD, such as cancer therapy-induced neutropenic fever and/or HCT-related GVHD and/or neutropenic fever therapy-induced GVHD, by modulating the microbiome and/or the activity of gut bacteria to prevent or reduce the severity of neutropenic fever and/or GVHD (e.g., HCT-related GVHD and/or neutropenic fever therapy-induced GVHD). The present disclosure also relates to microbiome activity metrics and bacterial abundance as biomarkers for predicting development of neutropenic fever and/or GVHD (e.g., HCT-related GVHD and/or neutropenic fever therapy-induced GVHD) in subjects.

The results encompassed herein demonstrate that novel approaches can prevent fevers in the setting of neutropenia following cancer therapy and/or can prevent GVHD in the setting of HCT therapy following cancer therapy and/or neutropenic fever treatment following cancer therapy. In particular embodiments, the composition and activity of the gut microbiome is analyzed or measured or determined or evaluated for a subject receiving a cancer therapy, and that analysis may occur by any suitable method. In some embodiments, a therapeutically effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, one or more mediators of organic acid metabolite levels produced following metabolism of mucin-derived carbohydrates by one or more genera of mucus-degrading bacteria in the gut microbiome of the subject, and/or one or more carbohydrate substrates metabolized by the one or more genera of mucus-degrading bacteria in the gut microbiome of the subject, are prophylactically utilized to prevent or reduce the severity of neutropenic fever and/or GVHD (e.g., HCT-related GVHD and/or neutropenic fever therapy-induced GVHD). In such cases, prior analysis of the composition and activity of the gut microbiome allows determination of which individuals would need the bacterial growth-suppressing agent, mucus-degrading enzyme inhibitor, mediator of organic acid metabolite levels, and/or carbohydrate substrates, allowing for informed clinical decision making.

### I. Neutropenic Fever

Neutropenia is an abnormally low concentration of neutrophils in the blood. Neutrophils make up the majority of circulating white blood cells and serve as the primary defense against infections by destroying bacteria, bacterial fragments, and immunoglobulin-bound viruses in the blood. People with neutropenia are more susceptible to bacterial infections and, without prompt medical attention, the condition may become life-threatening. The term “neutropenia” is sometimes used interchangeably with “leukopenia,” which refers to a deficit in the number of white blood cells.

Neutropenia can be the result of a variety of consequences, including from certain types of drugs, environmental toxins, vitamin deficiencies, metabolic abnormalities, and nutritional deficiencies, such as deficiency in vitamin B12, folate, copper or protein-calorie malnutrition, as well as cancer or infections, especially in people with underlying hematological diseases, which can deplete neutrophil reserves and lead to neutropenia. Acute neutropenia can be clinically common in oncology and immunocompromised individuals as a result of cancer therapy (cancer therapy-induced neutropenia), and in individuals recovering from a viral infection. Neutropenia that is developed in response to chemotherapy typically becomes evident in seven to fourteen days after treatment. Rarer, chronic forms of neutropenia include acquired (idiopathic) neutropenia, cyclic neutropenia, autoimmune neutropenia, and congenital neutropenia.

Signs and symptoms of neutropenia include fever, painful swallowing, gingival pain, skin abscesses, and otitis. These symptoms may exist because individuals with neutropenia often have infection.

The diagnosis of neutropenia is done via the low neutrophil count detection on a complete blood count. A bone marrow biopsy can identify abnormalities in myelopoiesis contributing to neutropenia such as the stage of arrest in the development of myeloid progenitor cells. Bone marrow biopsies can also be used to monitor the development of myelodysplastic syndrome (MDS) or acute myeloid leukemia (AML) in patients with chronic neutropenia (especially in those with severe congenital neutropenia (SCN) which carries a higher risk of MDS and AML)). Other tests commonly performed include serial neutrophil counts for suspected cyclic neutropenia, tests for anti-neutrophil antibodies, autoantibody screen, and vitamin B12 and folate assays.

The generally accepted reference range for absolute neutrophil count (ANC) in adults is 1500 to 8000 cells per microliter (μl) of blood. The Absolute Neutrophil Count (ANC) is a calculated parameter based on the total number of white blood cells, the percentage of neutrophils, and the percentage of band cells in a patient's blood sample at a given time. It has been used as a parameter to assess immune function and risk stratify patients for likelihood of acute bacterial infection. ANC is derived via the following formula:

$$ANC = \frac{( \% neutrophils + \% band cells ) \times ( WBC )}{100}$$

where % neutrophils refers to the percentage of neutrophils in a patient's blood sample, % band cells refers to the percentage of band cells in a patient's blood sample, and WBC refers to the total number of white blood cells in a patient's blood sample.

ANC levels have become a benchmark to assess the risk of opportunistic bacterial infections, in immunosuppressed patients with malignancy receiving chemotherapy, for example. The severity of neutropenia based on the ANC (expressed below in cells/μl) is as follows: mild neutropenia (1000≤ANC<1500)—minimal risk of infection; moderate neutropenia (500≤ANC<1000)—moderate risk of infection; severe neutropenia (ANC<500)—severe risk of infection.

A fever, when combined with profound neutropenia, referred to herein as “neutropenic fever,” “febrile neutropenia,” or “NPF.” is considered a medical emergency. Two categories of neutropenic fevers have been described. Microbiologically-based NPF infection is defined when the cultures isolate an organism. On the other hand clinically documented NPF is present when there is a high clinical suspicion for infection based on physical examination findings or radiological testing but there is a negative microbiologic work up. During the work up of NPF, an infectious origin can be identified either microbiologically and/or clinically in only 30-50% of the cases. The risk of febrile neutropenia not only depends on the duration and degree of neutropenia but also on other factors related to the demographics of the patient, for example the malignancy in question or the treatment regimen being delivered. The highest risk for NPF is in in patients with profound and protracted neutropenia after induction chemotherapy for acute leukemia and in the pre-engraftment stage following stem cell transplant infusion.

Treatment of NF requires broad spectrum antibiotics, including cefepime, carbapenems (meropenem and imipenem/cilastatin), piperacillin/tazobactam, amoxicillin-clavulanic acid, or ciprofloxacin. Generally, patients with febrile neutropenia are treated with empirical antibiotics until the neutrophil count has recovered (absolute neutrophil counts greater than 500/mm³) and the fever has abated; if the neutrophil count does not improve, treatment may need to continue for two weeks or occasionally more. In cases of recurrent or persistent fever, an antifungal agent such as amphotericin B can be added.

In addition to antibiotics, neutropenia can be treated with the hematopoietic growth factor granulocyte-colony stimulating factor (G-CSF). G-CSF is a cytokine promotes neutrophil recovery following anticancer therapy or in chronic neutropenia, for example. Recombinant G-CSF factor preparations, such as filgrastim, can be effective in people with congenital forms of neutropenia including severe congenital neutropenia and cyclic neutropenia. The administration of intravenous immunoglobulins (IVIGs) has also had some success in treating neutropenias of alloimmune and autoimmune origins with a response rate of about 50%.

In some embodiments disclosed herein, neutropenic fever results from administration of a cancer therapy. Thus, in some embodiments, the neutropenic fever is cancer therapy-induced neutropenic fever. The cancer therapy can include but is not limited to the following cytotoxic cancer therapies: chemotherapy, such as alkylating or other marrow-suppressive agents commonly used in the treatment of cancer patients; radiotherapy; and immunotherapy. Thus, in some embodiments, the cancer therapy-induced neutropenic fever is chemotherapy-induced neutropenic fever. In some embodiments, the chemotherapy-induced neutropenic fever is alkylating agent-induced neutropenic fever. In some embodiments, the chemotherapy-induced neutropenic fever is marrow-suppressive agent-induced neutropenic fever. In some embodiments, the cancer therapy-induced neutropenic fever is radiotherapy-induced neutropenic fever. In some embodiments, the cancer therapy-induced neutropenic fever is immunotherapy-induced neutropenic fever.

### A. Chemotherapy-Induced Neutropenic Fever

In some embodiments, neutropenic fever results from administration of a chemotherapy. Chemotherapeutic agents that can induce neutropenic fever include but are not limited to: (a) Alkylating Agents, such as nitrogen mustards (e.g., mechlorethamine, cyclophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozotocin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophyllotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydrazine derivatives (e.g., procarbazine), and adrenocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional chemotherapeutic agents include pyrimidine analogs, such as cytarabine (cytosine arabinoside), 5-fluorouracil (fluoruracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

### B. Radiotherapy-Induced Neutropenic Fever

In some embodiments, neutropenic fever results from administration of a radiotherapy, such as ionizing radiation. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

In some embodiments, the radiotherapy can comprise external radiotherapy, internal radiotherapy, radioimmunotherapy, or intraoperative radiation therapy (IORT). In some embodiments, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some embodiments, the internal radiotherapy comprises interstitial brachytherapy, intracavitary brachytherapy, or intraluminal radiation therapy. In some embodiments, the radiotherapy is administered to a primary tumor.

### C. Immunotherapy-Induced Neutropenic Fever

In some embodiments, neutropenic fever results from administration of an immunotherapy, such as ionizing radiation. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

Embodiments of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT.” “checkpoint blockade immunotherapy.” or “CBI”), refers to cancer therapy comprising providing one or more immune checkpoint inhibitors to a subject suffering from or suspected of having cancer.

PD-1 can act in the tumor microenvironment where T-cells encounter an infection or tumor. Activated T-cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T-cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T-cells and acts as an “off” switch when bound to B7-1 (CD80) or B7-2 (CD86) on the surface of antigen-presenting cells. CTLA4 is a member of the immunoglobulin superfamily that is expressed on the surface of Helper T-cells and transmits an inhibitory signal to T-cells. CTLA4 is similar to the T-cell co-stimulatory protein, CD28, and both molecules bind to B7-1 and B7-2 on antigen-presenting cells. CTLA-4 transmits an inhibitory signal to T-cells, whereas CD28 transmits a stimulatory signal. Intracellular CTLA-4 is also found in regulatory T-cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and YERVOY®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T-cells, natural killer cells, B cells, and plasmacytoid dendritic cells. LAG3's main ligand is MHC class II, and it negatively regulates cellular proliferation, activation, and homeostasis of T-cells, in a similar fashion to CTLA-4 and PD-1, and has been reported to play a role in Treg suppressive function. LAG3 also helps maintain CD8+ T-cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed disclosure can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1. CDR2, and CDR3 domains of the VH region of an anti-LAG3 antibody, and the CDR1, CDR2 and CDR3 domains of the VL region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed disclosure can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another embodiment, the antibody has at least about 70, 75, 80, 85, 90, 95, 97, or 99% (or any derivable range therein) variable region amino acid sequence identity with the above-mentioned antibodies.

### 2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T-cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T-cells create a link between an extracellular ligand recognition domain and an intracellular signaling molecule which in turn activates T-cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T-cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T-cells to tumor antigens.

### II. Graft-Versus-Host Disease

Graft-versus-host disease (GVHD) is a potentially serious complication of allogeneic stem cell transplantation. During allogeneic stem cell transplantation, a patient receives stem cells from a donor. GVHD may occur after stem cell transplantation when T-cells present in the graft, attack and damage the tissues of the transplant recipient after perceiving host tissues as antigenically foreign. The T-cells produce an excess of cytokines, including TNF-α and interferon-gamma (IFNγ). A wide range of host antigens can initiate graft-versus-host-disease, among them the human leukocyte antigens (HLA). However, graft-versus-host disease can occur even when HLA-identical siblings are the donors. HLA-identical siblings or HLA-identical unrelated donors may have genetically different minor histocompatibility antigens that can be presented by major histocompatibility complex (MHC) molecules to the donor T-cells, which see these antigens as foreign and so mount an immune response.

The pathophysiology of GVHD includes three phases: the afferent phase, characterized by activation of APC (antigen presenting cells); the efferent phase, characterized by activation, proliferation, differentiation and migration of effector cells; and the effector phase, characterized by target tissue destruction. Activation of APC occurs in the first stage of GVHD. Prior to hematopoietic stem cell transplantation, radiation or chemotherapy results in damage and activation of host tissues, especially intestinal mucosa.

Alternatively, as shown herein, administration of broad-spectrum antibiotics to treat or prevent neutropenic fever and infections may also result in damage to or thinning of intestinal mucosa. For example, in allogeneic hematopoietic stem cell transplantation, major microbiome shifts are seen in association with antibiotic administration (24). Certain classes of antibiotics including carbapenems have been particularly associated with increased intestinal GVHD (25-28). In some cases, intestinal microbiota, including Clostridia, can serve an important function in maintaining intestinal homeostasis, including intestinal mucosa thickness, and introduction of an experimental consortium of Clostridia was found to suppress intestinal inflammation in mice by a variety of mechanisms, including induction of regulatory T cells in the colon (29) and production of butyrate (20). Previous studies have shown that Clostridia play an important role in producing SCFAs, including butyrate which plays a role in maintaining the epithelial integrity (51, 52). Loss of Clostridia during GVHD results in increased epithelial injury (30). Thus, in some embodiments, clearance of commensal bacteria by broad-spectrum antibiotic administration may result in damage to or thinning of intestinal mucosa.

This allows the microbial products to enter and stimulate pro-inflammatory cytokines such as IL-1 and TNF-α. These pro-inflammatory cytokines increase the expression of MHC and adhesion molecules on APCs, thereby increasing the ability of APC to present antigen. Activation of effector donor T-cells further enhances the expression of MHC and adhesion molecules, chemokines, and the expansion of CD8⁺ and CD4⁺ T-cells and guest B-cells. In the final phase, these effector cells migrate to target organs and mediate tissue damage, resulting in multiorgan failure, in some cases.

GVHD disease can be mild, moderate or severe. In some cases, it can be life-threatening. There are two main categories of GVHD: acute graft-versus-host disease and chronic graft-versus-host disease. In some cases, each type affects different organs and tissues and has different signs and symptoms. Patients may develop one type, both types, or neither type.

Acute GVHD usually develops within the first 10 to 100 days after transplantation, although in some cases, it can occur later. Acute GVHD can affect the skin, the gastrointestinal tract, and/or the liver. Symptoms may include: a rash; burning, blistering, flaking, and/or redness of the skin; nausea, vomiting, abdominal cramps, loss of appetite, and/or diarrhea; and/or jaundice. In some cases, patients who develop acute GVHD are successfully treated with increased immunosuppression in the form of corticosteroids (medicines such as prednisone, methylprednisolone, dexamethasone, beclomethasone and budesonide).

Chronic GVHD may involve a single organ or several organs. It is one of the leading causes of medical problems and death after an allogeneic stem cell transplantation. Symptoms may include dry mouth; sensitivity to hot, cold, spicy and acidic foods, mint, and/or carbonated drinks, mouth ulcers that may extend down the throat; difficulty eating; gum disease and tooth decay; rash; dry, tight, itchy skin; thickening and tightening of the skin, which may result in restriction of joint movement; change in skin color; intolerance to temperature changes due to damaged sweat glands; changes in nail texture; hard, brittle nails; nail loss; hair loss; premature gray hair; loss of appetite; unexplained weight loss; nausea; vomiting; diarrhea; stomach pain; shortness of breath and difficulty breathing; persistent, chronic cough; wheezing; abdominal swelling; jaundice; abnormal liver function test results; muscle weakness and cramps; joint stiffness causing difficult full extension of fingers, wrists, elbows, knees, ankles; vaginal dryness; vaginal or penile itching, pain, and irritation; vaginal or urethral ulcerations and scarring; narrowing of the vagina or urethra; and difficult/painful intercourse.

Patients with mild symptoms of chronic GVHD, especially if the symptoms are limited to a single organ or site, can often be treated with close observation or with local/topical therapies. For example, mild cases of chronic skin GVHD may be treated with topical steroid ointments or immunosuppressants.

Patients with more severe symptoms or multi organ involvement chronic GVHD typically require “systemic” treatment using corticosteroids (medicines such as prednisone, methylprednisolone, dexamethasone, beclomethasone and budesonide).

Thus, in some embodiments, corticosteroids are administered in combination with or in addition to a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, one or more mediators of organic acid metabolite levels produced following metabolism of mucin-derived carbohydrates by one or more genera of mucus-degrading bacteria in the gut microbiome of the subject, and/or one or more carbohydrate substrates metabolized by the one or more genera of mucus-degrading bacteria in the gut microbiome of the subject.

### III. Mucins and Mucus-Degrading Bacteria

The present disclosure relates to methods and compositions for the treatment of neutropenic fever and/or GVHD, such as cancer therapy-induced neutropenic fever and/or HCT-related GVHD and/or neutropenic fever therapy-induced GVHD, by modulating the microbiome and/or the activity of mucus-degrading bacteria in the gut to prevent or reduce the severity of neutropenic fever and/or GVHD (e.g., HCT-related GVHD and/or neutropenic fever therapy-induced GVHD). The present disclosure also relates to mucus-degrading bacteria activity metrics and abundance of mucus-degrading bacteria as biomarkers for predicting development of neutropenic fever and/or GVHD (e.g., HCT-related GVHD and/or neutropenic fever therapy-induced GVHD) in subjects.

### A. Mucin

Mucins are the main structural components of mucus and play an integral and multifaceted role in the interaction between microbes and epithelial surfaces. The expression profile of mucins varies among host tissues and particularly within the GI tract, which displays the highest and most diverse levels of mucin expression in the body. To date, more than 20 genes encoding mucins have been identified in humans, with their classification based on the arrangement of their monomeric polypeptide domains. Mucins are broadly grouped as membrane-bound or secreted proteins. Common to each mucin are an N-terminal signal peptide and a proline-threonine-serine (PTS) domain. The signal peptide is required for the targeting of the polypeptide to the endoplasmic reticulum (ER) and either extracellular secretion or insertion of the synthesized mucin into the cell membrane. The PTS domain is the site of extensive O-glycosylation with carbohydrates accounting for up to 80% of the total mucin mass. These PTS domains, comprised of variable number of tandem repeat (VNTR) domains, allow for a great degree of heterogeneity in mucins, due to variability in both, mucin length and extent of glycan attachment at these sites. This characteristic filamentous protein backbone decorated with outwardly protruding oligosaccharides results in the typical “bottle-brush”-like appearance of mucins.

Membrane-bound mucins are essential contributors of the glycocalyx of mucosal surfaces where they play important biological roles in cell-cell and cell-matrix interactions, and in cell signaling. These mucins may be shed from the surface and integrate into the overlying mucus layer where they are able to influence the viscosity of the protective layer. Secreted mucins are the main structural components of the mucus gel. Along the GI tract, synthesis and secretion of these polymeric glycoproteins take place in the goblet cells of the small intestine and colon, or the surface mucous cells of the stomach. Characteristic properties of the secreted mucins are disulfide bridges which are formed among the cysteine-rich, cysteine knot, and von Willebrand C and D domains residing at the N- or C-termini of the glycoprotein monomers. MUC2 is the best characterized secreted mucin of the GI tract. Within the ER, newly synthesized MUC2 peptides immediately dimerize through the formation of disulfide bridges followed by transport to the Golgi apparatus. Here, the PTS domains of the mucin dimers are sites of elaborate glycosylation before further assembly into trimers in the trans-Golgi network and packaging into goblet cell vesicles in a pH- and Ca²⁺-dependent manner. As a monomer, fully glycosylated MUC2 exhibits a large size of approximately 2.5 MDa, while extensive polymerization may allow for sizes of greater than 100 MDa. Following secretion of the mucin granules at the mucosal surface, the densely-packed mucin structures are hydrated and rapidly expand to a size approximately 3000-fold greater than in the granules, thus providing a dynamic barrier.

In addition to their protective and lubricating activities, mucins facilitate microbial tropism through the presentation of glycans which may impact colonization, and act as a nutritional source for microorganisms. As such, mucin glycans have been proposed to play a key role in selecting microbial communities along and across the GI tract. Consistent with this hypothesis, recent studies in mouse models and humans showed an association between alteration in mucin glycosylation profile and deviations of overall community ecology along with altered abundances of specific microbes.

### B. Mucin-Degrading Bacteria

Embodiments of the disclosure concern one or more genera of mucus-degrading bacteria in the gut microbiome. In some embodiments, the one or more genera of mucus-degrading bacteria include Akkermansia and Bacteroides. In some embodiments, the one or more genera of mucus-degrading bacteria include Bacteroides, Akkermansia, Ruminococcus, and Bifidobacterium.

As used herein, “gut flora,” “gastrointestinal flora,” “intestinal flora,” “gut microbiome,” “intestinal microbiome,” “microbiome,” and the like are interchangeable and are intended to represent the normal, naturally occurring bacterial population present in the gastric and intestinal systems of healthy humans and animals. The terms are meant to reflect both the variety of bacterial species and the concentration of bacterial species found in a healthy human or animal.

As used herein, “gut.” “intestine,” “intestinal tract,” “colon,” and the like are used interchangeably and are intended to represent the gastrointestinal system of humans.

The GI tract is heavily colonized by bacteria that make up the gut microbiome with most species belonging to the phyla Firmicutes, Bacteroidetes, Actinobacteria, Proteobacteria, and Verrucomicrobia. The microbiota composition varies longitudinally along the GI tract but also transversally from the mucosa to the lumen.

In the colon, the epithelium is covered by a thick gel of mucus, divided into two layers, an inner layer firmly attached to the epithelium and a loose outer layer. The outer mucus layer is heavily colonized by bacteria, while the inner layer contains no or very few bacteria. It is thus believed that in healthy conditions mucosa-associated bacteria are not in direct contact with the epithelium but are restricted to the outer mucus layer. Although the molecular mechanisms underpinning the adaptation of gut bacteria to mucus remain unclear, it is likely that their ability to utilize mucin glycans as a source of nutrients would confer a competitive advantage to those bacteria with the required repertoire of hydrolytic enzymes. The first mucin-degrading (or mucinolytic) bacteria studied were pathogens, and thus for a long period mucin degradation had been associated with pathogenicity. However, it is now clear that mucin degradation is part of a normal turn-over process starting a few months after birth. To date, only a limited number of bacterial species/strains from the Bacteroidetes, Firmicutes, Actinobacteria, and Verrucomicrobia phyla have been studied for their ability to consume mucins.

The mucin-degrading ability of gut bacteria has been extensively studied in Bacteroidetes. An early study showed that all 22 strains of Bacteroides thetaiotaomicron tested were able to ferment glycosaminoglycans (GAG) but failed to ferment pig gastric mucin (PGM) or bovine submaxillary mucin (BSM). However, later, B. thetaiotaomicron VPI-5482 was shown to be able to grow on different fractions of glycans purified from pig gastric mucosa, including an O-glycan rich fraction. Transcriptomic analyses highlighted specific polysaccharide-utilization loci (PULs) including genes coding for putative glycoside hydrolases (GHs) such as α-L-fucosidase, endo-β-N-acetylglucosaminidase, endo-β-galactosidase and α-mannosidase, which were up-regulated when B. thetaiotaomicron was grown on mucin O-glycans or in monoxenic mice as compared to in vitro glucose control. Interestingly, these PULs were not up-regulated when B. thetaiotaomicron was grown on GAG, as compared to glucose. Colonization competition experiments demonstrated that B. thetaiotaomicron mutants for O-glycan PULs were able to colonize germ-free mice in a similar way as the wild-type strain when mice were fed a plant glycan-rich diet, but were outcompeted by the wild-type on a simple-sugar diet. This indicates that B. thetaiotaomicron relies on mucin and other host-derived glycans for colonization. Genome analysis of Bacteroides fragilis confirmed that Bacteroides species contain a much larger number of genes encoding carbohydrate-active enzymes (CAZymes) compared to other sequenced gut bacteria. In accordance with early studies demonstrating the ability of some B. fragilis strains to grow on mucin as sole carbon source, the B. fragilis genome contains a subset of PULs dedicated to host mucin O-glycan utilization; in particular, it has been shown that (i) loci involved in the binding, degradation, and transport of sialylated polysaccharides play an important role in the colonization of this bacterium in the gut and (ii) the genes involved in sialic acid utilization are up-regulated when B. fragilis is grown in the presence of mucin O-glycans as compared to glucose. Some strains of Bacteroides vulgatus have also been shown to moderately degrade PGM but failed to utilize human MUC2.

In the Firmicutes phylum, Ruminococcus torques and Ruminococcus gnavus, both members of the Lachnospiraceae family (belonging to the C. coccoides group/cluster XIVa) have been shown to degrade mucins. In an early study, six R. torques strains out of nine tested, but none of the R. gnavus strains tested, were shown to have the capacity to ferment PGM. A few years later, R. gnavus ATCC 35913 and two R. torques strains (ATCC 35915 and VIII-239) were among the five strains isolated from human fecal samples for their ability to degrade mucins. Both R. gnavus and R. torques species were able to degrade and utilize human MUC2 as a sole carbon source, providing further evidence of their adaptation to the human colonic mucosal environment. Several enzymatic activities were detected in the spent media of these strains grown with mucin that could explain their ability to degrade mucin. Recently, the ability of R. gnavus strains to utilize mucin was shown to be strain-dependent, as also supported by comparative genomic and transcriptomic analyses, and in agreement with earlier findings.

B. bifidum ATCC 35914 and B. longum subsp. infantis VIII-240, from the Actinobacteria phylum, were also isolated as mucin degraders, and several enzymatic activities possibly involved in mucin degradation were detected in the spent media of these strains grown with mucin as sole carbon source. Since then, B. longum subsp. infantis has been shown to grow on mucins, albeit moderately, and the ability of B. bifidum to utilize mucins has been confirmed for several strains using different types of mucins, including human MUC2. Transcriptomic analyses of B. bifidum L22 and PLR2010 suggest the involvement of several enzymes in the process, e.g., α-L-fucosidase and endo-α-N-acetylgalactosaminidase. Few other Bifidobacteria species, including some strains of B. longum subsp. longum and Bifidobacterium breve, have also been shown to degrade mucins in vitro. β-N-acetylglucosaminidase and β-glucuronidase activities were increased in the spent medium of B. longum subsp. longum NCIMB8809 grown with human intestinal mucus, suggesting a role of these activities in mucin degradation. More recently, detailed genome analyses of Bifidobacteria have identified metabolic pathways for the degradation of mucin-type O-glycans and human milk oligosaccharides (HMOs) and several GHs have been functionally characterized supporting these findings.

In the Verrucomicrobia phylum, Akkermansia muciniphila, a strictly anaerobic Gram-negative bacterial species, was recently identified as a key mucin degrader. Initially isolated from a human fecal sample due to its ability to utilize mucins as sole carbon and nitrogen source, A. muciniphila has since been shown to be a common member of the human gut with a high prevalence and variable abundance, present both in feces and at the mucosal surface. When A. muciniphila ATCC BAA-835 was grown with mucin as sole carbon source, several enzymatic activities potentially involved in mucin degradation were detected both in the spent medium and intracellularly. Numerous genes encoding putative mucinolytic enzymes were found in the genome of the ATCC BAA-835 strain.

### C. Mucus Degradation

Mucin degradation is achieved by a combination of mainly saccharolytic enzymes from the bacteria and proteolytic enzymes from the host and bacteria. As discussed above, the composition of O-linked glycans on the mucins, their size, linkages and terminal sugar-residues differs along the GI tract, being more neutral in the upper part, while more acidic in the lower part. Mucin-degrading bacteria can adapt to the host mucins by producing specific enzymes, which are able to degrade the histo-blood group antigens (oligosaccharides). Given the diversity and complexity of intestinal mucin glycan structures, strategies for metabolizing or degrading these molecules rely on the cooperative action of a number of enzymes, including proteases, sulfatases, and glycoside hydrolases (GHs), together designated as “mucinases,” encoded by the genome of mucin-degrading bacteria. These GHs include, but are not limited to, neuraminidases/sialidases (GH33), fucosidases (GH29 and GH95), exo- and endo-β-N-acetylglucosaminidases (GH84 and GH85), β-galactosidases (GH2, GH20, and GH42), α-N-acetylglucosaminidases (GH89), and α-N-acetylgalactosaminidases (GH101, GH129). In addition to their catalytic domains, GHs may have one or more carbohydrate binding modules (CBMs) which mediate the adherence of CAZymes to their carbohydrate substrate. Currently, CBMs that recognize mucin glycans have been reported in families 32, 40, 47, and 51. These CBMs show specificity for terminal glycan motifs, such as Gal, GlcNAc, sialic acid, fucose, and histo-blood group antigens. Other non-catalytic domains associated with these GHs include immunoglobulin domains, concanavalin A domains, or domains of unknown function.

Mucin degradation in vivo starts probably with cleavage of the non-glycosylated regions of the polypeptide backbone performed by proteolytic enzymes. Subsequently, the oligosaccharide chains are degraded by a panel of diverse glycosidases and finally followed by proteolytic degradation of the exposed protein core. Proteases are secreted by both host and bacteria, whereas glycosidases capable of degrading mucin-type O-linked glycans are only secreted by bacteria and not by the host tissues. The bacterial glycosidases include mainly β-N-acetyl-D-galactosaminidase. β-N-acetyl-D-glucosaminidase, α- and β-D-galactosidase and α-D-mannosidase; whereas the latter plays only a minor role in degradation of mucins that are relatively poor in N-linked glycans (mannose is only present in N-linked glycans). Sialidases (neuraminidases), sulfatases and α-fucosidases act on the terminal ends of the oligosaccharide chains and will often act as initiators of mucin oligosaccharide degradation, as sulfate, sialic acids and α-fucose (the latter in particular in the diverse blood group structures) form the usual terminal structures on the mucin-type O-glycans. Mucin degradation also requires the subsequent actions of several microbial enzymes, mainly glycosidases, each having the specificity to degrade a specific glycoside linkage.

The release of sialic acid from non-reducing ends is an initial step in the sequential degradation of mucins since the terminal location of sialic acid residues in the mucin oligosaccharide chains may prevent the action of other GHs. In bacteria, the genes involved in sialic acid metabolism are usually found clustered together forming what is denominated as a Nan cluster. Human gut bacteria that encode a Nan cluster include A. muciniphila, R. gnavus, Anaerotruncus colihominis, Dorea formicigenerans, Dorea longicatena, F. prausnitzii, Fusobacterium nucleatum, Lactobacillus sakei, Lactobacillus plantarum, and Lactobacillus salivarius, B. fragilis, and B. breve. Thus, the majority of the bacteria that harbor a Nan cluster colonize mucus regions of the human body, such as the gut, lung, bladder, or oral cavity, where sialic acid is highly abundant and can serve as a source of energy, carbon, and nitrogen.

GH33 sialidases encoded by human gut bacteria vary in terms of their substrate specificity and enzymatic reaction. Although most of them are hydrolytic sialidases, releasing sialic acid from sialylated substrates, some display transglycosylation activities. For example the sialidase from B. bifidum JCM 1254 can transfer Neu5Ac to 1-alkanols, that of R. gnavus ATCC 29149 (RgNanH) is an intramolecular trans-sialidase (IT-sialidase) which releases 2,7 anhydro-Neu5Ac specifically from α2,3-linked sialyl conjugates, and NanI from Clostridium perfringens str 13 can hydrate the inhibitor 2-deoxy-2,3-dehydro-Neu5Ac to Neu5Ac. Trans-sialidases show specificity for α2,3 linkages, whereas hydrolytic sialidases can often cleave α2,3, 2,6, or 2,8 linkages (e.g., B. thetaiotaomicron sialidase BTSA).

In mucins, fucosyl residues can be found at the extremity of the O-glycosidic chain linked to galactose by α1,2 linkages or to GlcNAc by α1,3 linkages, whereas it is most commonly linked α1,6 to the reducing, terminal β-GlcNAc in human N-linked glycans. Fucosidase-encoding genes are widely distributed in the genome of gut bacteria and generally belong to GH29 and GH95 families, which differ in their enzymatic mechanisms; GH29 enzymes retain the anomeric conformation of the glycosidic bond whereas GH95 enzymes proceed via the inverting mechanism. Transcriptional data suggest that GH29 and GH95 fucosidases play a key role in the ability of B. thetaiotaomicron VPI-5482, B. longum subspecies infantis ATCC 15697, B. bifidum JCM 1254, and R. gnavus ATCC 29149 to utilize mucins as a source of carbon. The substrate specificity of GH29 and GH95 fucosidases has been predominantly characterized in B. bifidum JCM 1254, where GH95 AfcA was shown to be specific for α1,2 linkages and GH29 AfcB for α1,3 and α1,4 linkages; together these enzymes can remove fucose at the non-reducing termini except for those that are α1,6-linked. AfcA and AfcB have been shown to be sufficient to confer B. longum 105-A with the ability to grow on 2-fucosyllactose (2′FL), 3-fucosyllactose (3′FL) and lacto-N-fucopentaose (LNFPII). The structural basis for the specificity of AfcA has been determined.

B. thetaiotaomicron produces multiple fucosidases that cleave fucose from host glycans, resulting in high fucose availability in the gut lumen. The genome of B. thetaiotaomicron VPI-5482 encodes five GH95 and nine GH29 genes. Two of the GH29 genes have been expressed and shown to have α-fucosidase activity and have been classified in separate sub-families, i.e., GH29-A (BT_2970) has a relaxed specificity that can accommodate pNP-fucose (pNP-Fuc), whereas GH29-B (BT_2192) is specific for branched fuco-oligosaccharides found in Lewis blood groups (also present in mucin structures). Structural analysis of BT_2192 elucidated the molecular mechanisms for the binding of the branched oligosaccharides and the unusual dual specificity of this enzyme, which also acts as a β-galactosidase.

Both the blood group A antigen and B antigen can be cleaved from mucin by GH98 endo-β1.4-galactosidases, these have been characterized in Clostridium perfringens strains 10543 and 13. The only structural information about this family of enzymes comes from Streptococcus pneumoniae str. and reveals a (α/β)8 barrel. Once the terminal sugars and blood group antigens are removed, the mucin core glycans are exposed to further enzymatic degradation.

Mucin glycan core structures are cleaved from the Ser/Thr amino acids of the mucin protein backbone by endo-α-N-acetylgalactosaminidases, with that of B. bifidum (EndoBF) being the founding member of GH101. These enzymes differ in their specificity toward core glycan structure types. For example, EndoBF is specific for the core 1 glycan (Ga1β1,3GalNAc) while endo-α-N-acetylgalactosaminidases from Enterococcus faecalis and C. perfringens have a broader specificity. The structural basis for this specificity has been elucidated for EndoBF. The α-N-acetylgalactosaminidase from B. bifidum JCM 1254 is the founding member of GH129 and differs from GH101 in that it targets the Tn antigen (GalNAcα1-Ser) found in gastroduodenal mucins. Many of the species encoding a GH129 are associated with the infant microbiota, although Bacillus sp. also contain GH129.

GH2, GH20, and GH42 β-galactosidases have been implicated in the degradation of type-1 and type-2 HMOs. Genome analysis of several Bifidobacteria species identified common metabolic pathways for the degradation of lacto-N-biose I (Galβ1,3GlcNAc, LNB) and galacto-N-biose (Galβ1,3GalNAc, GNB), a building block of the core 1 structure of mucin-type O-glycan, whereas the degradation of type-2 lacto-N-neo-tetraose (Galβ1,4GlcNAcβ1,3Galβ1,4Glc, LNnT), also present in the core 2 mucin-type O-glycans, involves a different pathway. In Bifidobacteria, the type-1 chain (Galβ1,3GlcNAcβ1) is likely eliminated by GH20 lacto-N-biosidases (LnbB), and the released LNB incorporated into the cytosol via a GNB/LNB transporter. Despite the quite rare occurrence in nature of β-galactosidases acting on type-1 chains, close lacto-N-biosidase homologs are present in the genomes of infant-gut associated Bifidobacteria that are known to consume type 1 Lacto-N-tetraose (Galβ1,3GlcNAcβ1,3Galβ1,4Glc, LNT), these are the GH42 LNT β-galactosidases. In Bifidobacteria, the type-2 chain (Galβ1,4GlcNAcβ1-) is sequentially degraded by GH2 β-galactosidase, BbgIII, acting on LacNAc and GH20 β-N-acetylhexosaminidases, BbhI and BbhII, specific for GlcNAcβ1,3Galβ1-R. Although GH2 is a very common glycosidase present in intestinal bacteria, the presence of membrane bound β-galactosidases is limited to several bifidobacterial strains. In contrast to β-galactosidases, GH20 β-N-acetylhexosaminidases are relatively rare in the genome of enteric bacteria. An unusual activity was reported for a GH20 enzyme from Prevotella strain RS2; the enzyme cleaved terminal 6-SO₃-GlcNAc from sulfated mucin glycans, representing a novel activity within the GH20 family. Other sulfatases have also been characterized.

GH84 and GH85 β-N-acetylglucosaminidases have also been implicated in mucin metabolism (e.g., C. perfringens str 13, and B. longum NCC2705).

The GH89, α-N-acetylglucosaminidase (AgnC), from C. perfringens (str 13124) has been studied structurally and the highly similar enzyme from C. perfringens str 13 has been demonstrated to be active against PGM and cell surface mucin (from adenocarcinoma AGSα4GnT cells stably expressing GlcNAcα1,4Gal as O-glycans on the cell surface). These studies suggest a role for AgnC in the release of terminal GlcNAc from “Class III” gastroduodenal mucins, similar to GH129.

### IV. Commensal Bacteria

The present disclosure relates to methods and compositions for the treatment of neutropenic fever and/or GVHD, such as cancer therapy-induced neutropenic fever and/or HCT-related GVHD and/or neutropenic fever therapy-induced GVHD, by modulating the microbiome and/or the activity of commensal bacteria in the gut to prevent or reduce the severity of neutropenic fever and/or GVHD (e.g., HCT-related GVHD and/or neutropenic fever therapy-induced GVHD). The present disclosure also relates to commensal bacteria activity metrics and abundance of commensal bacteria as biomarkers for predicting development of neutropenic fever and/or GVHD (e.g., HCT-related GVHD and/or neutropenic fever therapy-induced GVHD) in subjects.

Commensal bacteria (normal microflora, indigenous microbiota) are those micro-organisms present on body surfaces covered by epithelial cells and exposed to the external environment (gastrointestinal and respiratory tract, vagina, skin, etc.). The most abundant commensal bacteria are present in the distal parts of the gut. The majority of the intestinal bacteria are Gram-negative anaerobes.

Commensal bacteria can activate innate and adaptive immunity. The mucosal immune system has developed specialized regulatory, anti-inflammatory mechanisms for eliminating or tolerating non-dangerous, food and airborne antigens and commensal micro-organisms (oral, mucosal tolerance). However, at the same time the mucosal immune system must provide local defense mechanisms against environmental threats (e.g., invading pathogens). This important requirement is fulfilled by several mechanisms of mucosal immunity: strongly developed innate defense mechanisms ensuring appropriate function of the mucosal barrier, existence of unique types of lymphocytes and their products, transport of polymeric immunoglobulins through epithelial cells into secretions (sIgA) and migration and homing of cells originating from the mucosal organized tissues in mucosae and exocrine glands. The important role of commensal bacteria in development of optimally functioning mucosal immune system was demonstrated in germ-free animals (using gnotobiological techniques). Involvement of commensal microflora and its components with strong immunoactivating properties (e.g., LPS, peptidoglycans, superantigens, bacterial DNA, Hsp) in etiopathogenetic mechanism of various complex, multifactorial and multigenic diseases, including inflammatory bowel diseases, periodontal disease, rheumatoid arthritis, atherosclerosis, allergy, multiorgan failure, colon cancer has been recently suggested.

Embodiments of the disclosure concern one or more classes, orders, families, genera, or species of commensal bacteria in the gut microbiome. In some embodiments, the one or more classes, orders, families, genera, or species of commensal bacteria include but are not limited to: Bacteroides vulgatus, Faecalibacterium prausnitzii, Parabacteroides merdae, Eubacterium rectale, Escherichia coli, Roseburia faecis, Phascolarctobacterium faecium, Bacteroides ovatus, Alistipes shahii, Roseburia intestinalis, Parasutterella excrementihominis, Eubacterium eligens, Ruminococcus bromii, Lactobacillus rogosae, Prevotella copri, Anaerostipes hadrus, Bacteroides massilliensis, Bacteroides dorei, Ruminococcus faecis, Sphingomonas echinoides, Bacteroides stercoris, Dorea formicigenerans, Ochrobactrum cytisi, Fusicatenibacter saccharivorans, Sutterella wadswothensis, Sphingomonas melonis, Bacteroides thetaiotaomicron, Bacteroides coprophilus, Blautia obeum, Eubacterium hallii, Roseburia inulinivorans, Holdemanella biformis, Bacteroides cellulosilyticus, Eubacterium siraeum, Coprobacter fastidiosus, Intestinibacter bartlettii, Subdoligranulum variabile, Ruminococcus callidus, Tsukamurella spongiae, Blautia wexlerae, Phascolarctobacterium succinatutens, Ruminococcus lactaris, Bacteroides clarus, Blautia stercoris, Eubacterium hadrum, Butyrivibrio crossotus, Coprobacter secundus, Alistipes indistinctus, Romboutsia ilealis, Holdemania Catenibacterium mitsuokai, Serratia quinivorans, Parabacteroides johnsonii, Enterobacter muelleri, Anaerotruncus colihominis, Jeotgalicoccus halotolerans, Butyricimonas faecihominis, Pseudomonas gessardii, Parasutterella secunda, Bacteroides finegoldii, Barnesiella intestinihominis, Blautia luti, Citrobacter werkmanii, Citrobacter gillenii, Oxalobacter formigenes, Clostridium lactatifermentans, Streptococcus gordonii, Asaccharobacter celatus, Howardella ureilytica, Methylobacterium phyllosphaerae, Blautia hansenii, Coprobacillus cateniformis, Planomicrobium chinense, Parabacteroides faecis, Alistipes inops, Clostridium glycyrrhizinilyticum, Weissella confusa, Pelomonas aquatica, Acidaminococcus fermentans, Aggregatibacter segnis, Romboutsia lituseburensis, Sellimonas intestinalis, Pyramidobacter piscolens, Senegalimassillia anaerobia, Clostridium butyricum, Brachybacterium paraconglomeratum, Sutterella parvirubra, Hungatella effluvii, Kocuria carniohila, Microbacterium esteraomaticum, Aflipia broomeae, Pseudochrobactrums saccharolyticum, Eubacterium oxidoreducens, Bacteroides barnesiae, Butyricimonas paravirosa, Succinatimonas hippei, Enterobacter xiangfangensis, Enhydrobacter aerosaccus, Mesorhizobium jarvisii, Taonella mepensis, Eubacterium sulci, Clostridium citroniae, Clostridium scindens, Sphingomonas hankookensis, Bacteroides plebeius, Eubacterium desmolans, Micrococcus endophyticus, Eubacterium contortum, Dietzia cercidiphylli, Desulfovibrio legallii, Lactobacillus sanfranciscensis, Kluyvera georgiana, Pelomonas puraquae, Olsenella scatoligenes, Eubacterium dolichum, Bacteroides coagulans, Flavobacterium mizutaii, Lactobacillus crispatus, Treponema succinifaciens, Lactonifactor longoviformis, Brevundimonas aurantiaca, Microbacterium sediminicola, Phyllobacterium myrsinacearum, Bacteroides sartorii, Clostridium chartatabidum, Kocuria palustris, Anaerococcus murdochii, Sphingobacterium nematocida, Glutamicibacter protophormiae, Prevotella buccalis, Psychrobacter alimentarius, Herbaspirillum huttiense, Bradyrhizobium icense, Collinsella intestinalis, Desemzia incerta, Cloacibacillus porcorum, Comamonas denitrificans, Brachybacterium ginsengisoli, Bacteroides pectinophilus, Vampirovibrio chlorellavorus, Empedobacter falsenii, Aggregatibacter aphrophillus, Haemophilus sputorum, Aeromonas punctata, Methyloversatilis discipulorum, Paracoccus haeundaensis, Brevundimonas bullata, Phenylobacterium faslum, Sphingomonas glacialis, Rhodococcus degradans, Slackia piriformis, Staphylococcus captis, Lactobacillus delbrueckii, Cloacibacillus evryensis, Alistipes putredinis, Bacteroides galacturonicus, Leuconostoc citreum, Anaerostipes caccae, Clostridium hiranonis, Pseudomonas tolaasii, Aquabacterium citratiphium, Eubacterium xylanophilum, Peptoniphilus duerdenii, Psychrobacter namhaensis, Arthrobacter russicus, Pseudomonas parafulva, Moryella indoligenes, Clostridium ventriculi, Anaerococcus octavius, Prevotella timonensis, Bacteroides stercorirosoris, Cytophaga hutchinsonii, Vibrio natriegens, Thauera terpenica, Massilia norwichensis, Brevibacterium samyangense, Lactobacillus xiangfangensis, Streptococcus peroris, Sphingobacterium alimentarium, Pseudocitrobacter anthropi, Obesumbacterium proteus, Pseudomonas yamanorum, Pseudomonas fragi, Psychrobacter maritimus, Aquincola tertiaricarbonis, Herminiimonas contaminans, Uruburuella suis, Pseudoxanthomonas broegbernensis, Ensifer morelensis, Caulobacter vibrioides, Dietzia kunjamensis, Leucobacter aridicollis, Bacillus simplex, Bacillus altitudinis, Sporosatcina siberiensis, Planococcus koreense, Lactobacillus curvatus, Lactobacillus agilis, Weissella paramesenteroides, Thermus scotoductus, Murimonas intestini, Stomatobaculum longum, Anaerostipes rhamnosivorans, Clostridium sardiniense, Anaerofustis stercorihominis, Paeniclostridium bifermentans, Peptostreptococcus stomatis, Ezakiekka peruensis, Anaerococcus vaginalis, Ruminococcus champanellensis, Prochlorococcus marinus, Prevotella dentalis, Prevotella corporis, Bacteroides faecichinchillae, Pantoea rodasii, Pseudomonas zhaodongensis, Psychrobacter cibarius, Acidovorax wautersii, Variovorax paradoxus, Hydrogenophaga pesudoflava, Sphaerotilus natans, Aquabacterium parvum, Massilia varians, Massilia haematophila, Paraburkholderia fungorum, Neisseria oralis, Pseudoxanthomonas japonensis, Stenotrophomonas rhizophila, Thermomonas carbonis, Rhizobium metallidurans, Aureimonas ureilytica, Bradyrhizobium ganzhouense, Bradyrhizobium neotropicale, Rhodoplanes serenus, Methylobacterium oryzae, Methylobacterium adhaesivum, Microvirga aerophila, Bosea massiliensis, Paracoccus sphaerophysae, Devosia limi, Devosia chinhatensis, Devosia riboflavina, Hyphomicrobium facile, Sphingomonas pseudosanguinis, Sphingomonas aestuarii, Sphingomonas koreensis, Sphingomonas xenophagum, Sphingopyxis bauzanensis, Novosphingobium capsulatum, Belnapia soli, Bifidobacterium faecale, Microlunatus phosphovorus, Arthrobacter globiformis, Micrococcus cohnii, Kocuria salsicia, Pseudarthrobacter polychromogenes, Georgenia satyanarayanai, Microbacterium testaceum, Microbacterium deminutum, Microbacterium mitrae, Microbacterium ginsengisoli, Pseudoclavibacter helvolus, Janibacter anophelis, Bacillus flexus, Bacillus galliciensis, Chryseomicrobium amylolyticum, Planococcus okeanokoites, Listeria marthii, Exiguobacterium aurantiacum, Lactobacillus plantarum, Lactobacillus vaginalis, Lactobacillus mindensis, Lactobacillus farciminis, Enterococcus sulfureus, Carnobacterium inhibens, Facklamia tabacinasalis, Leuconostoc lactis, Weissella viridescens, Gemella asaccharolytica, Deinococcus caeni, Deinococcus reticulitermitis, Deinococcus wulumuqiensis, and/or Clostridium saccharogumia. In specific embodiments, the one or more commensal bacteria include Clostridia bacteria.

### V. Compositions of the Disclosure

### A. Bacterial Growth-Suppressing Agent Compositions

Embodiments of the present disclosure concern one or more bacterial growth-suppressing agent compositions for the treatment of neutropenic fever and/or GVHD, including cancer therapy-induced neutropenic fever and/or HCT-related GVHD and/or neutropenic fever therapy-induced GVHD. The one or more bacterial growth-suppressing agent compositions can comprise, for example, antibiotics and/or antimicrobial proteins or peptides, synthetic or natural compounds, and/or ruminal metabolites. In some embodiments, the bacterial growth-suppressing agents are capable of inhibiting the growth or expansion of one or more genera of bacteria in the microbiome of a subject's gut and/or reducing the number of one or more genera of bacteria in the microbiome of a subject's gut.

The compositions of the one or more bacterial growth-suppressing agent compositions may or may not be tailored to address any deficiency in a subject's gut microbiome or to enhance a subject's gut microbiome. The compositions may be given to a subject without having prior analysis of their gut microbiome. The bacterial growth-suppressing agent compositions may comprise any one or more bacterial growth-suppressing agents associated with efficacious therapy to treat or prevent neutropenic fever and/or GVHD (e.g., HCT-related GVHD and/or neutropenic fever therapy-induced GVHD).

The subject may be given one or more bacterial growth-suppressing agent compositions, including compositions that comprise one or more bacterial growth-suppressing agents that overcome any deficiencies in the subject's gut microbiome. The bacterial growth-suppressing agent(s) may be given to treat or prevent neutropenic fever and/or GVHD (e.g., HCT-related GVHD and/or neutropenic fever therapy-induced GVHD) and/or enhance therapy to treat or prevent neutropenic fever and/or GVHD (e.g., HCT-related GVHD and/or neutropenic fever therapy-induced GVHD).

The bacterial growth-suppressing agent composition can be administered alone or in combination with one or more additional therapeutic agents disclosed herein. Administration “in combination with” one or more additional therapeutic agents includes both simultaneous (at the same time) and consecutive administration in any order. The bacterial growth-suppressing agent composition and one or more additional therapeutic agents can be administered in one composition, or simultaneously as two separate compositions, or sequentially. Administration can be chronic or intermittent, as deemed appropriate by the supervising practitioner, including in view of any change in any undesirable side effects.

In some embodiments, the one or more bacterial growth-suppressing agents are antibiotics. The antibiotics can be of any class, including but not limited to the following classes: aminoglycosides, ansamycins, carbacephem, carbapenems, cephalosporins (first, second, third, fourth, or fifth generation), glycopeptides, linocsamides, lipopeptides, macrolides, monobactams, nitrofurans, oxazolidinones, penicillins, polypeptides, quinolones/fluoroquinolones, sulfonamides, tetracyclines, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, or trimethoprim. Aminoglycosides can include, but are not limited to: Amikacin, Gentamicin, Kanamycin, Neomycin, Netilmicin, Tobramycin, Paromomycin, Streptomycin, and Spectinomycin. Ansamycins can include but are not limited to: Geldanamycin, Herbimycin, and Rifaximin. Carbacephem can include but is not limited to Loracarbef. Carbapenems can include but are not limited to Ertapenem, Doripenem, Imipenem/Cilastatimn, and Meropenem. Cephalosporins can include but are not limited to: Cefadroxil, Cefazolin, Cephradine, Cephapirin, Cephalothin, Cefalexin, Cefaclor, Cefoxitin, Cefotetan, Cefotan, Cefamandole, Cefmetazole, Cefonicid, Loracarbef, Cefprozil, Cefzil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, and Ceftobiprole. Glycopeptides can include but are not limited to: Teicoplanin, Vancomycin, Telavancin, Dalbavancin, and Oritavancin. Lincosamides can include but are not limited to Clindamycin and Lincomycin. Lipopeptides can include but are not limited to Daptomycin. Macrolides can include but are not limited to: Azithromycin, Clarithromycin, Erythromycin, Roxithromycin, Telithromycin, Spiramycin, and Fidaxomicin. Monobactams can include but are not limited to Aztreonam. Nitrofurans can include but are not limited to: Furazolidone and Nitrofurantoin. Oxazolidinones can include but are not limited to: Linczolid, Posizolid, Radezolid, and Torezolid. Penicillins can include but are not limited to: Amoxicillin, Ampicillin, Azlocillin, Dicloxacillin, Flucloxxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, and Ticarcillin/clavulanate. Polypeptides can include but are not limited to: Bacitracin, Colistin, and Polymyxin B. Quinolones/fluoroquinolones can include but are not limited to: Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin. Sulfonamides can include but are not limited to: Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, and Sulfoamidochrysoidine. Tetracyclines can include but are not limited to: Demeclocycline, Doxycycline, Metacycline, Minocycline, Oxytetracycline, and Tetracycline. In some embodiments, the antibiotic is a macrolide. In some embodiment, the antibiotic is azithromycin.

In some embodiments, the one or more bacterial growth-suppressing agents are antimicrobial proteins or peptides. The antimicrobial proteins or peptides can be of any class, including but not limited to the following classes: anionic peptides (e.g., dermicidin), linear cationic α-helical peptides (e.g., LL37), cationic peptides enriched for proline, arginine, phenylalanine, glycine, or tryptophan, and anionic and cationic peptides that contain cysteine and form disulfide bonds (e.g., defensins). Defensins can include but are not limited to trans-defensins, cis-defensins, and related defensin-like proteins. Trans-defensins include but are not limited to α-defensins and β-defensins.

In some embodiments, the one or more bacterial growth-suppressing agents comprise one or more synthetic or natural compounds. In some embodiments, the one or more synthetic or natural compounds comprise bucine, methyl-β-D-galactopyranoside, resacetophenone, or serotonin.

In some embodiments, the one or more bacterial growth-suppressing agents comprise one or more ruminal metabolites. In some embodiments, the one or more ruminal metabolites comprise malic acid, 3-indole acetic acid, hydrocinnamic acid, methylmalonic acid, gluconic acid, galacturonic acid, or bis-hydroxy methyl propionic acid. These ruminal metabolite compounds have been found in human stool, as analyzed by MALDI-TOF mass spectrometry, and therefore, without wishing to be bound by theory, these metabolites would be expected to inhibit bacterial growth or expansion in the gut microbiome.

### B. Mucus-Degrading Enzyme Inhibitor Compositions

Embodiments of the present disclosure concern one or more mucus-degrading enzyme inhibitor compositions for the treatment of neutropenic fever and/or GVHD, including cancer therapy-induced neutropenic fever and/or HCT-related GVHD and/or neutropenic fever therapy-induced GVHD. The one or more mucus-degrading enzyme inhibitor compositions can comprise, for example, mucus-degrading enzyme inhibitors that target one or more enzymes involved in degrading mucus.

Mucin degradation is achieved by a combination of mainly saccharolytic enzymes from the bacteria and proteolytic enzymes from the host and bacteria, including proteases, sulfatases, and glycoside hydrolases (GHs), together designated as “mucinases,” encoded by the genome of mucin-degrading bacteria. These GHs include, but are not limited to, neuraminidases/sialidases (GH33), fucosidases (GH29 and GH95), exo- and endo-β-N-acetylglucosaminidases (GH84 and GH85), β-galactosidases (GH2, GH20, and GH42), α-N-acetylglucosaminidases (GH89), and α-N-acetylgalactosaminidases (GH101, GH129). In addition to their catalytic domains, GHs may have one or more carbohydrate binding modules (CBMs) which mediate the adherence of CAZymes to their carbohydrate substrate. Currently, CBMs that recognize mucin glycans have been reported in families 32, 40, 47, and 51. These CBMs show specificity for terminal glycan motifs, such as Gal, GlcNAc, sialic acid, fucose, and histo-blood group antigens. Other non-catalytic domains associated with these GHs include immunoglobulin domains, concanavalin A domains, or domains of unknown function.

The compositions of the one or more mucus-degrading enzyme inhibitor compositions may or may not be tailored to address any deficiency in a subject's gut microbiome or to enhance a subject's gut microbiome. The compositions may be given to a subject without having prior analysis of their gut microbiome. The mucus-degrading enzyme inhibitor compositions may comprise any one or more mucus-degrading enzyme inhibitors associated with efficacious therapy to treat or prevent neutropenic fever and/or GVHD (e.g., HCT-related GVHD and/or neutropenic fever therapy-induced GVHD).

The subject may be given one or more mucus-degrading enzyme inhibitor compositions, including compositions that comprise one or more mucus-degrading enzyme inhibitors that overcome any deficiencies in the subject's gut microbiome. The mucus-degrading enzyme inhibitor(s) may be given to treat or prevent neutropenic fever and/or GVHD (e.g., HCT-related GVHD and/or neutropenic fever therapy-induced GVHD) and/or enhance therapy to treat or prevent neutropenic fever and/or GVHD (e.g., HCT-related GVHD and/or neutropenic fever therapy-induced GVHD).

The mucus-degrading enzyme inhibitor composition can be administered alone or in combination with one or more additional therapeutic agents disclosed herein. Administration “in combination with” one or more additional therapeutic agents includes both simultaneous (at the same time) and consecutive administration in any order. The mucus-degrading enzyme inhibitor composition and one or more additional therapeutic agents can be administered in one composition, or simultaneously as two separate compositions, or sequentially. Administration can be chronic or intermittent, as deemed appropriate by the supervising practitioner, including in view of any change in any undesirable side effects.

In some embodiments, the one or more mucus-degrading enzyme inhibitors are inhibitors of proteases, sulfatases, and glycoside hydrolases (GHs). Many sulfatases require activation by a sulfatase maturing enzyme, also sometimes called a formylglycine converting enzyme, and thus, mucus-degrading enzyme inhibitors may also include inhibitors of sulfatase maturing enzyme, such as inhibitors of formylglycine converting enzyme. The GHs inhibited by the one or more mucus-degrading enzyme inhibitors can be of any class, including but not limited to: neuraminidases/sialidases (GH33), fucosidases (GH29 and GH95), exo- and endo-β-N-acetylglucosaminidases (GH84 and GH85), β-galactosidases (GH2, GH20, and GH42), α-N-acetylglucosaminidases (GH89), and α-N-acetylgalactosaminidases (GH101, GH129). Neuramidase/sialidase inhibitors include but are not limited to: siastatin B, zanamivir, peramivir, oseltamivir, and laninamivir.

### C. Compositions Comprising Mediators of Organic Acid Metabolite Levels

Embodiments of the present disclosure concern one or more compositions comprising one or more mediators of organic acid metabolite levels produced following metabolism of mucin-derived carbohydrates by one or more genera of mucus-degrading bacteria in the gut microbiome for the treatment of neutropenic fever and/or GVHD, including cancer therapy-induced neutropenic fever and/or HCT-related GVHD and/or neutropenic fever therapy-induced GVHD. The one or more compositions comprising one or more mediators of organic acid metabolite levels can comprise, for example, vitamins, such as vitamin B12, pre- or probiotics and/or organic acid metabolites.

### 1. Pre- and Probiotics

Embodiments of the present disclosure concern one or more compositions comprising one or more mediators of organic acid metabolite levels, said mediators comprising pre- and/or probiotic microbial compositions for the treatment of neutropenic fever and/or GVHD, including cancer therapy-induced neutropenic fever and/or HCT-related GVHD and/or neutropenic fever therapy-induced GVHD. The pre- and/or probiotics can regulate or mediate the levels of organic acid metabolites produced by metabolism of mucin-derived carbohydrates by one or more genera of mucus-degrading bacteria in the gut microbiome.

As used herein, “probiotic” refers to a composition containing at least one live probiotic bacterial strain. “Probiotics” are live bacteria or yeast that, when consumed, confer a health benefit to the host. Probiotics are said to restore the balance of bacteria in the gut after disruption due to long-term antibiotic use or gastrointestinal disease. Examples of probiotics include but are not limited to Bifidobacterium spp., Lactobacillus spp., Streptococcus thermophila, Bacillus coagulans, Bacillus laterosporus, Pediococcus acidilactici, and/or Saccharomyces boulardii. Probiotics include beneficial bacteria that perform fermentation of food or prebiotics to produce organic acids, for example, thereby regulating or mediating the levels of organic acid metabolites in the gut.

As used herein, “prebiotic” refers to a selectively fermented ingredient that induces specific changes to the composition and/or activity of gastrointestinal microflora to confer benefits upon host well-being and health. Examples of prebiotics include but are not limited to inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-galactooligosaccharides, glucooligosaccharides, isomaltooligosaccharides, lactosucrose, polydextrose, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and/or xylitol. Prebiotics are foods that are able to avoid being digested and absorbed in the upper GI tract and make it to the colonic lumen in sufficient quantities to be fermented by intestinal bacteria to produce organic acids, for example, thereby regulating or mediating the levels of organic acid metabolites in the gut.

The compositions of the one or more probiotic compositions may or may not be tailored to address any deficiency in a subject's gut microbiome or to enhance a subject's gut microbiome. In some cases, the probiotic is considered to be off-the-shelf and comprises a standard one or more microbes to enhance therapy of any kind, including therapy to treat or prevent neutropenic fever and/or GVHD, such as cancer therapy-induced neutropenic fever and/or HCT-related GVHD and/or neutropenic fever therapy-induced GVHD. Such a probiotic may be given to a subject without having prior analysis of their gut microbiome. The probiotic may comprise any one or more microbes associated with efficacious therapy to treat or prevent neutropenic fever and/or GVHD (e.g., HCT-related GVHD and/or neutropenic fever therapy-induced GVHD). In other cases, the probiotic composition is tailored to the specific deficiencies of the gut microbiome of the subject. In some cases, such a customized probiotic may or may not comprise all of the microbes that are considered to be deficient in the subject.

The subject may be given one or more probiotic compositions, including compositions that comprise one or more microbes that overcome any deficiencies in the subject's gut microbiome. The probiotic may be given to treat or prevent neutropenic fever and/or GVHD (e.g., HCT-related GVHD and/or neutropenic fever therapy-induced GVHD) and/or enhance therapy to treat or prevent neutropenic fever and/or GVHD (e.g., HCT-related GVHD and/or neutropenic fever therapy-induced GVHD).

In one embodiment, the probiotic composition is administered conjointly with a prebiotic that stimulates growth and/or activity of bacteria contained in the probiotic composition. Non-limiting examples of useful prebiotics include, e.g., fructooligosaccharides (FOS), galactooligosaccharides (GOS), human milk oligosaccharides (HMO), Lacto-N-neotetraose, D-Tagatose, xylo-oligosaccharides (XOS), arabinoxylan-oligosaccharides (AXOS), N-acetylglucosamine, N-acetylgalactosamine, glucose, arabinose, maltose, lactose, sucrose, cellobiose, amino acids, alcohols, resistant starch (RS), arabinan, pectin, fibers, sugar alcohols including sorbitol, poorly absorbed sugars including lactulose, fucose, and any mixtures thereof. In one specific embodiment, the probiotic and prebiotic are administered in one composition, or simultaneously as two separate compositions, or sequentially.

The composition comprising the desired microbe(s) can be administered alone or in combination with one or more additional probiotic, prebiotic, neutraceutical, or therapeutic agents. Administration “in combination with” one or more further additional probiotic, neutraceutical, or therapeutic agents includes both simultaneous (at the same time) and consecutive administration in any order. Administration can be chronic or intermittent, as deemed appropriate by the supervising practitioner, including in view of any change in any undesirable side effects.

The present disclosure provides pharmaceutical compositions comprising one or more probiotics. The bacterial species therefore are present in the dose form as live bacteria, whether in dried or lyophilized form. In specific embodiments, the probiotic comprises live microorganisms, which, when administered in adequate amounts, may treat or prevent neutropenic fever and/or enhance therapy to treat or prevent neutropenic fever. The probiotic compositions of the disclosure can comprise, without limitation, e.g., live bacterial cells, conditionally lethal bacterial cells, inactivated bacterial cells, killed bacterial cells, spores (e.g., germination-competent spores), recombinant carrier strains, cell extract, and bacterially-derived products (natural or synthetic bacterially-derived products such as, e.g., bacterial antigens or bacterial metabolic products). One or several different bacterial inoculants can be administered simultaneously or sequentially (including administering at different times). Such bacteria can be isolated from gastrointestinal (GI) microbiota and grown in culture. The present disclosure also comprises administering “bacterial analogues”, such as recombinant carrier strains expressing one or more heterologous genes derived from the relevant bacterial species. The use of such recombinant bacteria may allow the use of lower therapeutic amounts due to higher protein expression. In one embodiment of any of the methods involving administration of a probiotic composition, the probiotic composition is reconstituted from a lyophilized preparation. In one embodiment of any of the methods involving administration of a probiotic composition, said probiotic composition comprises a buffering agent to adjust pH to a suitable number, such as 7.0.

In one specific embodiment, the probiotic composition comprises an excipient or a carrier that optimizes the seeding of one or more bacterial strains contained in the probiotic composition.

In one embodiment of any of the above methods involving administration of a probiotic composition, the probiotic composition is directly or indirectly delivered to the digestive tract of the subject. In one embodiment, the probiotic composition is administered to the subject by a route selected from the group consisting of oral, rectal (e.g., by enema), and via naso/oro-gastric gavage. In one embodiment, the probiotic composition is delivered to the subject in a form of a liquid, foam, cream, spray, powder, or gel. In one embodiment, the probiotic composition comprises a buffering agent (e.g., sodium bicarbonate, infant formula or sterilized human milk, or other agents which allow bacteria to survive and grow (e.g., survive in the acidic environment of the stomach and to grow in the intestinal environment), along with preservatives, stabilizers, binders, compaction agents, lubricants, dispersion enhancers, disintegration agents, antioxidants, flavoring agents, sweeteners, and coloring agents.

The composition can be formulated as a frozen composition, e.g., flash frozen, dried or lyophilized for storage and/or transport. In addition, the composition can administered alone or in combination with a carrier, such as a pharmaceutically acceptable carrier or a biocompatible scaffold. Compositions of the disclosure may be conventionally administered rectally as a suppository, parenterally, by injection, for example, intravenously, subcutaneously, or intramuscularly.

Additional formulations that are suitable for other modes of administration include oral formulations. Oral formulations include such normally employed excipients such as, for example, pharmaceutical grades of mannitol, lactose, starch, magnesium stearate, sodium saccharine, cellulose, magnesium carbonate and the like. These compositions take the form of solutions, suppositories, suspensions, tablets, pills, capsules, sustained release formulations or powders and contain about 10% to about 95% of active ingredient, preferably about 25% to about 70%.

In some embodiments, the composition is formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like.

While the compositions of the present disclosure may be formulated for oral administration, other routes of administration can be employed, however, including, but not limited to, subcutaneous, intramuscular, intradermal, transdermal, intraocular, intraperitoneal, mucosal, vaginal, rectal, and intravenous.

In one aspect, the disclosed composition is prepared as a capsule. The capsule may be a hollow, generally cylindrical capsule formed from various substances, such as gelatin, cellulose, carbohydrate or the like.

In another aspect, the disclosed composition is prepared as a suppository. The suppository may include but is not limited to the bacteria and one or more carriers, such as polyethylene glycol, acacia, acetylated monoglycerides, carnuba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

In some aspects, the disclosed microbial composition is prepared as a tablet. The tablet may include the bacteria and one or more tableting agents, such as dibasic calcium phosphate, stearic acid, croscarmellose, silica, cellulose and cellulose coating. The tablets may be formed using a direct compression process, though those skilled in the art will appreciate that various techniques may be used to form the tablets.

In other aspects, the probiotic composition is formed as food, drink, or a dietary supplement (for example, but not limited to capsules, tablets, and powders, in some cases with an enteric coating, for oral treatment), or, alternatively, as an additive to food, drink, or a dietary supplement, wherein an appropriate quantity of bacteria is added to the food or drink to render the food or drink the carrier. Non-limiting examples of foods containing probiotics include dairy products such as yogurt, fermented and unfermented milk, smoothies, butter, cream, hummus, kombucha, salad dressing, miso, tempeh, nutrition bars, and some juices and soy beverages.

In some embodiments, the probiotic composition may further comprise a food or a nutritional supplement effective to stimulate the growth of bacteria present in the gastrointestinal tract of the subject. In some embodiments, the nutritional supplement is produced by another bacterium associated with a healthy human gut microbiome.

In some embodiments, a probiotic composition comprises a minimum amount of one or more isolated bacteria. In some embodiments, the one or more isolated bacteria include but are not limited to Parabacteroides distasonis and other bacteria in the class Bacteroidia, Veillonella and other bacteria in the class Negativicutes, and Lactobacillus rhamnosus and other bacteria in the class Bacilli. For example, a microbial composition may comprise at least, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9% of a particular type or particular types of bacteria, or any value or range derivable therein. Thus, in some embodiments, a probiotic composition comprises at least, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9%, or any value or range derivable therein, of Parabacteroides distasonis or other bacteria in the class Bacteroidia. In some embodiments, a probiotic composition comprises at least, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%. 93%, 94%, 95%, 96%, 97%, 98%, 99%, 99.5%, or 99.9%, or any value or range derivable therein, of Veillonella or other bacteria in the class Negativicutes. In some embodiments, a probiotic composition comprises at least, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9%, or any value or range derivable therein, of Lactobacillus rhamnosus or other bacteria in the class Bacilli. In some embodiments, a probiotic composition comprises at least, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9%, or any value or range derivable therein, of Parabacteroides distasonis or other bacteria in the class Bacteroidia, Veillonella or other bacteria in the class Negativicutes, Lactobacillus rhamnosus or other bacteria in the class Bacilli, or a combination thereof.

In cases wherein more than one microbe is in the probiotic, the ratio of the more than one microbe may or may not be substantially the same. For example, in some cases, two particular microbes in the composition may be at a ratio of 1:1, 1:2, 1:5, 1:10, 1:20, 1:50, 1:100, and so forth. In one embodiment, the probiotic composition comprises bacteria from at least two different bacterial species disclosed herein. Within a given composition, different bacterial strains can be contained in equal amounts (even combination) or in various proportions (uneven combinations) needed for achieving the maximal biological activity. For example, in a bacterial composition with two bacterial strains, the strains may be present in from a 1:10,000 ratio to a 1:1 ratio, from a 1:10,000 ratio to a 1:1,000 ratio, from a 1:1,000 ratio to a 1:100 ratio, from a 1:100 ratio to a 1:50 ratio, from a 1:50 ratio to a 1:20 ratio, from a 1:20 ratio to a 1:10 ratio, from a 1:10 ratio to a 1:1 ratio. For bacterial compositions comprising at least three bacterial strains, the ratio of strains may be chosen pairwise from ratios for bacterial compositions with two strains. For example, in a bacterial composition comprising bacterial strains A, B, and C, at least one of the ratios between strain A and B, the ratio between strain B and C, and the ratio between strain A and C may be chosen, independently, from the pairwise combinations above. In one specific embodiment, the disclosure encompasses administering two or more bacteria-containing compositions to the same subject. Such compositions can be administered simultaneously or sequentially.

In some embodiments, a probiotic composition does not comprise a detectable amount of one or more additional bacteria. In some embodiments, a microbial composition does not comprise more than a contaminating amount of one or more additional bacteria. A contaminating amount of bacteria in a composition may be at most 5%, 4%, 3%, 2%, 1%, 0.1%, 0.01%, 0.001%, or 0.0001% of the composition, or any range or value derivable therein.

The desired dose of the composition of the present disclosure may be presented in multiple (e.g., two, three, four, five, six, or more) sub-doses administered at appropriate intervals throughout the day, week, month or year.

### 2. Organic Acid Metabolites

Embodiments of the present disclosure concern one or more compositions comprising one or more mediators of organic acid metabolites, said mediators comprising organic acid metabolites produced following metabolism of mucin-derived carbohydrates by one or more genera of mucus-degrading bacteria in the gut microbiome for the treatment of neutropenic fever and/or GVHD, including cancer therapy-induced neutropenic fever and/or HCT-related GVHD and/or neutropenic fever therapy-induced GVHD. The one or more compositions comprising one or more organic acid metabolites can comprise, for example, propionate, acetate, butyrate, isovalerate, and/or valerate.

The compositions of the compositions comprising one or more organic acid metabolites may or may not be tailored to address any deficiency in a subject's gut microbiome or to enhance a subject's gut microbiome. The compositions may be given to a subject without having prior analysis of their gut microbiome. The compositions comprising one or more organic acid metabolites may comprise any one or more organic acid metabolites associated with efficacious therapy to treat or prevent neutropenic fever and/or GVHD (e.g., HCT-related GVHD and/or neutropenic fever therapy-induced GVHD).

The subject may be given one or more compositions comprising one or more organic acid metabolites, including compositions that comprise one or more organic acid metabolites that overcome any deficiencies in the subject's gut microbiome. The organic acid metabolites may be given to treat or prevent neutropenic fever and/or GVHD (e.g., HCT-related GVHD and/or neutropenic fever therapy-induced GVHD) and/or enhance therapy to treat or prevent neutropenic fever and/or GVHD (e.g., HCT-related GVHD and/or neutropenic fever therapy-induced GVHD).

The compositions comprising organic acid metabolites can be administered alone or in combination with one or more additional therapeutic agents disclosed herein. Administration “in combination with” one or more additional therapeutic agents includes both simultaneous (at the same time) and consecutive administration in any order. The compositions comprising organic acid metabolites and one or more additional therapeutic agents can be administered in one composition, or simultaneously as two separate compositions, or sequentially. Administration can be chronic or intermittent, as deemed appropriate by the supervising practitioner, including in view of any change in any undesirable side effects.

Metabolites of the gut microbiome can include but are not limited to organic acids, phytochemicals, and phenolic compounds. Gut microbial metabolites mostly stem from dietary components fermented by bacteria, such as SCFAs, unsaturated and saturated medium- and long-chain fatty acids (LCFAs), tryptophan metabolites, bile acids, amino acid derivatives including but not limited to indoles, monosaccharides and amino acids, and vitamins including but not limited to nicotinic acid and cobalamins. As used herein, “short chain fatty acids” (SCFAs) refer to a group of fatty acids with less than six carbons that are produced by anaerobes of the human large intestine. Certain types of gut bacteria ferment indigestible polysaccharides, resulting in the production of three major SCFAs: acetate, propionate, and butyrate. SCFAs are a major source of energy not only for enterocytes but also for the entire body. Microorganisms also modify endogenous metabolites, such as bile acids, intermediates of the citric acid cycle and cholesterol metabolites, and bacteria can de novo synthesize metabolites, such as adenosine triphosphate.

In some embodiments, organic acid metabolites include but are not limited to short chain fatty acids, including propionate, butyrate, acetate, isovalerate, and/or valerate. In some embodiments, the presence of organic acid metabolites in the gut microbiome serves as a feedback mechanism to suppress excessive utilization of mucin glycans, which would otherwise be metabolized by one or more genera of mucus-degrading bacteria in the gut microbiome. In some embodiments, the organic acid metabolites (e.g., SCFAs) are depleted upon reduced oral intake of nutrients.

In some embodiments, the organic acid metabolites are produced endogenously by metabolism of mucin-derived carbohydrates by one or more genera of mucus-degrading bacteria in the gut microbiome.

In some embodiments, the organic acid metabolites are produced exogenously and are directly or indirectly delivered to the digestive tract of the subject. In one embodiment, the organic acid metabolite composition is administered to the subject by a route selected from the group consisting of oral, rectal (e.g., by enema), and via naso/oro-gastric gavage. In one embodiment, the organic acid metabolite composition is delivered to the subject in a form of a liquid, foam, cream, spray, powder, or gel. In one embodiment, the organic acid metabolite composition comprises a buffering agent (e.g., sodium bicarbonate), along with preservatives, stabilizers, binders, compaction agents, lubricants, dispersion enhancers, disintegration agents, antioxidants, flavoring agents, sweeteners, and coloring agents.

In some embodiments, the one or more mediators of organic acid metabolite levels in the gut comprise one or more vitamins. In some embodiments, the one or more vitamins comprise vitamin B12. In some cases, the vitamin B12 promotes fermentation of substrate such as mucin-derived carbohydrates by endogenous bacteria in the gut to produce organic acid metabolites, such as propionate, thereby augmenting, or increasing, organic acid metabolite levels in the gut. In some cases, the vitamin B12 is co-administered with bacteria that ferment substrate such as mucin-derived carbohydrates in the gut to produce organic acid metabolites, such as propionate, thereby augmenting, or increasing, organic acid metabolite levels in the gut.

The organic acid metabolite composition can be formulated as a frozen composition, e.g., flash frozen, dried or lyophilized for storage and/or transport. In addition, the organic acid metabolite composition can administered alone or in combination with a carrier, such as a pharmaceutically acceptable carrier or a biocompatible scaffold. Compositions of the disclosure may be conventionally administered rectally as a suppository, parenterally, by injection, for example, intravenously, subcutaneously, or intramuscularly.

Additional formulations that are suitable for other modes of administration include oral formulations. Oral formulations include such normally employed excipients such as, for example, pharmaceutical grades of mannitol, lactose, starch, magnesium stearate, sodium saccharine, cellulose, magnesium carbonate and the like. These compositions take the form of solutions, suppositories, suspensions, tablets, pills, capsules, sustained release formulations or powders and contain about 10% to about 95% of active ingredient, preferably about 25% to about 70%.

In some embodiments, the organic acid metabolite composition is formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like.

While the compositions of the present disclosure may be formulated for oral administration, other routes of administration can be employed, however, including, but not limited to, subcutaneous, intramuscular, intradermal, transdermal, intraocular, intraperitoneal, mucosal, vaginal, rectal, and intravenous.

In one aspect, the disclosed organic acid metabolite composition is prepared as a capsule. The capsule may be a hollow, generally cylindrical capsule formed from various substances, such as gelatin, cellulose, carbohydrate or the like.

In another aspect, the disclosed organic acid metabolite composition is prepared as a suppository. The suppository may include but is not limited to the organic acid metabolite and one or more carriers, such as polyethylene glycol, acacia, acetylated monoglycerides, carnuba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

In some aspects, the disclosed organic acid metabolite composition is prepared as a tablet. The tablet may include the organic acid metabolite and one or more tableting agents, such as dibasic calcium phosphate, stearic acid, croscarmellose, silica, cellulose and cellulose coating. The tablets may be formed using a direct compression process, though those skilled in the art will appreciate that various techniques may be used to form the tablets.

In other aspects, the organic acid metabolite composition is formed as food, drink, or a dietary supplement (for example, but not limited to capsules, tablets, and powders, in some cases with an enteric coating, for oral treatment), or, alternatively, as an additive to food, drink, or a dietary supplement, wherein an appropriate quantity of organic acid metabolite is added to the food or drink to render the food or drink the carrier.

In some embodiments, the organic acid metabolite composition may further comprise a food or a nutritional supplement effective to stimulate the growth of bacteria present in the gastrointestinal tract of the subject, for example, a pre- and/or probiotic, and/or effective to stimulate organic acid production by bacteria in the gastrointestinal tract of the subject, for example, vitamins including but not limited to vitamin B12. In some embodiments, the nutritional supplement is produced by another bacterium associated with a healthy human gut microbiome.

### D. Carbohydrate Substrate Compositions

Embodiments of the present disclosure concern compositions comprising one or more carbohydrate substrates metabolized one or more genera of mucus-degrading bacteria in the gut microbiome for the treatment of neutropenic fever and/or GVHD, including cancer therapy-induced neutropenic fever and/or GVHD.

In some cases, metabolism of the carbohydrate substrates by one or more genera of mucus-degrading bacteria in the gut microbiome occurs preferentially over metabolism of the mucus lining the epithelium in the colon by one or more genera of mucus-degrading bacteria in the gut microbiome. In some cases, metabolism of the carbohydrate substrates is achieved by one or more saccharolytic enzymes from bacteria, as described elsewhere herein.

In some cases, the carbohydrate substrates or compositions thereof are administered to a subject to supplement or replace one or more carbohydrate substrates that become deficient or reduced in the gut of a subject to whom one or more broad-spectrum antibiotics are administered, for example, to treat neutropenic fever or infection following cancer therapy. In some cases, the one or more carbohydrate substrates become deficient or reduced in the gut of a subject following broad-spectrum antibiotic administration because, for example, the broad-spectrum antibiotics clear commensal bacteria. In some cases, the commensal bacteria metabolize dietary carbohydrates into carbohydrate substrates subsequently utilized by mucus-degrading bacteria in the gut, and upon clearance of the commensal bacteria from the gut, the carbohydrate substrates utilized by mucus-degrading bacteria in the gut are no longer produced, causing levels of carbohydrate substrates to therefore become deficient or reduced.

Thus, in some cases, the compositions of the one or more carbohydrate substrate compositions may or may not be tailored to address any deficiency in a subject's gut microbiome or to enhance a subject's gut microbiome. The compositions may be given to a subject without having prior analysis of their gut microbiome. The carbohydrate substrate compositions may comprise any one or more carbohydrate substrate associated with efficacious therapy to treat or prevent neutropenic fever and/or GVHD.

The subject may be given one or more carbohydrate substrate compositions, including compositions that comprise one or more carbohydrate substrate that overcome any deficiencies in the subject's gut microbiome. The carbohydrate substrate compositions may be given to treat or prevent neutropenic fever and/or GVHD and/or enhance therapy to treat or prevent neutropenic fever and/or GVHD.

The carbohydrate substrate composition can be administered alone or in combination with one or more additional therapeutic agents disclosed herein. Administration “in combination with” one or more additional therapeutic agents includes both simultaneous (at the same time) and consecutive administration in any order. The carbohydrate substrate composition and one or more additional therapeutic agents can be administered in one composition, or simultaneously as two separate compositions, or sequentially. Administration can be chronic or intermittent, as deemed appropriate by the supervising practitioner, including in view of any change in any undesirable side effects.

In some embodiments, the one or more carbohydrate substrates comprise, for example, mono- and/or polysaccharides. The carbohydrate substrates may be soluble, for example, soluble in the colonic lumen. In some embodiments, the carbohydrate substrate(s) comprise arabinose, fructose, fucose, galactose, galacturonic acid, glucuronic acid, glucosamine, glucose, mannose, N-acetylglucosamine, N-acetylgalactosamine, rhamnose, ribose, xylose, pullulan, glycogen, amylopectin, inulin, levan, heparin, hyaluronan, chondroitin sulfate, polygalacturonate, rhamnogalacturonan, pectic galactan, arabinogalactan, arabinan, xylan, arabinoxylan, galactomannan, glucomannan, xyloglucan, β-glucan, cellobiose, laminarin, lichenin, dextran, and/or α-mannan. In some embodiments, the carbohydrate substrate(s) are mannose, glucose, and/or xylose. In some embodiments, the carbohydrate substrate is glucose.

### VI. Methods of Treatment and Use of the Disclosure

The present disclosure encompasses methods and compositions related to the gut microbiome of a subject that has cancer, or that is suspected of having cancer, and is in need of intervention with a cancer therapy. The cancer may or may not be relapsed or refractory. The cancer may be solid tumors or hematological malignancies. The cancer may be of any stage or type or tissue of origin. The cancer may or may not be metastatic. The cancer may or may not be resistant to one or more types of therapies. In some cases, the subject is in need of a transplant therapy. In some cases, the subject has a leukemia, myeloma, or lymphoma and is in need of a hematopoietic stem cell transplant therapy.

In particular embodiments, the gut microbiome of a subject is analyzed or measured or determined or evaluated for the overall diversity of its microbes, irrespective of which microbes are actually present and/or absent. The overall diversity and/or activity of the gut microbiome may be measured in any suitable manner.

In particular cases, a subject having a gut microbiome having an increased abundance of one or more genera of mucus-degrading bacteria has an increased chance of developing neutropenic fever and/or GVHD (e.g., HCT-related GVHD and/or neutropenic fever therapy-induced GVHD). In particular cases, a subject having a gut microbiome having an increased abundance of one or more genera of mucus-degrading bacteria has a decreased chance of developing neutropenic fever and/or GVHD (e.g., HCT-related GVHD and/or neutropenic fever therapy-induced GVHD). In particular cases, a subject having a decreased abundance of one or more genera of mucus-degrading bacteria has an increased chance of developing neutropenic fever and/or GVHD (e.g., HCT-related GVHD and/or neutropenic fever therapy-induced GVHD). In particular cases, a subject having a decreased abundance of one or more genera of mucus-degrading bacteria has a decreased chance of developing neutropenic fever and/or GVHD (e.g., HCT-related GVHD and/or neutropenic fever therapy-induced GVHD).

In particular cases, a subject having a gut microbiome having an increased expression or activity of one or more mucus-degrading enzymes has an increased chance of developing neutropenic fever and/or GVHD (e.g., HCT-related GVHD and/or neutropenic fever therapy-induced GVHD). In particular cases, a subject having a gut microbiome having an increased expression or activity of one or more mucus-degrading enzymes has a decreased chance of developing neutropenic fever and/or GVHD (e.g., HCT-related GVHD and/or neutropenic fever therapy-induced GVHD). In particular cases, a subject having a decreased expression or activity of one or more mucus-degrading enzymes has an increased chance of developing neutropenic fever and/or GVHD (e.g., HCT-related GVHD and/or neutropenic fever therapy-induced GVHD). In particular cases, a subject having a decreased expression or activity of one or more mucus-degrading enzymes has a decreased chance of developing neutropenic fever and/or GVHD (e.g., HCT-related GVHD and/or neutropenic fever therapy-induced GVHD).

In particular cases, a subject having a gut microbiome having decreased levels of one or more organic acid metabolites produced by metabolism of mucin-derived carbohydrates by one or more genera of mucus-degrading bacteria in the gut microbiome has an increased chance of developing neutropenic fever and/or GVHD (e.g., HCT-related GVHD and/or neutropenic fever therapy-induced GVHD). In particular cases, a subject having a gut microbiome having decreased levels of one or more organic acid metabolites produced by metabolism of mucin-derived carbohydrates by one or more genera of mucus-degrading bacteria in the gut microbiome has a decreased chance of developing neutropenic fever and/or GVHD (e.g., HCT-related GVHD and/or neutropenic fever therapy-induced GVHD). In particular cases, a subject having increased levels of one or more organic acid metabolites produced by metabolism of mucin-derived carbohydrates by one or more genera of mucus-degrading bacteria in the gut microbiome has an increased chance of developing neutropenic fever and/or GVHD (e.g., HCT-related GVHD and/or neutropenic fever therapy-induced GVHD). In particular cases, a subject having increased levels of one or more organic acid metabolites produced by metabolism of mucin-derived carbohydrates by one or more genera of mucus-degrading bacteria in the gut microbiome has a decreased chance of developing neutropenic fever and/or GVHD (e.g., HCT-related GVHD and/or neutropenic fever therapy-induced GVHD).

In particular cases, a subject having a gut microbiome having increased levels of one or more carbohydrate substrates metabolized by one or more genera of mucus-degrading bacteria in the gut microbiome has an increased chance of developing neutropenic fever and/or GVHD (e.g., HCT-related GVHD and/or neutropenic fever therapy-induced GVHD). In particular cases, a subject having a gut microbiome having increased levels of one or more carbohydrate substrates metabolized by one or more genera of mucus-degrading bacteria in the gut microbiome has a decreased chance of developing neutropenic fever and/or GVHD (e.g., HCT-related GVHD and/or neutropenic fever therapy-induced GVHD). In particular cases, a subject having increased levels of one or more carbohydrate substrates metabolized by one or more genera of mucus-degrading bacteria in the gut microbiome has an increased chance of developing neutropenic fever and/or GVHD (e.g., HCT-related GVHD and/or neutropenic fever therapy-induced GVHD). In particular cases, a subject having increased levels of one or more carbohydrate substrates metabolized by one or more genera of mucus-degrading bacteria in the gut microbiome has a decreased chance of developing neutropenic fever and/or GVHD (e.g., HCT-related GVHD and/or neutropenic fever therapy-induced GVHD).

### A. Methods of Treating or Preventing Neutropenic Fever

Methods of the disclosure allow for the treatment or prevention of neutropenic fever, including cancer therapy-induced neutropenic fever, by administering a therapeutically effective amount of a composition comprising one or more agents targeting growth or expansion of one or more genera of mucus-degrading bacteria in the gut microbiome of the subject; one or more mucus-degrading enzyme inhibitors to inhibit mucus degradation by one or more genera of mucus-degrading bacteria in the gut microbiome of the subject; one or more mediators of organic acid metabolite levels produced following metabolism of mucin-derived carbohydrates by one or more genera of mucus-degrading bacteria in the gut microbiome of the subject; and/or one or more carbohydrate substrates metabolized by one or more genera of mucus-degrading bacteria in the gut microbiome of the subject.

Methods of the disclosure include methods of treating or preventing neutropenic fever, including cancer therapy-induced neutropenic fever, where risk of developing neutropenic fever, including cancer therapy-induced neutropenic fever, is increased. In some embodiments, the subject is at a higher risk than an average person in the general population receiving the cancer therapy of developing cancer therapy-induced neutropenic fever. In some embodiments, the cancer therapy-induced neutropenic fever poses a greater risk to the health or life of the subject than such a condition would pose to an average person in the general population receiving the cancer therapy. In some cases, the method is employed for a subject where it is uncertain whether or not risk of developing neutropenic fever, including cancer therapy-induced neutropenic fever, is increased, whereas in other cases the method is employed for a subject where it is known that the risk of developing neutropenic fever, including cancer therapy-induced neutropenic fever, is increased. In other cases, it has been determined that the risk of developing neutropenic fever, including cancer therapy-induced neutropenic fever, is increased for the subject, but the methods of the disclosure are still employed as a routine matter or in the general therapeutic interest of the subject.

The disclosure encompasses methods and compositions for modulating the gut microbiome activity and/or composition of a subject to treat or prevent neutropenic fever, including cancer therapy-induced neutropenic fever. The modulation may or may not be as a result of analysis of the gut microbiome activity and/or composition prior to or after diagnosing the subject with neutropenia. In some cases, the modulation is a result of analysis of the gut microbiome prior to diagnosing the subject with neutropenia, and the outcome of the analysis determines the nature of the resultant modulation of the gut microbiome. For example, the modulation may comprise providing a therapeutically effective amount of one or more agents targeting growth or expansion of one or more genera of mucus-degrading bacteria in the gut microbiome; one or more mucus-degrading enzyme inhibitors to inhibit mucus degradation by one or more genera of mucus-degrading bacteria in the gut microbiome; one or more mediators of organic acid metabolite levels produced following metabolism of mucin-derived carbohydrates by one or more genera of mucus-degrading bacteria in the gut microbiome; and/or one or more carbohydrate substrates metabolized by one or more genera of mucus-degrading bacteria in the gut microbiome of the subject.

In some cases, the subject receiving a cancer therapy and having or at risk of having neutropenia and/or neutropenic fever was determined to have an increased abundance of mucus-degrading bacteria in the gut microbiome compared to a control or reference sample, and the modulation may comprise providing an effective amount of one or more bacterial growth-suppressing agents that would reduce levels of one or more microbes that were determined to be excessive in the gut microbiome of a subject. In some cases, the subject was determined to have an increase in functional activity and/or expression levels of one or more mucus-degrading enzymes secreted by one or more genera of mucus-degrading bacteria in the gut microbiome compared to a control or reference sample, and the modulation may comprise providing an effective amount of one or more one or more mucus-degrading enzyme inhibitors that would inhibit mucus degradation by enzymes produced by one or more genera of mucus-degrading bacteria in the gut microbiome that were determined to be excessive in the gut microbiome of a subject. In some cases, the subject was determined to have decreased levels of one or more organic acid metabolites produced following metabolism of mucin-derived carbohydrates by one or more genera of mucus-degrading bacteria in the gut microbiome compared to a control or reference sample, and the modulation may comprise providing an effective amount of one or more mediators of organic acid metabolite levels produced following metabolism of mucin-derived carbohydrates by one or more genera of mucus-degrading bacteria in the gut microbiome that serve as a feedback mechanism to suppress excessive utilization of mucin glycans, which would otherwise be metabolized by one or more genera of mucus-degrading bacteria in the gut microbiome. In some cases, the subject was determined to have decreased levels of one or more carbohydrate substrates metabolized by one or more genera of mucus-degrading bacteria in the gut microbiome compared to a control or reference sample, and the modulation may comprise providing an effective amount of one or more carbohydrate substrates for metabolism by one or more genera of mucus-degrading bacteria in the gut microbiome. In some cases, the subject receiving a cancer therapy and/or a neutropenic fever therapy and having or at risk of having neutropenia and/or neutropenic fever was determined to have a decreased abundance of one or more classes, orders, families, genera, or species of commensal bacteria in the gut microbiome compared to a control or reference sample; one or more commensal bacteria may comprise Clostridia bacteria, and the modulation may comprise any method of modulation disclosed herein.

Though these methods of modulating the gut microbiome of a subject receiving a cancer therapy and having or at risk of having neutropenia and/or neutropenic fever are described with reference to a particular dysbiosis (e.g., increased abundance of one or more genera of mucus-degrading bacteria, decreased abundance of one or more commensal bacteria, increased functional activity and/or expression levels of one or more mucus-degrading enzymes, decreased levels of one or more organic acid metabolites, and/or decreased levels of one or more carbohydrate substrates), one of skill in the art would understand that any method of modulation disclosed herein (e.g., administration of one or more bacterial growth-suppressing agents, one or more one or more mucus-degrading enzyme inhibitors, one or more mediators of organic acid metabolite levels, and/or one or more carbohydrate substrates) may be applied to treat, prevent, or reduce the severity of any dysbiosis (e.g., increased abundance of one or more genera of mucus-degrading bacteria, decreased abundance of one or more commensal bacteria, increased functional activity and/or expression levels of one or more mucus-degrading enzymes, have decreased levels of one or more organic acid metabolites, and/or decreased levels of one or more carbohydrate substrates) disclosed herein.

In some cases, the control or reference sample is a sample from a healthy subject. In some cases the control or reference sample is a sample from a subject who is diagnosed with neutropenia but who does not become febrile or develop neutropenic fever. In some cases the control or reference sample is a sample from a subject who is diagnosed with neutropenia after administration of the cancer therapy but who does not become febrile or develop neutropenic fever. In some cases the control or reference sample is a sample from a subject who is diagnosed with neutropenia who becomes febrile or develops neutropenic fever. In some cases the control or reference sample is a sample from a subject who is diagnosed with neutropenia after administration of the cancer therapy who becomes febrile or develops neutropenic fever. In some cases, the control or reference sample is used to identify normal and/or abnormal ranges for the abundance of one or more genera of mucus-degrading and/or commensal bacteria in the gut microbiome; the functional activity and/or expression levels of one or more mucus-degrading enzymes secreted by one or more genera of mucus-degrading bacteria in the gut microbiome; levels of one or more organic acid metabolites produced following metabolism of mucin-derived carbohydrates by one or more genera of mucus-degrading bacteria in the gut microbiome; and/or levels of one or more carbohydrate substrates metabolized by one or more genera of mucus-degrading bacteria in the gut microbiome.

In some cases, the subject does not exhibit symptoms of cancer therapy-induced neutropenic fever when the composition comprising one or more agents targeting growth or expansion of one or more genera of mucus-degrading bacteria in the gut microbiome of the subject; one or more mucus-degrading enzyme inhibitors to inhibit mucus degradation by one or more genera of mucus-degrading bacteria in the gut microbiome of the subject; one or more mediators of organic acid metabolite levels produced following metabolism of mucin-derived carbohydrates by one or more genera of mucus-degrading bacteria in the gut microbiome of the subject; and/or one or more carbohydrate substrates metabolized by one or more genera of mucus-degrading bacteria in the gut microbiome of the subject is administered. In some embodiments, the subject has been diagnosed with neutropenia. In some embodiments, the composition is administered after the subject has been diagnosed with neutropenia and/or neutropenic fever, and the composition may be administered to the subject every day until the subject is no longer neutropenic and/or no longer has neutropenic fever. In some embodiments, the composition is administered multiple times per day. In some embodiments, the composition is administered 1, 2, 3, 4, 5, or 6 times per day.

In some cases, the subject is neutropenic and/or develops neutropenic fever due to the chemotherapy treatment received by the subject. The chemotherapy treatment received by the subject can comprise alkylating agents, marrow-suppressive agents, reduced intensity conditioning, myeloablative conditioning, non-myeloablative conditioning, and immunosuppressive drugs.

In some cases, the subject is neutropenic and/or develops neutropenic fever due to a radiotherapy treatment received by the subject. The radiotherapy treatment received by the subject can comprise external radiotherapy, internal radiotherapy, radioimmunotherapy, or intraoperative radiation therapy (IORT).

In some cases, the subject is neutropenic and/or develops neutropenic fever due to an immunotherapy treatment received by the subject. The immunotherapy treatment received by the subject can comprise a checkpoint inhibitor, an inhibitor of a co-stimulatory molecule, dendritic cell therapy, CAR-T cell therapy, cytokine therapy, or adoptive T cell therapy.

In some embodiments, the abundance of mucus-degrading and/or commensal bacteria in the gut microbiome, the functional activity and/or expression levels of one or more mucus-degrading enzymes secreted by one or more genera of mucus-degrading bacteria in the gut microbiome, the levels of one or more organic acid metabolites produced following metabolism of mucin-derived carbohydrates by one or more genera of mucus-degrading bacteria in the gut microbiome, and/or levels of one or more carbohydrate substrates metabolized by one or more genera of mucus-degrading bacteria in the gut microbiome were determined from a fecal sample from the subject. Mucus-degrading and/or commensal bacteria can be quantified by the relative abundance of 16S gene copies of bacterial taxa known to harbor mucolytic genes, the absolute abundance of 16S gene copies of these taxa, as well as whole metagenomic shotgun sequencing of DNA or RNA to identify and quantify mucolytic genes. Enzymatic activity of these bacteria could also be functionally quantified, by quantifying breakdown of mucin, or by activity of specific enzymes that participate in various steps of mucus breakdown.

In some cases, both a deficiency in the gut microbiome and an excess in the gut microbiome are both handled prior to or after diagnosing the subject with neutropenia. In particular embodiments, such actions improve the efficacy of therapeutic strategies to treat or prevent neutropenic fever, including cancer therapy-induced neutropenic fever.

### B. Methods of Predicting Neutropenic Fever

In particular embodiments, the disclosure concerns methods of predicting development of neutropenic fever, including cancer therapy-induced neutropenic fever, in a subject receiving a cancer therapy treatment based on analyzing one or more of the following biomarkers: (1) abundance of one or more genera of mucus-degrading bacteria in the gut microbiome of the subject; (2) abundance of one or more commensal bacteria in the gut microbiome of the subject; (3) the functional activity and/or expression levels of one or more mucus-degrading enzymes secreted by one or more genera of mucus-degrading bacteria in the gut microbiome of the subject; (4) levels of one or more organic acid metabolites produced following metabolism of mucin-derived carbohydrates by one or more genera of mucus-degrading bacteria in the gut microbiome of the subject; (5) levels of one or more carbohydrate substrates metabolized by one or more genera of mucus-degrading bacteria in the gut microbiome of the subject; and/or (6) levels of one or more ruminal metabolites that target the growth or expansion of one or more genera of mucus-degrading bacteria in the gut microbiome of the subject.

In some embodiments, the subject has been diagnosed with neutropenia. In some cases, the subject is neutropenic and/or develops neutropenic fever due to a cancer therapy received by the subject. In some cases, the cancer therapy comprises a chemotherapy treatment received by the subject. The chemotherapy treatment received by the subject can comprise alkylating agents, marrow-suppressive agents, reduced intensity conditioning, myeloablative conditioning, non-myeloablative conditioning, and immunosuppressive drugs. In some cases, the cancer therapy comprises a radiotherapy treatment received by the subject. The radiotherapy treatment received by the subject can comprise external radiotherapy, internal radiotherapy, radioimmunotherapy, or intraoperative radiation therapy (IORT). In some cases, the cancer therapy comprises an immunotherapy treatment received by the subject. The immunotherapy treatment received by the subject can comprise a checkpoint inhibitor, an inhibitor of a co-stimulatory molecule, dendritic cell therapy, CAR-T cell therapy, cytokine therapy, or adoptive T cell therapy.

In some embodiments, the disclosure concerns methods of predicting a therapy outcome for a subject in need of a cancer therapy, including the likelihood of developing neutropenic fever, including cancer therapy-induced neutropenic fever, such as when compared to a standard or a subject with a different microbiome. Such analysis of (1), (2), (3), (4), (5), (6) of the above compared to a control or reference sample results in a determination of whether or how best to treat or prevent the development of neutropenic fever, including cancer therapy-induced neutropenic fever, in the subject receiving chemotherapy. In some cases, the control or reference sample is a sample from a healthy subject. In some cases the control or reference sample is a sample from a subject who is diagnosed with neutropenia but who does not become febrile or develop neutropenic fever. In some cases the control or reference sample is a sample from a subject who is diagnosed with neutropenia after administration of the cancer therapy but who does not become febrile or develop neutropenic fever. In some cases the control or reference sample is a sample from a subject who is diagnosed with neutropenia who becomes febrile or develops neutropenic fever. In some cases the control or reference sample is a sample from a subject who is diagnosed with neutropenia after administration of the cancer therapy who becomes febrile or develops neutropenic fever. In some cases, the control or reference sample is used to identify normal and/or abnormal ranges for the abundance of one or more genera of mucus-degrading and/or commensal bacteria in the gut microbiome; the functional activity and/or expression levels of one or more mucus-degrading enzymes secreted by one or more genera of mucus-degrading bacteria in the gut microbiome; levels of one or more organic acid metabolites produced following metabolism of mucin-derived carbohydrates by one or more genera of mucus-degrading bacteria in the gut microbiome; levels of one or more carbohydrate substrates metabolized by one or more genera of mucus-degrading bacteria in the gut microbiome of the subject; and/or levels of one or more ruminal metabolites that target the growth or expansion of one or more genera of mucus-degrading bacteria in the gut microbiome of the subject.

For example, when the analysis of the gut microbiome indicates that the abundance of one or more genera of mucus-degrading bacteria in the gut microbiome of the subject is increased compared to a control or reference sample, the subject has an increased likelihood of developing cancer therapy-induced neutropenic fever, and the subject may then be provided a therapeutically effective amount of one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, one or more mediators of organic acid metabolite levels produced following metabolism of mucin-derived carbohydrates by one or more genera of mucus-degrading bacteria in the gut microbiome of the subject, and/or one or more carbohydrate substrates metabolized by one or more genera of mucus-degrading bacteria in the gut microbiome of the subject. When the analysis of the gut microbiome indicates that the abundance of one or more genera of mucus-degrading bacteria in the gut microbiome of the subject is similar to or decreased compared to a control or reference sample, the subject is not at risk or has a reduced risk of developing cancer therapy-induced neutropenic fever.

The abundance of the one or more genera of mucus-degrading bacteria in the gut microbiome of the subject is considered to be increased compared to a control or reference sample when the abundance of the one or more genera of mucus-degrading bacteria in the gut microbiome of the subject comprises more than equal to any one of, at least any one of, at most any one of, or between any two of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%. 8.0%. 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, or any range or value derivable therein, of the total gut microbiome bacterial population compared to a control or reference sample.

When the analysis of the gut microbiome indicates that the abundance of one or more commensal bacteria in the gut microbiome of the subject is decreased compared to a control or reference sample, the subject has an increased likelihood of developing cancer therapy-induced neutropenic fever, and the subject may then be provided a therapeutically effective amount of one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, one or more mediators of organic acid metabolite levels produced following metabolism of mucin-derived carbohydrates by one or more genera of mucus-degrading bacteria in the gut microbiome of the subject, and/or one or more carbohydrate substrates metabolized by one or more genera of mucus-degrading bacteria in the gut microbiome of the subject. When the analysis of the gut microbiome indicates that the abundance of one or more commensal bacteria in the gut microbiome of the subject is similar to or increased compared to a control or reference sample, the subject is not at risk or has a reduced risk of developing cancer therapy-induced neutropenic fever.

The abundance of the one or more commensal bacteria in the gut microbiome of the subject is considered to be decreased compared to a control or reference sample when the abundance of the one or more commensal bacteria in the gut microbiome of the subject comprises more than equal to any one of, at least any one of, at most any one of, or between any two of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%. 2.3%. 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, or any range or value derivable therein, of the total gut microbiome bacterial population compared to a control or reference sample.

When the analysis of the gut microbiome indicates that the functional activity or expression levels of one or more mucus-degrading enzymes secreted by one or more genera of mucus-degrading bacteria are increased compared to a control or reference sample or control, the subject has an increased likelihood of developing cancer therapy-induced neutropenic fever, and the subject may be provided an effective amount of one or more mucus-degrading enzyme inhibitors to inhibit mucus degradation by one or more genera of mucus-degrading bacteria in the gut microbiome of the subject, one or more agents targeting growth or expansion of one or more genera of mucus-degrading bacteria in the gut microbiome of the subject, one or more mediators of organic acid metabolite levels produced following metabolism of mucin-derived carbohydrates by one or more genera of mucus-degrading bacteria in the gut microbiome of the subject, and/or one or more carbohydrate substrates metabolized by one or more genera of mucus-degrading bacteria in the gut microbiome of the subject. When the analysis of the gut microbiome indicates that the functional activity or expression levels of one or more mucus-degrading enzymes secreted by one or more genera of mucus-degrading bacteria are similar to or decreased compared to a control or reference sample, the subject is not at risk or has a reduced risk of developing cancer therapy-induced neutropenic fever.

The functional activity or expression levels of one or more mucus-degrading enzymes secreted by one or more genera of mucus-degrading bacteria in the gut microbiome of the subject are considered to be increased compared to a control or reference sample when the functional activity or expression levels of one or more mucus-degrading enzymes secreted by one or more genera of mucus-degrading bacteria in the gut microbiome of the subject are increased to greater than equal to any one of, at least any one of, at most any one of, or between any two of 1-fold, 10-fold, 20-fold, 30-fold, 40-fold, 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 410-fold, 420-fold, 430-fold, 440-fold, 450-fold, 460-fold, 470-fold, 480-fold, 490-fold, 500-fold, 510-fold, 520-fold, 530-fold, 540-fold, 550-fold, 560-fold, 570-fold, 580-fold, 590-fold, 600-fold, 610-fold, 620-fold, 630-fold, 640-fold, 650-fold, 660-fold, 670-fold, 680-fold, 690-fold, 700-fold, 710-fold, 720-fold, 730-fold, 740-fold, 750-fold, 760-fold, 770-fold, 780-fold, 790-fold, 800-fold, 810-fold, 820-fold, 830-fold, 840-fold, 850-fold, 860-fold, 870-fold, 880-fold, 890-fold, 900-fold, 910-fold, 920-fold, 930-fold, 940-fold, 950-fold, 960-fold, 970-fold, 980-fold, 990-fold, 1000-fold, 1100-fold, 1200-fold, 1300-fold, 1400-fold, 1500-fold, 1600-fold, 1700-fold, 1800-fold, 1900-fold, 2000-fold, 2100-fold, 2200-fold, 2300-fold, 2400-fold, 2500-fold, 2600-fold, 2700-fold, 2800-fold, 2900-fold, 3000-fold, 3100-fold, 3200-fold, 3300-fold, 3400-fold, 3500-fold, 3600-fold, 3700-fold, 3800-fold, 3900-fold, 4000-fold, 4100-fold, 4200-fold, 4300-fold, 4400-fold, 4500-fold, 4600-fold, 4700-fold, 4800-fold, 4900-fold, 5000-fold, 5100-fold, 5200-fold, 5300-fold, 5400-fold, 5500-fold, 5600-fold, 5700-fold, 5800-fold, 5900-fold, 6000-fold, 6100-fold, 6200-fold, 6300-fold, 6400-fold, 6500-fold, 6600-fold, 6700-fold, 6800-fold, 6900-fold, 7000-fold, 7100-fold, 7200-fold, 7300-fold, 7400-fold, 7500-fold, 7600-fold, 7700-fold, 7800-fold, 7900-fold, 8000-fold, 8100-fold, 8200-fold, 8300-fold, 8400-fold, 8500-fold, 8600-fold, 8700-fold, 8800-fold, 8900-fold, 9000-fold, 9100-fold, 9200-fold, 9300-fold, 9400-fold, 9500-fold, 9600-fold, 9700-fold, 9800-fold, 9900-fold, 10000-fold, 11000-fold, 12000-fold, 13000-fold, 14000-fold, 15000-fold, 16000-fold, 17000-fold, 18000-fold, 19000-fold, 20000-fold, 21000-fold, 22000-fold, 23000-fold, 24000-fold, 25000-fold, 26000-fold, 27000-fold, 28000-fold, 29000-fold, 30000-fold, 31000-fold, 32000-fold, 33000-fold, 34000-fold, 35000-fold, 36000-fold, 37000-fold, 38000-fold, 39000-fold, 40000-fold, 41000-fold, 42000-fold, 43000-fold, 44000-fold, 45000-fold, 46000-fold, 47000-fold, 48000-fold, 49000-fold, 50000-fold, 51000-fold, 52000-fold, 53000-fold, 54000-fold, 55000-fold, 56000-fold, 57000-fold, 58000-fold, 59000-fold, 60000-fold, 61000-fold, 62000-fold, 63000-fold, 64000-fold, 65000-fold, 66000-fold, 67000-fold, 68000-fold, 69000-fold, 70000-fold, 71000-fold, 72000-fold, 73000-fold, 74000-fold, 75000-fold, 76000-fold, 77000-fold, 78000-fold, 79000-fold, 80000-fold, 81000-fold, 82000-fold, 83000-fold, 84000-fold, 85000-fold, 86000-fold, 87000-fold, 88000-fold, 89000-fold, 90000-fold, 91000-fold, 92000-fold, 93000-fold, 94000-fold, 95000-fold, 96000-fold, 97000-fold, 98000-fold, 99000-fold, 100000-fold, or any range or value derivable therein, compared to a control or reference sample.

When the analysis of the gut microbiome indicates that levels of one or more organic acid metabolites produced following metabolism of mucin-derived carbohydrates by one or more genera of mucus-degrading bacteria are decreased compared to a control or reference sample, the subject has an increased likelihood of developing cancer therapy-induced neutropenic fever, and the subject may be provided an effective amount of one or more mediators of organic acid metabolite levels produced following metabolism of mucin-derived carbohydrates by one or more genera of mucus-degrading bacteria in the gut microbiome of the subject, one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more carbohydrate substrates metabolized by one or more genera of mucus-degrading bacteria in the gut microbiome of the subject. When the analysis of the gut microbiome indicates that levels of one or more organic acid metabolites produced following metabolism of mucin-derived carbohydrates by one or more genera of mucus-degrading bacteria are similar to or increased compared to a control or reference sample, the subject is not at risk or has a reduced risk of developing cancer therapy-induced neutropenic fever.

The levels of one or more organic acid metabolites produced following metabolism of mucin-derived carbohydrates by one or more genera of mucus-degrading bacteria in the gut microbiome of the subject are considered to be decreased compared to a control or reference sample when the levels of one or more organic acid metabolites produced following metabolism of mucin-derived carbohydrates by one or more genera of mucus-degrading bacteria in the gut microbiome of the subject are decreased to less than equal to any one of, at least any one of, at most any one of, or between any two of 0.1 mM, 0.2 mM, 0.3 mM, 0.4 mM. 0.5 mM, 0.6 mM, 0.7 mM. 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, 2.5 mM. 2.6 mM, 2.7 mM, 2.8 mM, 2.9 mM, 3.0 mM. 3.1 mM, 3.2 mM, 3.3 mM, 3.4 mM, 3.5 mM. 3.6 mM, 3.7 mM, 3.8 mM. 3.9 mM, 4.0 mM, 4.1 mM, 4.2 mM, 4.3 mM, 4.4 mM, 4.5 mM. 4.6 mM. 4.7 mM, 4.8 mM, 4.9 mM, 5.0 mM, 5.1 mM, 5.2 mM, 5.3 mM, 5.4 mM, 5.5 mM. 5.6 mM, 5.7 mM. 5.8 mM, 5.9 mM, 6.0 mM, 6.1 mM. 6.2 mM, 6.3 mM, 6.4 mM, 6.5 mM. 6.6 mM. 6.7 mM, 6.8 mM, 6.9 mM, 7.0 mM, 7.1 mM, 7.2 mM, 7.3 mM, 7.4 mM, 7.5 mM. 7.6 mM, 7.7 mM. 7.8 mM, 7.9 mM, 8.0 mM. 8.1 mM. 8.2 mM, 8.3 mM. 8.4 mM, 8.5 mM. 8.6 mM, 8.7 mM. 8.8 mM. 8.9 mM, 9.0 mM, 9.1 mM, 9.2 mM. 9.3 mM. 9.4 mM. 9.5 mM, 9.6 mM, 9.7 mM. 9.8 mM. 9.9 mM, 10.0 mM, 10.1 mM, 10.2 mM, 10.3 mM, 10.4 mM, 10.5 mM, 10.6 mM, 10.7 mM, 10.8 mM, 10.9 mM, 11.0 mM, 11.1 mM, 11.2 mM, 11.3 mM, 11.4 mM, 11.5 mM, 11.6 mM, 11.7 mM, 11.8 mM, 11.9 mM, 12.0 mM, 12.1 mM, 12.2 mM, 12.3 mM, 12.4 mM, 12.5 mM, 12.6 mM, 12.7 mM, 12.8 mM, 12.9 mM, 13.0 mM, 13.1 mM. 13.2 mM. 13.3 mM, 13.4 mM, 13.5 mM, 13.6 mM, 13.7 mM, 13.8 mM, 13.9 mM, 14.0 mM, 14.1 mM, 14.2 mM, 14.3 mM, 14.4 mM, 14.5 mM, 14.6 mM, 14.7 mM, 14.8 mM, 14.9 mM, 15.0 mM, 15.1 mM, 15.2 mM, 15.3 mM, 15.4 mM, 15.5 mM, 15.6 mM, 15.7 mM, 15.8 mM, 15.9 mM, 16.0 mM, 16.1 mM, 16.2 mM, 16.3 mM, 16.4 mM, 16.5 mM, 16.6 mM, 16.7 mM, 16.8 mM, 16.9 mM, 17.0 mM, 17.1 mM, 17.2 mM, 17.3 mM, 17.4 mM, 17.5 mM, 17.6 mM, 17.7 mM, 17.8 mM, 17.9 mM, 18.0 mM, 18.1 mM, 18.2 mM, 18.3 mM, 18.4 mM, 18.5 mM. 18.6 mM, 18.7 mM, 18.8 mM, 18.9 mM, 19.0 mM, 19.1 mM, 19.2 mM, 19.3 mM, 19.4 mM, 19.5 mM, 19.6 mM, 19.7 mM, 19.8 mM, 19.9 mM, 20.0 mM, or any range or value derivable therein, compared to a control or reference sample.

When the analysis of the gut microbiome indicates that the levels of one or more carbohydrate substrates metabolized by one or more genera of mucus-degrading bacteria are decreased compared to a control or reference sample or control, the subject has an increased likelihood of developing cancer therapy-induced neutropenic fever, and the subject may be provided an effective amount of one or more carbohydrate substrates to for metabolism by one or more genera of mucus-degrading bacteria in the gut microbiome of the subject, one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject. When the analysis of the gut microbiome indicates that the levels of one or more carbohydrate substrates metabolized by one or more genera of mucus-degrading bacteria are similar to or increased compared to a control or reference sample, the subject is not at risk or has a reduced risk of developing cancer therapy-induced neutropenic fever.

The levels of one or more carbohydrate substrates metabolized by one or more genera of mucus-degrading bacteria in the gut microbiome of the subject are considered to be decreased compared to a control or reference sample when the levels of one or more carbohydrate substrates metabolized by one or more genera of mucus-degrading bacteria in the gut microbiome of the subject are decreased to less than equal to any one of, at least any one of, at most any one of, or between any two of 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, 2.5 mM, 2.6 mM, 2.7 mM, 2.8 mM, 2.9 mM, 3.0 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 4.6 mM, 4.7 mM, 4.8 mM, 4.9 mM, 5.0 mM, 5.1 mM, 5.2 mM, 5.3 mM, 5.4 mM, 5.5 mM, 5.6 mM, 5.7 mM, 5.8 mM, 5.9 mM, 6.0 mM, 6.1 mM, 6.2 mM, 6.3 mM, 6.4 mM, 6.5 mM, 6.6 mM, 6.7 mM, 6.8 mM, 6.9 mM, 7.0 mM, 7.1 mM, 7.2 mM, 7.3 mM, 7.4 mM, 7.5 mM, 7.6 mM, 7.7 mM, 7.8 mM, 7.9 mM, 8.0 mM, 8.1 mM, 8.2 mM, 8.3 mM, 8.4 mM, 8.5 mM, 8.6 mM, 8.7 mM, 8.8 mM, 8.9 mM, 9.0 mM, 9.1 mM, 9.2 mM, 9.3 mM, 9.4 mM, 9.5 mM, 9.6 mM, 9.7 mM, 9.8 mM, 9.9 mM, 10.0 mM, 10.1 mM, 10.2 mM, 10.3 mM, 10.4 mM, 10.5 mM, 10.6 mM, 10.7 mM, 10.8 mM, 10.9 mM, 11.0 mM, 11.1 mM, 11.2 mM, 11.3 mM, 11.4 mM, 11.5 mM, 11.6 mM, 11.7 mM, 11.8 mM, 11.9 mM, 12.0 mM, 12.1 mM, 12.2 mM, 12.3 mM, 12.4 mM, 12.5 mM, 12.6 mM, 12.7 mM, 12.8 mM, 12.9 mM, 13.0 mM, 13.1 mM, 13.2 mM, 13.3 mM, 13.4 mM, 13.5 mM, 13.6 mM, 13.7 mM, 13.8 mM, 13.9 mM, 14.0 mM, 14.1 mM, 14.2 mM, 14.3 mM, 14.4 mM, 14.5 mM, 14.6 mM, 14.7 mM, 14.8 mM, 14.9 mM, 15.0 mM, 15.1 mM, 15.2 mM, 15.3 mM, 15.4 mM, 15.5 mM, 15.6 mM, 15.7 mM, 15.8 mM, 15.9 mM, 16.0 mM, 16.1 mM, 16.2 mM, 16.3 mM, 16.4 mM, 16.5 mM, 16.6 mM, 16.7 mM, 16.8 mM, 16.9 mM, 17.0 mM, 17.1 mM, 17.2 mM, 17.3 mM, 17.4 mM, 17.5 mM, 17.6 mM, 17.7 mM, 17.8 mM, 17.9 mM, 18.0 mM, 18.1 mM, 18.2 mM, 18.3 mM, 18.4 mM, 18.5 mM, 18.6 mM, 18.7 mM, 18.8 mM, 18.9 mM, 19.0 mM, 19.1 mM, 19.2 mM, 19.3 mM, 19.4 mM, 19.5 mM, 19.6 mM, 19.7 mM, 19.8 mM, 19.9 mM, 20.0 mM, or any range or value derivable therein, compared to a control or reference sample.

When the analysis of the gut microbiome indicates that levels of one or more ruminal metabolites that target the growth or expansion of one or more genera of mucus-degrading bacteria in the gut microbiome of the subject are decreased compared to a control or reference sample, the subject has an increased likelihood of developing cancer therapy-induced neutropenic fever, and the subject may be provided an effective amount of one or more mediators of organic acid metabolite levels produced following metabolism of mucin-derived carbohydrates by one or more genera of mucus-degrading bacteria in the gut microbiome of the subject, one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more carbohydrate substrates metabolized by one or more genera of mucus-degrading bacteria in the gut microbiome of the subject. When the analysis of the gut microbiome indicates that levels of one or more ruminal metabolites that target the growth or expansion of one or more genera of mucus-degrading bacteria in the gut microbiome of the subject are similar to or increased compared to a control or reference sample, the subject is not at risk or has a reduced risk of developing cancer therapy-induced neutropenic fever.

The levels of one or more one or more ruminal metabolites that target the growth or expansion of one or more genera of mucus-degrading bacteria in the gut microbiome of the subject are considered to be decreased compared to a control or reference sample when the levels of one or more ruminal metabolites that target the growth or expansion of one or more genera of mucus-degrading bacteria in the gut microbiome of the subject are decreased to less than equal to any one of, at least any one of, at most any one of, or between any two of 0.1 mM. 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, 2.5 mM, 2.6 mM, 2.7 mM, 2.8 mM, 2.9 mM, 3.0 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 4.6 mM, 4.7 mM, 4.8 mM, 4.9 mM, 5.0 mM, 5.1 mM, 5.2 mM, 5.3 mM, 5.4 mM, 5.5 mM, 5.6 mM, 5.7 mM, 5.8 mM, 5.9 mM, 6.0 mM, 6.1 mM, 6.2 mM, 6.3 mM, 6.4 mM, 6.5 mM, 6.6 mM, 6.7 mM, 6.8 mM, 6.9 mM, 7.0 mM, 7.1 mM, 7.2 mM, 7.3 mM, 7.4 mM, 7.5 mM, 7.6 mM, 7.7 mM, 7.8 mM, 7.9 mM, 8.0 mM, 8.1 mM, 8.2 mM, 8.3 mM, 8.4 mM, 8.5 mM, 8.6 mM, 8.7 mM, 8.8 mM, 8.9 mM, 9.0 mM, 9.1 mM, 9.2 mM, 9.3 mM, 9.4 mM, 9.5 mM, 9.6 mM, 9.7 mM, 9.8 mM, 9.9 mM, 10.0 mM, 10.1 mM, 10.2 mM, 10.3 mM, 10.4 mM, 10.5 mM, 10.6 mM, 10.7 mM, 10.8 mM, 10.9 mM, 11.0 mM, 11.1 mM, 11.2 mM, 11.3 mM, 11.4 mM, 11.5 mM, 11.6 mM, 11.7 mM, 11.8 mM, 11.9 mM, 12.0 mM, 12.1 mM, 12.2 mM, 12.3 mM, 12.4 mM, 12.5 mM, 12.6 mM, 12.7 mM, 12.8 mM, 12.9 mM, 13.0 mM, 13.1 mM, 13.2 mM, 13.3 mM, 13.4 mM, 13.5 mM, 13.6 mM, 13.7 mM, 13.8 mM, 13.9 mM, 14.0 mM, 14.1 mM, 14.2 mM, 14.3 mM, 14.4 mM, 14.5 mM, 14.6 mM, 14.7 mM, 14.8 mM, 14.9 mM, 15.0 mM, 15.1 mM, 15.2 mM, 15.3 mM, 15.4 mM, 15.5 mM, 15.6 mM, 15.7 mM, 15.8 mM, 15.9 mM, 16.0 mM, 16.1 mM, 16.2 mM, 16.3 mM, 16.4 mM, 16.5 mM, 16.6 mM, 16.7 mM, 16.8 mM, 16.9 mM, 17.0 mM, 17.1 mM, 17.2 mM, 17.3 mM, 17.4 mM, 17.5 mM, 17.6 mM, 17.7 mM, 17.8 mM, 17.9 mM, 18.0 mM, 18.1 mM, 18.2 mM, 18.3 mM, 18.4 mM, 18.5 mM, 18.6 mM, 18.7 mM, 18.8 mM, 18.9 mM, 19.0 mM, 19.1 mM, 19.2 mM, 19.3 mM, 19.4 mM, 19.5 mM, 19.6 mM, 19.7 mM, 19.8 mM, 19.9 mM, 20.0 mM, or any range or value derivable therein, compared to a control or reference sample.

In some embodiments, following analysis of the activity and/or composition of the gut microbiome of a subject, it is determined that a subject is at risk of developing neutropenic fever and is in need of a modification of the gut microbiome, including prior to or after being diagnosed with neutropenia, to prevent the development of neutropenic fever, including cancer therapy-induced neutropenic fever. In some cases, the modification comprises administering to the subject an effective amount of one or more compositions that modify the gut microbiome such that the presence and/or level and/or activity of one or more microbes are modified.

In some embodiments, the subject is provided an effective amount of a composition comprising one or more bacterial growth-suppressing agents that would reduce levels of one or more microbes that were determined to be excessive in the gut microbiome of a subject, and then following this administration (whether it be by one or more administrations), the subject's gut microbiome is then modified to a sufficient level such that the subject is no longer at risk or is at a lesser risk of developing neutropenic fever, including cancer therapy-induced neutropenic fever.

In some embodiments, the subject is provided an effective amount of a composition comprising one or more mucus-degrading enzyme inhibitors that would inhibit mucus degradation by enzymes produced by one or more genera of mucus-degrading bacteria in the gut microbiome that were determined to be excessive in the gut microbiome of a subject, and then following this administration (whether it be by one or more administrations), the activity of bacterial enzymes in individual's gut microbiome is then modified to a sufficient level such that the subject is no longer at risk or is at a lesser risk of developing neutropenic fever, including cancer therapy-induced neutropenic fever.

In some embodiments, the subject is provided an effective amount of a composition comprising one or more mediators of organic acid metabolite levels produced following metabolism of mucin-derived carbohydrates by one or more genera of mucus-degrading bacteria in the gut microbiome, and then following this administration (whether it be by one or more administrations), the metabolites in the subject's gut microbiome that serve as a feedback mechanism to suppress excessive utilization of mucin glycans, which would otherwise be metabolized by one or more genera of mucus-degrading bacteria in the gut microbiome, are modified to a sufficient level such that the subject is no longer at risk or is at a lesser risk of developing neutropenic fever, including cancer therapy-induced neutropenic fever.

In some embodiments, the subject is provided an effective amount of a composition comprising one or more carbohydrate substrates metabolized by one or more genera of mucus-degrading bacteria in the gut microbiome that were determined to be deficient or reduced in the gut microbiome of a subject, and then following this administration (whether it be by one or more administrations), the carbohydrate substrate levels in individual's gut microbiome is then modified to a sufficient level such that the subject is no longer at risk or is at a lesser risk of developing neutropenic fever, including cancer therapy-induced neutropenic fever.

In some embodiments, the likelihood of developing neutropenic fever, including cancer therapy-induced neutropenic fever, is determined based on the gut microbiome of the subject, including prior to or after the subject is diagnosed with neutropenia. In such cases, as a result of the gut microbiome activity and/or composition, targeted therapeutic strategies to treat or prevent neutropenic fever, including cancer therapy-induced neutropenic fever, are administered to the subject. For example, the subject may be given a therapeutically effective amount of one or more agents targeting growth or expansion of one or more genera of mucus-degrading bacteria in the gut microbiome; one or more mucus-degrading enzyme inhibitors to inhibit mucus degradation by one or more genera of mucus-degrading bacteria in the gut microbiome; one or more mediators of organic acid metabolite levels produced following metabolism of mucin-derived carbohydrates by one or more genera of mucus-degrading bacteria in the gut microbiome; and/or one or more carbohydrate substrates metabolized by one or more genera of mucus-degrading bacteria in the gut microbiome.

The disclosure also encompasses the determination or prediction whether, in response to a cancer therapy, the outcome of the cancer therapy will be development of neutropenic fever, including cancer therapy-induced neutropenic fever, by a subject by analyzing the microbiome. If the analysis determines the subject is in need of modification of the gut microbiome to treat or prevent neutropenic fever, including cancer therapy-induced neutropenic fever, the subject is provided an effective amount of a composition that addresses the deficiency of the microbiome. As one example, the subject may be provided an effective amount of a composition that comprises one or more bacterial growth-suppressing agents, one or more one or more mucus-degrading enzyme inhibitors, one or more mediators of organic acid metabolite levels, and/or one or more carbohydrate substrates.

### C. Methods of Treating or Preventing Graft-Versus-Host Disease

Methods of the disclosure allow for the treatment or prevention of graft-versus-host disease (GVHD), including HCT-related and/or neutropenic fever therapy-induced GVHD, by administering a therapeutically effective amount of a composition comprising one or more agents targeting growth or expansion of one or more genera of mucus-degrading bacteria in the gut microbiome of the subject; one or more mucus-degrading enzyme inhibitors to inhibit mucus degradation by one or more genera of mucus-degrading bacteria in the gut microbiome of the subject; one or more mediators of organic acid metabolite levels produced following metabolism of mucin-derived carbohydrates by one or more genera of mucus-degrading bacteria in the gut microbiome of the subject; and/or one or more carbohydrate substrates metabolized by one or more genera of mucus-degrading bacteria in the gut microbiome of the subject.

Methods of the disclosure include methods of treating or preventing GVHD, including HCT-related and/or neutropenic fever therapy-induced GVHD, where risk of developing GVHD, including HCT-related and/or neutropenic fever therapy-induced GVHD, is increased. In some embodiments, the subject is at a higher risk than an average person in the general population receiving the cancer therapy prior to HCT therapy and/or neutropenic fever therapy of developing HCT-related and/or neutropenic fever therapy-induced GVHD. In some embodiments, the HCT-related and/or neutropenic fever therapy-induced GVHD poses a greater risk to the health or life of the subject than such a condition would pose to an average person in the general population receiving the HCT therapy and/or neutropenic fever therapy. In some cases, the method is employed for a subject where it is uncertain whether or not risk of developing GVHD, including HCT-related and/or neutropenic fever therapy-induced GVHD, is increased, whereas in other cases the method is employed for a subject where it is known that the risk of developing GVHD, including HCT-related and/or neutropenic fever therapy-induced GVHD, is increased. In other cases, it has been determined that the risk of developing GVHD, including HCT-related and/or neutropenic fever therapy-induced GVHD, is increased for the subject, but the methods of the disclosure are still employed as a routine matter or in the general therapeutic interest of the subject.

The disclosure encompasses methods and compositions for modulating the gut microbiome activity and/or composition of a subject to treat or prevent GVHD, including HCT-related and/or neutropenic fever therapy-induced GVHD. The modulation may or may not be as a result of analysis of the gut microbiome activity and/or composition prior to or after diagnosing the subject with neutropenic fever, infection, and/or GVHD. In some cases, the modulation is a result of analysis of the gut microbiome prior to diagnosing the subject with neutropenic fever, infection, and/or GVHD, and the outcome of the analysis determines the nature of the resultant modulation of the gut microbiome. For example, the modulation may comprise providing a therapeutically effective amount of one or more agents targeting growth or expansion of one or more genera of mucus-degrading bacteria in the gut microbiome; one or more mucus-degrading enzyme inhibitors to inhibit mucus degradation by one or more genera of mucus-degrading bacteria in the gut microbiome; one or more mediators of organic acid metabolite levels produced following metabolism of mucin-derived carbohydrates by one or more genera of mucus-degrading bacteria in the gut microbiome; and/or one or more carbohydrate substrates metabolized by one or more genera of mucus-degrading bacteria in the gut microbiome of the subject.

In some cases, the subject receiving an HCT therapy and/or a neutropenic fever therapy and having or at risk of having GVHD was determined to have an increased abundance of mucus-degrading bacteria in the gut microbiome compared to a control or reference sample, and the modulation may comprise providing an effective amount of one or more bacterial growth-suppressing agents that would reduce levels of one or more microbes that were determined to be excessive in the gut microbiome of a subject. In some cases, the subject was determined to have an increase in functional activity and/or expression levels of one or more mucus-degrading enzymes secreted by one or more genera of mucus-degrading bacteria in the gut microbiome compared to a control or reference sample, and the modulation may comprise providing an effective amount of one or more one or more mucus-degrading enzyme inhibitors that would inhibit mucus degradation by enzymes produced by one or more genera of mucus-degrading bacteria in the gut microbiome that were determined to be excessive in the gut microbiome of a subject. In some cases, the subject was determined to have decreased levels of one or more organic acid metabolites produced following metabolism of mucin-derived carbohydrates by one or more genera of mucus-degrading bacteria in the gut microbiome compared to a control or reference sample, and the modulation may comprise providing an effective amount of one or more mediators of organic acid metabolite levels produced following metabolism of mucin-derived carbohydrates by one or more genera of mucus-degrading bacteria in the gut microbiome that serve as a feedback mechanism to suppress excessive utilization of mucin glycans, which would otherwise be metabolized by one or more genera of mucus-degrading bacteria in the gut microbiome. In some cases, the subject was determined to have decreased levels of one or more carbohydrate substrates metabolized by one or more genera of mucus-degrading bacteria in the gut microbiome compared to a control or reference sample, and the modulation may comprise providing an effective amount of one or more carbohydrate substrates for metabolism by one or more genera of mucus-degrading bacteria in the gut microbiome. In some cases, the subject receiving a HCT therapy and/or a neutropenic fever therapy and having or at risk of having GVHD was determined to have a decreased abundance of one or more classes, orders, families, genera, or species of commensal bacteria in the gut microbiome compared to a control or reference sample; one or more commensal bacteria may comprise Clostridia bacteria, and the modulation may comprise any method of modulation disclosed herein.

Though these methods of modulating the gut microbiome of a subject receiving a HCT therapy and/or a neutropenic fever therapy and having or at risk of having GVHD are described with reference to a particular dysbiosis (e.g., increased abundance of one or more genera of mucus-degrading bacteria, decreased abundance of one or more commensal bacteria, increased functional activity and/or expression levels of one or more mucus-degrading enzymes, decreased levels of one or more organic acid metabolites, and/or decreased levels of one or more carbohydrate substrates), one of skill in the art would understand that any method of modulation disclosed herein (e.g., administration of one or more bacterial growth-suppressing agents, one or more one or more mucus-degrading enzyme inhibitors, one or more mediators of organic acid metabolite levels, and/or one or more carbohydrate substrates) may be applied to treat, prevent, or reduce the severity of any dysbiosis (e.g., increased abundance of one or more genera of mucus-degrading bacteria, decreased abundance of one or more commensal bacteria, increased functional activity and/or expression levels of one or more mucus-degrading enzymes, have decreased levels of one or more organic acid metabolites, and/or decreased levels of one or more carbohydrate substrates) disclosed herein.

In some cases, the control or reference sample is a sample from a healthy subject. In some cases the control or reference sample is a sample from a subject to whom the HCT therapy and/or neutropenic fever therapy is not administered. In some cases, the control or reference sample is used to identify normal and/or abnormal ranges for the abundance of one or more genera of mucus-degrading bacteria in the gut microbiome; the abundance of one or more commensal bacteria in the gut microbiome; the functional activity and/or expression levels of one or more mucus-degrading enzymes secreted by one or more genera of mucus-degrading bacteria in the gut microbiome; levels of one or more organic acid metabolites produced following metabolism of mucin-derived carbohydrates by one or more genera of mucus-degrading bacteria in the gut microbiome; and/or levels of one or more carbohydrate substrates metabolized by one or more genera of mucus-degrading bacteria in the gut microbiome.

In some cases, the subject does not exhibit symptoms of HCT-related and/or neutropenic fever therapy-induced GVHD when the composition comprising one or more agents targeting growth or expansion of one or more genera of mucus-degrading bacteria in the gut microbiome of the subject; one or more mucus-degrading enzyme inhibitors to inhibit mucus degradation by one or more genera of mucus-degrading bacteria in the gut microbiome of the subject; one or more mediators of organic acid metabolite levels produced following metabolism of mucin-derived carbohydrates by one or more genera of mucus-degrading bacteria in the gut microbiome of the subject; and/or one or more carbohydrate substrates metabolized by one or more genera of mucus-degrading bacteria in the gut microbiome of the subject is administered. In some embodiments, the subject has been diagnosed with GVHD. In some embodiments, the composition is administered after the subject has been diagnosed with neutropenia, neutropenic fever, infection, and/or GVHD, and the composition may be administered to the subject every day until the subject no longer exhibits symptoms of GVHD and/or is determined to be cured of GVHD. In some embodiments, the composition is administered multiple times per day. In some embodiments, the composition is administered 1, 2, 3, 4, 5, or 6 times per day.

In some cases, the subject is diagnosed with GVHD due to infusion of allogeneic donor cells following administration of a chemotherapy received by the subject. The chemotherapy treatment received by the subject can comprise alkylating agents, marrow-suppressive agents, reduced intensity conditioning, myeloablative conditioning, non-myeloablative conditioning, and immunosuppressive drugs.

In some cases, the subject is diagnosed with GVHD due to infusion of allogeneic donor cells following administration of a radiotherapy treatment received by the subject. The radiotherapy treatment received by the subject can comprise external radiotherapy, internal radiotherapy, radioimmunotherapy, or intraoperative radiation therapy (IORT).

In some cases, the subject is diagnosed with GVHD due to infusion of allogeneic donor cells following administration of an immunotherapy treatment received by the subject. The immunotherapy treatment received by the subject can comprise a checkpoint inhibitor, an inhibitor of a co-stimulatory molecule, dendritic cell therapy, CAR-T cell therapy, cytokine therapy, or adoptive T cell therapy.

In some cases, the subject is diagnosed with GVHD due to a neutropenic fever therapy received by the subject. The subject may have neutropenic fever for any reason, including but not limited to neutropenia following receipt of a cancer therapy disclosed herein by the subject and subsequence microbial infection. The neutropenic fever therapy received by the subject can comprise one or more broad spectrum antibiotics, including cefepime and/or one or more carbapenems. The one or more carbapenems can include meropenem, imipenem/cilastatin, panipenem/betamipron, biapenem, ertapenem, and doripenem.

In some embodiments, the abundance of mucus-degrading and/or commensal bacteria in the gut microbiome, the functional activity and/or expression levels of one or more mucus-degrading enzymes secreted by one or more genera of mucus-degrading bacteria in the gut microbiome, the levels of one or more organic acid metabolites produced following metabolism of mucin-derived carbohydrates by one or more genera of mucus-degrading bacteria in the gut microbiome, and/or levels of one or more carbohydrate substrates metabolized by one or more genera of mucus-degrading bacteria in the gut microbiome were determined from a fecal sample from the subject. Mucus-degrading and/or commensal bacteria can be quantified by the relative abundance of 16S gene copies of bacterial taxa known to harbor mucolytic genes, the absolute abundance of 16S gene copies of these taxa, as well as whole metagenomic shotgun sequencing of DNA or RNA to identify and quantify mucolytic genes. Enzymatic activity of these bacteria could also be functionally quantified, by quantifying breakdown of mucin, or by activity of specific enzymes that participate in various steps of mucus breakdown.

In some cases, both a deficiency in the gut microbiome and an excess in the gut microbiome are both handled prior to or after diagnosing the subject with GVHD. In particular embodiments, such actions improve the efficacy of therapeutic strategies to treat or prevent GVHD, including HCT-related GVHD and/or neutropenic fever therapy-induced GVHD.

### D. Methods of Predicting Graft-Versus-Host Disease

In particular embodiments, the disclosure concerns methods of predicting development of graft-versus-host disease (GVHD), including HCT-related and/or neutropenic fever therapy-induced GVHD, in a subject receiving a cancer therapy followed by a HCT therapy and/or neutropenic fever therapy treatment based on analyzing one or more of the following biomarkers: (1) abundance of one or more genera of mucus-degrading bacteria in the gut microbiome of the subject; (2) abundance of one or more commensal bacteria in the gut microbiome of the subject; (3) the functional activity and/or expression levels of one or more mucus-degrading enzymes secreted by one or more genera of mucus-degrading bacteria in the gut microbiome of the subject; (4) levels of one or more organic acid metabolites produced following metabolism of mucin-derived carbohydrates by one or more genera of mucus-degrading bacteria in the gut microbiome of the subject; (5) levels of one or more carbohydrate substrates metabolized by one or more genera of mucus-degrading bacteria in the gut microbiome of the subject; and/or (6) levels of one or more ruminal metabolites that target the growth or expansion of one or more genera of mucus-degrading bacteria in the gut microbiome of the subject.

In some embodiments, the subject has been diagnosed with GVHD. In some cases, the subject develops GVHD due to a HCT therapy and/or a neutropenic therapy received by the subject.

In some cases, the HCT therapy and/or a neutropenic therapy follows a chemotherapy treatment received by the subject. The chemotherapy treatment received by the subject can comprise alkylating agents, marrow-suppressive agents, reduced intensity conditioning, myeloablative conditioning, non-myeloablative conditioning, and immunosuppressive drugs.

In some cases, the HCT therapy and/or a neutropenic therapy follows a radiotherapy treatment received by the subject. The radiotherapy treatment received by the subject can comprise external radiotherapy, internal radiotherapy, radioimmunotherapy, or intraoperative radiation therapy (IORT).

In some cases, the HCT therapy and/or a neutropenic therapy follows an immunotherapy treatment received by the subject. The immunotherapy treatment received by the subject can comprise a checkpoint inhibitor, an inhibitor of a co-stimulatory molecule, dendritic cell therapy, CAR-T cell therapy, cytokine therapy, or adoptive T cell therapy.

In some cases, the neutropenic fever therapy comprises one or more broad spectrum antibiotics, including cefepime and/or one or more carbapenems. The one or more carbapenems can include meropenem, imipenem/cilastatin, panipenem/betamipron, biapenem, ertapenem, and doripenem.

In some embodiments, the disclosure concerns methods of predicting a therapy outcome for a subject in need of a cancer therapy followed by a HCT therapy and/or a neutropenic fever therapy, including the likelihood of developing GVHD, including HCT-related GVHD and/or neutropenic fever therapy-induced GVHD, such as when compared to a standard or a subject with a different microbiome. Such analysis of (1), (2), (3), (4), (5), or (6) of the above compared to a control or reference sample results in a determination of whether or how best to treat or prevent the development of GVHD, including HCT-related and/or neutropenic fever therapy-induced neutropenic fever, in the subject receiving the HCT therapy and/or the neutropenic fever therapy. In some cases, the control or reference sample is a sample from a healthy subject. In some cases the control or reference sample is a sample from a subject to whom the HCT therapy and/or neutropenic fever therapy is not administered. In some cases, the control or reference sample is used to identify normal and/or abnormal ranges for the abundance of one or more genera of mucus-degrading and/or commensal bacteria in the gut microbiome; the functional activity and/or expression levels of one or more mucus-degrading enzymes secreted by one or more genera of mucus-degrading bacteria in the gut microbiome; levels of one or more organic acid metabolites produced following metabolism of mucin-derived carbohydrates by one or more genera of mucus-degrading bacteria in the gut microbiome; levels of one or more carbohydrate substrates metabolized by one or more genera of mucus-degrading bacteria in the gut microbiome of the subject; and/or levels of one or more ruminal metabolites that target the growth or expansion of one or more genera of mucus-degrading bacteria in the gut microbiome of the subject.

For example, when the analysis of the gut microbiome indicates that the abundance of one or more genera of mucus-degrading bacteria in the gut microbiome of the subject is increased compared to a control or reference sample, the subject has an increased likelihood of developing HCT-related and/or neutropenic fever therapy-induced GVHD, and the subject may then be provided a therapeutically effective amount of one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, one or more mediators of organic acid metabolite levels produced following metabolism of mucin-derived carbohydrates by one or more genera of mucus-degrading bacteria in the gut microbiome of the subject, and/or one or more carbohydrate substrates metabolized by one or more genera of mucus-degrading bacteria in the gut microbiome of the subject. When the analysis of the gut microbiome indicates that the abundance of one or more genera of mucus-degrading bacteria in the gut microbiome of the subject is similar to or decreased compared to a control or reference sample, the subject is not at risk or has a reduced risk of developing HCT-related and/or neutropenic fever therapy-induced GVHD.

The abundance of the one or more genera of mucus-degrading bacteria in the gut microbiome of the subject is considered to be increased compared to a control or reference sample when the abundance of the one or more genera of mucus-degrading bacteria in the gut microbiome of the subject comprises more than equal to any one of, at least any one of, at most any one of, or between any two of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%. 19.1%, 19.2%, 19.3%, 19.4%, 19.5%, 19.6%, 19.7%, 19.8%, 19.9%, 20.0%, or any range or value derivable therein, of the total gut microbiome bacterial population compared to a control or reference sample.

When the analysis of the gut microbiome indicates that the abundance of one or more commensal bacteria in the gut microbiome of the subject is decreased compared to a control or reference sample, the subject has an increased likelihood of developing HCT-related and/or neutropenic fever therapy-induced GVHD, and the subject may then be provided a therapeutically effective amount of one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, one or more mediators of organic acid metabolite levels produced following metabolism of mucin-derived carbohydrates by one or more genera of mucus-degrading bacteria in the gut microbiome of the subject, and/or one or more carbohydrate substrates metabolized by one or more genera of mucus-degrading bacteria in the gut microbiome of the subject.

When the analysis of the gut microbiome indicates that the abundance of one or more classes, orders, families, genera, or species of commensal bacteria in the gut microbiome of the subject is similar to or increased compared to a control or reference sample, the subject is not at risk or has a reduced risk of developing HCT-related and/or neutropenic fever therapy-induced GVHD. In some cases, the one or more commensal bacteria include Clostridia bacteria.

The abundance of the one or more commensal bacteria in the gut microbiome of the subject is considered to be decreased compared to a control or reference sample when the abundance of the one or more genera of mucus-degrading bacteria in the gut microbiome of the subject comprises more than equal to any one of, at least any one of, at most any one of, or between any two of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, or any range or value derivable therein, of the total gut microbiome bacterial population compared to a control or reference sample.

When the analysis of the gut microbiome indicates that the functional activity or expression levels of one or more mucus-degrading enzymes secreted by one or more genera of mucus-degrading bacteria are increased compared to a control or reference sample or control, the subject has an increased likelihood of developing HCT-related and/or neutropenic fever therapy-induced GVHD, and the subject may be provided an effective amount of one or more mucus-degrading enzyme inhibitors to inhibit mucus degradation by one or more genera of mucus-degrading bacteria in the gut microbiome of the subject, one or more agents targeting growth or expansion of one or more genera of mucus-degrading bacteria in the gut microbiome of the subject, one or more mediators of organic acid metabolite levels produced following metabolism of mucin-derived carbohydrates by one or more genera of mucus-degrading bacteria in the gut microbiome of the subject, and/or one or more carbohydrate substrates metabolized by one or more genera of mucus-degrading bacteria in the gut microbiome of the subject. When the analysis of the gut microbiome indicates that the functional activity or expression levels of one or more mucus-degrading enzymes secreted by one or more genera of mucus-degrading bacteria are similar to or decreased compared to a control or reference sample, the subject is not at risk or has a reduced risk of developing HCT-related and/or neutropenic fever therapy-induced GVHD.

The functional activity or expression levels of one or more mucus-degrading enzymes secreted by one or more genera of mucus-degrading bacteria in the gut microbiome of the subject are considered to be increased compared to a control or reference sample when the functional activity or expression levels of one or more mucus-degrading enzymes secreted by one or more genera of mucus-degrading bacteria in the gut microbiome of the subject are increased to greater than equal to any one of, at least any one of, at most any one of, or between any two of 1-fold, 10-fold, 20-fold, 30-fold, 40-fold, 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 410-fold, 420-fold, 430-fold, 440-fold, 450-fold, 460-fold, 470-fold, 480-fold, 490-fold, 500-fold, 510-fold, 520-fold, 530-fold, 540-fold, 550-fold, 560-fold, 570-fold, 580-fold, 590-fold, 600-fold, 610-fold, 620-fold, 630-fold, 640-fold, 650-fold, 660-fold, 670-fold, 680-fold, 690-fold, 700-fold, 710-fold, 720-fold, 730-fold, 740-fold, 750-fold, 760-fold, 770-fold, 780-fold, 790-fold, 800-fold, 810-fold, 820-fold, 830-fold, 840-fold, 850-fold, 860-fold, 870-fold, 880-fold, 890-fold, 900-fold, 910-fold, 920-fold, 930-fold, 940-fold, 950-fold, 960-fold, 970-fold, 980-fold, 990-fold, 1000-fold, 1100-fold, 1200-fold, 1300-fold, 1400-fold, 1500-fold, 1600-fold, 1700-fold, 1800-fold, 1900-fold, 2000-fold, 2100-fold, 2200-fold, 2300-fold, 2400-fold, 2500-fold, 2600-fold, 2700-fold, 2800-fold, 2900-fold, 3000-fold, 3100-fold, 3200-fold, 3300-fold, 3400-fold, 3500-fold, 3600-fold, 3700-fold, 3800-fold, 3900-fold, 4000-fold, 4100-fold, 4200-fold, 4300-fold, 4400-fold, 4500-fold, 4600-fold, 4700-fold, 4800-fold, 4900-fold, 5000-fold, 5100-fold, 5200-fold, 5300-fold, 5400-fold, 5500-fold, 5600-fold, 5700-fold, 5800-fold, 5900-fold, 6000-fold, 6100-fold, 6200-fold, 6300-fold, 6400-fold, 6500-fold, 6600-fold, 6700-fold, 6800-fold, 6900-fold, 7000-fold, 7100-fold, 7200-fold, 7300-fold, 7400-fold, 7500-fold, 7600-fold, 7700-fold, 7800-fold, 7900-fold, 8000-fold, 8100-fold, 8200-fold, 8300-fold, 8400-fold, 8500-fold, 8600-fold, 8700-fold, 8800-fold, 8900-fold, 9000-fold, 9100-fold, 9200-fold, 9300-fold, 9400-fold, 9500-fold, 9600-fold, 9700-fold, 9800-fold, 9900-fold, 10000-fold, 11000-fold, 12000-fold, 13000-fold, 14000-fold, 15000-fold, 16000-fold, 17000-fold, 18000-fold, 19000-fold, 20000-fold, 21000-fold, 22000-fold, 23000-fold, 24000-fold, 25000-fold, 26000-fold, 27000-fold, 28000-fold, 29000-fold, 30000-fold, 31000-fold, 32000-fold, 33000-fold, 34000-fold, 35000-fold, 36000-fold, 37000-fold, 38000-fold, 39000-fold, 40000-fold, 41000-fold, 42000-fold, 43000-fold, 44000-fold, 45000-fold, 46000-fold, 47000-fold, 48000-fold, 49000-fold, 50000-fold, 51000-fold, 52000-fold, 53000-fold, 54000-fold, 55000-fold, 56000-fold, 57000-fold, 58000-fold, 59000-fold, 60000-fold, 61000-fold, 62000-fold, 63000-fold, 64000-fold, 65000-fold, 66000-fold, 67000-fold, 68000-fold, 69000-fold, 70000-fold, 71000-fold, 72000-fold, 73000-fold, 74000-fold, 75000-fold, 76000-fold, 77000-fold, 78000-fold, 79000-fold, 80000-fold, 81000-fold, 82000-fold, 83000-fold, 84000-fold, 85000-fold, 86000-fold, 87000-fold, 88000-fold, 89000-fold, 90000-fold, 91000-fold, 92000-fold, 93000-fold, 94000-fold, 95000-fold, 96000-fold, 97000-fold, 98000-fold, 99000-fold, 100000-fold, or any range or value derivable therein, compared to a control or reference sample.

When the analysis of the gut microbiome indicates that levels of one or more organic acid metabolites produced following metabolism of mucin-derived carbohydrates by one or more genera of mucus-degrading bacteria are decreased compared to a control or reference sample, the subject has an increased likelihood of developing HCT-related and/or neutropenic fever therapy-induced GVHD, and the subject may be provided an effective amount of one or more mediators of organic acid metabolite levels produced following metabolism of mucin-derived carbohydrates by one or more genera of mucus-degrading bacteria in the gut microbiome of the subject, one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more carbohydrate substrates metabolized by one or more genera of mucus-degrading bacteria in the gut microbiome of the subject. When the analysis of the gut microbiome indicates that levels of one or more organic acid metabolites produced following metabolism of mucin-derived carbohydrates by one or more genera of mucus-degrading bacteria are similar to or increased compared to a control or reference sample, the subject is not at risk or has a reduced risk of developing HCT-related and/or neutropenic fever therapy-induced GVHD.

The levels of one or more organic acid metabolites produced following metabolism of mucin-derived carbohydrates by one or more genera of mucus-degrading bacteria in the gut microbiome of the subject are considered to be decreased compared to a control or reference sample when the levels of one or more organic acid metabolites produced following metabolism of mucin-derived carbohydrates by one or more genera of mucus-degrading bacteria in the gut microbiome of the subject are decreased to less than equal to any one of, at least any one of, at most any one of, or between any two of 0.1 mM, 0.2 mM, 0.3 mM. 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM. 1.6 mM. 1.7 mM, 1.8 mM, 1.9 mM. 2.0 mM. 2.1 mM, 2.2 mM, 2.3 mM. 2.4 mM, 2.5 mM, 2.6 mM, 2.7 mM. 2.8 mM. 2.9 mM, 3.0 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM. 4.6 mM. 4.7 mM, 4.8 mM, 4.9 mM, 5.0 mM, 5.1 mM, 5.2 mM. 5.3 mM, 5.4 mM, 5.5 mM. 5.6 mM, 5.7 mM, 5.8 mM, 5.9 mM, 6.0 mM, 6.1 mM, 6.2 mM, 6.3 mM. 6.4 mM, 6.5 mM. 6.6 mM. 6.7 mM. 6.8 mM, 6.9 mM, 7.0 mM, 7.1 mM, 7.2 mM, 7.3 mM. 7.4 mM, 7.5 mM. 7.6 mM. 7.7 mM, 7.8 mM, 7.9 mM. 8.0 mM. 8.1 mM. 8.2 mM. 8.3 mM, 8.4 mM, 8.5 mM. 8.6 mM. 8.7 mM, 8.8 mM. 8.9 mM, 9.0 mM. 9.1 mM. 9.2 mM. 9.3 mM. 9.4 mM, 9.5 mM. 9.6 mM. 9.7 mM. 9.8 mM. 9.9 mM. 10.0 mM, 10.1 mM, 10.2 mM, 10.3 mM, 10.4 mM, 10.5 mM, 10.6 mM, 10.7 mM, 10.8 mM, 10.9 mM, 11.0 mM, 11.1 mM, 11.2 mM, 11.3 mM, 11.4 mM. 11.5 mM, 11.6 mM, 11.7 mM, 11.8 mM, 11.9 mM, 12.0 mM, 12.1 mM, 12.2 mM, 12.3 mM. 12.4 mM. 12.5 mM. 12.6 mM. 12.7 mM, 12.8 mM, 12.9 mM, 13.0 mM, 13.1 mM, 13.2 mM, 13.3 mM, 13.4 mM, 13.5 mM, 13.6 mM, 13.7 mM, 13.8 mM, 13.9 mM, 14.0 mM, 14.1 mM, 14.2 mM, 14.3 mM, 14.4 mM, 14.5 mM, 14.6 mM, 14.7 mM, 14.8 mM, 14.9 mM, 15.0 mM, 15.1 mM, 15.2 mM, 15.3 mM, 15.4 mM, 15.5 mM, 15.6 mM, 15.7 mM, 15.8 mM, 15.9 mM, 16.0 mM, 16.1 mM, 16.2 mM, 16.3 mM, 16.4 mM, 16.5 mM, 16.6 mM, 16.7 mM, 16.8 mM, 16.9 mM, 17.0 mM, 17.1 mM, 17.2 mM, 17.3 mM, 17.4 mM, 17.5 mM, 17.6 mM, 17.7 mM, 17.8 mM. 17.9 mM, 18.0 mM, 18.1 mM, 18.2 mM, 18.3 mM, 18.4 mM, 18.5 mM, 18.6 mM, 18.7 mM, 18.8 mM, 18.9 mM, 19.0 mM. 19.1 mM, 19.2 mM, 19.3 mM, 19.4 mM. 19.5 mM, 19.6 mM, 19.7 mM, 19.8 mM, 19.9 mM. 20.0 mM, or any range or value derivable therein, compared to a control or reference sample.

When the analysis of the gut microbiome indicates that the levels of one or more carbohydrate substrates metabolized by one or more genera of mucus-degrading bacteria are decreased compared to a control or reference sample or control, the subject has an increased likelihood of developing HCT-related and/or neutropenic fever therapy-induced GVHD, and the subject may be provided an effective amount of one or more carbohydrate substrates to for metabolism by one or more genera of mucus-degrading bacteria in the gut microbiome of the subject, one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject. When the analysis of the gut microbiome indicates that the levels of one or more carbohydrate substrates metabolized by one or more genera of mucus-degrading bacteria are similar to or increased compared to a control or reference sample, the subject is not at risk or has a reduced risk of developing HCT-related and/or neutropenic fever therapy-induced GVHD.

The levels of one or more carbohydrate substrates metabolized by one or more genera of mucus-degrading bacteria in the gut microbiome of the subject are considered to be decreased compared to a control or reference sample when the levels of one or more carbohydrate substrates metabolized by one or more genera of mucus-degrading bacteria in the gut microbiome of the subject are decreased to less than equal to any one of, at least any one of, at most any one of, or between any two of 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, 2.5 mM, 2.6 mM, 2.7 mM, 2.8 mM, 2.9 mM, 3.0 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 4.6 mM, 4.7 mM, 4.8 mM, 4.9 mM, 5.0 mM, 5.1 mM, 5.2 mM, 5.3 mM, 5.4 mM, 5.5 mM, 5.6 mM, 5.7 mM, 5.8 mM, 5.9 mM, 6.0 mM, 6.1 mM, 6.2 mM, 6.3 mM, 6.4 mM, 6.5 mM, 6.6 mM, 6.7 mM, 6.8 mM, 6.9 mM, 7.0 mM, 7.1 mM, 7.2 mM, 7.3 mM, 7.4 mM, 7.5 mM, 7.6 mM, 7.7 mM, 7.8 mM, 7.9 mM, 8.0 mM, 8.1 mM, 8.2 mM, 8.3 mM, 8.4 mM, 8.5 mM, 8.6 mM, 8.7 mM, 8.8 mM, 8.9 mM, 9.0 mM, 9.1 mM, 9.2 mM, 9.3 mM, 9.4 mM, 9.5 mM, 9.6 mM, 9.7 mM, 9.8 mM, 9.9 mM, 10.0 mM, 10.1 mM, 10.2 mM, 10.3 mM, 10.4 mM, 10.5 mM, 10.6 mM, 10.7 mM, 10.8 mM, 10.9 mM, 11.0 mM, 11.1 mM, 11.2 mM, 11.3 mM, 11.4 mM, 11.5 mM, 11.6 mM, 11.7 mM, 11.8 mM, 11.9 mM, 12.0 mM, 12.1 mM, 12.2 mM, 12.3 mM, 12.4 mM, 12.5 mM, 12.6 mM, 12.7 mM, 12.8 mM, 12.9 mM, 13.0 mM, 13.1 mM, 13.2 mM, 13.3 mM, 13.4 mM, 13.5 mM, 13.6 mM, 13.7 mM, 13.8 mM, 13.9 mM, 14.0 mM, 14.1 mM, 14.2 mM, 14.3 mM, 14.4 mM, 14.5 mM, 14.6 mM, 14.7 mM, 14.8 mM, 14.9 mM, 15.0 mM, 15.1 mM, 15.2 mM, 15.3 mM, 15.4 mM, 15.5 mM, 15.6 mM, 15.7 mM, 15.8 mM, 15.9 mM, 16.0 mM, 16.1 mM, 16.2 mM, 16.3 mM, 16.4 mM, 16.5 mM, 16.6 mM, 16.7 mM, 16.8 mM, 16.9 mM, 17.0 mM, 17.1 mM, 17.2 mM, 17.3 mM, 17.4 mM, 17.5 mM, 17.6 mM, 17.7 mM, 17.8 mM, 17.9 mM, 18.0 mM, 18.1 mM, 18.2 mM, 18.3 mM, 18.4 mM, 18.5 mM, 18.6 mM, 18.7 mM, 18.8 mM, 18.9 mM, 19.0 mM, 19.1 mM, 19.2 mM, 19.3 mM, 19.4 mM, 19.5 mM, 19.6 mM, 19.7 mM, 19.8 mM, 19.9 mM, 20.0 mM, or any range or value derivable therein, compared to a control or reference sample.

When the analysis of the gut microbiome indicates that levels of one or more ruminal metabolites that target the growth or expansion of one or more genera of mucus-degrading bacteria in the gut microbiome of the subject are decreased compared to a control or reference sample, the subject has an increased likelihood of developing HCT-related and/or neutropenic fever therapy-induced GVHD, and the subject may be provided an effective amount of one or more mediators of organic acid metabolite levels produced following metabolism of mucin-derived carbohydrates by one or more genera of mucus-degrading bacteria in the gut microbiome of the subject, one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more carbohydrate substrates metabolized by one or more genera of mucus-degrading bacteria in the gut microbiome of the subject. When the analysis of the gut microbiome indicates that levels of one or more ruminal metabolites that target the growth or expansion of one or more genera of mucus-degrading bacteria in the gut microbiome of the subject are similar to or increased compared to a control or reference sample, the subject is not at risk or has a reduced risk of developing HCT-related and/or neutropenic fever therapy-induced GVHD.

The levels of one or more ruminal metabolites that target the growth or expansion of one or more genera of mucus-degrading bacteria in the gut microbiome of the subject are considered to be decreased compared to a control or reference sample when the levels of one or more ruminal metabolites that target the growth or expansion of one or more genera of mucus-degrading bacteria in the gut microbiome of the subject are decreased to less than equal to any one of, at least any one of, at most any one of, or between any two of 0.1 mM, 0.2 mM. 0.3 mM. 0.4 mM. 0.5 mM, 0.6 mM, 0.7 mM, 0.8 mM, 0.9 mM, 1.0 mM, 1.1 mM, 1.2 mM, 1.3 mM. 1.4 mM, 1.5 mM. 1.6 mM, 1.7 mM, 1.8 mM, 1.9 mM, 2.0 mM, 2.1 mM, 2.2 mM, 2.3 mM. 2.4 mM, 2.5 mM, 2.6 mM. 2.7 mM, 2.8 mM. 2.9 mM, 3.0 mM, 3.1 mM. 3.2 mM, 3.3 mM. 3.4 mM, 3.5 mM, 3.6 mM, 3.7 mM, 3.8 mM, 3.9 mM, 4.0 mM, 4.1 mM, 4.2 mM, 4.3 mM. 4.4 mM. 4.5 mM, 4.6 mM, 4.7 mM, 4.8 mM, 4.9 mM, 5.0 mM, 5.1 mM, 5.2 mM. 5.3 mM, 5.4 mM, 5.5 mM. 5.6 mM. 5.7 mM, 5.8 mM, 5.9 mM, 6.0 mM, 6.1 mM, 6.2 mM, 6.3 mM. 6.4 mM. 6.5 mM, 6.6 mM, 6.7 mM, 6.8 mM, 6.9 mM, 7.0 mM, 7.1 mM, 7.2 mM. 7.3 mM. 7.4 mM, 7.5 mM, 7.6 mM, 7.7 mM, 7.8 mM, 7.9 mM, 8.0 mM, 8.1 mM, 8.2 mM, 8.3 mM. 8.4 mM. 8.5 mM. 8.6 mM, 8.7 mM, 8.8 mM, 8.9 mM, 9.0 mM, 9.1 mM, 9.2 mM, 9.3 mM. 9.4 mM. 9.5 mM. 9.6 mM. 9.7 mM, 9.8 mM, 9.9 mM, 10.0 mM, 10.1 mM, 10.2 mM. 10.3 mM, 10.4 mM, 10.5 mM, 10.6 mM, 10.7 mM. 10.8 mM, 10.9 mM, 11.0 mM, 11.1 mM, 11.2 mM, 11.3 mM. 11.4 mM, 11.5 mM, 11.6 mM, 11.7 mM, 11.8 mM, 11.9 mM, 12.0 mM. 12.1 mM, 12.2 mM, 12.3 mM, 12.4 mM, 12.5 mM, 12.6 mM, 12.7 mM, 12.8 mM, 12.9 mM, 13.0 mM, 13.1 mM, 13.2 mM, 13.3 mM, 13.4 mM, 13.5 mM, 13.6 mM, 13.7 mM, 13.8 mM, 13.9 mM, 14.0 mM, 14.1 mM, 14.2 mM, 14.3 mM, 14.4 mM, 14.5 mM, 14.6 mM, 14.7 mM, 14.8 mM, 14.9 mM, 15.0 mM, 15.1 mM, 15.2 mM, 15.3 mM, 15.4 mM, 15.5 mM, 15.6 mM, 15.7 mM, 15.8 mM, 15.9 mM, 16.0 mM, 16.1 mM, 16.2 mM, 16.3 mM, 16.4 mM, 16.5 mM, 16.6 mM, 16.7 mM, 16.8 mM, 16.9 mM, 17.0 mM, 17.1 mM, 17.2 mM, 17.3 mM, 17.4 mM, 17.5 mM, 17.6 mM, 17.7 mM, 17.8 mM, 17.9 mM, 18.0 mM, 18.1 mM, 18.2 mM, 18.3 mM, 18.4 mM, 18.5 mM, 18.6 mM, 18.7 mM, 18.8 mM, 18.9 mM, 19.0 mM. 19.1 mM, 19.2 mM, 19.3 mM. 19.4 mM, 19.5 mM. 19.6 mM, 19.7 mM, 19.8 mM, 19.9 mM, 20.0 mM, or any range or value derivable therein, compared to a control or reference sample.

In some embodiments, following analysis of the activity and/or composition of the gut microbiome of a subject, it is determined that a subject is at risk of developing GVHD and is in need of a modification of the gut microbiome, including prior to or after being diagnosed with neutropenia, neutropenic fever, and/or GVHD, to prevent the development of GVHD, including HCT-related and/or neutropenic fever therapy-induced GVHD. In some cases, the modification comprises administering to the subject an effective amount of one or more compositions that modify the gut microbiome such that the presence and/or level and/or activity of one or more microbes are modified.

In some embodiments, the subject is provided an effective amount of a composition comprising one or more bacterial growth-suppressing agents that would reduce levels of one or more microbes that were determined to be excessive in the gut microbiome of a subject, and then following this administration (whether it be by one or more administrations), the subject's gut microbiome is then modified to a sufficient level such that the subject is no longer at risk or is at a lesser risk of developing GVHD, including HCT-related and/or neutropenic fever therapy-induced GVHD.

In some embodiments, the subject is provided an effective amount of a composition comprising one or more mucus-degrading enzyme inhibitors that would inhibit mucus degradation by enzymes produced by one or more genera of mucus-degrading bacteria in the gut microbiome that were determined to be excessive in the gut microbiome of a subject, and then following this administration (whether it be by one or more administrations), the activity of bacterial enzymes in individual's gut microbiome is then modified to a sufficient level such that the subject is no longer at risk or is at a lesser risk of developing GVHD, including HCT-related and/or neutropenic fever therapy-induced GVHD.

In some embodiments, the subject is provided an effective amount of a composition comprising one or more mediators of organic acid metabolite levels produced following metabolism of mucin-derived carbohydrates by one or more genera of mucus-degrading bacteria in the gut microbiome, and then following this administration (whether it be by one or more administrations), the metabolites in the subject's gut microbiome that serve as a feedback mechanism to suppress excessive utilization of mucin glycans, which would otherwise be metabolized by one or more genera of mucus-degrading bacteria in the gut microbiome, are modified to a sufficient level such that the subject is no longer at risk or is at a lesser risk of developing GVHD, including HCT-related and/or neutropenic fever therapy-induced GVHD.

In some embodiments, the subject is provided an effective amount of a composition comprising one or more carbohydrate substrates metabolized by one or more genera of mucus-degrading bacteria in the gut microbiome that were determined to be deficient or reduced in the gut microbiome of a subject, and then following this administration (whether it be by one or more administrations), the carbohydrate substrate levels in individual's gut microbiome is then modified to a sufficient level such that the subject is no longer at risk or is at a lesser risk of developing GVHD, including HCT-related and/or neutropenic fever therapy-induced GVHD.

In some embodiments, the likelihood of developing GVHD, including HCT-related and/or neutropenic fever therapy-induced GVHD, is determined based on the gut microbiome of the subject, including prior to or after the subject is diagnosed with neutropenia, neutropenic fever, and/or GVHD. In such cases, as a result of the gut microbiome activity and/or composition, targeted therapeutic strategies to treat or prevent GVHD, including HCT-related and/or neutropenic fever therapy-induced GVHD, are administered to the subject. For example, the subject may be given a therapeutically effective amount of one or more agents targeting growth or expansion of one or more genera of mucus-degrading bacteria in the gut microbiome; one or more mucus-degrading enzyme inhibitors to inhibit mucus degradation by one or more genera of mucus-degrading bacteria in the gut microbiome; one or more mediators of organic acid metabolite levels produced following metabolism of mucin-derived carbohydrates by one or more genera of mucus-degrading bacteria in the gut microbiome; and/or one or more carbohydrate substrates metabolized by one or more genera of mucus-degrading bacteria in the gut microbiome.

The disclosure also encompasses the determination or prediction whether, in response to a cancer therapy followed by HCT therapy and/or neutropenic fever therapy, the outcome of the cancer therapy followed by HCT therapy and/or neutropenic fever therapy will be development of GVHD, including HCT-related therapy and/or neutropenic fever therapy-induced GVHD, by a subject by analyzing the microbiome. If the analysis determines the subject is in need of modification of the gut microbiome to treat or prevent GVHD, including HCT-related and/or neutropenic fever therapy-induced GVHD, the subject is provided an effective amount of a composition that addresses the deficiency of the microbiome. As one example, the subject may be provided an effective amount of a composition that comprises one or more bacterial growth-suppressing agents, one or more one or more mucus-degrading enzyme inhibitors, one or more mediators of organic acid metabolite levels, and/or one or more carbohydrate substrates.

### VII. Methods of Analyzing the Gut Microbiome

The analysis of the gut microbiome to determine its content may be performed by any suitable method. The analysis may begin with collection of a suitable sample, such as stool, tissue biopsy, or a combination thereof. In specific cases wherein stool is the sample of choice, one may collect the whole stool, homogenize it immediately (e.g., with a blender or a tissue homogenizer), then flash freeze the homogenate in liquid nitrogen or in dry ice/ethanol slurry, with an aliquot preserved in a certain percentage of glycerol in suitable media for culturing. The subject that obtains the sample may or may not be the subject that performs the analysis. In some cases, the sample is stored prior to analysis, whereas in other cases the sample is analyzed without storage.

In particular embodiments, the gut microbiome is analyzed based on shotgun sequencing of nucleic acid of the microbe(s), including shotgun metagenomics sequencing, such as to provide more in-depth reads. In specific embodiments, the majority or substantially all of the genomic DNA for a microbe is analyzed instead of a specific region of DNA. However, in certain embodiments, analysis of a specific region of DNA is utilized, such as with 16S rRNA sequencing.

Other analysis methods may be utilized, either alone or with other methods. As one example, for known organisms with well-characterized selective culture conditions, culturing may be utilized as a detection method. Assay panels that target a set of known microbes or genes thereof may be utilized. Stool samples may be processed through nucleic acid extraction followed by complementary DNA synthesis and subsequent amplification using mixtures of primers specific for a given range of organisms. Either genomic DNA or PCR product may then be qualified and quantified, such as through a hybridization array using a fluorescence-based measure or a melt curve analysis. In specific embodiments, quantitative PCR and reverse-transcription quantitative PCR may be utilized.

In particular embodiments, amplicon analyses are employed in which a specific region of DNA is amplified by orders of magnitude using various methods including PCR. In specific cases, the PCR primers match a specific region, such as the 16S rRNA for bacteria. Bacterial 16S rRNA genes contain 9 hypervariable regions (V1-V9) that show sequence diversity and can be used as a barcode-like method to differentiate many bacterial taxa, including at the species level. In some cases, next-generation sequencing may be performed to read the sequences. In other cases, instead of using one gene, such as 16S rRNA, shotgun metagenomics is utilized that fragments all the DNA from a sample into small pieces, sequences these fragments, and then the sequenced fragments are arranged accordingly to provide information on a grander scale for the microbe identification.

### VIII. Sample Collection and Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy, or skin biopsy. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the check, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of an esophageal or a suspected esophageal tumor or neoplasm. In some cases, the fine needle aspirate sampling procedure may be guided by the use of an ultrasound, X-ray, or other imaging device.

In some embodiments of the present methods, the biological sample may be obtained from a subject directly, from a medical professional, from a third party, or from a kit provided by a third party. In some cases, the subject, a medical professional, or a third party may be provided with suitable containers and excipients for storage and transport of the biological sample.

In some embodiments of the methods described herein, a medical professional need not be involved in the initial sample acquisition. A subject may alternatively obtain a sample through the use of an over the counter (OTC) kit. An OTC kit may contain a means for obtaining said sample as described herein, a means for storing said sample for inspection, and instructions for proper use of the kit. A sample suitable for use may be any material containing tissues, cells, nucleic acids, genes, gene fragments, expression products, gene expression products, or gene expression product fragments of a subject to be tested. Methods for determining sample suitability and/or adequacy are known in the art.

In some embodiments, the subject may be referred to a specialist such as an oncologist, surgeon, or endocrinologist. The specialist may likewise obtain a biological sample for testing or refer the subject to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IX. Administration of Therapeutic Compositions

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

In some embodiments, the therapy provided herein comprises administration of a combination of therapeutic agents, such as a combination of one or more bacterial growth-suppressing agent compositions, one or more mucus-degrading enzyme inhibitor compositions, or one or more compositions comprising mediators of organic acid metabolite levels in the gut. In some embodiments, the therapy comprises administration of a combination or one or more bacterial growth-suppressing agent compositions and one or more mucus-degrading enzyme inhibitor compositions. In some embodiments, the therapy comprises administration of a combination of one or more bacterial growth-suppressing agent compositions and or one or more compositions comprising mediators of organic acid metabolite levels in the gut. In some embodiments, the therapy comprises administration of a combination of one or more mucus-degrading enzyme inhibitor compositions and or one or more compositions comprising mediators of organic acid metabolite levels in the gut. For example, a therapy may comprise administration of one or more antibiotics and one or more probiotics. The therapy may be administered in any suitable manner known in the art. For example, a bacterial growth-suppressing agent, a mucus-degrading enzyme inhibitor compositions, and a mediator of organic acid metabolite levels in the gut may be administered sequentially (at different times) or concurrently (at the same time).

Embodiments of the disclosure relate to compositions and methods comprising bacterial growth-suppressing agents, mucus-degrading enzyme inhibitors, and mediators of organic acid metabolite levels in the gut. The bacterial agents, mucus-degrading enzyme inhibitors, and mediators of organic acid metabolite levels in the gut may be administered in one composition or in more than one composition, such as 2 compositions, 3 compositions, or 4 compositions. Various combinations of the agents may be employed, for example, a bacterial growth-suppressing agent (or composition comprising a bacterial growth-suppressing agent) is “A” and a mucus-degrading enzyme inhibitor or mediator of organic acid metabolite levels in the gut is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In some embodiments, the bacterial growth-suppressing agent is administered prior to the mucus-degrading enzyme inhibitor, the mediator of organic acid metabolite levels in the gut, and/or the carbohydrate substrate(s) metabolized by mucus-degrading bacteria in the gut. In some embodiments, the bacterial growth-suppressing agent is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) prior to the mucus-degrading enzyme inhibitor, the mediator of organic acid metabolite levels in the gut, and/or the carbohydrate substrate(s) metabolized by mucus-degrading bacteria in the gut. In some embodiments, at least 1, 2, 3, 4, 5, 6, or 7 doses (or any derivable range therein) of the bacterial growth-suppressing agent is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) prior to the mucus-degrading enzyme inhibitor, the mediator of organic acid metabolite levels in the gut, and/or the carbohydrate substrate(s) metabolized by mucus-degrading bacteria in the gut. In some embodiments, the bacterial growth-suppressing agent is administered after the mucus-degrading enzyme inhibitor, the mediator of organic acid metabolite levels in the gut, and/or the carbohydrate substrate(s) metabolized by mucus-degrading bacteria in the gut. In some embodiments, the bacterial growth-suppressing agent is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) after the mucus-degrading enzyme inhibitor, the mediator of organic acid metabolite levels in the gut, and/or the carbohydrate substrate(s) metabolized by mucus-degrading bacteria in the gut. In some embodiments, at least 1, 2, 3, 4, 5, 6, or 7 doses (or any derivable range therein) of the bacterial growth-suppressing agent is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) after the mucus-degrading enzyme inhibitor, the mediator of organic acid metabolite levels in the gut, and/or the carbohydrate substrate(s) metabolized by mucus-degrading bacteria in the gut.

In some embodiments, the mucus-degrading enzyme inhibitor is administered prior to the bacterial growth-suppressing agent, the mediator of organic acid metabolite levels in the gut, and/or the carbohydrate substrate(s) metabolized by mucus-degrading bacteria in the gut. In some embodiments, the mucus-degrading enzyme inhibitor is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) prior to the bacterial growth-suppressing agent, the mediator of organic acid metabolite levels in the gut, and/or the carbohydrate substrate(s) metabolized by mucus-degrading bacteria in the gut. In some embodiments, at least 1, 2, 3, 4, 5, 6, or 7 doses (or any derivable range therein) of the mucus-degrading enzyme inhibitor is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) prior to the bacterial growth-suppressing agent, the mediator of organic acid metabolite levels in the gut, and/or the carbohydrate substrate(s) metabolized by mucus-degrading bacteria in the gut. In some embodiments, the mucus-degrading enzyme inhibitor is administered after the bacterial growth-suppressing agent, the mediator of organic acid metabolite levels in the gut, and/or the carbohydrate substrate(s) metabolized by mucus-degrading bacteria in the gut. In some embodiments, the mucus-degrading enzyme inhibitor is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) after the bacterial growth-suppressing agent, the mediator of organic acid metabolite levels in the gut, and/or the carbohydrate substrate(s) metabolized by mucus-degrading bacteria in the gut. In some embodiments, at least 1, 2, 3, 4, 5, 6, or 7 doses (or any derivable range therein) of the mucus-degrading enzyme inhibitor is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) after the bacterial growth-suppressing agent, the mediator of organic acid metabolite levels in the gut, and/or the carbohydrate substrate(s) metabolized by mucus-degrading bacteria in the gut.

In some embodiments, the mediator of organic acid metabolite levels in the gut is administered prior to the bacterial growth-suppressing agent, the mucus-degrading enzyme inhibitor, and/or the carbohydrate substrate(s) metabolized by mucus-degrading bacteria in the gut. In some embodiments, the mediator of organic acid metabolite levels in the gut is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) prior to the bacterial growth-suppressing agent, the mucus-degrading enzyme inhibitor, and/or the carbohydrate substrate(s) metabolized by mucus-degrading bacteria in the gut. In some embodiments, at least 1, 2, 3, 4, 5, 6, or 7 doses (or any derivable range therein) of the mediator of organic acid metabolite levels in the gut is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) prior to the bacterial growth-suppressing agent, the mucus-degrading enzyme inhibitor, and/or the carbohydrate substrate(s) metabolized by mucus-degrading bacteria in the gut. In some embodiments, the mediator of organic acid metabolite levels in the gut is administered after the bacterial growth-suppressing agent, the mucus-degrading enzyme inhibitor, and/or the carbohydrate substrate(s) metabolized by mucus-degrading bacteria in the gut. In some embodiments, the mediator of organic acid metabolite levels in the gut is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) after the bacterial growth-suppressing agent, the mucus-degrading enzyme inhibitor, and/or the carbohydrate substrate(s) metabolized by mucus-degrading bacteria in the gut. In some embodiments, at least 1, 2, 3, 4, 5, 6, or 7 doses (or any derivable range therein) of mediator of organic acid metabolite levels in the gut is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) after the bacterial growth-suppressing agent, the mucus-degrading enzyme inhibitor, and/or the carbohydrate substrate(s) metabolized by mucus-degrading bacteria in the gut.

In some embodiments, the carbohydrate substrate(s) metabolized by mucus-degrading bacteria in the gut is administered prior to the bacterial growth-suppressing agent, the mucus-degrading enzyme inhibitor, and/or the mediator of organic acid metabolite levels in the gut. In some embodiments, the carbohydrate substrate(s) metabolized by mucus-degrading bacteria in the gut is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) prior to the bacterial growth-suppressing agent, the mucus-degrading enzyme inhibitor, and/or the mediator of organic acid metabolite levels in the gut. In some embodiments, at least 1, 2, 3, 4, 5, 6, or 7 doses (or any derivable range therein) of the carbohydrate substrate(s) metabolized by mucus-degrading bacteria in the gut is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) prior to the bacterial growth-suppressing agent, the mucus-degrading enzyme inhibitor, and/or the mediator of organic acid metabolite levels in the gut. In some embodiments, the carbohydrate substrate(s) metabolized by mucus-degrading bacteria in the gut is administered after the bacterial growth-suppressing agent, the mucus-degrading enzyme inhibitor, and/or the mediator of organic acid metabolite levels in the gut. In some embodiments, the carbohydrate substrate(s) metabolized by mucus-degrading bacteria in the gut is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) after the bacterial growth-suppressing agent, the mucus-degrading enzyme inhibitor, and/or the mediator of organic acid metabolite levels in the gut. In some embodiments, at least 1, 2, 3, 4, 5, 6, or 7 doses (or any derivable range therein) of carbohydrate substrate(s) metabolized by mucus-degrading bacteria in the gut is administered at least, at most, or about 1, 2, 3, 5, 6, 12, 24 hours or 2, 3, 4, 6, 8, 10, days or 2, 3, 4, 5, 6, 7, or 8 weeks (or any derivable range therein) after the bacterial growth-suppressing agent, the mucus-degrading enzyme inhibitor, and/or the mediator of organic acid metabolite levels in the gut.

In some embodiments, the compositions comprising one or more bacterial agents, one or more mucus-degrading enzyme inhibitors, one or more mediators of organic acid metabolite levels in the gut are formulated for oral administration, and/or one or more carbohydrate substrates metabolized by mucus-degrading bacteria in the gut. The skilled artisan knows a variety of formulas which can encompass living or killed microorganisms and which can present as food supplements (e.g., pills, tablets, powders, and the like) or as functional food such as drinks or fermented yogurts.

The agents of the disclosure may be administered by the same route of administration or by different routes of administration. In some embodiments, the prebiotic is administered intravenously, intramuscularly, subcutaneously, topically, orally, transdermally, intraperitoneally, intraorbitally, by implantation, by inhalation, intrathecally, intraventricularly, or intranasally. In some embodiments, the microbial composition is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the subject, the subject's clinical history and response to the treatment, and the discretion of the attending physician.

Embodiments of the disclosure relate to compositions and methods comprising one or more bacterial growth-suppressing agents. In some embodiments, disclosed are compositions comprising one or more bacterial growth-suppressing agents. In some embodiments, the one or more bacterial growth-suppressing agents comprise one or more antibiotics. In some embodiments, the one or more antibiotics comprise azithromycin. In some embodiments, the one or more bacterial growth-suppressing agents comprise one or more antimicrobial proteins or peptides. In some embodiments, the one or more bacterial growth-suppressing agents comprise bucine, methyl-β-D-galactopyranoside, resacetophenone, or serotonin. In some embodiments, the one or more bacterial growth-suppressing agents comprise one or more ruminal metabolites. In some embodiments, the one or more ruminal metabolites comprise malic acid, 3-indole acetic acid, hydrocinnamic acid, methylmalonic acid, gluconic acid, galacturonic acid, or bis-hydroxy methyl propionic acid.

Embodiments of the disclosure relate to compositions and methods comprising one or more mediators of organic acid metabolite levels in the gut. In some embodiments, disclosed are compositions comprising one or more mediators of organic acid metabolite levels in the gut. In some embodiments, the one or more mediators of organic acid metabolite levels in the gut comprise one or more vitamins. In some embodiments, the one or more vitamins comprise vitamin B12.

In some embodiments, the one or more mediators of organic acid metabolite levels in the gut comprise one or more prebiotics. In some embodiments, the one or more mediators of organic acid metabolite levels in the gut comprise one or more probiotics. In some embodiments, a prebiotic and/or a probiotic composition may comprise a therapeutically effective amount of one or more bacteria. As used here, a “therapeutically effective” amount of a bacterium describes an amount sufficient to be effective in treating a desired condition, for example, neutropenic fever. In some embodiments, a therapeutically effective amount of isolated or purified populations of bacteria administered to a human will be at least about 1×10³ colony forming units (CFU) of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU (or any derivable range therein). In some embodiments, a single dose will contain bacteria (such as a specific bacteria or species, genus, or family described herein) present in an amount of least, at most, or about 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or more CFU (or any derivable range therein). In some embodiments, a single dose will contain at least, at most, or about 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ CFU (or any derivable range therein) of total bacteria.

In some embodiments, a therapeutically effective amount of each isolated or purified population of bacteria that is administered to a human will be at least about 1×10³ cells of bacteria or at least about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ cells (or any derivable range therein). In some embodiments, a single dose will contain bacteria (such as a specific bacteria or species, genus, or family described herein) present in an amount of at least, at most, or about 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or more cells (or any derivable range therein). In some embodiments, a single dose will contain at least, at most, or about 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ cells (or any derivable range therein) of total bacteria.

In some embodiments, the one or more mediators of organic acid metabolite levels in the gut comprise one or more metabolites from a metabolic pathway. In some embodiments, the metabolic pathway comprises degradation of mucin and/or mucin-derived carbohydrates by bacteria in the gut microbiome. In some embodiments, the disclosed compositions comprise one or more metabolites from the metabolic pathway comprising degradation of mucin and/or mucin-derived carbohydrates by bacteria in the gut microbiome. In some embodiments, the disclosed compositions comprise propionate. In some embodiments, the disclosed compositions comprise butyrate. In some embodiments, the disclosed compositions comprise acetate. In some embodiments, the disclosed compositions comprise isovalerate. In some embodiments, the disclosed compositions comprise valerate.

Also contemplated are compositions comprising inhibitors of the metabolic pathway comprising degradation of mucin and/or mucin-derived carbohydrates by bacteria in the gut microbiome. For example, in some embodiments, a composition comprises an inhibitor of one or more enzymes involved in the metabolism of or degradation of mucin and/or mucin-derived carbohydrates in the gut microbiome. In some embodiments, the one or more mucus-degrading enzyme inhibitors comprise inhibitors of glycosidase, sulfatase, neuraminidase, cysteine protease, Vat protease, α- and β-galactosidases, α-fucosidases, α- and β-N-acetylgalactosaminidases, β-N-acetylglucosaminidases, or mucinases.

Embodiments of the disclosure relate to compositions and methods comprising one or more carbohydrate substrates metabolized by one or more genera of mucus-degrading bacteria in the gut. In some embodiments, disclosed are compositions comprising one or more carbohydrate substrates metabolized by one or more genera of mucus-degrading bacteria. In some embodiments, the one or more carbohydrate substrates comprise one or more mono- or polysaccharides. In some embodiments, the one or more mono-saccharides comprise arabinose, fructose, fucose, galactose, galacturonic acid, glucuronic acid, glucosamine, glucose, mannose, N-acetylglucosamine, N-acetylgalactosamine, rhamnose, ribose, and/or xylose. In some embodiments, the one or more polysaccharides comprise pullulan, glycogen, amylopectin, inulin, levan, heparin, hyaluronan, chondroitin sulfate, polygalacturonate, rhamnogalacturonan, pectic galactan, arabinogalactan, arabinan, xylan, arabinoxylan, galactomannan, glucomannan, xyloglucan, β-glucan, cellobiose, laminarin, lichenin, dextran, and/or α-mannan. In some embodiments, the one or more mono- or polysaccharides comprise glucose, mannose, and/or xylose. In some embodiments, the one or more mono- or polysaccharides comprise xylose.

The treatments may include various “unit doses.” Unit dose is defined as containing a predetermined-quantity of the therapeutic composition calculated to produce the desired responses discussed above in association with its administration, i.e., the appropriate route and regimen. The quantity to be administered, and the particular route and formulation, is within the skill of determination of those in the clinical arts and depends on the result and/or protection desired. A unit dose need not be administered as a single injection but may comprise continuous infusion over a set period of time. In some embodiments, a unit dose comprises a single administrable dose.

Typically, compositions are administered in a manner compatible with the dosage formulation, and in such amount as will be therapeutically or prophylactically effective for the subject being treated. Precise amounts of the therapeutic composition also depend on the judgment of the practitioner and are peculiar to each individual. Suitable regimes for initial administration and boosters are also variable, but are typified by an initial administration followed by subsequent administrations. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

The compositions will be pharmaceutically acceptable or pharmacologically acceptable. The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

The carrier may be a solvent or dispersion medium containing, for example, water, ethanol, polyol (for example, glycerol, propylene glycol, and liquid polyethylene glycol), and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of undesirable microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

### X. Kits

Certain aspects of the present disclosure also concern kits containing compositions of the disclosure or compositions to implement methods of the disclosure. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a sample or a cell. The kit may also include components for collecting said sample or cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker. For example, in some cases, kits include components for measuring 16S gene copies of bacterial taxa. In some cases, kits may also include in addition to, in combination with, or separate from components for measuring 16S gene copies of bacterial taxa components for measuring enzymatic activity of bacteria, such enzymatic activity including but not limited to breakdown of mucin.

In certain aspects, negative and/or positive control nucleic acids, probes, and inhibitors are included in some kit embodiments. In addition, a kit may include a sample that is a negative or positive control for one or more biomarkers.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Diet-Derived Metabolites and Mucus Link the Gut Microbiome to Fever after Cytotoxic Cancer Treatment

A. Mucus-Degrading Intestinal Bacteria are Associated with Development of Fever Following Onset of Neutropenia in HCT Patients

A cohort of 119 patients who all developed neutropenia following HCT conditioning was examined. Of these, 56 (47%) remained afebrile over the next 4 days, while 63 (53%) developed a fever, of which 7 were found to have a bloodstream infection, including 5 with Enterobacteriaceae and 2 with oral streptococci. In the microbiome analyses described below, patients with bloodstream infections were included with those who developed fever, though analyses excluding these patients found very similar results. Prior to stool collection, patients were treated daily with prophylactic levofloxacin. Additional patient characteristics are provided below in Table 1.

TABLE 1
Patient characteristics

| | Combined | | No fever | | Fever | | p value |
|---|---|---|---|---|---|---|---|
| Total patients | n = 119 | | n = 56 | | n = 63 | | |
| Median age (range) | 58 years | | 62 years | | 56 years | | 0.12 |
| | (23-80) | | (23-80) | | (23-75) | | |
| Gender | | | | | | | |
| Female | n = 49 | (41.2%) | n = 22 | (39.3%) | n = 27 | (42.9%) | 0.69 |
| Male | n = 70 | (58.8%) | n = 34 | (60.7%) | n = 36 | (57.1%) | |
| Graft type | | | | | | | |
| Autologous HCT | n = 62 | (52.1%) | n = 22 | (39.3%) | n = 40 | (63.5%) | 0.008 |
| Allogeneic HCT | n = 57 | (47.9%) | n = 34 | (60.7%) | n = 23 | (36.5%) | |
| Neutropenia depth | | | | | | | |
| WBC 100-500/μL | n = 34 | (28.6%) | n = 15 | (26.8%) | n = 19 | (30.2%) | 0.84 |
| WBC <100/μL | n = 85 | (71.4%) | n = 41 | (73.2%) | n = 44 | (60.8%) | |
| Disease | | | | | | | |
| Multiple myeloma/PCD | n = 43 | (36.1%) | n = 16 | (28.6%) | n = 27 | (42.9%) | 0.11 |
| Acute myeloid leukemia | n = 23 | (19.3%) | n = 12 | (21.4%) | n = 11 | (17.5%) | 0.58 |
| Non-Hodgkin lymphoma | n = 20 | (16.8%) | n = 9 | (16.1%) | n = 11 | (17.5%) | 0.84 |
| MDS/MPN/MF | n = 12 | (10.1%) | n = 8 | (14.3%) | n = 4 | (6.3%) | 0.22 |
| Acute lymphocytic leukemia | n = 11 | (9.2%) | n = 7 | (12.5%) | n = 4 | (6.3%) | 0.34 |
| Hodgkin lymphoma | n = 4 | (3.4%) | n = 0 | (0%) | n = 4 | (6.3%) | 0.12 |
| Other | n = 6 | (5%) | n = 4 | (7.1%) | n = 2 | (3.2%) | 0.42 |
| Conditioning regimen | | | | | | | |
| Busulfan-based | n = 33 | (27.7%) | n = 18 | (32.1%) | n = 15 | (23.8%) | 0.41 |
| Melphalan-based | n = 64 | (53.8%) | n = 32 | (57.1%) | n = 32 | (50.8%) | 0.58 |
| Busulfan and melphalan-based | n = 17 | (14.3%) | n = 2 | (3.6%) | n = 15 | (23.8%) | 0.0015 |
| Other | n = 5 | (4.2%) | n = 4 | (7.1%) | n = 1 | (1.6%) | 0.19 |
| Conditioning intensity | | | | | | | |
| Myeloablative | n = 101 | (84.9%) | n = 44 | (78.6%) | n = 57 | (90.5%) | 0.08 |
| Nonmyeloablative | n = 18 | (15.1%) | n = 12 | (21.4%) | n = 6 | (9.5%) | |

Abbreviations:
HCT (hematopoietic cell transplantation), WBC (white blood cell), PCD (plasma cell disorder), MDS (myelodysplastic syndrome), MPN (myeloproliferative neoplasm), MF (myelofibrosis). “Other” includes blastic plasmacytoid dendritic cell neoplasm (n = 2), chronic myelogenous leukemia (n = 1), germ-cell tumor (n = 1), systemic sclerosis (n = 1), and T-cell-prolymphocytic leukemia (n = 1).

Receiving an autologous HCT, or a HCT following a preparative regimen based on both busulfan and melphalan, was associated with increased fever, likely reflecting increased conditioning intensity.

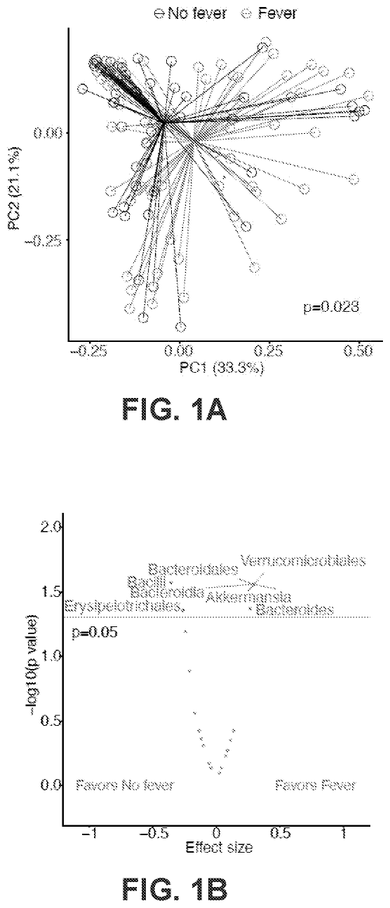

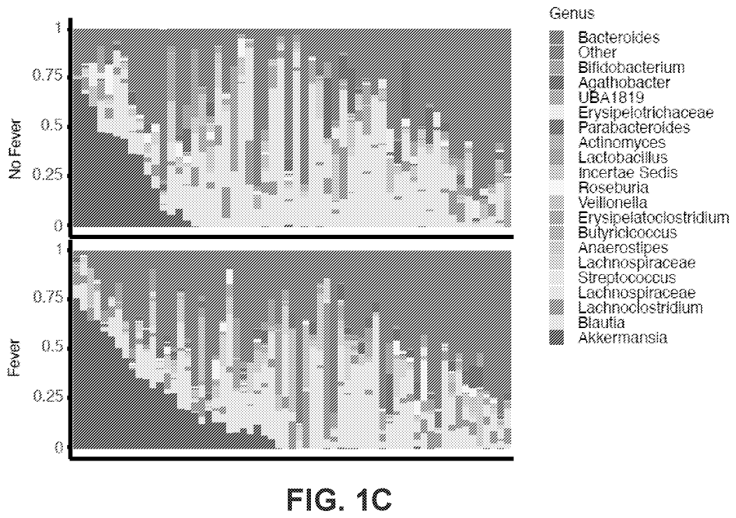

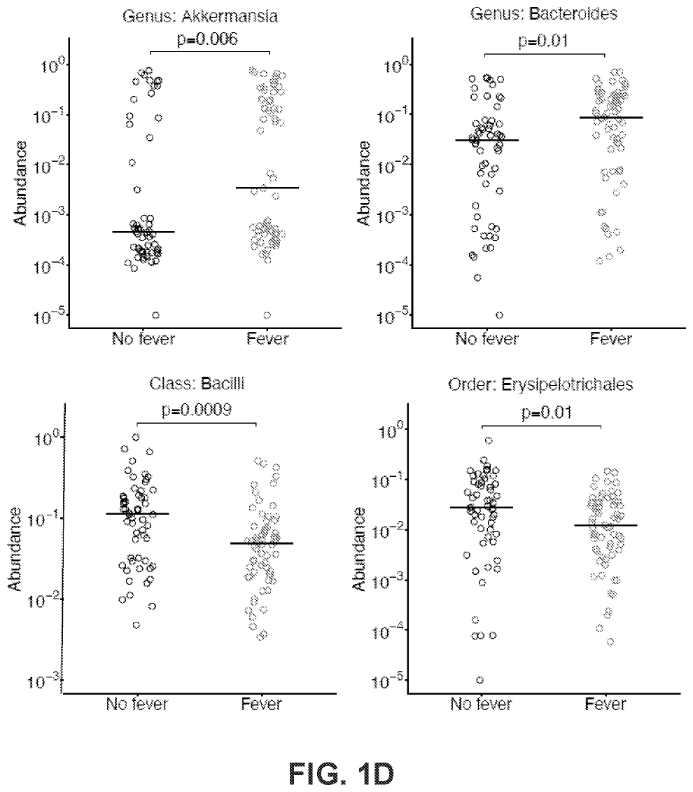

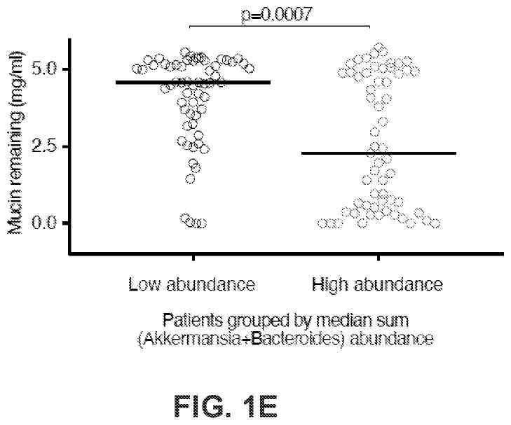

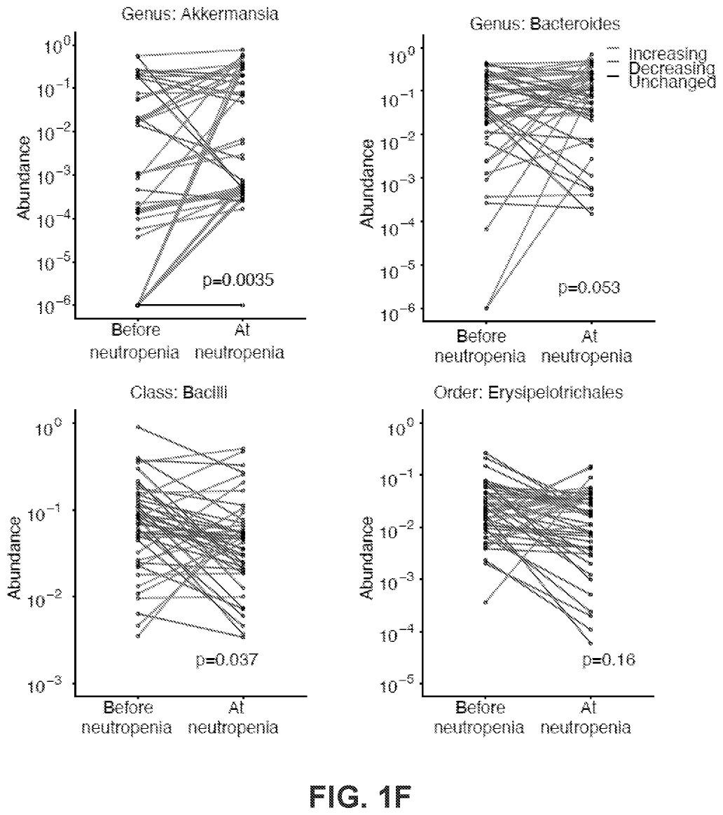

When analyzing stool samples collected at the onset of neutropenia, microbiome differences were observed between patients who did or did not develop fever over the next 4 days. There was a significant difference in beta-diversity (p=0.02, permutational MANOVA, FIG. 1A). Patients who later developed fever had increased relative abundance of bacteria from the genus Akkermansia (p=0.006, adjusted for multiple comparisons), as well as bacteria from the genus Bacteroides (p=0.01), while bacteria from the class Bacilli and from the order Erysipelotrichales were increased in patients that were afebrile (FIGS. 1B, 1C, and 1D). Bacterial taxa associated with bloodstream infections, included Enterobacteriales, Streptococcus, and Enterococcus (7) were not associated with fever.

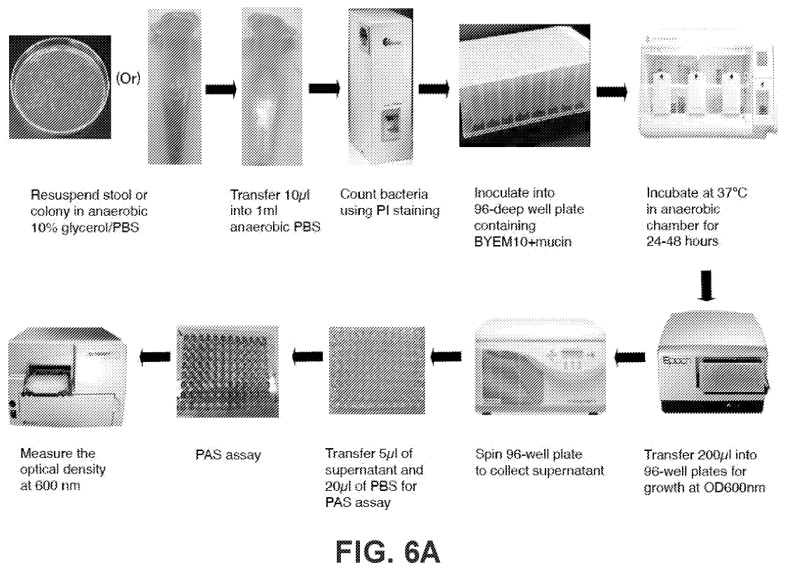

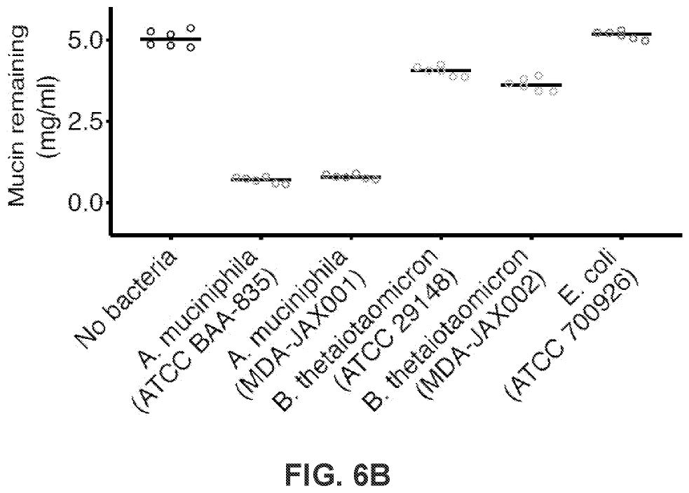

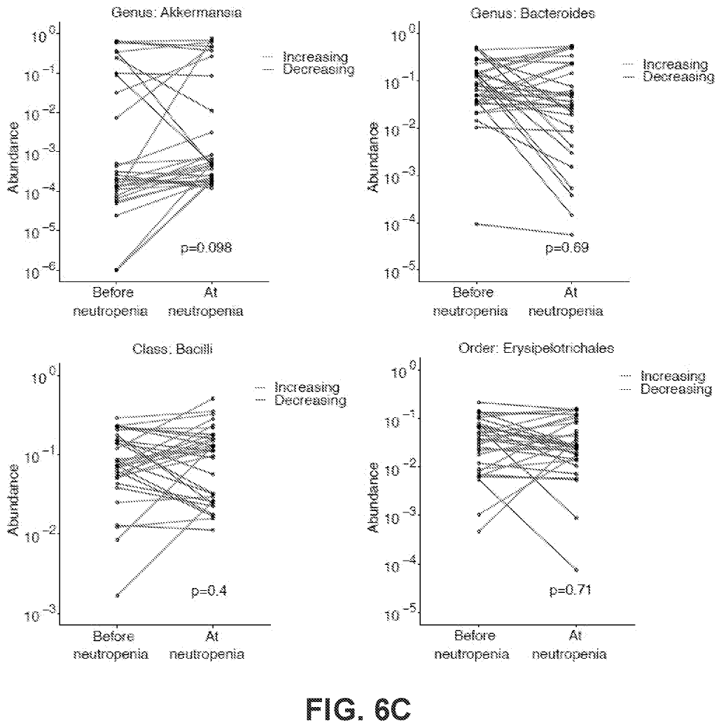

The genus Akkermansia currently includes only one species, Akkermansia muciniphila (A. muciniphila), while Bacteroides is quite diverse. A. muciniphila and several members of Bacteroides are known to have mucus-degrading capabilities (8), and the ability of intestinal bacteria from patients with febrile neutropenia to degrade mucin glycans was investigated. Certain carbohydrates including mucin glycans can be quantified from liquid samples using periodic acid-Schiff's reagent (FIG. 6A) (9), and such a method can quantify the concentration of glycans derived from commercially available porcine gastric mucin (PGM) in media. Following a 48-hour culture, a reduction in glycan concentration in media inoculated with isolates of A. muciniphila (ATCC BAA-835) and Bacteroides thetaiotaomicron (B. thetaiotaomicron, ATCC 29148) was quantifiable, but glycan levels were not altered by a non-mucolytic isolate of E. coli (ATCC 700926, FIG. 6B). Application of this assay to aliquots of patient samples from FIG. 1 determined that samples from patients with higher combined abundances of Akkermansia and Bacteroides were more effective at consuming mucin glycans (FIG. 1E).

In a majority of patients from the inventors' cohort (32 of 56 without fever and 44 of 63 with fever), baseline stool samples had been collected at least 4 days prior to onset of neutropenia and were available for comparison. Patients who later developed fever had significantly increased Akkermansia and reduced Bacilli at onset of neutropenia compared to baseline, while afebrile patients had no significant changes over time in any of the bacteria associated with fever (FIG. 1F, FIG. 6C).

In summary, evaluation of the microbiome in the setting of clinical neutropenia onset identified an increase in the abundance of bacteria with mucolytic properties in patients who later developed fever, and Akkermansia in particular was significantly increased from baseline in patients who later developed fever. Together, these results demonstrate a relationship between the composition of intestinal bacteria and fever in patients who developed neutropenia in the setting of HCT.

### B. Systemic Cytotoxic Therapy Increases the Relative Abundance of Mucus-Degrading Intestinal Bacteria in Mice

Previous analyses were performed in patients not receiving broad-spectrum antibiotics, suggesting a non-antimicrobial mechanism mediating the dysbiotic expansion of mucolytic bacteria. Thus, total body radiotherapy of C57BL/6 mice was performed to investigate whether transplant conditioning changes the composition of the intestinal microbiome in a murine model for evaluating of the impact of cytotoxic therapy.

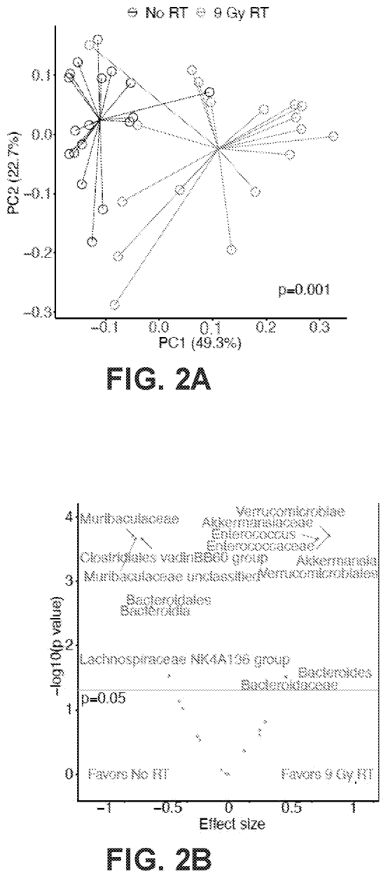

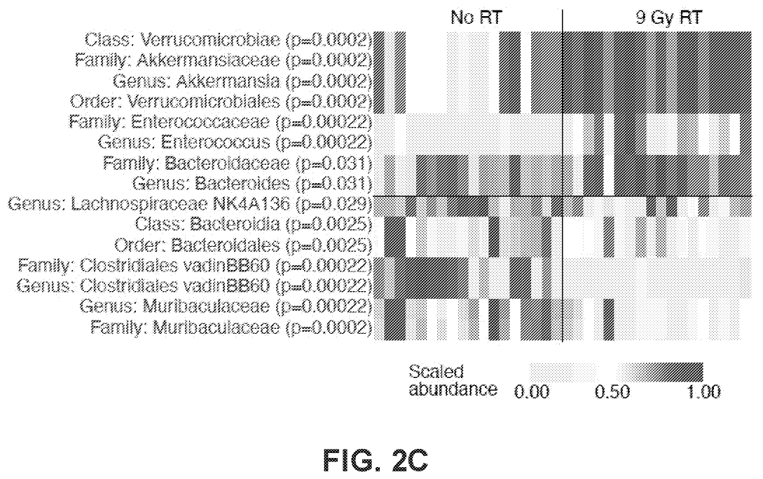

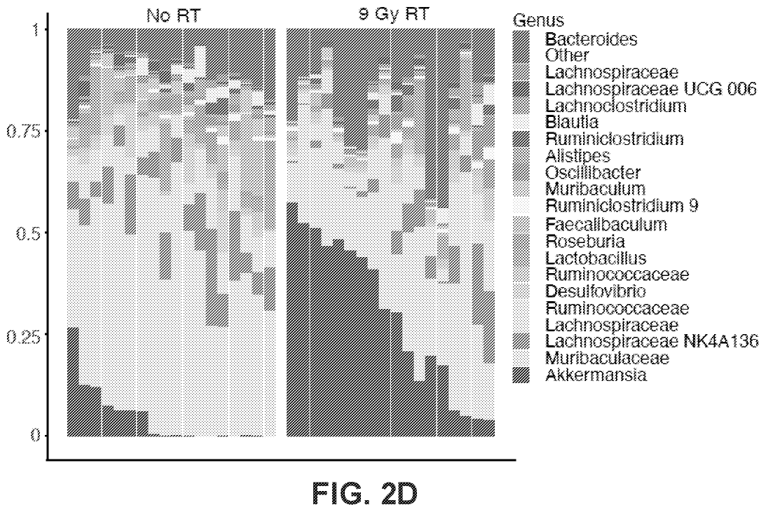

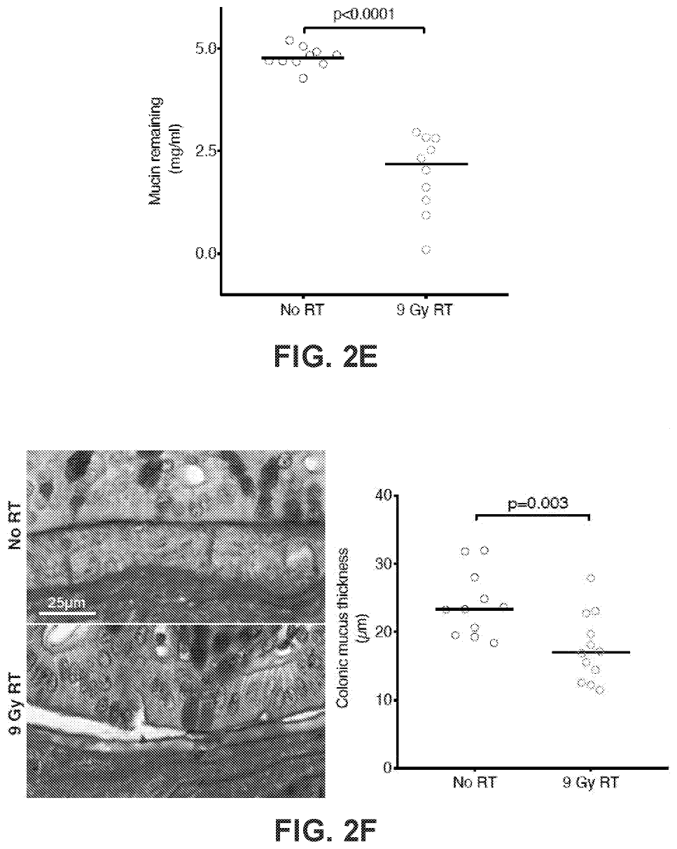

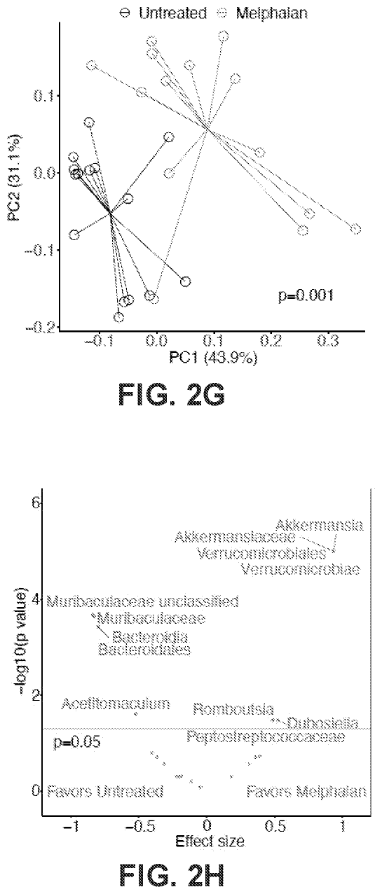

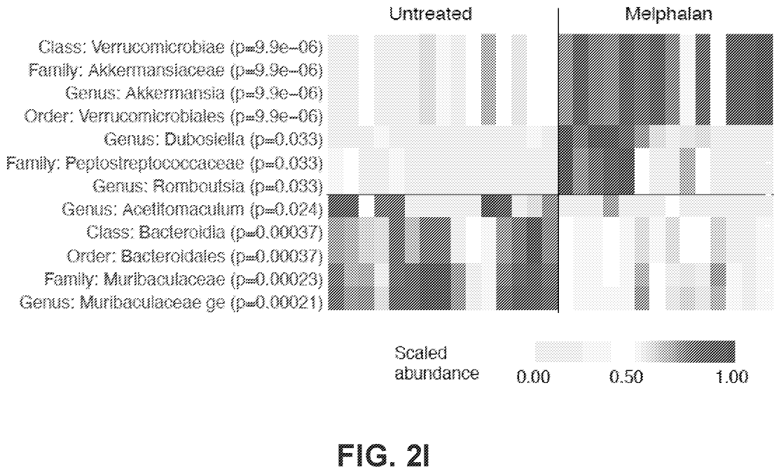

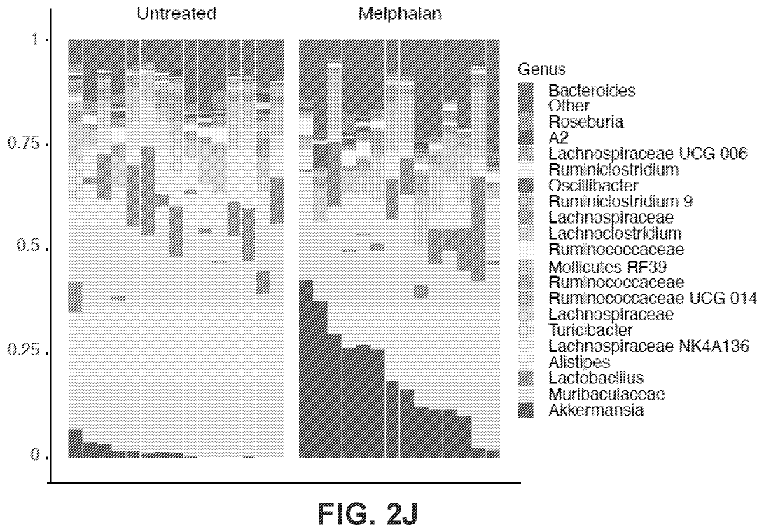

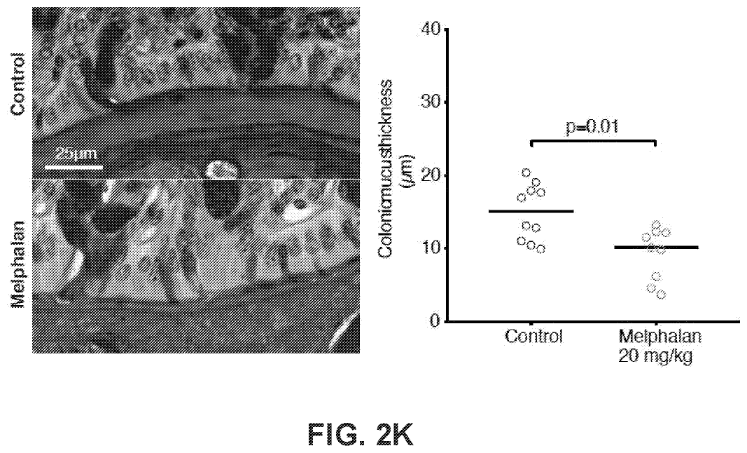

C57BL/6 mice were exposed to a single myeloablative dose of total body radiotherapy (9 Gy RT), and stool samples were evaluated 6 days later by 16S rRNA gene sequencing. This time point was chosen because mice would often become moribund by day 7. The microbiome of mice on day 6 was markedly changed compared to un-irradiated mice (FIG. 2A). Moreover, the profile was highly reminiscent of that of HCT patients with febrile neutropenia, showing increases in the abundance of Akkermansia, and to a lesser degree, Bacteroides (FIGS. 2B-2D). No compensatory reductions in Bacilli or Erysipelotrichales were observed, however. Rather, reduced were bacteria from the family Muribaculaceae, a recently-named group of bacteria commonly found in high abundance in mice but one that is usually a minor contributor in the intestinal tract of humans (10). Bacteria derived from stool samples of irradiated mice more efficiently degraded mucin glycans than bacteria from un-irradiated mice in vitro (FIG. 2E), indicating that changes in bacterial composition are accompanied by functional changes. Colonic tissue samples were cut cross-sectionally and mucus layer thickness was averaged across 8 equally-spaced circumferential sites (FIG. 7) to evaluate whether the dense colonic mucus layer, which normally separates bacteria-rich luminal contents from the colonic epithelium, was affected by irradiation in mice. Using this systematic approach, the mucus layer was found to be significantly thinner in irradiated mice compared to normal mice (FIG. 2F).

Myeloablative RT, previously a foundational pillar that made HCT possible, has been progressively replaced by chemotherapy, particularly alkylating agents (11). Thus, mice were treated with the alkylating agent melphalan, and treatment was found to lead to significant changes in the microbiome, marked particularly by an increase in the abundance of Akkermansia, similar to that seen after RT (FIGS. 2G-2J). An expansion of Bacteroides was also seen, though this was not statistically significant after correction for multiple comparisons, and was accompanied by a loss of Muribaculaceae, similar to that following RT. Histological analysis demonstrated that the mucus layer was significantly thinner in melphalan-treated mice, similar to RT (FIG. 2K).

### C. Dietary Restriction Increases the Relative Abundance of Mucus-Degrading Intestinal Bacteria in Mice

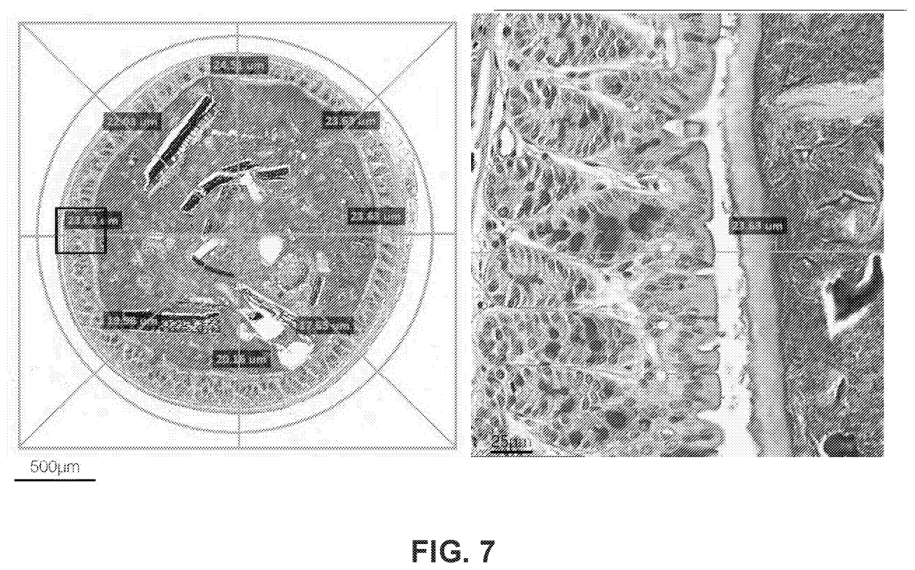

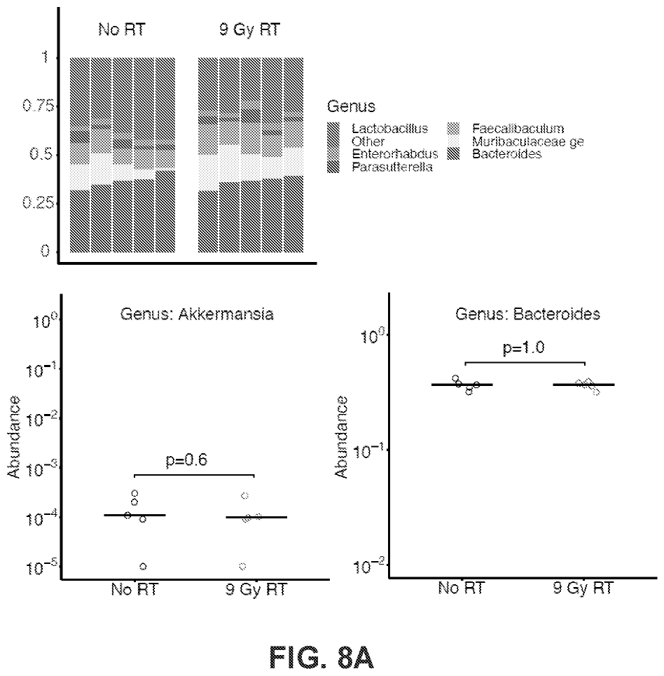

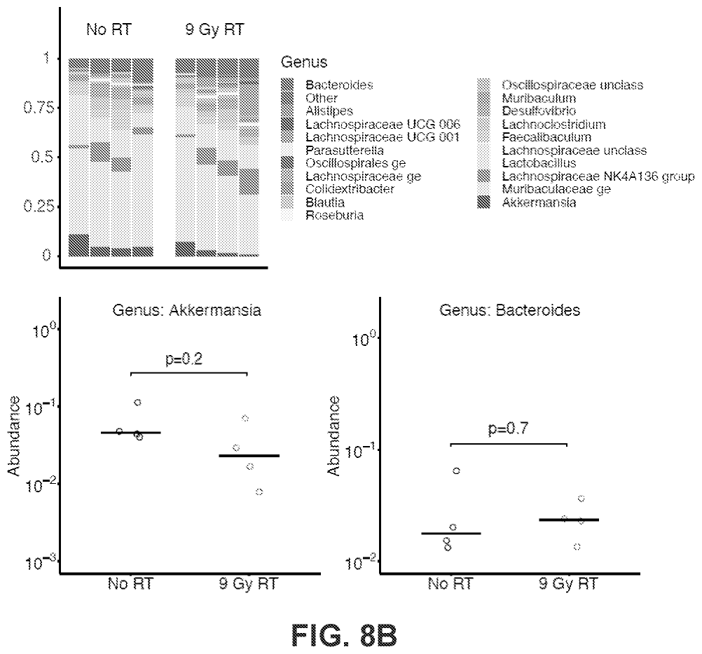

To investigate why the intestinal microbiome appeared to be impacted similarly in response to different cytotoxic therapies, the direct effects of RT on intestinal bacterial composition were evaluated by irradiating mouse fecal pellets and cultivating bacteria on agar plates. All bacterial colonies that grew were swabbed, and taxonomical composition of the swabbed colonies was evaluated by 16S rRNA gene sequencing. Exposure to irradiation resulted in no enrichment for Akkermansia or Bacteroides, though Akkermansia abundance was low due to its relatively slow growth rate in vitro (FIG. 8A). Bacteria from irradiated fecal pellets were also introduced orally to mice previously treated with an oral decontaminating antibiotic cocktail. Exposure to irradiation had no discernible effect on the composition of bacterial populations, including no enrichment for Akkermansia or Bacteroides (FIG. 8B).

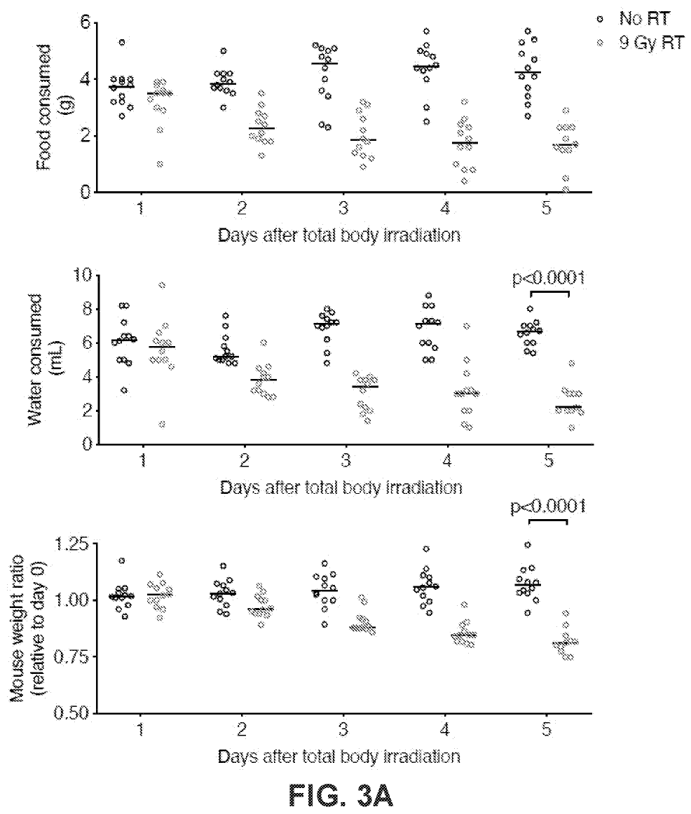

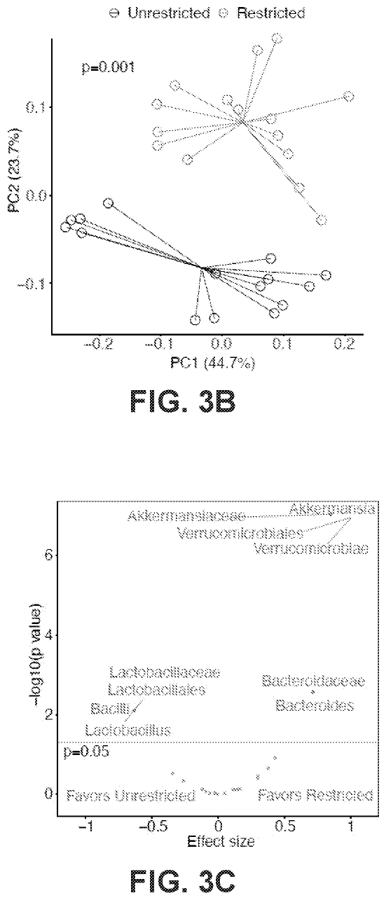

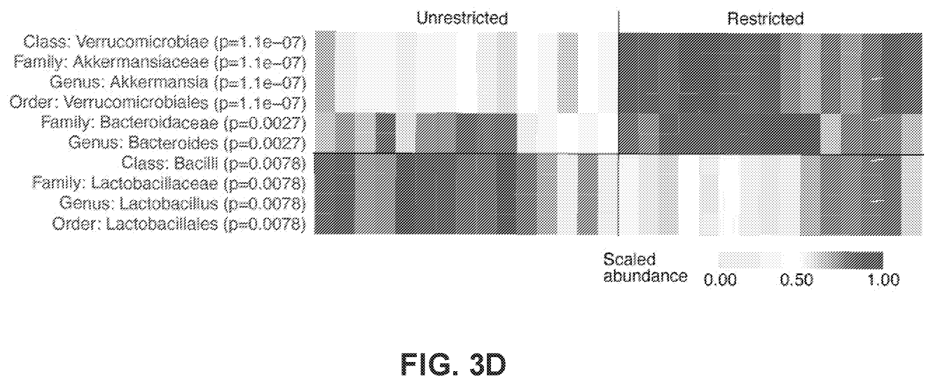

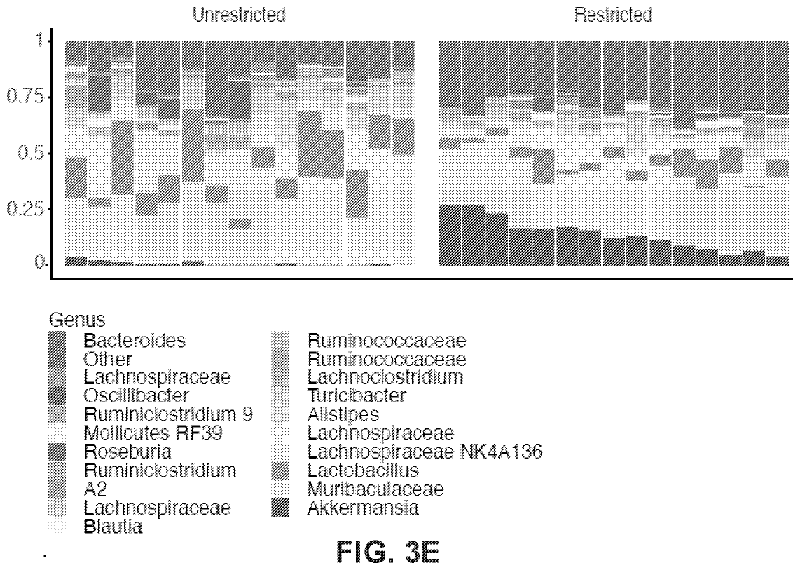

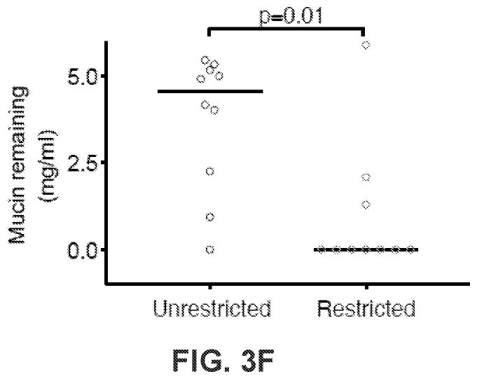

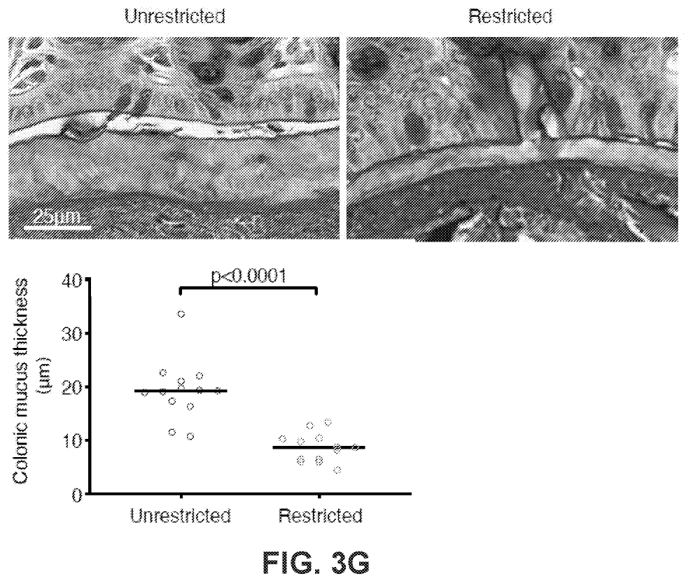

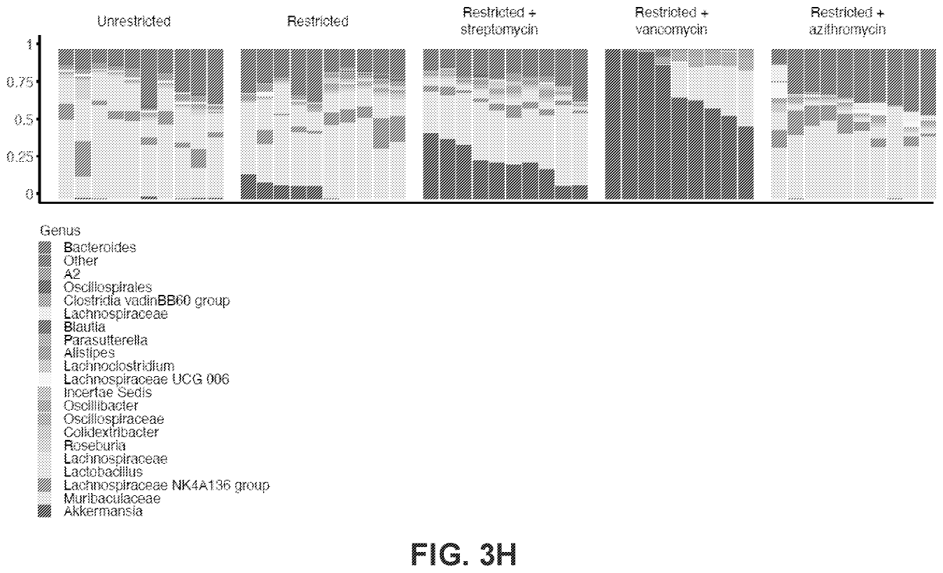

Diet is a major determinant of intestinal microbiome composition, and the indirect impact of RT on microbiome composition due to a reduction in intake of food in mice was investigated. Mice were individually housed in metabolic cages following RT to quantify effects of RT on food and water intake. Within 2 days following RT, mice had reduced their oral intake to approximately 2 grams a day, or a 50% reduction (FIG. 3A). To evaluate whether dietary restriction (DR) could impact intestinal microbiome composition and the colonic mucus layer, normal, un-irradiated mice were limited 2 grams of chow per mouse per day for 7 days. DR resulted in marked changes in the microbiome, characterized primarily by expansion of Akkermansia and, to a lesser extent, an expansion of Bacteroides and loss of Bacilli (FIGS. 3B-3E). Mucin glycan degradation was more robust after DR (FIG. 3F), and the colonic mucus layer was also significantly thinner (FIG. 3G).

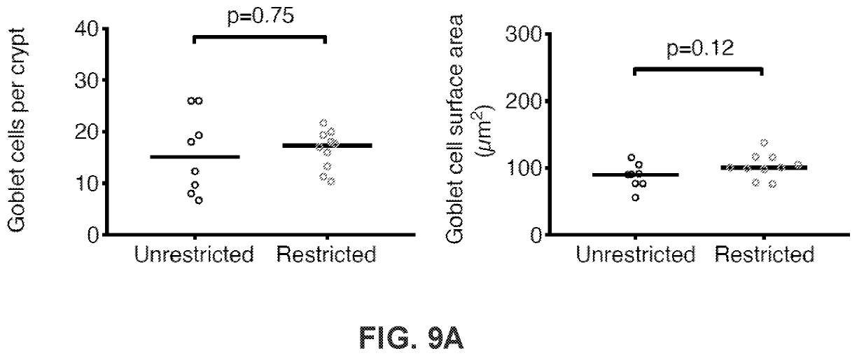

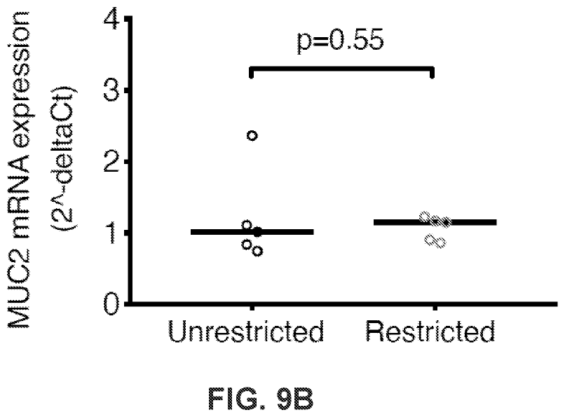

To evaluate if DR was impacting mucin production, goblet cells, specialized epithelial cells that are the primary producers of mucin in the colon, were evaluated. Neither the number of goblet cells per crypt nor the combined surface area of goblet cells in a cross section of colonic tissue were impacted by DR (FIG. 9A). RNA expression of the gene encoding the predominant mucin in the small and large intestine, Muc2, was also not significantly changed in colonic tissue homogenates from mice following DR (FIG. 9B). Altogether, these results indicated that, in some embodiments, a reduction in oral nutrition may be sufficient to produce a thinner colonic mucus layer. Without wishing to be bound by theory, such a reduction in the mucus layer may be due to an increase in mucin degradation leading to increased consumption of mucin, since the production of mucin appeared to be intact.

Because commercially-available mice lacking Akkermansia are not easy to find (12), narrow-spectrum antibiotics were used to evaluate the specific contribution of Akkermansia to mucus thinning during DR. Streptomycin depleted certain gram-positive bacteria, vancomycin depleted both gram-positive bacteria and some Bacteroidetes, and azithromycin depleted Akkermansia as well as some gram positive populations (FIG. 3H). Neither streptomycin nor vancomycin had a significant effect on mucus barrier loss, while azithromycin treatment prevented thinning of the colonic mucus layer (FIG. 3I), indicating that, in some embodiments, Akkermansia is correlated with increased mucolysis during dietary restriction.

### D. Bacterial Metabolites Link Dietary Restriction to Mucolytic Bacteria

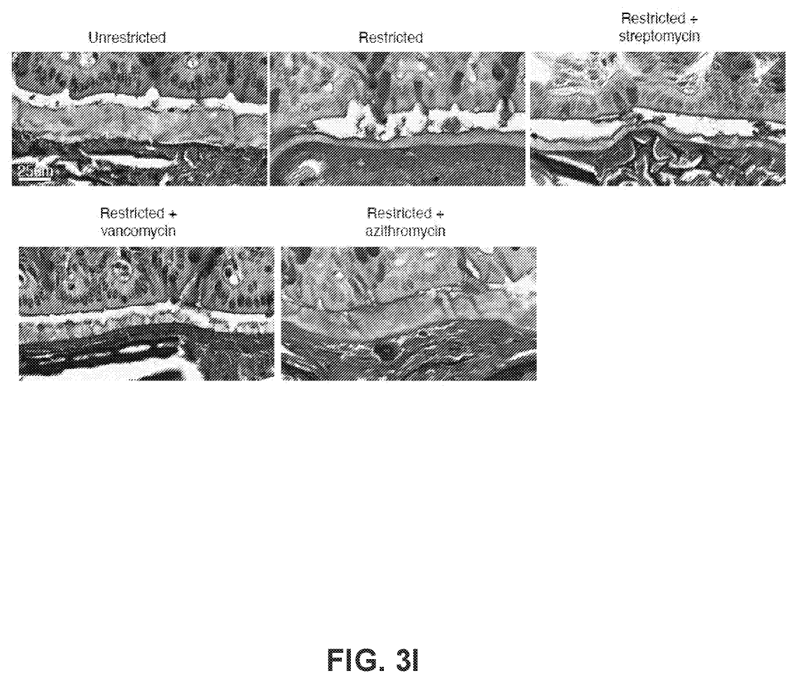

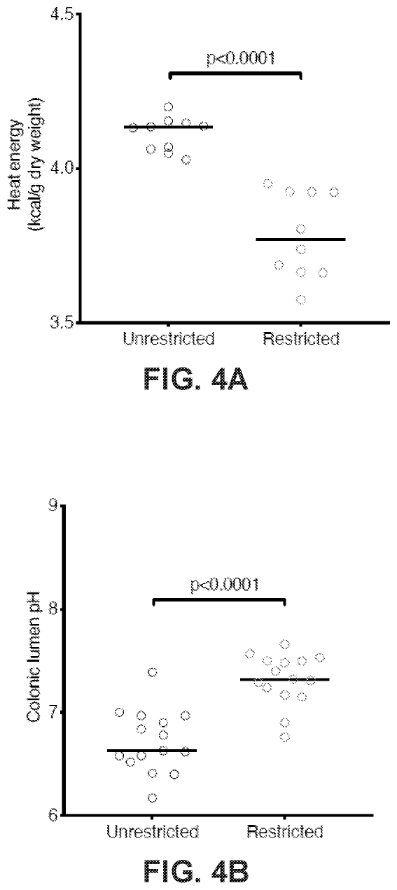

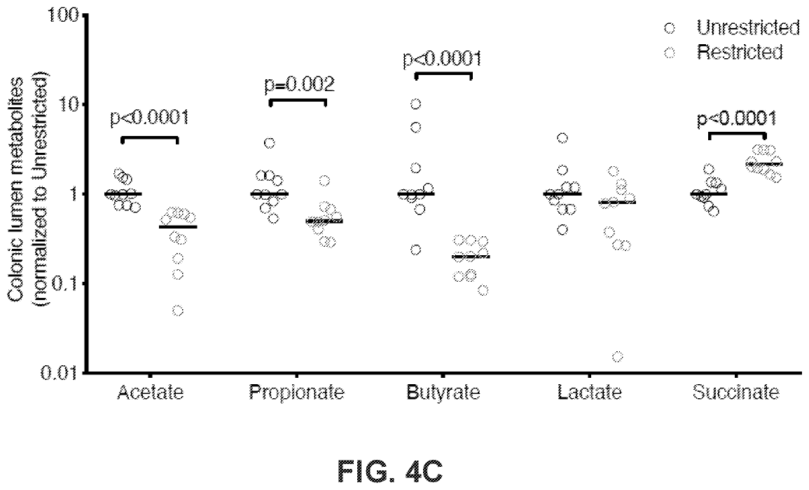

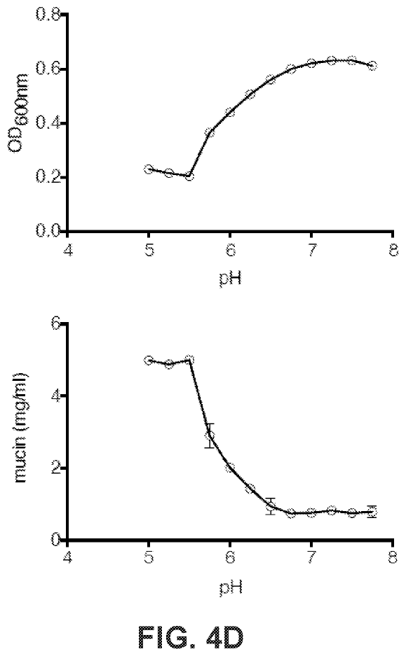

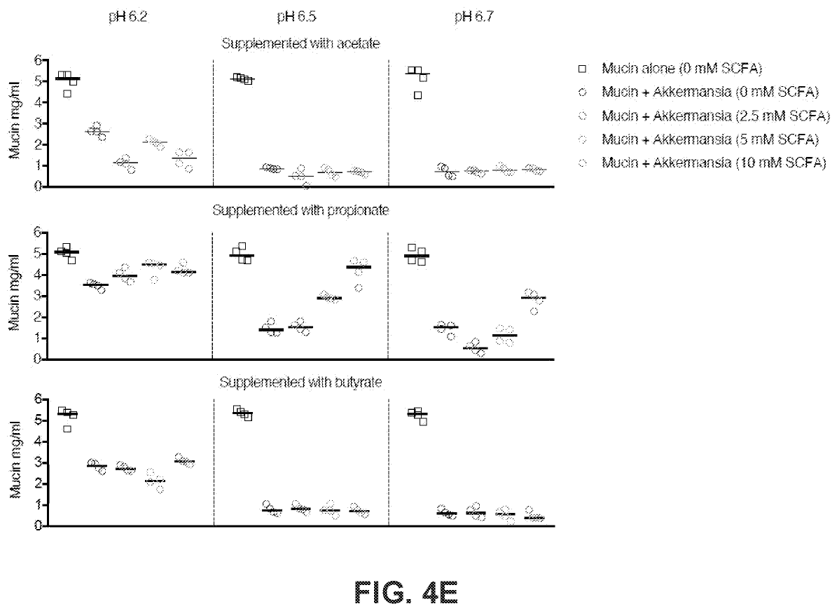

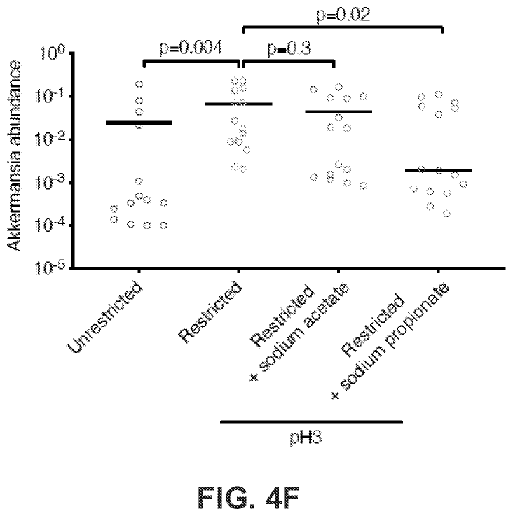

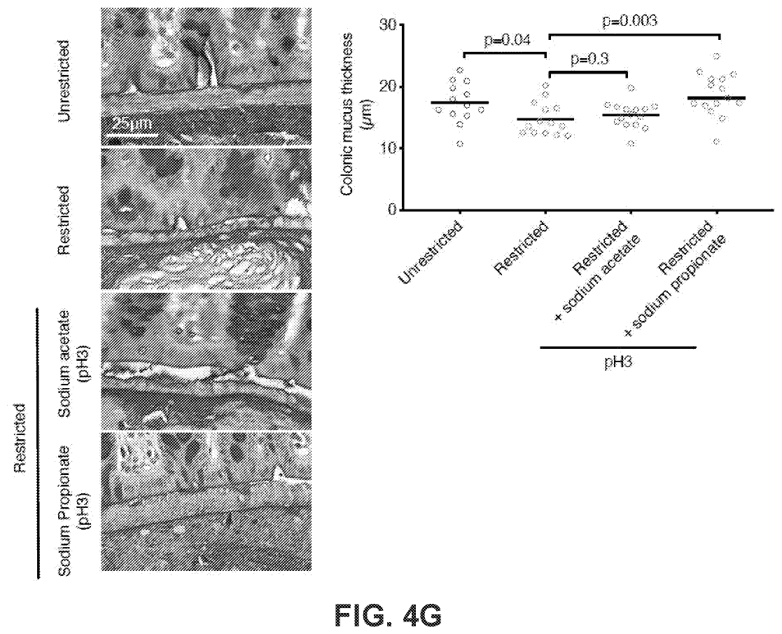

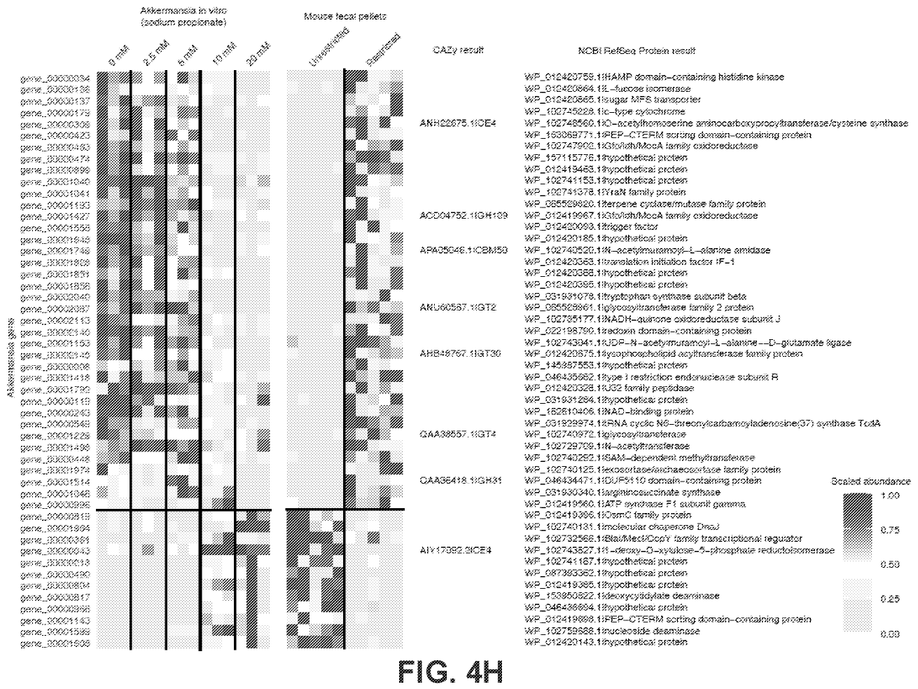

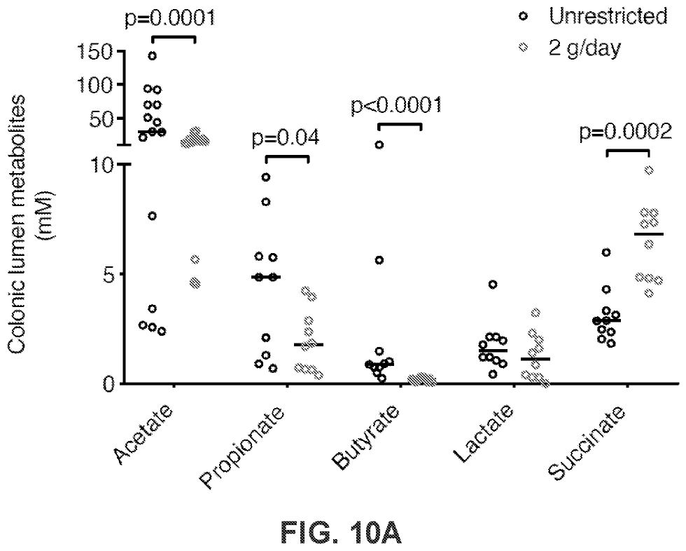

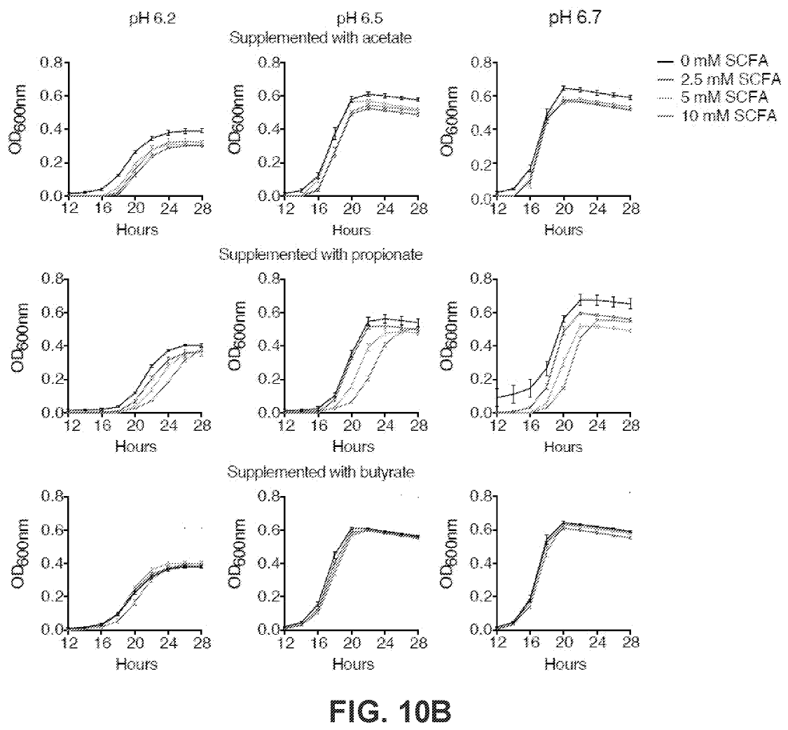

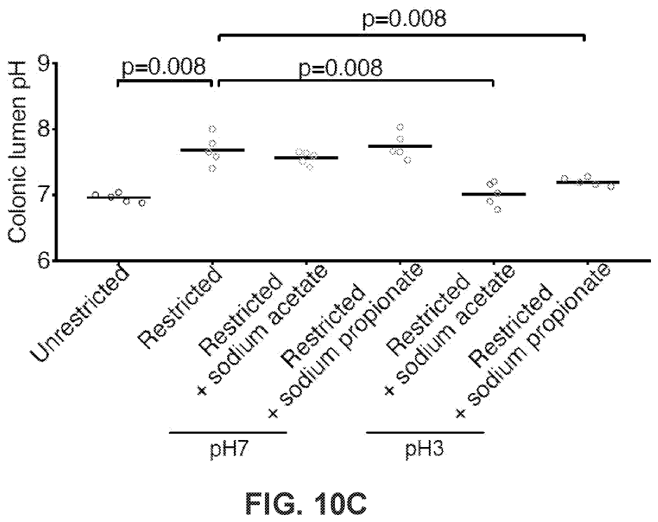

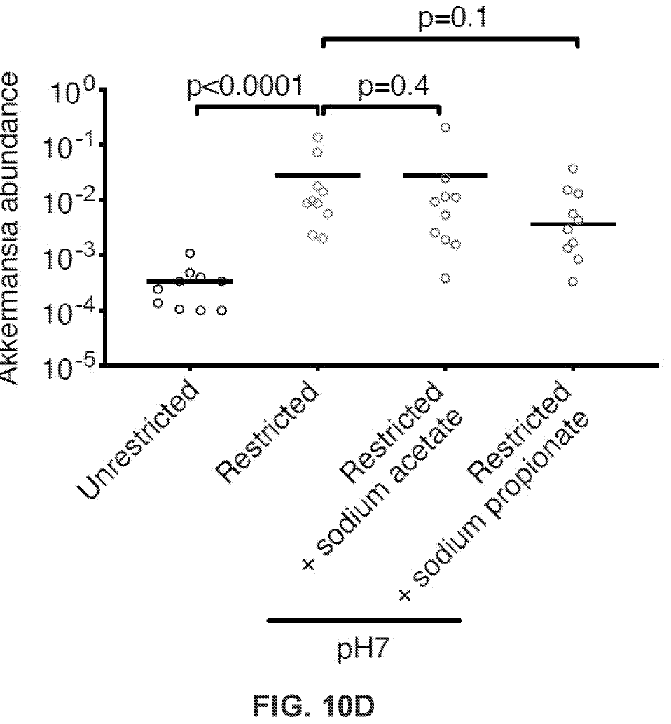

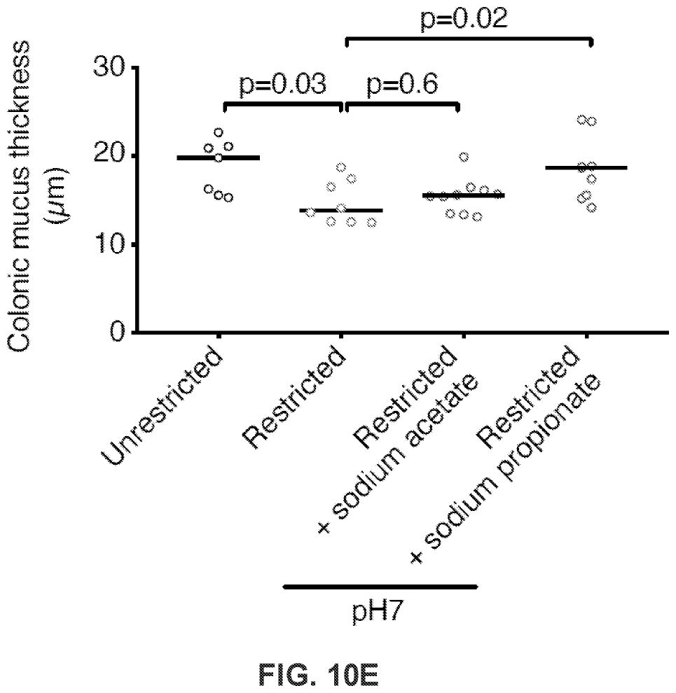

To identify mechanistic links between diet and microbiome composition and whether reduced oral dietary intake perturbs normal commensal bacterial metabolism in the intestinal lumen, the impact of DR on metabolic substrates entering the colon was evaluated by bomb calorimetry on cecal contents of mice. DR mice were found to have less calories entering the colon (FIG. 4A). Among the most abundant products of intestinal bacterial metabolism are organic acids, and quantification of pH in the colonic lumen showed that DR resulted in a raised pH, indicating overall reduced metabolism (FIG. 4B). To better characterize this rise in pH, specific bacterial metabolites were quantified using ion-chromatography mass spectrometry, and DR was found to lead to reduced concentrations of acetate, propionate and butyrate, and increased succinate (normalized results in FIG. 4C, raw results in FIG. 10A). Succinate is a metabolic precursor of propionate (13), suggesting that, in some embodiments, DR inhibits enzymatic conversion of succinate to propionate.

The impact of DR-induced metabolic changes in the colonic lumen on A. muciniphila behavior was also studied using an in vitro mucin glycan consumption assay. A novel A. muciniphila (MDA-JAX AM001) strain was isolated from the feces of C57BL6 mice and introduced to liquid media supplemented with PGM, and the effects of varying pH either alone or combined with the presence of physiological concentrations of acetate, propionate, and butyrate were evaluated. Progressively lowering the pH of bacterial media below 7 led to increased inhibition of A. muciniphila in terms of both growth and mucin glycan degradation (FIG. 4D), and higher levels of propionate had inhibitory effects on mucin glycan utilization by A. muciniphila (FIG. 4E) and also led to delays in growth (FIG. 10B), while acetate and butyrate each had negligible effects on A. muciniphila behavior.

Mice were supplemented during DR with acidified sodium propionate in the drinking water to investigate the combination of acidity and propionate on suppression of A. muciniphila growth in vivo. Such supplementation reduced fecal pH (FIG. 10C), mitigated expansion of Akkermansia (FIG. 4F) and prevented thinning of the mucus layer (FIG. 4G). Similar treatment with acidified sodium acetate, despite lowering the pH in the colonic lumen, had no such preventative effect (FIGS. 4F-4G), while drinking water with sodium propionate at neutral pH was sufficient to prevent mucus thinning and trended towards preventing Akkermansia expansion (FIGS. 10D-10E). Altogether, these results indicate that, in some embodiments, a reduced level of propionate following DR and a higher pH together can support increased mucolytic activity.

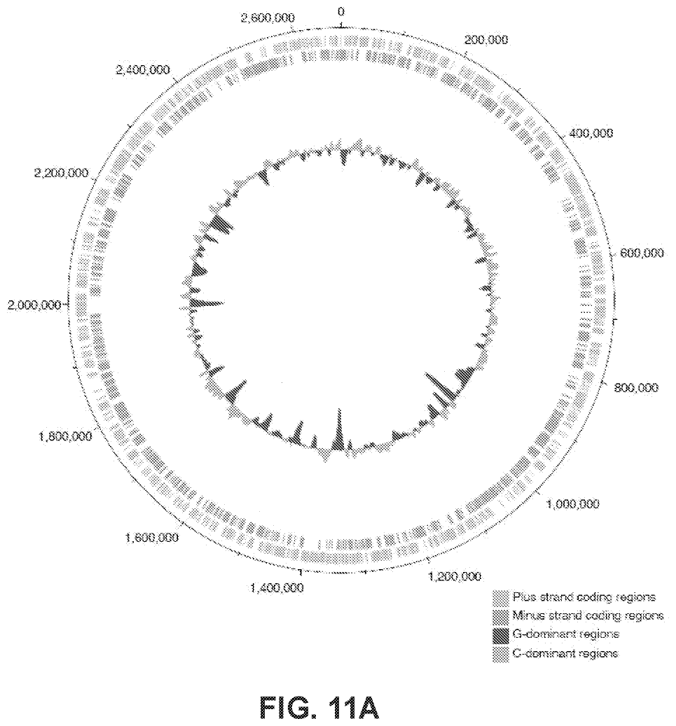

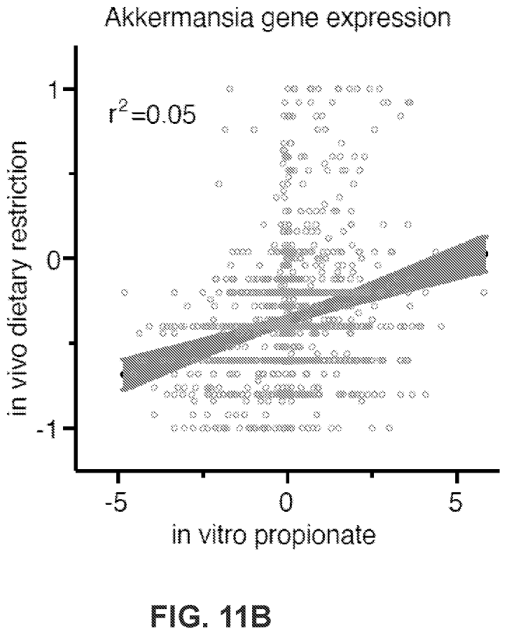

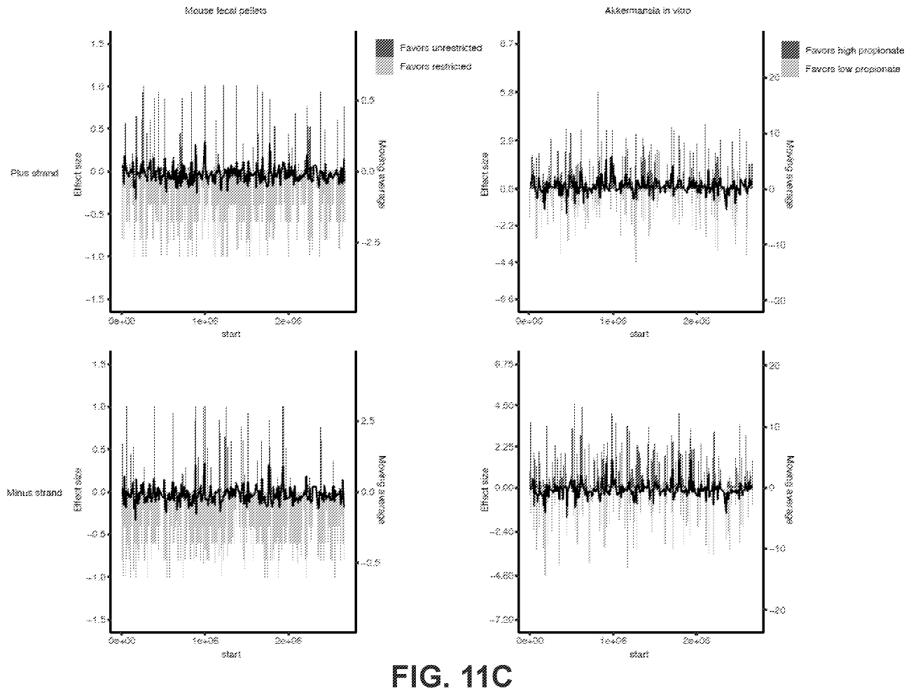

To explore how DR and propionate can modulate growth and mucin utilization by A. muciniphila, the A. muciniphila transcriptome was profiled under various conditions in vivo and in vitro. The circularized genomic sequence of the murine A. muciniphila isolate (MDA-JAX AM001) was determined, and identified 1935 putative proteins (FIG. 11A) were identified. RNA sequencing was performed on stool samples from mice, as well as A. muciniphila in vitro, and 186 genes were determined to be modulated by DR in vivo (Mann-Whitney U test unadjusted <0.05), while 392 genes were determined to be modulated by propionate in vitro (Kruskal-Wallis unadjusted <0.05). Evaluating the correlation of effect sizes in these two settings, propionate-related effects explained changes seen in DR at a proportion of 0.05, though the slope was significantly non-zero (p<0.0001, FIG. 11B). Fifty genes were identified that concordantly changed in vivo during DR and in vitro in low concentrations of propionate (FIG. 4H), and representations of gene expression with respect to the genome are depicted in FIG. 11C).

Mucins are glycoproteins predominately capped by fucose and sialic acid residues at their branching terminals. Upregulated genes in the settings of DR and low propionate included L-fucose isomerase which interconverts fucose and fucolose, as well as a member of the glycosyl hydrolase enzyme family 109, which have been shown to cleave oligosaccharide chains on glycoproteins and glycolipids found on the surface of erythrocytes that determine ABO blood types (14). Also upregulated was a member of the Idh/MocA family of oxidoreductases, which can play a part in sialic acid utilization (15). In contrast, some of the genes upregulated in the settings of an unrestricted diet or high propionate include enzymes critical for producing nucleotides, such as deoxycytidylate deaminase and nucleoside deaminase, indicating a relative downregulation of carbohydrate utilization genes relative to housekeeping functions such as synthesizing DNA and RNA components.

### E. Strategies Targeting Mucolytic Bacteria in Mice Receiving RT Preserve Colonic Mucus, Reduce Hypothermia, and Reduce Colonic Inflammation

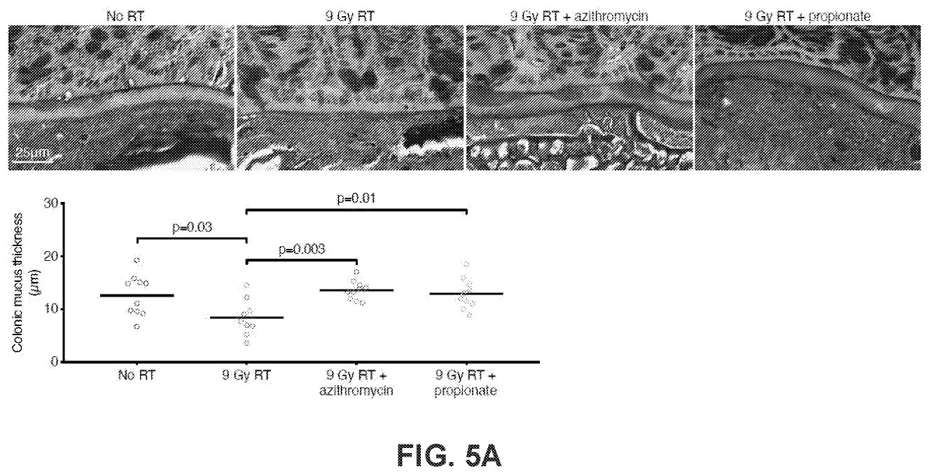

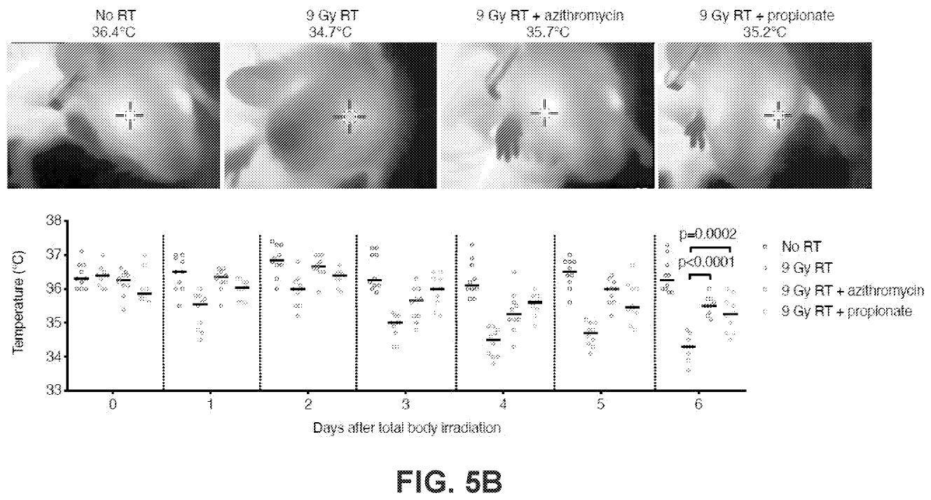

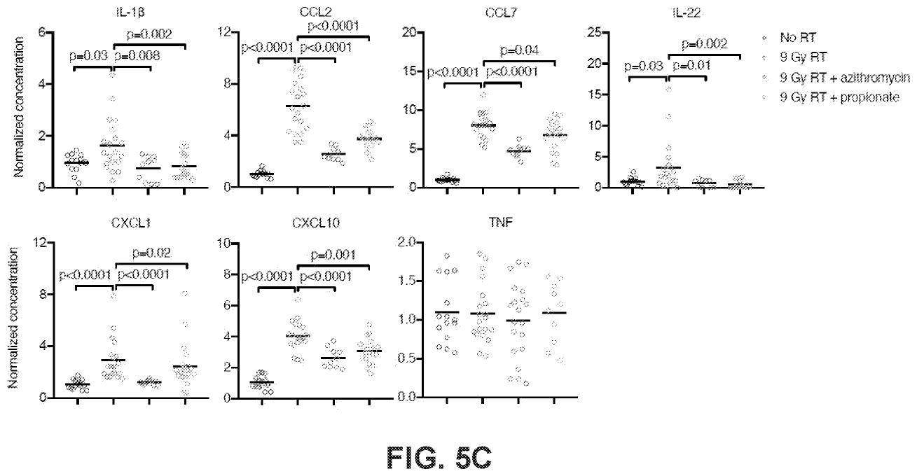

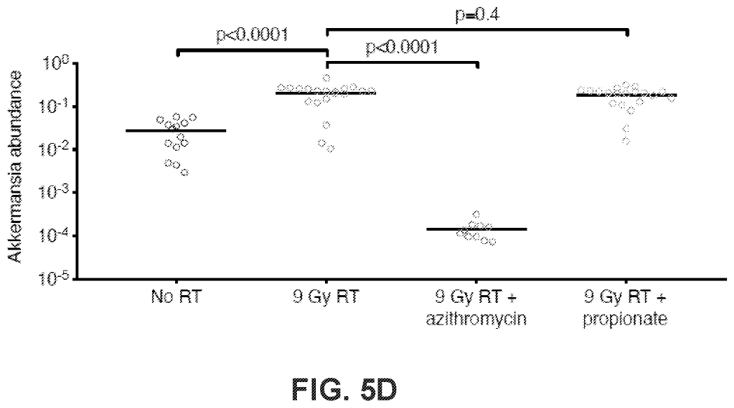

Finally, the two strategies identified to be effective in preventing loss of colonic mucin during dietary restriction, azithromycin or propionate, were also tested for their ability to reduce systemic inflammation following cytotoxic therapy. The addition of azithromycin or propionate to the total body RT model was tested, and each strategy was found to be effective in preserving colonic mucus layer thickness (FIG. 5A). Additionally, ocular surface temperatures were non-invasively measured in mice using a published method (16) because in contrast to humans, mice are develop hypothermia in response to exposure to inflammatory ligands such as LPS (17) and in models of sepsis (18). Total body RT produced significant hypothermia detectable as early as 1 day following RT, with temperatures of irradiated mice further decreasing over the next several days, while the temperatures of irradiated mice supplemented with azithromycin or propionate were less depressed at day 6 (FIG. 5B). These results suggested that, in some embodiments, strategies to inhibit mucolytic activity of A. muciniphila can be effective at reducing inflammation in irradiated mice.

The degree of inflammation in colonic tissue was directed measured by quantifying levels of a panel of cytokines (FIG. 5C). IL-1b, CCL2, CCL7, IL-22, CXCL1, and CXCL10 were all elevated in colonic tissues of mice following RT, but were reduced with the addition of azithromycin treatment. Propionate treatment also prevented elevation of all of these cytokines, with the exception of CXCL1. No elevation of TNF was observed following RT, nor were effects of azithromycin or propionate on TNF levels. Corroborating a less complete suppression of colonic inflammation by propionate, azithromycin was very effective at preventing outgrowth of Akkermansia in mice following RT, while propionate was not effective (FIG. 5D). Altogether, results from interventional experiments in mice following RT indicated that, in some embodiments, azithromycin and/or propionate therapy can be effective at eliminating intestinal Akkermansia, preserving colonic mucus, and preventing colonic inflammation and hypothermia.

### F. Levels of Vitamin B12 are an Important Determinant of Propionate Levels

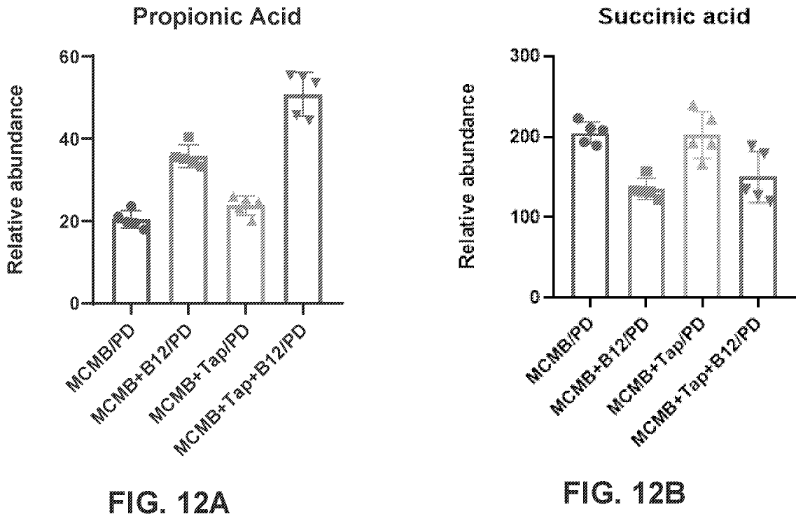

Parabacteroides distasonis (PD) coupled with a metabolic substrate, tapioca starch (Tap), was also found to increase propionate levels (FIG. 12). The relative abundance of short-chain fatty acids, propionate and succinate, from Parabacteroides distasonis (PD)-grown cell-free culture supernatant (CFCS) was quantified using ion chromatography-mass spectrometry (IC-MS). PD was grown in modified chopped meat broth (MCMB) with/without tapioca for three days anaerobically, and cell-free culture supernatant was filter-sterilized through 0.22-um filters and stored at −30° C. until metabolite analysis.

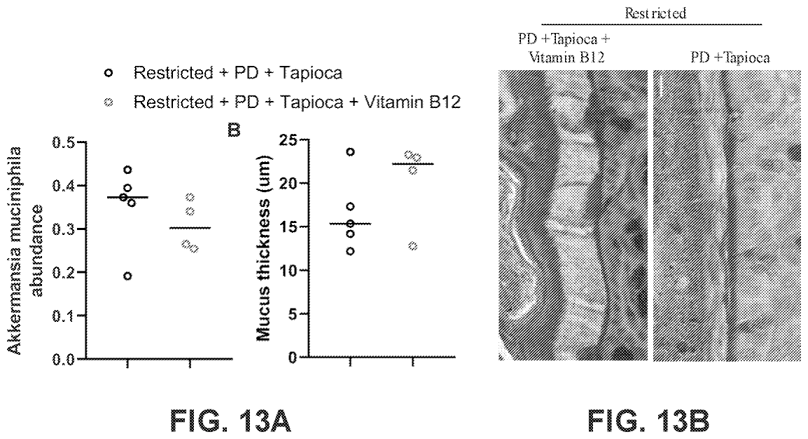

While propionate levels were increased, a related metabolite, succinate, was even more elevated. Succinate is a propionate precursor and can be converted to propionate by an enzymatic pathway that depends on vitamin B12 as a cofactor (Louis, P. and H. J. Flint, Environ Microbiol, 2017. 19(1): p. 29-41), and a higher level of succinate has been detected in the culture of PD (Wang K et al., Cell Reports 2019; 26, 222-235). Thus, the growth of PD with non-digestible carbohydrates (tapioca) was also tested in the presence of vitamin B12 to increase the production of propionate levels in the CFCS and to assess the inhibitory effect on mucolytic bacteria. As shown in FIG. 13, oral administration of Parabacteroides distasonis (PD) and Tapioca starch together with vitamin B12 reduced Akkermansia expansion and prevented thinning of colonic mucus during dietary restriction.

The impact of the addition of B12 to PD and tapioca starch in vitro (FIG. 12) and in vivo (FIG. 13) indicates that, in some embodiments, strategies to augment propionate in the intestinal lumen that depend on bacterial fermentation to produce propionate may benefit by the addition of providing vitamin B12, and that delivery of B12 to the intestinal lumen may provide a benefit in patients at risk for neutropenic fever or intestinal inflammation.

### G. Identification of Additional Compounds to Inhibit Akkermansia Growth

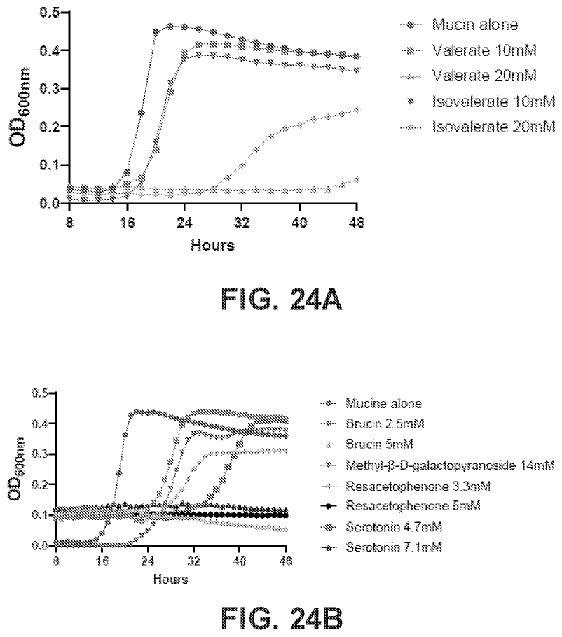

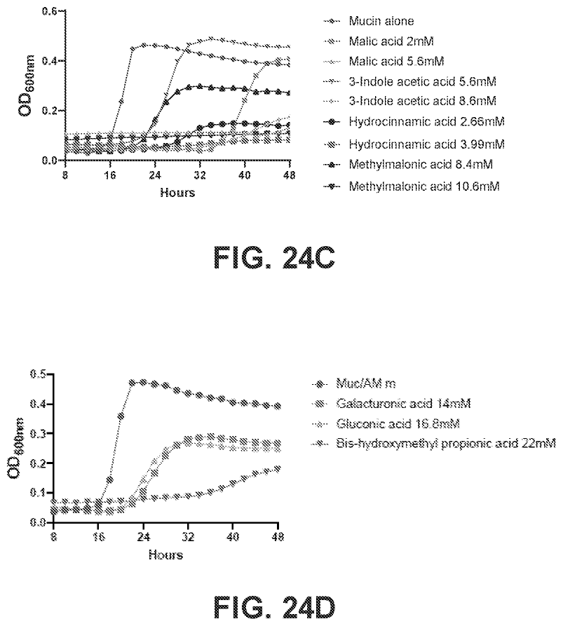

In addition to propionate, acetate, and butyrate, two concentrations of the SCFAs isovalerate and valerate were tested for their ability to delay and/or suppress growth of the novel A. muciniphila strain (MDA-JAX AM001) isolated from the feces of C57BL6 mice. SCFAs were added in 10 and 20 mM final concentration to bacterial culture. Growth of the bacteria at 37° C. in BYEM10+ mucin medium was read at OD600 nm continuously up to 48 hrs. Control Akkermansia culture was grown in absence of compounds. Both 10 mM and 20 mM isovalerate and valerate were found to suppress and/or delay MDA-JAX AM001 growth compared to a mucin-only control (FIG. 24A).

Using a high-throughput screening strategy analyzing ˜1800 compounds, various other natural compounds in addition to SCFAs were identified for their potential to delay and/or suppress growth of MDA-JAX AM001. Several compounds identified by the screen were tested, including Brucine (2.5 mM and 5 mM), Methyl-β-D-galactopyranoside (14 mM), Resacetophenone (3.3 mM and 5 mM), and Serotonin (4.7 mM and 7.1 mM) and were found to delay and suppress the growth of MDA-JAX AM001 (FIG. 24B). Growth of the bacteria at 37° C. in BYEM10+ mucin medium was read at OD600 nm continuously up to 48 hrs. Control Akkermansia culture was grown in absence of compounds.

Several active ruminal metabolites were also tested for their ability to delay and/or suppress growth of MDA-JAX AM001. Compounds were selected based on initial testing of Akkermansia growth in medium containing 1× to 3× concentrated ruminal fluid, followed by analysis of metabolite content by MALDI-TOF mass-spectrometry. Ruminal metabolite compounds tested included Malic acid (2 mM and 5.6 mM), 3-Indole acetic acid (5.6 mM and 8.6 mM), Hydrocinnamic acid (2.66 mM and 3.99 mM), Methylmalonic acid (8.4 mM and 10.6 mM), Glucoranic acid (16.8 mM), Galacturonic acid (14 mM), and Bis-hydroxy methyl propionic acid (22 mM). These ruminal metabolite compounds have also been found in human stool, as analyzed by MALDI-TOF mass spectrometry. Each of these compounds were found to delay and inhibit MDA-JAX AM001 growth (FIGS. 24C, 24D). Growth of the bacteria at 37° C. in BYEM10+ mucin medium was read at OD600 nm continuously up to 48 hrs. Control Akkermansia culture was grown in absence of compounds.

### Example 2

### Mucus-Degrading Bacteroides Link Carbapenem Antibiotics to Aggravated Graft-Versus-Host Disease

A. Meropenem Treatment During Allo-HSCT is Associated with Increased Intestinal GVHD in Both Allo-HSCT Patients and Mice

Retrospective clinical studies have found that allo-HSCT patients receiving antibiotics with activity against commensal anaerobes was associated with increased GVHD (25-28). In some cases, institutional guidelines recommend first-line therapy for neutropenic fever with cefepime, an antibiotic that is relatively sparing of commensal anaerobes, while second-line therapy is with meropenem, which is highly active against commensal anaerobes. In some cases, the use of meropenem is associated with a difference in incidence of intestinal GVHD.

In this study, data from 295 patients with acute myeloid leukemia (AML) or myelodysplastic syndrome (MDS) who underwent allo-HSCT with tacrolimus and methotrexate as GVHD prophylaxis following conditioning therapy with fludarabine and 5 busulfan from 2011 to 2016 were retrospectively examined. The incidence of acute intestinal GVHD until day 100 after transplant was evaluated and compared were patients who received neither cefepime nor meropenem, those who received cefepime alone, those who received meropenem alone, and those who received both cefepime and meropenem during the period from days −10 to 30 relative to allo-HSCT infusion. A summary of clinical characteristics is provided in Table 2; there were no significant differences in major clinical transplant parameters between each antibiotic-exposure group.

TABLE 2
Patient characteristics of allo-HSCT patients
who examined incidence of intestinal GVHD.

| | None | | Cefepime | | Meropenem | | Both | | |
|---|---|---|---|---|---|---|---|---|---|
| | (n = 80) | | (n = 124) | | (n = 32) | | (n = 59) | | P value |
| Median age (range), y | 57 | (20-72) | 55 | (18-77) | 57 | (25-72) | 54 | (19-74) | 0.8355 |
| Male, n (%) | 45 | (56%) | 69 | (57%) | 18 | (56%) | 40 | (68%) | 0.4332 |
| Donor type, n (%) | | | | | | | | | 0.0741 |
| MRD | 44 | (55%) | 59 | (48%) | 14 | (44%) | 14 | (24%) | |
| MUD | 33 | (41%) | 62 | (50%) | 18 | (56%) | 43 | (73%) | |
| MMRD | 2 | (3%) | 0 | (0%) | 0 | (0%) | 0 | (0%) | |
| MMUD | 1 | (1%) | 3 | (2%) | 0 | (0%) | 2 | (3%) | |
| Cell source | | | | | | | | | 0.2637 |
| Bone marrow | 21 | (26%) | 35 | (28%) | 5 | (16%) | 22 | (37%) | |
| Peripheral blood | 59 | (74%) | 89 | (72%) | 27 | (84%) | 37 | (63%) | |
| Conditioning, n (%) | | | | | | | | | 0.6650 |
| Myeloablative | 78 | (98%) | 118 | (95%) | 31 | (97%) | 54 | (92%) | |
| Non-myeloablative | 2 | (3%) | 6 | (5%) | 1 | (3%) | 5 | (9%) | |
| Acute GVHD | | | | | | | | | 0.9746 |
| Grade 0 | 33 | (41%) | 57 | (46%) | 13 | (41%) | 22 | (37%) | |
| Grade I | 18 | (23%) | 26 | (21%) | 5 | (16%) | 10 | (17%) | |
| Grade II | 23 | (29%) | 34 | (27%) | 10 | (31%) | 21 | (36%) | |
| Grade III-IV | 6 | (8%) | 7 | (6%) | 4 | (13%) | 6 | (10%) | |

Continuous variables are presented as the median and range, while categorical variables are presented as number and percentages. Non-repeated ANOVA was used to compare continuous variables, while chi-square or Fisher's exact tests were used to analyze the frequency distribution between categorical variables. P-values under 0.05 were considered to be statistically significant. MRD, HLA-matched related donor; MUD, HLA-matched unrelated donor; MMRD, HLA-mismatched related donor; MMUD, HLA-mismatched unrelated donor.

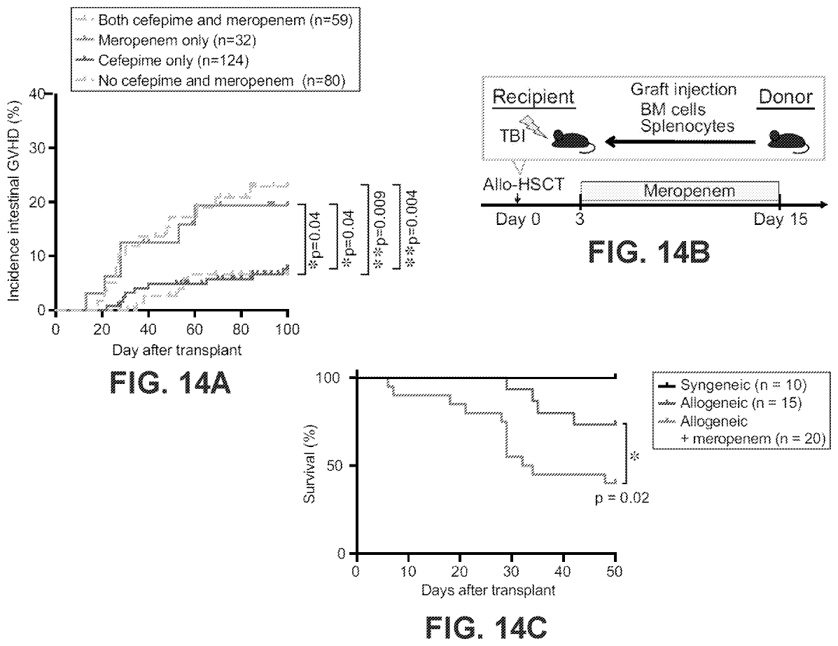

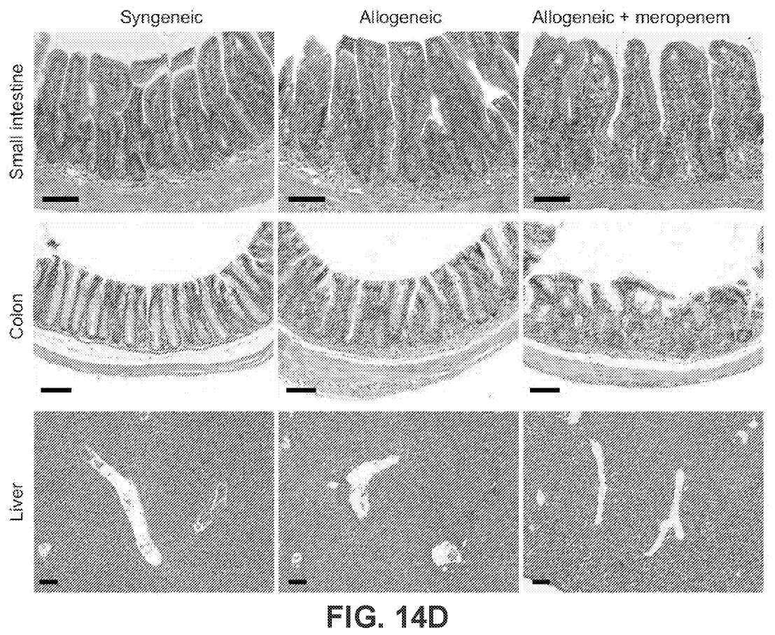

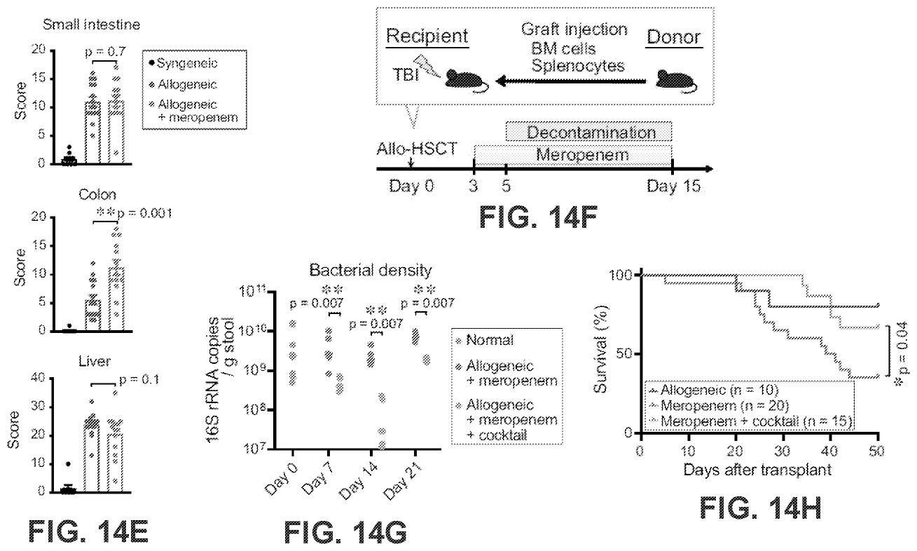

However, the incidence of acute intestinal GVHD was significantly higher in patients who received either meropenem alone or both cefepime and meropenem, while the acute intestinal GVHD incidence in patients receiving only cefepime was similar to that of patients who received neither antibiotic (FIG. 14A). These results indicated that, in some embodiments, meropenem exposure in allo-HSCT patients is associated with an increased incidence of acute intestinal GVHD, consistent with previous reports (25-28), and thus, in some embodiments, meropenem may aggravate GVHD by modulating the intestinal microbiota.

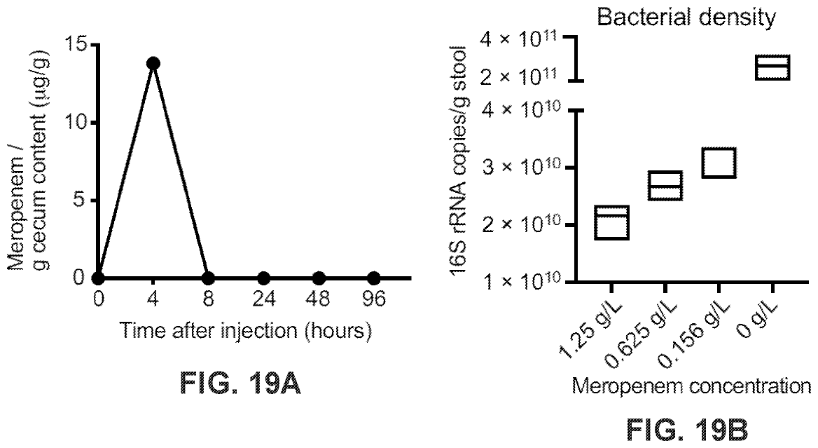

To further explore a potential causal relationship between meropenem and intestinal GVHD, the effects of meropenem on GVHD in a mouse model of allo-HSCT were examined. A method of administering meropenem to mice was first developed that would better mimic the effects of meropenem on the intestinal microbiome of patients, which is typically given every 8 hours intravenously. Previously, antibiotics were administered subcutaneously to mice (25), and meropenem concentrations were quantified in the cecal lumen of mice 4, 8, 24, 48 and 96 hours after subcutaneous injection. Subcutaneous injection produced an increase in meropenem concentrations in the cecum at 4 hours after injection, which rapidly declined thereafter (FIG. 19A). Next, drinking water was evaluated as a means of continuously administering meropenem to mice, using bacterial density quantified by 16S ribosomal RNA (16S rRNA) gene quantitative PCR (qPCR) to gauge reductions of the intestinal microbiota. A concentration of 0.625 g/L of meropenem was selected as an experimental drinking water dose, which produced a detectable meropenem concentration in fecal samples (median±SE, 0.11±0.07 μg/g stool) and a substantial one-log reduction in fecal bacterial density (FIG. 19B).

Lethally irradiated B6D2F1 (H-2b/d) mice were intravenously injected with 5×10⁶ bone marrow (BM) cells and 5×10⁶ splenocytes from major histocompatibility complex (MHC)-mismatched B6 (H-2b) or syngeneic donors on day 0. Meropenem was additionally administered to allo-HSCT recipient mice in drinking water from days 3 to 15 relative to allo-HSCT infusion (FIG. 14B). Mice treated with meropenem after allo-HSCT had significantly worsened survival (FIG. 14C), with severe epithelial damage in the colon (FIG. 14D) and significantly higher GVHD histological scores in the colon compared to control allo-HSCT recipient mice (FIG. 14E). In contrast, GVHD histology in the small intestine and liver were not substantially different in mice treated with meropenem (FIG. 14D, FIG. 14E). Thus, in some embodiments, not only does meropenem aggravate GVHD in mice, but it does so in a manner that is localized to the large intestine.

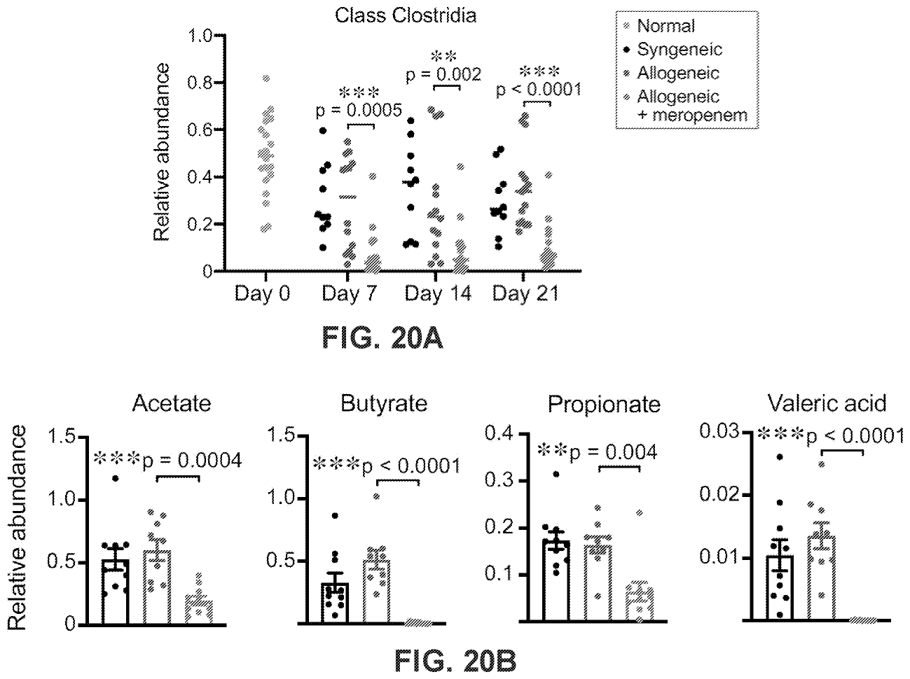

In some cases, meropenem-induced intestinal dysbiosis contributes to aggravated GVHD via loss of beneficial commensal bacteria. In some embodiments, meropenem-treated allogeneic mice show a loss of Clostridia abundance and a reduction in SCFA levels, including butyrate. On day 7, or 4 days following the start of meropenem treatment, Clostridia were depleted in meropenem-treated mice and remained so at 6 days after stopping 5 meropenem (FIG. 20A). These mice also had reduced fecal levels of SCFAs, with particularly dramatic reductions in butyrate and valerate (FIG. 20B).

To determine whether loss of Clostridia during meropenem treatment is mechanistically sufficient to aggravate intestinal GVHD, the effects of intestinal microbiome decontamination in meropenem-treated mice with GVHD were examined. In addition to meropenem, an oral cocktail of piperacillin/tazobactam and nystatin was administered in drinking water from days 5 to 15 after transplantation (FIG. 14F), similar to an oral decontamination regimen given to pediatric allo-HSCT patients (32). Bacterial density was significantly reduced by this decontamination cocktail (FIG. 14G). Intestinal decontamination improved survival in meropenem-treated allo-HSCT recipient mice (FIG. 14H), suggesting that in some embodiments, meropenem treatment leads to worsened GVHD not only due to depletion of beneficial bacteria, but also via expansion of pro-inflammatory bacteria.

### B. Meropenem Treatment During Allo-HSCT Results in Loss of Clostridia and Expansion of Bacteroides in Both Patients and Mice

Next, the effects of meropenem on the composition of the colonic microbiota of transplanted patients and mice were investigated. Fecal specimens were collected from HSCT patients transplanted from 2014 to 2019 and 44 patients were identified who were treated with allo-HSCT following conditioning with fludarabine and busulfan. Of these, 26 patients did not receive meropenem treatment between days −10 to 14 while 18 patients did receive meropenem treatment. A comparison of clinical characteristics demonstrated no significant differences in major clinical transplant parameters between these groups as shown in Table 3.

TABLE 3
Patient characteristics of allo-HSCT patients
who underwent intestinal microbiome profiling.

| | Meropenem-untreated | | Meropenem-treated | | |
|---|---|---|---|---|---|
| | (n = 26) | | (n = 18) | | P value |
| Median age (range), y | 53 | (39-72) | 58 | (38-70) | 0.0573 |
| Male, n (%) | 14 | (53%) | 8 | (44%) | 0.7591 |
| Disease, n (%) | | | | | 0.8961 |
| AML | 11 | (42%) | 9 | (50%) | |
| MDS | 4 | (15%) | 4 | (22%) | |
| MPD | 6 | (23%) | 3 | (16%) | |
| CML | 4 | (15%) | 0 | (0%) | |
| ALL | 0 | (0%) | 1 | (5%) | |
| Others | 1 | (3%) | 1 | (5%) | |
| Donor type, n (%) | | | | | 0.7327 |
| MRD | 8 | (30%) | 4 | (22%) | |
| MUD | 18 | (69%) | 14 | (77%) | |
| Cell source | | | | | 0.4390 |
| Bone marrow | 6 | (23%) | 2 | (11%) | |
| Peripheral blood | 20 | (76%) | 16 | (88%) | |
| Conditioning, n (%) | | | | | 1.0000 |
| Myeloablative | 26 | (100%) | 18 | (100%) | |
| Non-myeloablative | 0 | (0%) | 0 | (0%) | |
| Acute GVHD | | | | | 0.5531 |
| Grade 0 | 9 | (34%) | 7 | (38%) | |
| Grade I | 4 | (15%) | 0 | (0%) | |
| Grade II | 11 | (42%) | 7 | (38%) | |
| Grade III-IV | 2 | (7%) | 4 | (22%) | |

Continuous variables are presented as the median and range, while categorical variables are presented as number and percentages. Non-repeated ANOVA was used to compare continuous variables, while chi-square or Fisher's exact tests were used to analyze the frequency distribution between categorical variables. P-values under 0.05 were considered to be statistically significant. ALL, acute lymphoid leukemia; AML, acute myeloid leukemia; CML, chronic myeloid leukemia; MPD, myeloproliferative disorder; MRD, HLA-matched related donor; MDS, myelodysplastic syndrome; MUD, HLA-matched unrelated donor; MMRD, HLA-mismatched related donor; MMUD, HLA-mismatched unrelated donor.

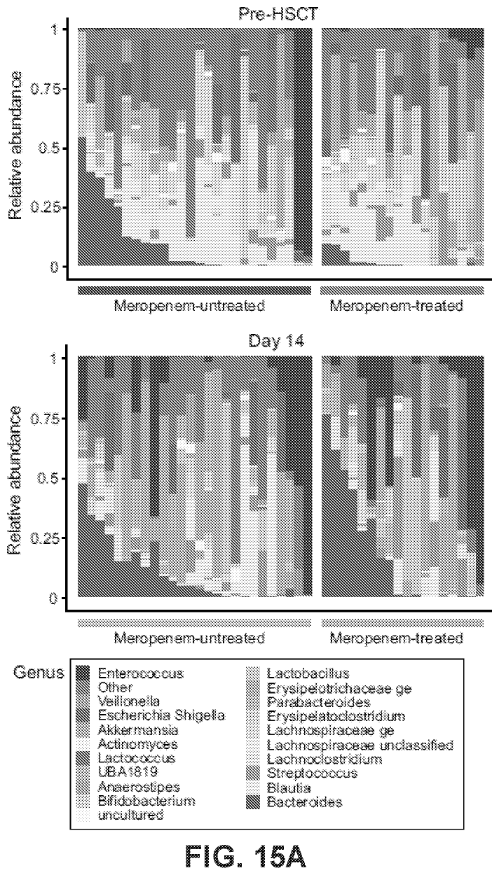

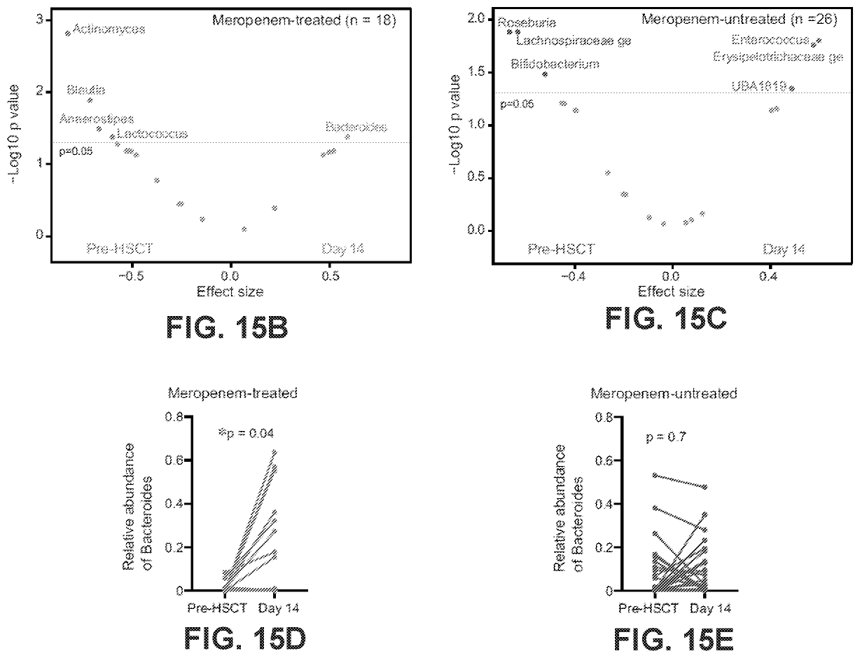

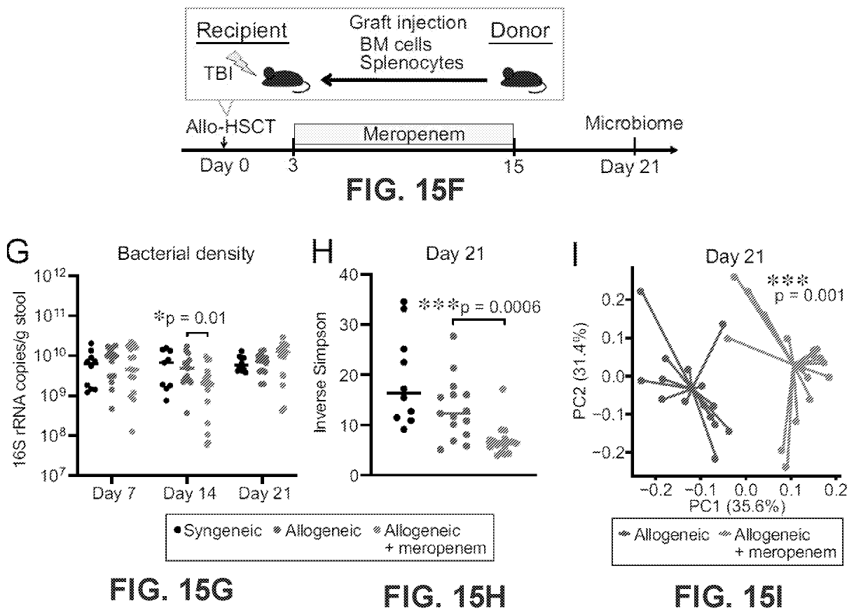

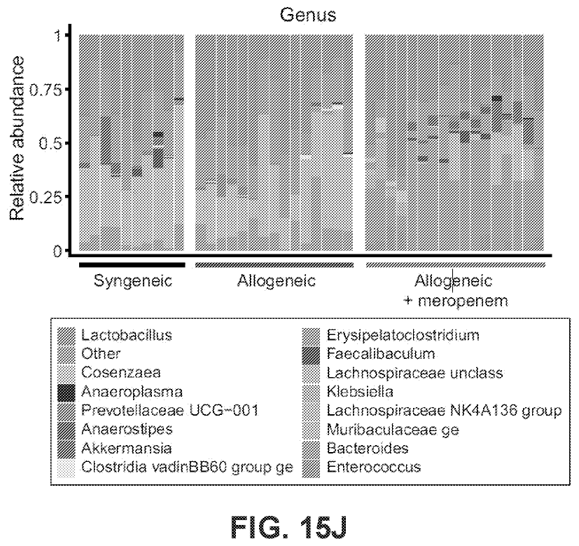

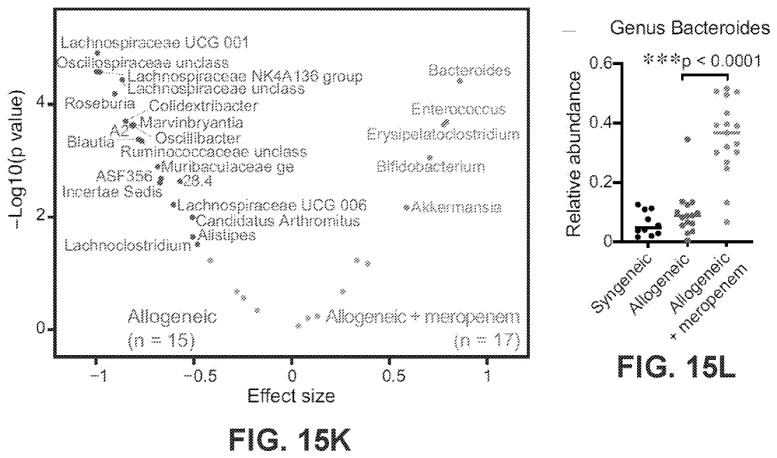

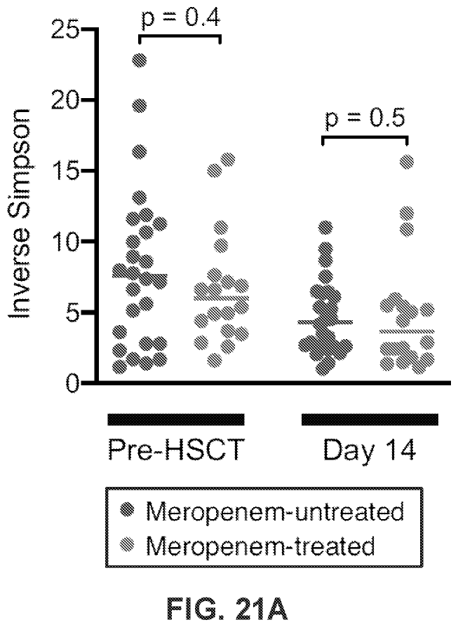

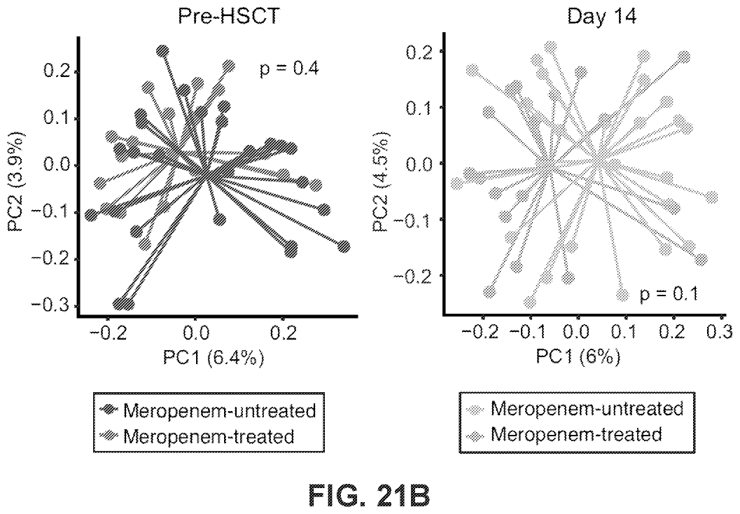

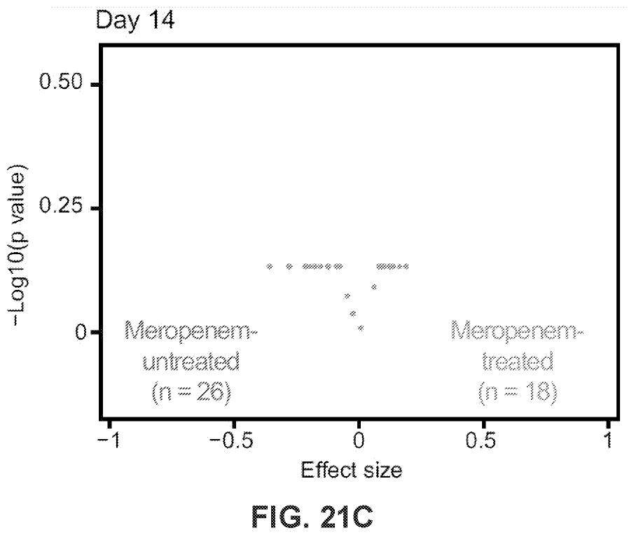

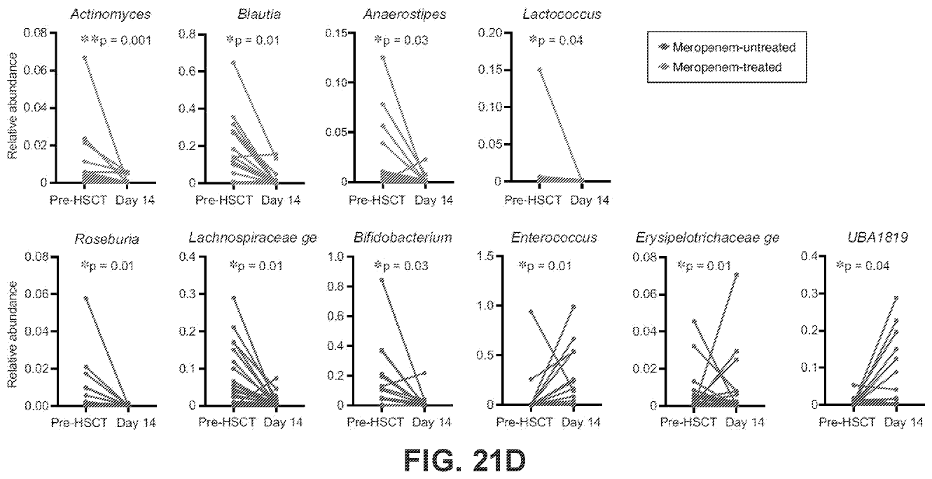

Fecal samples collected at baseline following hospital admission around day −7 as well as on day 14 following allo-HSCT were examined (FIG. 15A). Using PERMANOVA testing of weighted UniFrac beta diversity measures, baseline samples from meropenem-untreated and treated patients were not found to be significantly different, nor were samples on day 14 from meropenem-untreated and treated patients significantly different (FIGS. 21A-21C). A paired differential abundance analysis was then performed, asking which bacterial genera were the most changed from baseline in the two subgroups of patients. Using this approach, patients treated with meropenem were demonstrated to show significant expansion of bacteria from the genus Bacteroides (FIGS. 15A-15C and FIG. 21D). In contrast, patients not treated with meropenem showed expansion of the genus Enterococcus, a genus belonging to Erysipelotrichaceae, and the genus UBA1819 (within the Family Ruminococcaceae), but not Bacteroides (FIGS. 15A, 15C, 15D and FIG. 21D).

Effects of meropenem treatment on the composition of the intestinal microbiota of allo-HSCT mice were also examined (FIG. 15F). Using 16S rRNA gene qPCR, fecal bacterial densities were quantified and meropenem-treated mice were found to show significantly decreased bacterial density on day 14 after allo-HSCT during meropenem treatment, but by day 21, or 6 days after stopping meropenem, fecal bacterial densities had recovered to a level similar to that of untreated syngeneic and allogeneic mice (FIG. 15G). Most mice treated with meropenem began to succumb to aggravated GVHD approximately 3 weeks after allo-HSCT (FIG. 14C), and so the fecal microbiome was characterized on day 21 and alpha diversity, quantified using the inverse Simpson index, was significantly reduced in meropenem-treated mice (FIG. 15H). PERMANOVA testing demonstrated significant compositional differences between meropenem-untreated and treated mice (FIG. 15I). The most impacted bacterial genera in allo-HSCT mice were examined, and meropenem treatment was found to lead to significantly higher abundances of bacteria from several genera, including most substantially Bacteroides, as well as Enterococcus, Erysipelatoclostridium, Bifidobacterium and Akkermansia (FIGS. 15J-15L). Simultaneously, many genera were depleted, including Blautia, Lachnoclostridium, and other members of Lachnospiraceae, which belong to the class Clostridia (FIGS. 15J-15K).

### C. Bacteroides Thetaiotaomicron Contributes to Meropenem-Exacerbated Colonic GVHD in Mice

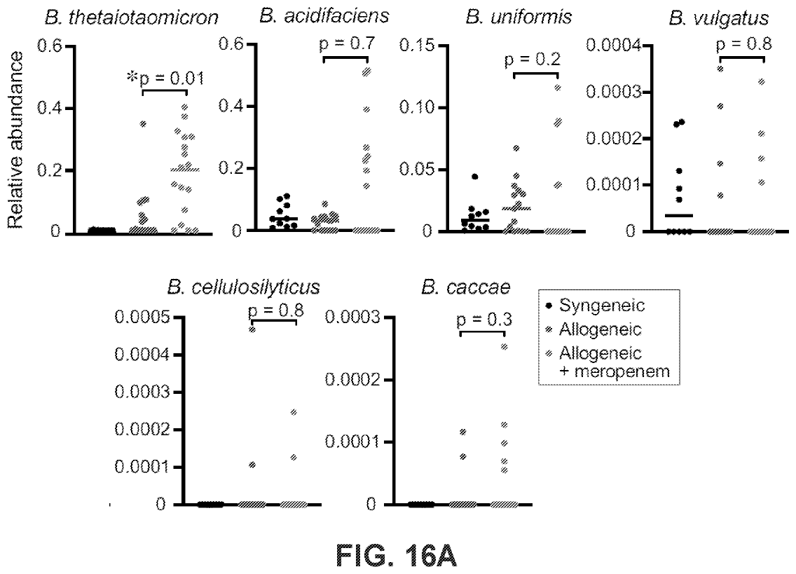

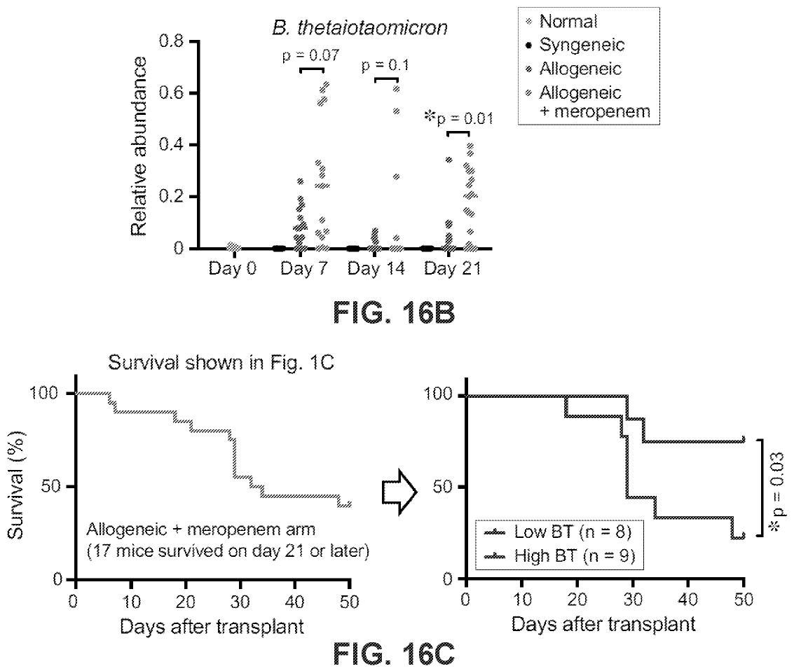

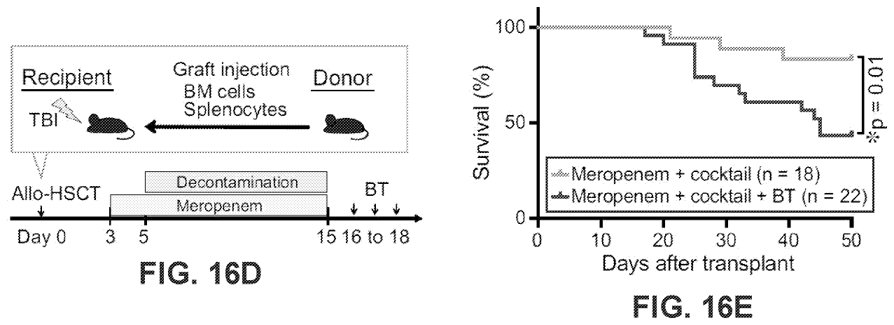

The effects of meropenem treatment on Bacteroides subsets was examined by sequencing the V4 region of the 16S rRNA gene (34). A single Bacteroides sequence variant was identified that was significantly expanded in meropenem-treated mice, and it had 100% identity with the 16S sequences of BT, Bacteroides faecis, and Bacteroides faecichinchillae, while other Bacteroides strains had 98.8% identity or less (FIG. 16A). The predominant murine Bacteroides isolate was isolated, and it was confirmed by whole genome sequencing that it was a strain of BT, with 97.4% genomic identity to the ATCC type strain of BT (ATCC 29148), and only 89.2% and 80.8% genomic identity to Bacteroides faecis and Bacteroides faecichinchillae, respectively. This isolate was named MDA-JAX BT001, or murine BT. Murine BT was suppressed by meropenem-supplemented drinking water but quickly expanded after cessation of meropenem therapy, in contrast to Clostridia which remained depleted (FIG. 16B and FIG. 20A). Thus, in some embodiments, murine BT is less sensitive to meropenem than Clostridia.

The minimum inhibitory concentration (MIC) of meropenem against murine BT was quantified using MIC test strips (Table 4).

TABLE 4
Quantification of the MIC of bacteria against meropenem.

| | MIC (μg/mL) |
|---|---|
| Bacteroides thetaiotaomicron (MDA-JAX BT001) | 4 |
| Bacteroides thetaiotaomicron (ATCC 29148) | 6 |
| Enterococcus faecalis (MDA-JAX EF001) | 12 |
| Clostridium disporicum (MDA-JAX CD001) | 0.094 |
| Clostridium saudiense (MDA-JAX CS001) | 0.38 |
| Lachnospiraceae unclassified (MDA-JAX LS001) | 0.38 |

Murine isolates of Enterococcus faecalis (MDA-JAX EF001), Clostridium disporicum (MDA-JAX CD001), Clostridium saudiense (MDA-JAX CS001), and Lachnospiraceae unclassified (MDA-JAX LS001) were also tested. Both mouse-derived and human-derived (ATCC 29148) BT strains showed only moderate sensitivity to meropenem with MICs of 4 μg/mL and 6 μg/mL, respectively. Mouse-derived Enterococcus faecalis was more resistant with a MIC of 12 μg/ml, while MICs of mouse-derived Clostridium disporicum, Clostridium saudiense, and Lachnospiraceae unclassified, which belong to the class Clostridia, had MICs of 0.094 μg/mL, 0.38 μg/mL, and 0.38 μg/mL, respectively, showing high sensitivity. Stool collected 48 hours after starting meropenem treatment in normal specific-pathogen-free (SPF) mice had a meropenem concentration of 0.11±0.07 μg/g, suggesting that, in some embodiments, BT is capable of surviving during meropenem treatment and then has a selective advantage after discontinuation of meropenem.

To further evaluate for an association between murine BT and aggravated GVHD in meropenem-treated mice, mice from 3 experiments shown in FIG. 14C were retrospectively stratified by their median relative abundance of BT. A comparison of these two cohorts showed that mice with higher abundances of BT had worsened overall survival (FIG. 16C). Next, the effects of murine BT on GVHD severity were studied experimentally. Meropenem-treated GVHD mice that had completed treatment with a decontamination cocktail were orally inoculated with 2×10⁷ colony-forming units (CFUs) of murine BT, and GVHD severity and survival was monitored (FIG. 16D). Mice administered murine BT showed worsened survival (FIG. 16E), indicating that, in some embodiments, murine BT was sufficient to aggravate GVHD in allo-HSCT mice that had been previously decontaminated.

D. Meropenem Treatment Induces Thinning of the Colonic Mucus Layer and Impairment of Epithelial Barrier Integrity in Mice with GVHD

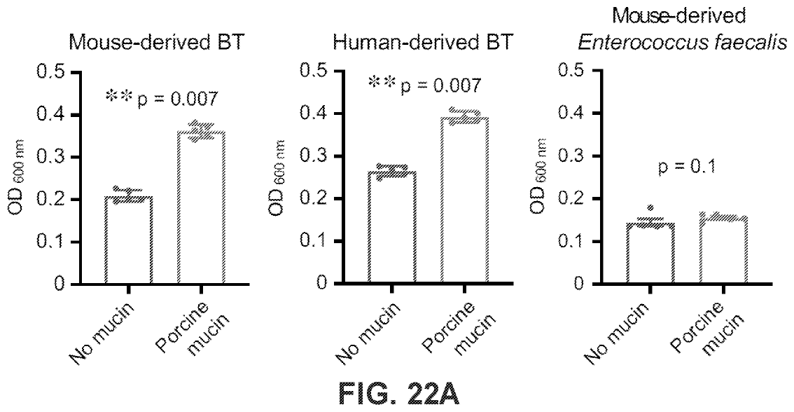

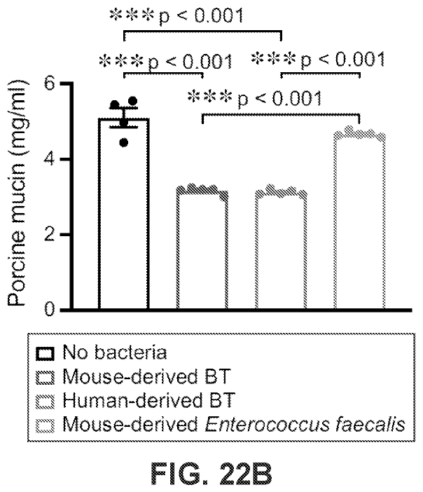

BT is a gram-negative obligate anaerobe with a broad ability to degrade dietary polysaccharides as well as host-derived glycans, including mucins (35, 36), and the effect of meropenem treatment during GVHD on the colonic mucus layer was investigated. The ability of murine BT isolate MDA-JAX BT001 to utilize mucin as a carbohydrate source was evaluated, as well as a human-derived BT strain (ATCC 29148) and, as a comparison, non-mucolytic mouse-derived Enterococcus faecalis (MDA-JAX 10 EF001). Supplementation of carbohydrate-poor media with porcine gastric mucin resulted in augmented growth of both BT strains, in contrast to no growth benefit for murine E. faecalis (FIG. 22A), indicating that mucin-utilization enzymes were expressed by both BT strains. Also quantified was degradation of mucin-derived carbohydrates using a PAS-based colorimetric assay, which confirmed that both BT strains displayed degradation of mucin-derived carbohydrates (FIG. 22B).

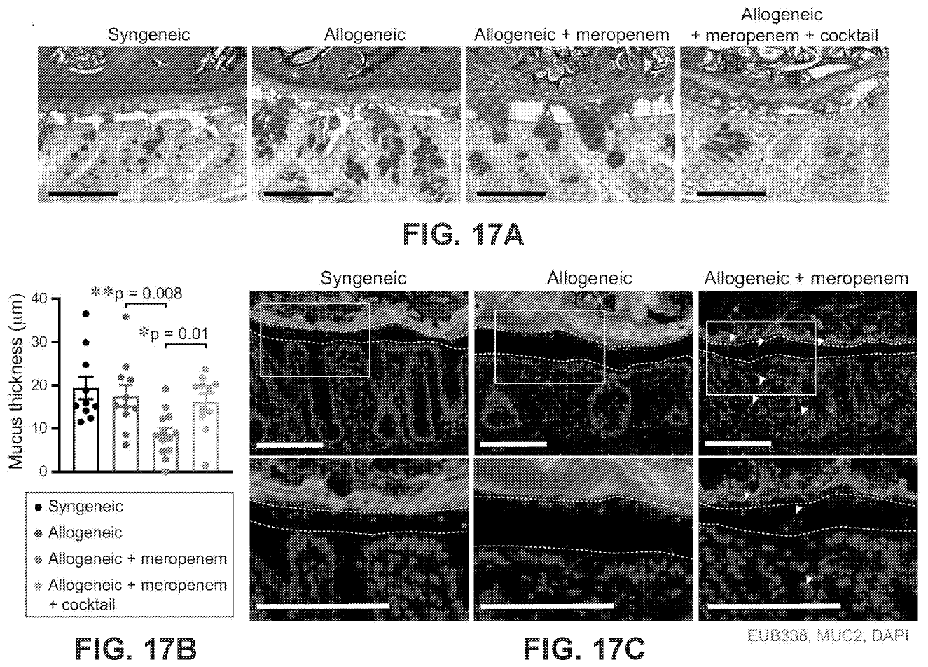

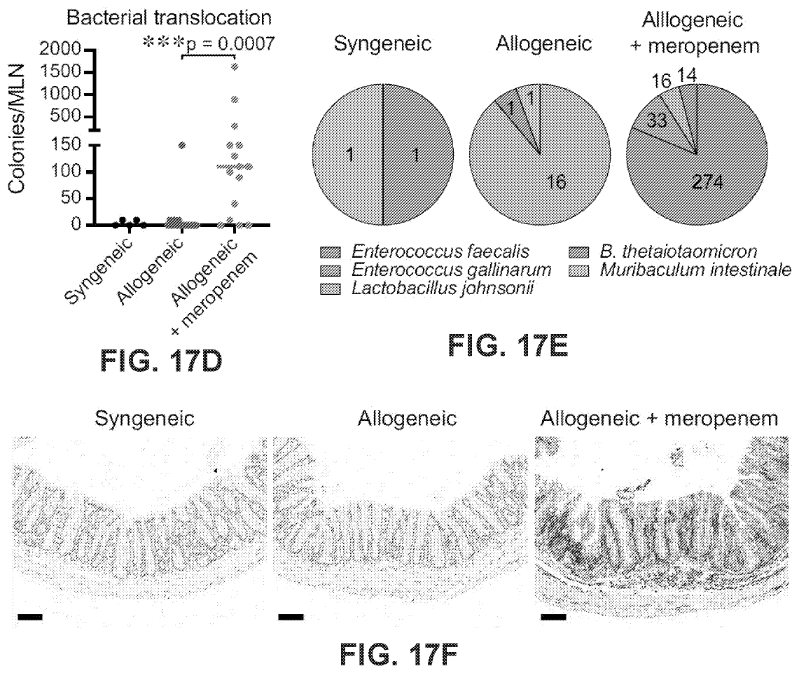

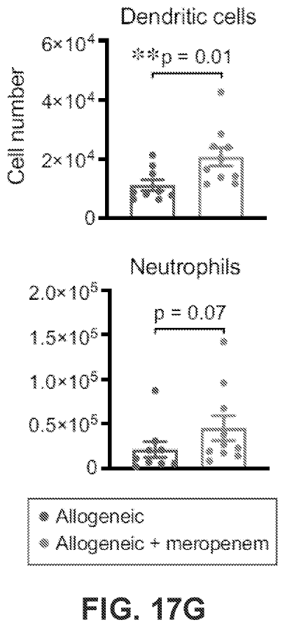

Next, assessed was the impact of meropenem treatment on the dense colonic mucus layer of mice with GVHD. PAS staining of histological colon sections demonstrated a significantly thinned colonic mucus layer in meropenem-treated mice compared to untreated GVHD mice and syngeneic mice (FIGS. 17A-17B). Decontamination, in contrast, led to preservation of the colonic mucus layer in meropenem-treated mice, suggesting that, in some embodiments, meropenem leads to increased mucus degradation by colonic bacteria (FIGS. 17A-17B).

The mucus layer provides a critical contribution to the intestinal barrier by excluding bacteria from the intestinal lumen adjacent to the intestinal epithelium. Histological sections were stained with 16S fluorescence in situ hybridization (FISH) probes, and dissemination of bacteria into the mucus layer and lamina propria of the colon in meropenem-treated mice with GVHD was visualized (FIG. 17C). To better quantify effects of meropenem on bacterial translocation, MLNs were cultivated microbiologically, and higher bacterial loads were found in meropenem-treated mice. Translocating bacteria included Enterococcus faecalis, Enterococcus gallinarum, Lactobacillus johnsonii, and BT (FIGS. 17D-17E).

Bacterial translocation has previously been found to aggravate GVHD via at least two mechanisms, including recruitment of neutrophils, which can compound tissue damage, as well as by enhancing antigen presentation by dendritic cells through activation of pathogen-associated molecular pattern signaling pathways (39, 40). In some embodiments, this barrier compromise leads to an inflammatory response in meropenem-treated mice, and observed marked colonic tissue infiltration by neutrophils and dendritic cells is observed (FIGS. 17F-17G). Altogether, these findings indicate that, in some cases, meropenem treatment leads to compromise of the colonic mucus layer, increased bacterial translocation and an aggravated inflammatory response.

### E. Meropenem Treatment During GVHD Upregulates In Vivo Expression of Mucus-Degrading Enzymes by BT

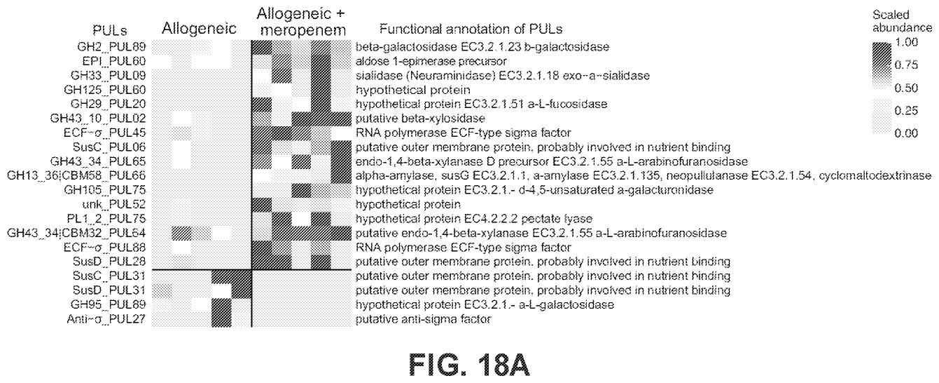

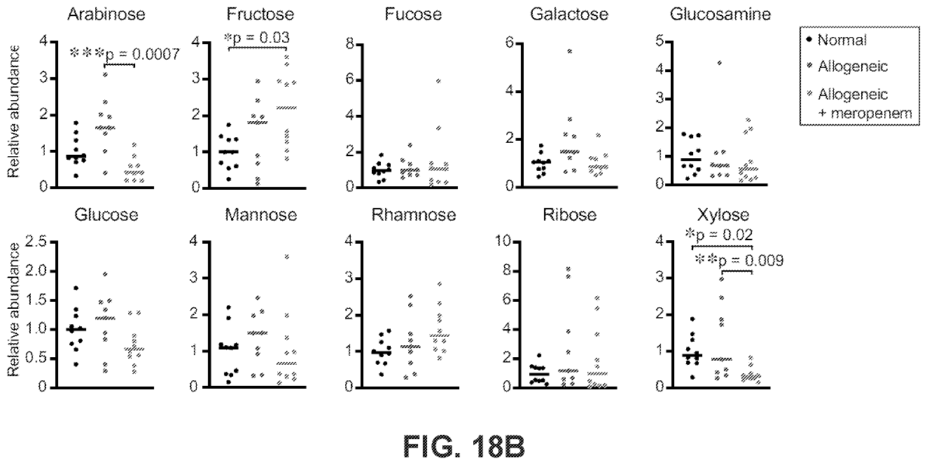

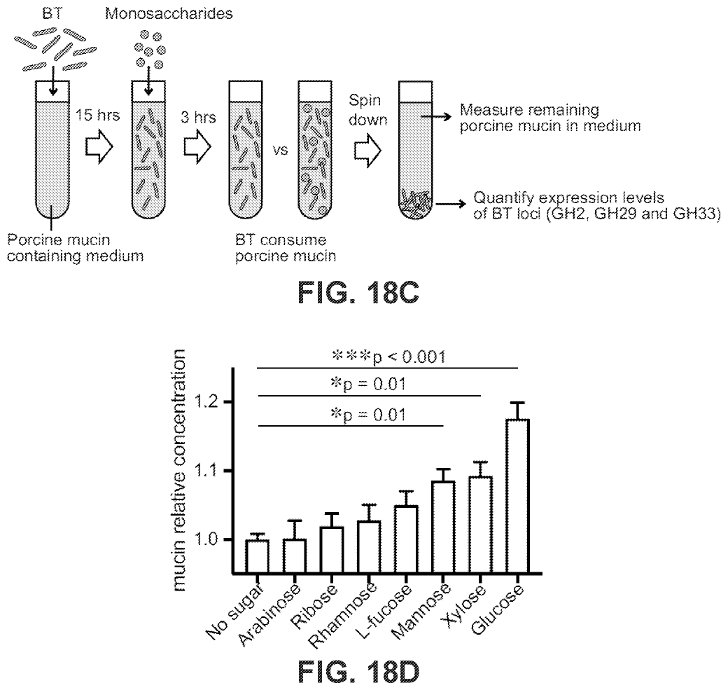

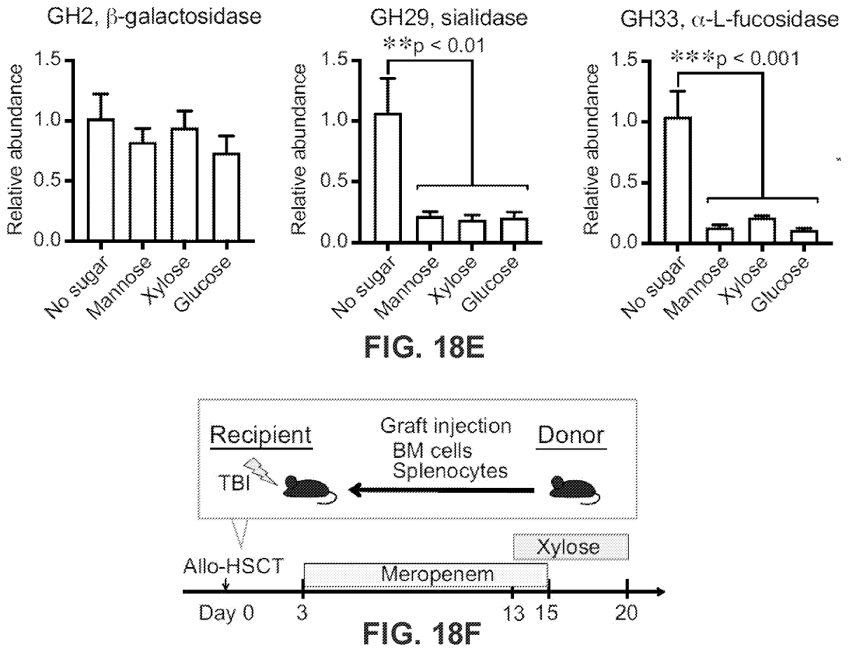

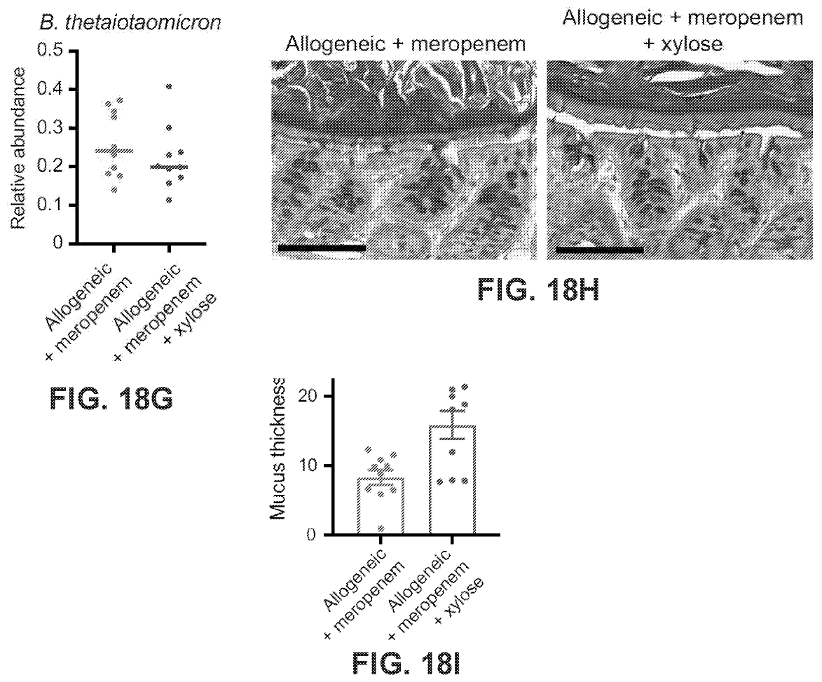

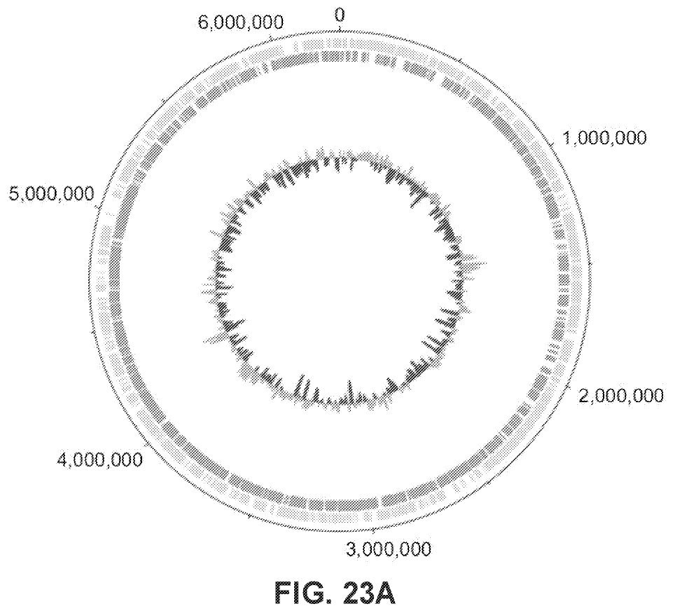

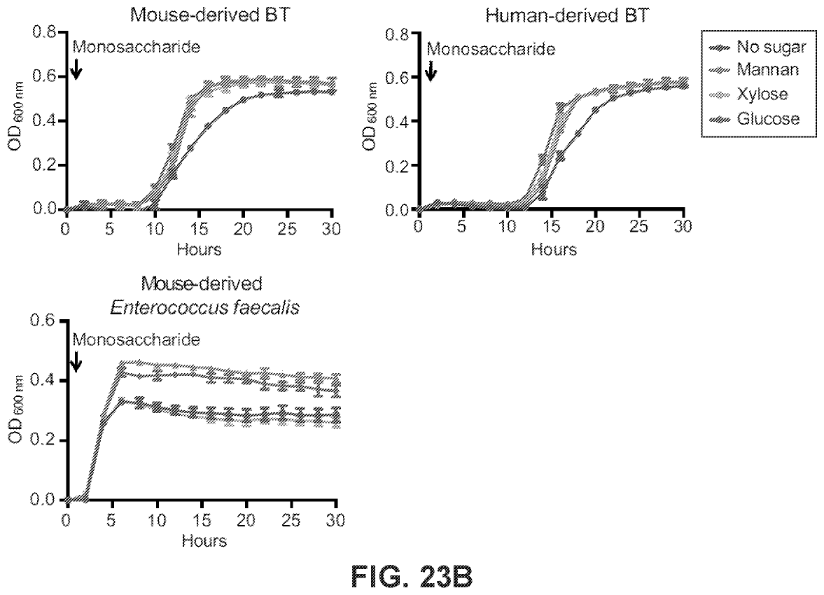

While meropenem treatment led to higher abundances of BT in mice with GVHD, it was also found that GVHD itself, without meropenem treatment, also resulted in moderate increases in abundances of BT compared to syngeneic HSCT recipient mice (FIG. 16B). GVHD mice, however, did not display a notable thinning of the mucus layer or barrier compromise, despite increased intestinal abundances of BT. Thus, in some cases, meropenem treatment during GVHD may lead to alterations in the behavior of BT. To evaluate this further, RNA sequencing of stool samples from allo-HSCT mice without and with meropenem treatment was performed. Also examined was the genome of mouse-derived BT isolate MDA-JAX BT001 (FIG. 23), and open reading frames (ORF) were identified, which were then assigned to polysaccharide utilization loci (PUL) using the Polysaccharide-Utilization Loci DataBase (PULDB) (41). RNA reads were examined that aligned to the BT genome, and it was found that meropenem treatment led to upregulated expression in murine BT of GH2 β-galactosidase, GH33 sialidase, and GH29 α-L-fucosidase, all of which participate in the degradation of host mucin glycans (FIG. 18A).

BT is known to be a versatile utilizer of a variety of carbohydrate sources derived from the diet, including xylose via the xylose isomerase pathway, as well as host glycans. In the presence of multiple suitable carbohydrate substrates, BT has been found to preferentially consume certain carbohydrates first, and only after depleting these will it then upregulate utilization genes targeting other available polysaccharides (42). Host mucin glycans are particularly low on the metabolic hierarchy and are typically targeted only after other dietary polysaccharides have been depleted (43). Both ambient carbohydrates as well as metabolic byproducts have been found to be modulators of the BT transcriptional profile (44). Thus, in some embodiments, levels of soluble carbohydrates in the colonic lumen may be perturbed by meropenem treatment, given that it is shown herein that meropenem depletes the abundance of commensal Clostridia, which function to metabolize dietary fibers and starches (45). Using acid hydrolysis followed by ion chromatography-mass spectrometry (IC-MS), levels of monosaccharide subunits comprising soluble luminal polysaccharides were characterized in the colon of mice. It was found that meropenem-treatment led to significantly lower concentrations of arabinose- and xylose-derived polysaccharides in mice with GVHD (FIG. 18B).

To examine the effects of the presence of monosaccharides on mucin utilization by BT, BT was cultivated in bacterial media containing porcine gastric mucin, a panel of monosaccharides was subsequently added, including arabinose and xylose, and levels of remaining mucin were quantified using a colorimetric assay (FIG. 18C). In the absence of additional monosaccharides, BT readily metabolized porcine gastric mucin. Mucin utilization by BT in the presence of certain monosaccharides, however, was significantly suppressed, including in particular mannose, glucose, or xylose (FIG. 18D). Expression levels of GH2 β-galactosidase, GH33 sialidase and GH29 α-L-fucosidase, were quantified by real-time PCR, and it was found that expression of GH33 sialidase and GH29 α-L-fucosidase by murine BT were suppressed by these monosaccharides, but expression of GH2 β-galactosidase was not significantly impacted (FIG. 18C, FIG. 18E). Altogether, these data indicate that, in some embodiments, following meropenem treatment, reductions in colonic soluble polysaccharides, particularly those comprised of xylose, leads to increased mucus-degrading behavior by BT in the colon of mice with GVHD, and in some cases, oral supplementation with xylose could mediate a benefit in mice with GVHD aggravated by meropenem.

Allo-HSCT recipient mice were treated with meropenem as in FIG. 15A and with xylose from days 13 to 20 (FIG. 18F). The relative abundances of BT were not significantly different in meropenem-treated mice with or without oral xylose supplementation (FIG. 18G). Interestingly, xylose supplementation prevented thinning of the colonic mucus thickness compared to GVHD mice receiving meropenem alone (FIG. 18H, FIG. 18I). Importantly, in vitro, growth of mouse and human-derived BT in carbohydrate-poor media was augmented by xylose (FIG. 23B). Together, these results suggest that, in some cases, xylose supplementation does not suppress growth of BT, but rather can inhibit expression of mucus-degrading enzymes by BT, leading to better preservation of the mucus barrier, and in some embodiments, xylose supplementation can be a novel strategy to ameliorate compromise of the epithelial barrier in the setting of an injured commensal microbiota following antibiotic treatment.

### Example 3

### Exemplary Materials & Methods

Retrospective study design. 295 patients with acute myeloid leukemia (AML) or myelodysplastic syndrome (MDS) underwent allogeneic hematopoietic stem cell transplantation (allo-HSCT) with fludarabine plus busulfan as conditioning therapy and tacrolimus and methotrexate as GVHD prophylaxis from 2011 and 2016 and were analyzed retrospectively. Patients were classified by antibiotic exposures, including those who received neither cefepime nor meropenem, those who received cefepime alone, those who received meropenem alone and those who received both cefepime and meropenem from day −10 to day 30 after allo-HSCT. Acute GVHD was diagnosed by clinical and/or pathological findings, and graded according to standard criteria (56). For patient microbiome analyses, identified were 26 meropenem-unexposed patients and 18 meropenem-exposed patients who underwent allo-HSCT with fludarabine plus busulfan as conditioning therapy from 2014 to 2019 and provided stool samples for a biorepository on day 14 after allo-HSCT.

Human samples. Samples were collected from patients undergoing stem cell transplantation and stored at 4° C. for 24-48 hours until aliquoted for long-term storage at −80 ºC.

Mice. Female C57BL/6J (B6: H-2b) and B6D2F1 (H-2b/d, CD45.2+) were purchased from The Jackson Laboratory (Bar Harbor, ME). All animal experiments were performed under the Guide for the Care and Use of Laboratory Animals Published by the US National Institutes of Health and was approved by the Institutional Animal Care and Use Committee. Experiments disclosed herein were performed in a non-blinded fashion.

Antibiotics administration. For subcutaneous administration, meropenem was dissolved with PBS and given at a concentration of 10 mg/day. For oral administration, meropenem was dissolved with phosphate buffer pH 8.0 and given at a concentration of 0.625 g/L in the drinking water from day 3 to day 15 after transplant. Piperacillin/tazobactam and nystatin were given at a concentration of 3.2 g/L and 320,000 IU/L respectively in combination with meropenem in the drinking water from days 5 to 15 after transplant.

Xylose administration. D-(+)-xylose (X3877, SIGMA-ALDRICH®) was dissolved in phosphate buffer pH 8.0 with meropenem or in water without meropenem and given at a concentration of 0.5% from days 13 to 20 after allo-HSCT.

HSCT. Mice were transplanted as previously described (57). In brief, after receiving myeloablative total body irradiation (11 Gray) delivered in 2 doses at 4 hour intervals, B6D2F1 (H-2b/d) mice were i.v. injected with 5×10⁶ bone marrow (BM) cells and 5×10⁶ splenocytes from allogeneic B6 (H-2b) or syngeneic B6D2F1 donors. Female mice that were 8 to 12-weeks-old were allocated randomly to each experimental group, ensuring the mean body weight in each group was similar. Total body radiotherapy was performed using a Shepherd Mark I. Model 30, 137Cs irradiator. Mice were maintained in specific pathogen-free (SPF) condition and received normal chow (LABDIET® PICOLAB® Rodent Diet 20 5053, Lab Supply) after HSCT. Survival after HSCT was monitored daily, and the degree of clinical GVHD was assessed weekly by using an established scoring system (58).

Histological and immunohistochemistry analysis. For pathological analysis, samples of the small intestine, colon and liver were fixed in 10% formalin, embedded in paraffin, sectioned, and stained with hematoxylin and cosin (H&E). Pathology scores were quantified by a blinded pathologist. For evaluation of mucus thickness, colonic sections containing stool pellets were fixed in methanol-Carnoy's fixative composed of methanol 60%, chloroform 30% and glacial acetic acid 10%, and 5 μM sections were made and stained with Periodic acid-Schiff (PAS). Sections were imaged using an APERIO® AT2. Mucus thickness of the colonic sections were measured using eSlide Manager Version 12.4.3.5008. Eight measurements per image were taken and averaged over the entire usable colon surface. Immunohistochemistry was performed using primary antibodies of rabbit anti-CD11b (ab75476, Abcam), visualized using 3,3′-diaminobenzidine (DAB) and counterstained using hematoxylin.

Immunostaining and fluorescence in situ hybridization. Colon containing stool pellets were fixed in methanol-Carnoy's fixative and the 5 μM thin sections were made as described above. Paraffin-embedded sections were dewaxed and hydrated. Sections were incubated with 1 μg Alexa Fluor 594-conjugated EUB338 (5′-GCTGCCTCCCGTAGGAGT-3′; SEQ ID NO:1) for detection of all bacteria in 200 μL of hybridization buffer (750 mM NaCl, 100 mM Tris-HCl (pH 7.4), 5 mM EDTA, 0.01% BSA, 10% dextran sulfate) at 40° C. for 16 hours. Sections were rinsed in wash buffer (50 mM NaCl, 4 mM Tris-HCl (pH 7.4), 0.02 mM EDTA), washed at 45° ° C. for 20 min, stained with anti-Muc2 antibody [C3] (GTX100664, GeneTex) and counterstained with DAPI (Vector Laboratories). Photographs of sections were obtained using a fluorescent microscope (Nikon NIS Elements, Advanced Research version 4.20).

Sequencing of 16S rRNA gene amplicons. Fecal samples from patients and mice were weighed before DNA isolation. In brief, genomic DNA was isolated using the QIAAMP® DNA mini kit (51306, QIAGEN®) according to the manufacturer's protocol that was modified to include an intensive bead-beating lysis step. The V4 region of 16S rRNA gene was amplified by PCR from 100 ng of each of extracted and purified genomic DNA using 515 forward and 806 reverse primer pairs (59). The quality and quantity of the barcoded amplicons were assessed on an Agilent 4200 TapeStation system (Agilent) and QUBIT™ Fluorometer (THERMO FISHER SCIENTIFIC™), and libraries were prepared after pooling at equimolar ratios. The final libraries were purified using QIAQUICK® gel extraction kit (28706X4, QIAGEN®) and sequenced with a 2×250 base pair paired-end protocol on the ILLUMINA® MISEQ™ platform.

Microbiome data analysis. Sequencing data from paired-end reads were de-multiplexed using QIIME 2 (60). Merging of paired-end reads, dereplicating, and length filtering was performed using VSEARCH 2.17.1 (61). Following de-noising and chimera calling using the unoise3 command (62), unique sequences were taxonomically classified with mothur (63) using the Silva database (64) version 138. Weighted UniFrac distances (65) were determined using QIIME 2, visualized using principal coordinate analysis, and evaluated for statistical significance using permutational multivariate analysis of variance (PERMANOVA) testing. For differential abundance analysis, abundances of sequences belonging to taxonomical groups were included for analysis using the Mann-Whitney U test and adjusted for multiple comparisons using the method of Benjamini and Hochberg. Paired samples were analyzed using the Wilcoxon signed rank test with adjustment for multiple comparisons.

Quantification of fecal bacterial density. Genomic DNA was isolated from stool as described above. qPCR was performed as previously described (66). In brief, 16S rRNA gene sequences were amplified from total fecal DNA using the primers 926F (5′-AAACTCAAAKGAATTGACGG-3′, where K is a G or a T; SEQ ID NO:2) and 1062R (5′-CTCACRRCACGAGCTGAC-3′, where R is an A or a G; SEQ ID NO:3). Real-time PCR were carried out in 96-well optical plates on QUANTSTUDIO™ Flex 6 RT PCR (APPLIED BIOSYSTEMS™) and KAPA SYBR® FAST Master Mix (Roche). The PCR conditions included one initial denaturing step of 10 min at 95° C. and 40 cycles of 95° C. for 20 sec and 60 ºC for 1 min. Melting-curve analysis was performed after amplification. To determined bacterial density, a plasmid with a 16S rRNA gene of a murine Blautia isolate was generated in the pCR4 backbone and used as a standard.

Lamina propria hematopoietic cell dissociation. Murine colons were isolated, dissected longitudinally and then on a shaker in 2% fetal bovine serum in PBS with 1 mM DL-dithiothreitol (Bioworld) at 37 ºC for 20 min and subsequently incubated with 1.3 mM EDTA at 37° C. for 40 min. They were rinsed twice and digested with 0.3 mg/ml of type IV collagenase (C5138, SIGMA-ALDRICH®) at 37 ºC for 45 min, homogenized, filtered, and washed.

Flow cytometric analysis. Monoclonal antibodies conjugated with fluorescein isothiocynate, phycoerythrin, phycoerythrin-Cy7, peridinin-chlorophyll protein complexes, allophycocyanin, or allophycocyanin-Cy7 were purchased from Tonbo Biosciences (San Diego, CA), EBIOSCIENCE™ (San Diego, CA), or BIOLEGEND® (San Diego, CA.). Lamina propria cells in colon were stained with the antibodies against murine CD45 (30-F11, BIOLEGEND®), CD11b (M1/70, BIOLEGEND®), CD11c (N418, BIOLEGEND®), CD103 (2E7, BIOLEGEND®), Ly6G (1A8, BIOLEGEND®), MHC-II (M5/114.15.2, BIOLEGEND®) and F4/80 (BM8, BIOLEGEND®) and Zombie Aqua Fixable viability kit (423101, BIOLEGEND®). In flow cytometric analysis, at least 100,000 live samples were analyzed using BD LSRFORTESSA™ X-20 (BD Biosciences) and FlowJo software (Trec Star, OR). The CD45+ cells were classified into neutrophils (CD11b+Ly6G+) and dendritic cells (CD11c+MHC-II+CD103+).

Culturing of bacteria. Mouse-derived BT (MDA-JAX BT001), Enterococcus faecalis (MDA-JAX EF001) and Clostridium disporicum (MDA-JAX CD001), Clostridium saudiense (MDA-JAX CS001), and Lachnospiraceae unclassified (MDA-JAX LS001) were isolated and cultured from mouse stool samples suspended in 1 ml of chilled 20% anaerobic glycerol in a Whitley anaerobic chamber (10% H₂, 5% CO₂ and 85% N₂). Human-derived BT (ATCC 29148) was purchased from ATCC. Bacterial number was quantified using a Nexcelom Cellometer cell counter with SYTO™ BC dye and propidium iodide. For measuring MICs against meropenem, bacteria were cultured on BYE plates including 5% sterilized rumen fluid (Fisher Scientific) with MIC test strips (LIOFILCHEM™ MTS™ Meropenem [MRP] 0.016-256 μg/mL, THERMO FISHER SCIENTIFIC™)). Bacterial growth experiments were in liquid media were performed in a novel bacterial media, BYEM10, composed of a hybrid of BHI and M10 supplemented with yeast extract (Table 5).

TABLE 5
Formula per liter of BYEM10 broth.

| | | |
|---|---|---|
| KH₂PO₄ (P5655, SIGMA-ALDRICH ®) | 0.06 | g |
| K₂HPO₄ (RES20765, SIGMA-ALDRICH ®) | 0.06 | g |
| (NH₄)₂SO₄ (221260, SIGMA-ALDRICH ®) | 0.06 | g |
| NaCl (S3014, SIGMA-ALDRICH ®) | 0.12 | g |
| MgSO₄ (230391, SIGMA-ALDRICH ®) | 0.025 | g |
| CaCl₂ (746495, SIGMA-ALDRICH ®) | 0.01 | g |
| Yeast Extract (DF0127-17-9, THERMO | 4.0 | g |
| FISHER SCIENTIFIC ™) | | |
| Peptone from casein, pancreatic digest | 0.5 | g |
| (70169, SIGMA-ALDRICH ®) | | |
| Meat extract (70164, SIGMA-ALDRICH ®) | 0.375 | g |
| Brain Heart Infusion broth without dextrose | 26 | g |
| (71-C00079, Microbiology International) | | |
| Hemin (51280, SIGMA-ALDRICH ®) | 0.01 | g |
| L-Cysteine (30120, SIGMA-ALDRICH ®) | 1 | g |
| Vitamin Supplement (MD-VS, ATCC) | 10 | mL |
| Mineral Supplement (MDTMS, ATCC) | 10 | mL |
| Vitamin K3 (M5625, SIGMA-ALDRICH ®) | 1 | mg |

Bacteria were cultured up to 48 hours at a starting concentration of 1×10⁶ bacteria/ml in BYEM10 broth (pH 7.2) with and without 5 mg/ml of porcine gastric mucin (M1778, Sigma-Aldrich). Optical densities (OD600 nm) of bacterial cultures were measured with a BioTek EPOCH™ 2 plate reader.

Microbiologic analysis of bacterial translocation. Mesenteric lymph nodes (MLNs) were harvested from mice and homogenized in PBS and cultured anaerobically on BHI plates containing yeast extract and 5% sterilized rumen fluid (THERMO FISHER SCIENTIFIC™)) and Columbia blood agar plates (BD) for 4 days at 37° ° C. Colony-forming units (CFUs) were counted and adjusted per organ. Bacteria were identified by MALDI BIOTYPER®.

Mucin degradation assay. Levels of mucin glycans in culture supernatants were determined by a PAS-based colorimetric assay as previously described (67) with minor modifications. Briefly, culture supernatants were centrifuged at 20,000 g, 4° C. for 10 minutes and collected. To perform mucin precipitation, 500 μL of culture supernatants were mixed with 1 mL of molecular grade ethanol and incubated at −30° C. overnight. Culture supernatants were centrifuged at 20,000 g, 4° C. for 10 minutes. Mucin-containing pellets were washed with 1 mL of molecular grade ethanol twice and resuspended in 500 μL of PBS. 10 μL of washed culture supernatants were transferred into round bottom 96-well plate containing 15 μL of PBS. Serially diluted porcine gastric mucin (SIGMA-ALDRICH®) standards were prepared. Freshly prepared 0.06% periodic acid in 7% acetic acid was added, and incubated at 37° C. for 90 min, followed by 100 μL of Schiff's reagent (84655, SIGMA-ALDRICH®) and incubation at room temperature for 40 min. Absorbance was measured at 550 nm using a BioTek SYNERGY™ HTX plate reader.

Short Chain Fatty Acids profiling by ion chromatography-mass spectrometry (IC-MS). To determine the relative abundance of short chain fatty acids in mouse feces samples, extracts were prepared and analyzed by ultra-high resolution mass spectrometry (HRMS). Fecal pellets were homogenized with a PRECELLYS® Tissue Homogenizer. Metabolites were extracted using 1 mL ice-cold 0.1% Ammonium hydroxide in 80/20 (v/v) methanol/water. Extracts were centrifuged at 17,000 g for 5 min at 4° C., and supernatants were transferred to clean tubes, followed by evaporation to dryness under nitrogen. Dried extracts were reconstituted in deionized water, and 5 μL was injected for analysis by IC-MS. IC mobile phase A (MPA; weak) was water, and mobile phase B (MPB; strong) was water containing 100 mM KOH. A THERMO SCIENTIFIC™ DIONEX™ ICS-5000+ system included a THERMO FISHER SCIENTIFIC™ IONPAC™ AS11 column (4 μM particle size, 250×2 mm) with column compartment kept at 30° C. The autosampler tray was chilled to 4° C. The mobile phase flow rate was 360 μL/min, and the gradient elution program was: 0-5 min, 1% MPB; 5-25 min, 1-35% MPB; 25-39 min, 35-99% MPB; 39-49 min, 99% MPB; 49-50, 99-1% MPB. The total run time was 50 min. To assist the desolvation for better sensitivity, methanol was delivered by an external pump and combined with the eluent via a low dead volume mixing tec. Data were acquired using a THERMO FISHER SCIENTIFIC™ ORBITRAP FUSION™ TRIBRID™ Mass Spectrometer under ESI negative ionization mode at a resolution of 240,000. Raw data files were imported to THERMO FISHER SCIENTIFIC™ TRACEFINDER™ and COMPOUND DISCOVERER™ software for spectrum database analysis. The relative abundance of each metabolite was normalized by sample weight.

Pharmacokinetics of meropenem by Triple Quadruple liquid chromatography-mass spectrometry (LC-MS). C57BL/6 mice were treated with 10 mg of meropenem by subcutaneously injection. Cecal contents were collected prior to, 4, 8, 24, 48, and 96 hours after meropenem injection. To determine the relative abundance of meropenem in mouse cecum samples, extracts were prepared and analyzed with a THERMO SCIENTIFIC™ TSQ QUANTIVA™ triple quadruple mass spectrometer coupled with a DIONEX™ ULTIMATE™ 3000 HPLC system. Approximately 600 mgs of mouse cecal contents were homogenized with a PRECELLYS® Tissue Homogenizer. Metabolites were extracted using 100% acetonitrile. The tissue lysates were vortexed, centrifuged at 17,000 g for 5 min at 4° C., and organic layers were transferred to clean tubes, followed by evaporation to dryness under nitrogen. Dried extracts were reconstituted in 50/50 (v/v) water/Acetonitrile, and 5 μL was injected for analysis by LC-MS. The mobile phase A is 100% water and mobile phase B is 0.1% Formic Acid in acetonitrile. Separation of meropenem was achieved on an Agilent SB-C18, 1.8 UM, 100×3 mm column. The flow rate was 250 μL/min at 35° C., and the gradient elution program was: 0-1 min, 5% MPB; 1-5 min, 5-50% MPB; 5-6 min, 50-95% MPB; 6-10 min, 95% MPB; 10-10.1 min, 95-5% MPB. The total run time was 15 min. The mass spectrometer was operated in the MRM positive ion electrospray mode with the transition m/z 384.1->68.0. Raw data files were imported to THERMO FISHER SCIENTIFIC™ TRACEFINDER™ software for final analysis. The relative abundance of meropenem was normalized by sample weight.

Carbohydrates analysis by IC-MS. To determine the relative abundance of carbohydrates in mouse feces samples, extracts were prepared and analyzed by ultra-HRMS. Fecal pellets were homogenized with a PRECELLYS® Tissue Homogenizer. Metabolites were extracted using 1 mL ice-cold 80/20 (v/v) methanol/water. Extracts were centrifuged at 17,000 g for 5 min at 4° C., and supernatants were transferred to clean tubes, followed by evaporation to dryness under nitrogen. Dried extracts were reconstituted in deionized water, and 5 μL was injected for analysis by IC-MS. IC mobile phase A (MPA; weak) was water, and mobile phase B (MPB; strong) was water containing 100 mM KOH. A THERMO SCIENTIFIC™ DIONEX™ ICS-5000+ system included a THERMO FISHER SCIENTIFIC™ CARBOPAC™ PA-20-Fast column (4 μM particle size, 100×2 mm) with column compartment kept at 30° C. The autosampler tray was chilled to 4° C. The mobile phase flow rate was 200 μL/min, and the gradient elution program was: 0-0.5 min, 1% MPB; 0.5-10 min, 1-5% MPB; 10-15 min, 5-95% MPB; 15-20 min, 95% MPB; 20.5-25, 95-1% MPB. The total run time was 25 min. To assist the desolvation for better sensitivity, methanol was delivered by an external pump and combined with the eluent via a low dead volume mixing tec. Data were acquired using a THERMO FISHER SCIENTIFIC™ ORBITRAP FUSION™ TRIBRID™ Mass Spectrometer under ESI negative ionization mode at a resolution of 240,000. Raw data files were imported to THERMO FISHER SCIENTIFIC™ TRACEFINDER™ and COMPOUND DISCOVERER™ software for spectrum database analysis. The relative abundance of each metabolite was normalized by sample weight.

Whole genome sequencing of BT (MDA-JAX BT001). BT (MDA-JAX BT001) genomic DNA was isolated and purified using a QIAGEN® Genomic-tip 20/G column, according to the manufacturer's instructions. For short-read ILLUMINA® sequencing, libraries were constructed with a NEXTERA™ DNA Flex Library Prep Kit (ILLUMINA®, San Diego, CA, USA), according to the manufacturer's protocol. All libraries were quantified with a TapeStation and pooled in equal molar ratios. The final libraries were sequenced with the NovaSeq 6000 platform (ILLUMINA®) to produce 2×150 bp paired-end reads, resulting in ˜5 Gb per sample. For long-read Nanopore sequencing, 500 ng of genomic DNA was used for library preparation using the Rapid Sequencing Kit (SQK-RAD004, Oxford Nanopore Technologies). Libraries were loaded into a FLO-MIN106 flow-cell for 24 h sequencing run on a MINION™ sequencer platform (Oxford Nanopore Technologies, Oxford, UK). Data acquisition and real-time base calling were carried out by the MINKNOW™ software version 3.6.5. The fastq files were generated from basecalled sequencing fast5 reads.

Hybrid assembly and genome annotation of BT (MDA-JAX BT001). To assemble the complete genome of BT, Flye version 2.8.2. (68) was used with long (Nanopore) reads and short (NovaSeq) reads combined using default settings. The genome was compared to a reference genome (BT 5 VPI-5482) using Rebaler version 0.2.0 (github.com/rrwick/Rebaler). The similarities of the genome of MDA-JAX BT001 to other reference genomes was calculated using blastn for Bacteroides faecichinchillae (GCF_004801645.1_ASM480164v1), Bacteroides faecis (GCF_000226135.1_ASM22613v2), and BT (VPI-5482), respectively (69). Open reading frames (ORFs) of BT (MDA-JAX BT001) were identified using the Sequence Manipulation Suite (70) and annotated with polysaccharide utilization loci (PUL) (41) using BWA version 0.7.17 (71). The genome of BT and ORFs were depicted using DNA plotter software (72).

RNA sequencing and analysis. Approximately 30 mg of stool was freshly collected in 700 μL of ice cold QIAZOL® containing 200 μL of 0.1 mm diameter Zirconia Silica beads (11079101z, BioSpec). Samples were bead beaten twice for 2 min with a 30 second interval recovery. Samples were then centrifuged at 12,000 g for 1 min and the supernatant was collected for RNA isolation using the RNeasy mini kit (74104, QIAGEN®). RNA was treated on column with DNase I (79254, QIAGEN®) to eliminate contaminating genomic DNA. RNA quantity and quality was determined using an Agilent 4200 TapeStation system (Agilent). 250 ng of total RNA from mouse stools was used to construct libraries using the NUGEN® OVATION® Complete Prokaryotic RNA-Seq Systems (NUGEN®), following the manufacturer's protocol. The cDNA libraries were sequenced on the ILLUMINA® MISEQ™ system to produce 1×300 bp single-end reads, resulting in ˜1 million reads per sample. Sequence data were demultiplexed using QIIME 2 (60) and their qualities were checked using VSEARCH 2.17.1 (61). Data were filtered and truncated by quality with VSEARCH default settings. Adapter sequences were removed using QIIME. The total reads of mouse stool samples were 950923±113406 (mean±standard deviation). Sequences of ribosomal RNA were removed using BWA software against prokaryotic ribosomal RNA sequences from prokaryotic RefSeq genomes (73). Sequences of interest were further identified using DIAMOND software version 0.9.24 (74) to align against PULs. Features with percent identity less than 80% were excluded. The total counts of bacterial isolated samples were 104172±101292. Aligned mRNA expression changes were calculated using the Mann-Whitney U test in R software version 3.6.0 via RStudio version 1.2.1335. P values <0.05 were considered statistically significant.

Quantitative real-time PCR analysis of BT loci. Total RNA was isolated from in vitro cultured BT as described above. The cDNA was synthesized using a High-Capacity cDNA Reverse Transcription Kit (4368814, APPLIED BIOSYSTEMS™). The mRNA levels of selected targets were quantified by qPCR using KAPA SYBR® FAST Master Mix (Roche) and specific probes (GH2, 5′-CGCACTCTTCTTGCATCTGC-3′ (SEQ ID NO:4) for the forward primer, 5′-TACCAACGGCTCACATTGGG-3′ (SEQ ID NO:5) for the reverse primer; GH29, 5′-GATGCTGGAAAAGGCAACGG-3′ (SEQ ID NO:6) for the forward primer, 5′-AGCGTGCCTTTTCCTTCTGA-3′ (SEQ ID NO:7) for the reverse primer; GH33, 5′-GGTCACCGAAAGACATTATTCATCG-3′ (SEQ ID NO:8) for the forward primer, 5′-GCCGTTTGATACAGATCCATTCC-3′ (SEQ ID NO:9) for the reverse primer) and were normalized to BT specific probes (5′-CACAACAGCCATAGCGTTCCA-3′ (SEQ ID NO:10) for the forward primer, 5′-ATCGCAAAAATAAGATGGGCAAA-3′ (SEQ ID NO:11) for the reverse primer) (75).

Statistical analysis. Data were checked for normality and similar variances between groups and Student's t-tests were used when appropriate. Mann-Whitney U tests were used to compare data between two groups when the data did not follow a normal distribution. The Kaplan-Meier curves were used to depict survival probabilities and the log-rank test was applied to compare survival curves. For clinical data analysis, non-repeated ANOVA was used to compare continuous variables, while chi-square or Fisher's exact tests were used to analyze the frequency distribution between categorical variables. Analyses were performed using R software version 3.6.0 and Prism version 7.0 (GraphPad Software, San Diego, CA). P values <0.05 were considered statistically significant.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
1. N. M. Kuderer, D. C. Dale, J. Crawford, L. E. Cosler, G. H. Lyman, Mortality, morbidity, and cost associated with febrile neutropenia in adult cancer patients. Cancer 106, 2258-2266 (2006).
2. E. Tai, G. P. Guy, A. Dunbar, L. C. Richardson, Cost of Cancer-Related Neutropenia or Fever Hospitalizations, United States, 2012. J Oncol Pract 13, e552-e561 (2017).
3. R. A. Taplitz et al., Antimicrobial Prophylaxis for Adult Patients With Cancer-RelateD Immunosuppression: ASCO and IDSA Clinical Practice Guideline Update. J Clin Oncol 36, 3043-3054 (2018).
4. G. P. Bodey, The changing face of febrile neutropenia—from monotherapy to moulds to mucositis. Fever and neutropenia: the early years. J Antimicrob Chemother 63 Suppl 1, 13-13 (2009).
5. A. G. Freifeld et al., Clinical practice guideline for the use of antimicrobial agents in neutropenic patients with cancer: 2010 update by the infectious diseases society of America. Clin Infect Dis 52, e56-93 (2011).
6. F. B. Tamburini et al., Precision identification of diverse bloodstream pathogens in the gut microbiome. Nat Med 24, 1809-1814 (2018).
7. Y. Taur et al., Intestinal Domination and the Risk of Bacteremia in Patients Undergoing Allogeneic Hematopoietic Stem Cell Transplantation. Clin Infect Dis, (2012).
8. J. F. Sicard, G. Le Bihan, P. Vogeleer, M. Jacques, J. Harel, Interactions of Intestinal Bacteria with Components of the Intestinal Mucus. Front Cell Infect Microbiol 7, 387 (2017).
9. M. Kilcoyne, J. Q. Gerlach, M. P. Farrell, V. P. Bhavanandan, L. Joshi, Periodic acid-Schiff's reagent assay for carbohydrates in a microtiter plate format. Anal Biochem 416, 18-26 (2011).
10. I. Lagkouvardos et al., Sequence and cultivation study of Muribaculaceae reveals novel species, host preference, and functional potential of this yet undescribed family. Microbiome 7, 28 (2019).
11. B. Gyurkocza, B. M. Sandmaier, Conditioning re 1 gimens for hematopoietic cell transplantation: one size does not fit all. Blood 124, 344-353 (2014).
12. E. Ansaldo et al., Akkermansia muciniphila induces intestinal adaptive immune responses during homeostasis. Science 364, 1179-1184 (2019).
13. J. M. Macy, L. G. Ljungdahl, G. Gottschalk, Pathway of succinate and propionate formation in Bacteroides fragilis. J Bacteriol 134, 84-91 (1978).
14. Q. P. Liu et al., Bacterial glycosidases for the production of universal red blood cells. Nat Biotechnol 25, 454-464 (2007).
15. E. H. Crost et al., The mucin-degradation strategy of Ruminococcus gnavus: The importance of intramolecular trans-sialidases. Gut Microbes 7, 302-312 (2016).
16. B. Vogel et al., Touch-free measurement of body temperature using close-up thermography of the ocular surface. MethodsX 3, 407-416 (2016).
17. A. A. Romanovsky et al., Fever and hypothermia in systemic inflammation: recent discoveries and revisions. Front Biosci 10, 2193-2216 (2005).
18. W. Tao, D. J. Deyo, D. L. Traber, W. E. Johnston, E. R. Sherwood, Hemodynamic and cardiac contractile function during sepsis caused by cecal ligation and puncture in mice. Shock 21, 31-37 (2004).
19. L. V. Hooper, D. R. Littman, A. J. Macpherson, Interactions between the microbiota and the immune system. Science 336, 1268-1273 (2012).
20. R. R. Jenq et al., Regulation of intestinal inflammation by microbiota following allogeneic bone marrow transplantation. J Exp Med 209, 903-911 (2012).
21. R. E. Ley, C. A. Lozupone, M. Hamady, R. Knight, J. I. Gordon, Worlds within worlds: evolution of the vertebrate gut microbiota. Nat Rev Microbiol 6, 776-788 (2008).
22. G. R. Hill, J. L. Ferrara, The primacy of the gastrointestinal tract as a target organ of acute graft-versus-host disease: rationale for the use of cytokine shields in allogeneic bone marrow transplantation. Blood 95, 2754-2759 (2000).
23. Y. Taur et al., The effects of intestinal tract bacterial diversity on mortality following allogeneic hematopoietic stem cell transplantation. Blood 124, 1174-1182 (2014).
24. Holler et al., Metagenomic analysis of the stool microbiome in patients receiving allogeneic stem cell transplantation: loss of diversity is associated with use of systemic antibiotics and more pronounced in gastrointestinal graft-versus-host disease. Biol Blood Marrow Transplant 20, 640-645 (2014).
25. Y. Shono et al., Increased GVHD-related mortality with broad-spectrum antibiotic use after allogeneic hematopoietic stem cell transplantation in human patients and mice. Sci Transl Med 8, 339ra371 (2016).
26. D. Hidaka et al., The association between the incidence of intestinal graft-vs-host disease and antibiotic use after allogeneic hematopoietic stem cell transplantation. Clin Transplant 32, e13361 (2018).
27. S. E. Lee et al., Alteration of the Intestinal Microbiota by Broad-Spectrum Antibiotic Use Correlates with the Occurrence of Intestinal Graft-versus-Host Disease. Biol Blood Marrow Transplant 25, 1933-1943 (2019).
28. C. W. Elgarten et al., Broad spectrum antibiotics and risk of graft-versus-host disease in pediatric patients transplanted for acute leukemia: association of carbapenem use with risk of acute GVHD. Transplant Cell Ther 27, 177 e171-177 e178 (2021).
29. K. Atarashi et al., Induction of colonic regulatory T cells by indigenous Clostridium species. Science 331, 337-341 (2011).
30. N. D. Mathewson et al., Gut microbiome-derived metabolites modulate intestinal epithelial cell damage and mitigate graft-versus-host disease. Nat Immunol 17, 505-513 (2016).
31. T. R. Simms-Waldrip et al., Antibiotic-Induced Depletion of Anti-inflammatory Clostridia Is Associated with the Development of Graft-versus-Host Disease in Pediatric Stem Cell Transplantation Patients. Biol Blood Marrow Transplant 23, 820-829 (2017).
32. V. Bekker et al., Dynamics of the Gut Microbiota in Children Receiving Selective or Total Gut Decontamination Treatment during Hematopoietic Stem Cell Transplantation. Biol Blood Marrow Transplant 25, 1164-1171 (2019).
33. J. U. Peled et al., Microbiota as Predictor of Mortality in Allogeneic Hematopoietic-Cell Transplantation. N Engl J Med 382, 822-834 (2020).
34. J. Jovel et al., Characterization of the Gut Microbiome Using 16S or Shotgun Metagenomics. Front Microbiol 7, 459 (2016).
35. K. S. Bergstrom, L. Xia, Mucin-type O-glycans and their roles in intestinal homeostasis. Glycobiology 23, 1026-1037 (2013).
36. L. E. Tailford, E. H. Crost, D. Kavanaugh, N. Juge, Mucin glycan foraging in the human gut microbiome. Front Genet 6, 81 (2015).
37. S. M. Bloom et al., Commensal Bacteroides species induce colitis in host-genotypespecific fashion in a mouse model of inflammatory bowel disease. Cell Host Microbe 9, 390-403 (2011).
38. C. A. Hickey et al., Colitogenic Bacteroides thetaiotaomicron Antigens Access Host Immune Cells in a Sulfatase-Dependent Manner via Outer Membrane Vesicles. Cell Host Microbe 17, 672-680 (2015).
39. L. Schwab et al., Neutrophil granulocytes recruited upon translocation of intestinal bacteria enhance graft-versus-host disease via tissue damage. Nat Med 20, 648-654 (2014).
40. M. Koyama et al., Donor colonic CD103+ dendritic cells determine the severity of acute graft-versus-host disease. J Exp Med 212, 1303-1321 (2015).
41. N. Terrapon et al., PULDB: the expanded database of Polysaccharide Utilization Loci. Nucleic Acids Res 46, D677-D683 (2018).
42. T. E. Rogers et al., Dynamic responses of Bacteroides thetaiotaomicron during growth on glycan mixtures. Mol Microbiol 88, 876-890 (2013).
43. M. A. TerAvest et al., Regulated expression of polysaccharide utilization and capsular biosynthesis loci in biofilm and planktonic Bacteroides thetaiotaomicron during growth in chemostats. Biotechnol Bioeng 111, 165-173 (2014).
44. W. B. Schofield, M. Zimmermann-Kogadeeva, M. Zimmermann, N. A. Barry, A. L. Goodman, The Stringent Response Determines the Ability of a Commensal Bacterium to Survive Starvation and to Persist in the Gut. Cell Host Microbe 24, 120-132 e126 (2018).
45. D. Chinda et al., The fermentation of different dietary fibers is associated with fecal clostridia levels in men. J Nutr 134, 1881-1886 (2004).
46. R. A. Taplitz et al., Antimicrobial Prophylaxis for Adult Patients With Cancer-Related Immunosuppression: ASCO and IDSA Clinical Practice Guideline Update. J Clin Oncol 36, 3043-3054 (2018).
47. G. Freifeld et al., Clinical practice guideline for the use of antimicrobial agents in neutropenic patients with cancer: 2010 Update by the Infectious Diseases Society of America. Clin Infect Dis 52, 427-431 (2011).
48. J. M. Vossen et al., Prevention of infection and graft-versus-host disease by suppression of intestinal microflora in children treated with allogeneic bone marrow transplantation. Eur J Clin Microbiol Infect Dis 9, 14-23 (1990).
49. D. W. Beelen, A. Elmaagacli, K. D. Muller, H. Hirche, U. W. Schaefer, Influence of intestinal bacterial decontamination using metronidazole and ciprofloxacin or ciprofloxacin alone on the development of acute graft-versus-host disease after marrow transplantation in patients with hematologic malignancies: final results and long-term follow-up of an open-label prospective randomized trial. Blood 93, 3267-3275 (1999).
50. J. M. Vossen et al., Complete suppression of the gut microbiome prevents acute graftversus—host disease following allogeneic bone marrow transplantation. PLOS One 9, e105706 (2014).
51. P. M. Smith et al., The microbial metabolites, short-chain fatty acids, regulate colonic Treg cell homeostasis. Science 341, 569-573 (2013).
52. N. Arpaia et al., Metabolites produced by commensal bacteria promote peripheral regulatory T-cell generation. Nature 504, 451-455 (2013).
53. L. Wrzosek et al., Bacteroides thetaiotaomicron and Faecalibacterium prausnitzii influence the production of mucus glycans and the development of goblet cells in the colonic epithelium of a gnotobiotic model rodent. BMC Biol 11, 61 (2013).
54. van der Hee, J. M. Wells, Microbial Regulation of Host Physiology by Short-chain Fatty Acids. Trends Microbiol 29, 700-712 (2021).
55. Dodd, R. I. Mackie, I. K. Cann, Xylan degradation, a metabolic property shared by rumen and human colonic Bacteroidetes. Mol Microbiol 79, 292-304 (2011).
56. Przepiorka et al., 1994 Consensus Conference on Acute GVHD Grading. Bone Marrow Transplant 15, 825-828 (1995).
57. Hayase et al., R-Spondin1 expands Paneth cells and prevents dysbiosis induced by graftversus host disease. J Exp Med 214, 3507-3518 (2017).
58. K. R. Cooke et al., An experimental model of idiopathic pneumonia syndrome after bone marrow transplantation: I. The roles of minor H antigens and endotoxin. Blood 88, 3230-3239 (1996).
59. J. G. Caporaso et al., Ultra-high-throughput microbial community analysis on the Illumina HiSeq and MiSeq platforms. ISME J 6, 1621-1624 (2012).
60. J. G. Caporaso et al., QIIME allows analysis of high-throughput community sequencing data. Nat Methods 7, 335-336 (2010).
61. T. Rognes, T. Flouri, B. Nichols, C. Quince, F. Mahe, VSEARCH: a versatile open source tool for metagenomics. PeerJ 4, e2584 (2016).
62. R. C. Edgar, UNOISE2: improved error-correction for Illumina 16S and ITS amplicon sequencing. bioRxiv, 081257 (2016).
63. P. D. Schloss et al., Introducing mothur: open-source, platform-independent, community supported software for describing and comparing microbial communities. Appl Environ Microbiol 75, 7537-7541 (2009).
64. C. Quast et al., The SILVA ribosomal RNA gene database project: improved data processing and web-based tools. Nucleic Acids Res 41, D590-596 (2013).
65. C. Lozupone, M. E. Lladser, D. Knights, J. Stombaugh, R. Knight, UniFrac: an effective distance metric for microbial community comparison. ISME J 5, 169-172 (2011).
66. Y. W. Yang et al., Use of 16S rRNA Gene-Targeted Group-Specific Primers for Real-Time PCR Analysis of Predominant Bacteria in Mouse Feces. Appl Environ Microbiol 81, 6749-6756 (2015).
67. M. Kilcoyne, J. Q. Gerlach, M. P. Farrell, V. P. Bhavanandan, L. Joshi, Periodic acid-Schiff's reagent assay for carbohydrates in a microtiter plate format. Anal Biochem 416, 18-26 (2011).
68. M. Kolmogorov, J. Yuan, Y. Lin, P. A. Pevzner, Assembly of long, error-prone reads using repeat graphs. Nat Biotechnol 37, 540-546 (2019).
69. S. F. Altschul, W. Gish, W. Miller, E. W. Myers, D. J. Lipman, Basic local alignment search tool. J Mol Biol 215, 403-410 (1990).
70. P. Stothard, The sequence manipulation suite: JavaScript programs for analyzing and formatting protein and DNA sequences. Biotechniques 28, 1102, 1104 (2000).
71. H. Li, R. Durbin, Fast and accurate short read alignment with Burrows-Wheeler transform. Bioinformatics 25, 1754-1760 (2009).
72. T. Carver, N. Thomson, A. Bleasby, M. Berriman, J. Parkhill, DNAPlotter: circular and linear interactive genome visualization. Bioinformatics 25, 119-120 (2009).
73. T. Tatusova et al., NCBI prokaryotic genome annotation pipeline. Nucleic Acids Res 44, 6614-6624 (2016).
74. B. Buchfink, C. Xie, D. H. Huson, Fast and sensitive protein alignment using DIAMOND. Nat Methods 12, 59-60 (2015).
75. Benjdia, E. C. Martens, J. L Gordon, O. Bertean, Sulfatases and a radical S-adenosyl-L-methionine (AdoMet) enzyme are key for mucosal foraging and fitness of the prominent human gut symbiont, Bacteroides thetaiotaomicron. J Biol Chem 286, 25973-25982 (2011).

What is claimed is:

1. A method of preventing or reducing the severity of cancer therapy-induced neutropenic fever, the method comprising prophylactically administering to a subject receiving a cancer therapy a therapeutically effective amount of a composition comprising one or more of the following:
a) one or more agents targeting growth or expansion of one or more genera of mucus-degrading bacteria in the gut microbiome of the subject;
b) one or more mucus-degrading enzyme inhibitors to inhibit mucus degradation by one or more genera of mucus-degrading bacteria in the gut microbiome of the subject; and/or
c) one or more mediators of organic acid metabolite levels produced following metabolism of mucin-derived carbohydrates by one or more genera of mucus-degrading bacteria in the gut microbiome of the subject;
wherein the one or more genera of mucus-degrading bacteria comprise Akkermansia or Bacteroides.

2. The method of claim 1, wherein subject is at a higher risk than an average person in the general population receiving the cancer therapy of developing cancer therapy-induced neutropenic fever.

3. The method of claim 1 or claim 2, wherein the cancer therapy-induced neutropenic fever poses a greater risk to the health or life of the subject than such a condition would pose to an average person in the general population receiving the cancer therapy.

4. The method of any one of claims 1-3, wherein the subject was determined to have an increased abundance of mucus-degrading bacteria in the gut microbiome compared to a control or reference sample.

5. The method of claim 4, wherein the increased abundance of mucus-degrading bacteria in the gut microbiome was determined from a fecal sample from the subject.

6. The method of any one of claims 1-5, wherein the subject was determined to have an increase in functional activity and/or expression levels of one or more mucus-degrading enzymes secreted by one or more genera of mucus-degrading bacteria in the gut microbiome compared to a control or reference sample.

7. The method of claim 6, wherein the increase in functional activity and/or expression levels of one or more mucus-degrading enzymes secreted by one or more genera of mucus-degrading bacteria in the gut microbiome was determined from a fecal sample from the subject.

8. The method of any one of claims 1-7, wherein the subject was determined to have an decrease in the levels of one or more organic acid metabolites produced following metabolism of mucin-derived carbohydrates by one or more genera of mucus-degrading bacteria in the gut microbiome compared to a control or reference sample.

9. The method of claim 8, wherein the decrease in the levels of one or more organic acid metabolites produced following metabolism of mucin-derived carbohydrates by one or more genera of mucus-degrading bacteria in the gut microbiome was determined from a fecal sample from the subject.

10. A method of treating cancer therapy-induced neutropenic fever in a subject receiving a cancer therapy and having an increased abundance of the one or more genera of mucus-degrading bacteria in the gut microbiome of the subject compared to a control or reference sample, the method comprising administering to the subject a therapeutically effective amount of a composition comprising one or more of the following:
a) one or more agents targeting growth or expansion of one or more genera of mucus-degrading bacteria in the gut microbiome of the subject;
b) one or more mucus-degrading enzyme inhibitors to inhibit mucus degradation by one or more genera of mucus-degrading bacteria in the gut microbiome of the subject; and/or
c) one or more mediators of organic acid metabolite levels produced following metabolism of mucin-derived carbohydrates by one or more genera of mucus-degrading bacteria in the gut microbiome of the subject;
wherein the one or more genera of mucus-degrading bacteria comprise Akkermansia or Bacteroides.

11. A method of treating cancer therapy-induced neutropenic fever in a subject receiving a cancer therapy and having increased functional activity or expression levels of one or more mucus-degrading enzymes secreted by one or more genera of mucus-degrading bacteria in the gut microbiome of the subject compared to a control or reference sample, the method comprising administering to the subject a therapeutically effective amount of a composition comprising one or more of the following:
a) one or more agents targeting growth or expansion of one or more genera of mucus-degrading bacteria in the gut microbiome of the subject;
b) one or more mucus-degrading enzyme inhibitors to inhibit mucus degradation by one or more genera of mucus-degrading bacteria in the gut microbiome of the subject; and/or
c) one or more mediators of organic acid metabolite levels produced following metabolism of mucin-derived carbohydrates by one or more genera of mucus-degrading bacteria in the gut microbiome of the subject;
wherein the one or more genera of mucus-degrading bacteria comprise Akkermansia or Bacteroides.

12. A method of treating cancer therapy-induced neutropenic fever in a subject receiving a cancer therapy and having decreased levels of one or more organic acid metabolites produced following metabolism of mucin-derived carbohydrates by one or more genera of mucus-degrading bacteria in the gut microbiome of the subject compared to a control or reference sample, the method comprising administering to the subject a therapeutically effective amount of a composition comprising one or more of the following:
a) one or more agents targeting growth or expansion of one or more genera of mucus-degrading bacteria in the gut microbiome of the subject;
b) one or more mucus-degrading enzyme inhibitors to inhibit mucus degradation by one or more genera of mucus-degrading bacteria in the gut microbiome of the subject; and/or
c) one or more mediators of organic acid metabolite levels produced following metabolism of mucin-derived carbohydrates by one or more genera of mucus-degrading bacteria in the gut microbiome of the subject;
wherein the one or more genera of mucus-degrading bacteria comprise Akkermansia or Bacteroides.

13. The method of any one of claims 4-12, wherein the control or reference sample is a sample from a healthy subject.

14. The method of any one of claims 4-12, wherein the control or reference sample is a sample from a subject who is diagnosed with neutropenia but who does not become febrile or develop neutropenic fever.

15. The method of any one of claims 4-12, wherein the control or reference sample is a sample from a subject who is diagnosed with neutropenia after administration of the cancer therapy but who does not become febrile or develop neutropenic fever.

16. The method of any one of claims 4-12, wherein the control or reference sample is a sample from a subject who is diagnosed with neutropenia who becomes febrile or develops neutropenic fever.

17. The method of any one of claims 4-12, wherein the control or reference sample is a sample from a subject who is diagnosed with neutropenia after administration of the cancer therapy who becomes febrile or develops neutropenic fever.

18. The method of any one of claims 1-17, wherein the subject does not exhibit symptoms of cancer therapy-induced neutropenic fever when the composition is administered.

19. The method of any one of claims 1-18, wherein the subject has been diagnosed with neutropenia.

20. The method of claim 19, wherein the composition is administered after the subject has been diagnosed with neutropenia.

21. The method of claim 19 or claim 20, wherein the composition is administered to the subject every day until the subject is no longer neutropenic.

22. The method of claim 21, wherein the composition is administered multiple times per day.

23. The method of claim 22, wherein the composition is administered 2, 3, 4, 5, or 6 times per day.

24. The method of any one of claims 19-21, wherein the subject is neutropenic due to the cancer therapy received by the subject.

25. The method of claim 24, wherein the cancer therapy received by the subject comprises one or more chemotherapies, radiotherapies, and/or immunotherapies.

26. The method of claim 25, wherein the one or more chemotherapies comprise alkylating agents, marrow-suppressive agents, reduced intensity conditioning, myeloablative conditioning, non-myeloablative conditioning, or immunosuppressive drugs.

27. The method of claim 25, wherein the one or more radiotherapies comprise external radiotherapy, internal radiotherapy, radioimmunotherapy, or intraoperative radiation therapy (IORT).

28. The method of claim 25, wherein the one or more immunotherapies comprise checkpoint inhibitors, inhibitors of co-stimulatory molecules, dendritic cell therapy, CAR-T cell therapy, cytokine therapy, or adoptive T cell therapy.

29. The method of any one of the preceding claims, wherein the one or more agents targeting growth or expansion of one or more genera of mucus-degrading bacteria comprise antibiotics or antimicrobial proteins or peptides.

30. The method of claim 29, wherein the antibiotics comprise azithromycin.

31. The method of any one of claims 1-28, wherein the one or more agents targeting growth or expansion of one or more genera of mucus-degrading bacteria comprise bucine, methyl-β-D-galactopyranoside, resacetophenone, or serotonin.

32. The method of any one of claims 1-28, wherein the one or more agents targeting growth or expansion of one or more genera of mucus-degrading bacteria comprise one or more ruminal metabolites.

33. The method of claim 32, wherein the one or more ruminal metabolites comprise malic acid, 3-indole acetic acid, hydrocinnamic acid, methylmalonic acid, gluconic acid, galacturonic acid, or bis-hydroxy methyl propionic acid.

34. The method of any one of the preceding claims, wherein the one or more mucus-degrading enzyme inhibitors comprise inhibitors of proteases, sulfatases, mucinases, or glycoside hydrolases.

35. The method of claim 34, wherein the glycoside hydrolases comprise neuraminidases/sialidases, fucosidases, N-acetylglucosaminidases, galactosidases, N-acetylglucosaminidases, or N-acetylgalactosaminidases.

36. The method of claim 35, wherein the neuramidase/sialidase inhibitors comprise siastatin B, zanamivir, peramivir, oseltamivir, or laninamivir.

37. The method of any one of the preceding claims, wherein the one or more mediators of organic acid metabolite levels comprise one or more vitamins, probiotics, prebiotics, or direct or indirect delivery of organic acid metabolites.

38. The method of claim 37, wherein the one or more vitamins comprise vitamin B12.

39. The method of claim 37, wherein the one or more organic acid metabolites comprise propionate, acetate, butyrate, isovalerate, or valerate.

40. The method of any one of claims 1-39, wherein the composition is orally administered.

41. The method of claim 40, wherein the composition is encapsulated.

42. The method of any one of the preceding claims, further comprising administering to the subject a therapeutically effective amount of a composition comprising one or more broad-spectrum antibiotics to treat, prevent, or reduce the severity of cancer therapy-induced neutropenic fever in the subject.

43. The method of claim 43, wherein the one or more broad-spectrum antibiotics comprise one or more cefepime and/or carbapenems.

44. The method of claim 42 or claim 43, wherein the carbapenems comprise meropenem, imipenem/cilastatin, panipenem/betamipron, biapenem, ertapenem, and/or doripenem.

45. The method of any one of claims 42-44, wherein administration of the one or more broad-spectrum antibiotics increases the risk of graft-versus-host disease (GVHD) to the subject compared to a subject to whom the one or more broad-spectrum antibiotics are not administered.

46. The method of claim 45, wherein the GVHD poses a greater risk to the health or life of the subject than such a condition would pose to an average person in the general population receiving the cancer therapy and/or the one or more broad-spectrum antibiotics.

47. The method of any one of claims 42-46, wherein the subject was determined to have decreased levels of one or more carbohydrate substrates metabolized by the one or more genera of mucus-degrading bacteria in the gut microbiome of the subject, an increased abundance of the one or more genera of mucus-degrading bacteria in the gut microbiome of the subject, and/or a decreased abundance of one or more commensal bacteria in the gut microbiome of the subject compared to a control or reference sample.

48. The method of claim 47, wherein the decreased levels of one or more carbohydrate substrates metabolized by the one or more genera of mucus-degrading bacteria in the gut microbiome of the subject, increased abundance of the one or more genera of mucus-degrading bacteria in the gut microbiome of the subject, and/or decreased abundance of one or more commensal bacteria in the gut microbiome of the subject were determined from a fecal sample from the subject.

49. The method of any one of claims 42-48, wherein the control or reference sample is a sample from a healthy subject or a subject to whom the one or more broad-spectrum antibiotics are not administered.

50. The method of any one of claims 42-49, further comprising administering to the subject a therapeutically effective amount of a composition comprising one or more carbohydrate substrates metabolized by the one or more genera of mucus-degrading bacteria in the gut microbiome to treat, prevent, and/or reduce the severity of GVHD.

51. The method of claim 50, wherein the one or more carbohydrate substrates metabolized by the one or more genera of mucus-degrading bacteria in the gut microbiome comprise arabinose, fructose, fucose, galactose, galacturonic acid, glucuronic acid, glucosamine, glucose, mannose, N-acetylglucosamine, N-acetylgalactosamine, rhamnose, ribose, xylose, pullulan, glycogen, amylopectin, inulin, levan, heparin, hyaluronan, chondroitin sulfate, polygalacturonate, rhamnogalacturonan, pectic galactan, arabinogalactan, arabinan, xylan, arabinoxylan, galactomannan, glucomannan, xyloglucan, β-glucan, cellobiose, laminarin, lichenin, dextran, and/or α-mannan.

52. The method of claim 50 or claim 51, wherein the one or more carbohydrate substrates metabolized by the one or more genera of mucus-degrading bacteria in the gut microbiome comprise mannose, glucose, and/or xylose.

53. The method of any one of claims 50-52, wherein the one or more carbohydrate substrates metabolized by the one or more genera of mucus-degrading bacteria in the gut microbiome comprise xylose.

54. The method of any one of claims 50-53, wherein the subject does not exhibit symptoms of GVHD when the composition is administered.

55. The method of any one of claims 50-54, wherein the subject has been diagnosed with GVHD.

56. The method of claim 55, wherein the composition is administered after the subject has been diagnosed with GVHD.

57. The method of claim 55 or claim 56, wherein the composition is administered to the subject every day until the subject no longer exhibits symptoms of GVHD and/or is determined to be cured of GVHD.

58. The method of claim 57, wherein the composition is administered multiple times per day.

59. The method of claim 58, wherein the composition is administered 2, 3, 4, 5, or 6 times per day.

60. The method of any one of claims 50-59, wherein the composition comprising one or more carbohydrate substrates metabolized by the one or more genera of mucus-degrading bacteria in the gut microbiome is orally administered.

61. The method of claim 60, wherein the composition is encapsulated.

62. The method of any one of claims 55-61, wherein the subject has GVHD due to the one or more broad-spectrum antibiotics received by the subject to treat, prevent, and/or reduce the severity of cancer therapy-induced neutropenic fever in the subject.

63. A method of predicting development of cancer therapy-induced neutropenic fever in a subject receiving a cancer therapy, the method comprising measuring an abundance of one or more genera of mucus-degrading bacteria in the gut microbiome of the subject, wherein:
a) the subject is likely to develop cancer therapy-induced neutropenic fever or is at risk of developing cancer therapy-induced neutropenic fever when the abundance of the one or more genera of mucus-degrading bacteria in the gut microbiome of the subject is increased compared to a control or reference sample; and/or
b) the subject is not at risk or is at reduced risk of developing cancer therapy-induced neutropenic fever when the abundance of the one or more genera of mucus-degrading bacteria in the gut microbiome of the subject are similar to or decreased compared to a control or reference sample; and
wherein the one or more genera of mucus-degrading bacteria comprise Akkermansia or Bacteroides.

64. The method of claim 63, wherein the subject is likely to develop cancer therapy-induced neutropenic fever or is at risk of developing cancer therapy-induced neutropenic fever when the abundance of the one or more genera of mucus-degrading bacteria in the gut microbiome of the subject comprises more than 0.5%, more than 0.6%, more than 0.7%, more than 0.8%, more than 0.9%, more than 1.0%, more than 1.1%, more than 1.2%, more than 1.3%, more than 1.4%, or more than 1.5% of the total gut microbiome bacterial population compared to a control or reference sample.

65. The method of claim 63 or claim 64, wherein when the abundance of the one or more genera of mucus-degrading bacteria in the gut microbiome of the subject is increased, the subject is provided a therapeutically effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

66. A method of predicting a therapy outcome for a subject in need of a cancer therapy, the method comprising measuring an abundance of one or more genera of mucus-degrading bacteria in the gut microbiome of the subject,
wherein when the abundance of the one or more genera of mucus-degrading bacteria in the gut microbiome of the subject is increased compared to control or reference sample, the subject has an increased likelihood of developing cancer therapy-induced neutropenic fever, and
wherein the one or more genera of mucus-degrading bacteria comprise Akkermansia or Bacteroides.

67. The method of claim 66, wherein the subject has an increased likelihood of developing cancer therapy-induced neutropenic fever when the abundance of the one or more genera of mucus-degrading bacteria in the gut microbiome of the subject comprises more than 0.5%, more than 0.6%, more than 0.7%, more than 0.8%, more than 0.9%, more than 1.0%, more than 1.1%, more than 1.2%, more than 1.3%, more than 1.4%, or more than 1.5% of the total gut microbiome bacterial population compared to a control or reference sample.

68. The method of claim 66 or claim 67, wherein when the abundance of the one or more genera of mucus-degrading bacteria in the gut microbiome of the subject is increased, the subject is provided a therapeutically effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

69. A method of predicting development of cancer therapy-induced neutropenic fever in a subject receiving a cancer therapy, the method comprising measuring functional activity and/or expression levels of one or more mucus-degrading enzymes secreted by one or more genera of mucus-degrading bacteria in the gut microbiome of the subject, wherein:
a) the subject is likely to develop cancer therapy-induced neutropenic fever or is at risk of developing cancer therapy-induced neutropenic fever when the functional activity or expression levels of one or more mucus-degrading enzymes secreted by one or more genera of mucus-degrading bacteria in the gut microbiome of the subject are increased compared to a control or reference sample; and/or
b) the subject is not at risk or is at reduced risk of developing cancer therapy-induced neutropenic fever when the functional activity or expression levels of one or more mucus-degrading enzymes secreted by one or more genera of mucus-degrading bacteria in the gut microbiome of the subject are similar to or decreased compared to a control or reference sample; and
wherein the one or more genera of mucus-degrading bacteria comprise Akkermansia or Bacteroides.

70. The method of claim 69, wherein the subject is likely to develop cancer therapy-induced neutropenic fever or is at risk of developing cancer therapy-induced neutropenic fever when the functional activity or expression levels of one or more mucus-degrading enzymes secreted by one or more genera of mucus-degrading bacteria in the gut microbiome of the subject are increased greater than 1-fold to greater than 100000-fold compared to a control or reference sample.

71. The method of claim 69 or claim 70, wherein when the functional activity or expression levels of one or more mucus-degrading enzymes secreted by one or more genera of mucus-degrading bacteria are increased, the subject is provided an effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

72. A method of predicting a therapy outcome for a subject in need of a cancer therapy, the method comprising measuring functional activity or expression levels of one or more mucus-degrading enzymes secreted by one or more genera of mucus-degrading bacteria in the gut microbiome of the subject,
wherein when the functional activity or expression levels of one or more mucus-degrading enzymes secreted by one or more genera of mucus-degrading bacteria are increased compared to a control or reference sample, the subject has an increased likelihood of developing cancer therapy-induced neutropenic fever, and
wherein the one or more genera of mucus-degrading bacteria comprise Akkermansia or Bacteroides.

73. The method of claim 72, wherein the subject has an increased likelihood of developing cancer therapy-induced neutropenic fever when the functional activity or expression levels of one or more mucus-degrading enzymes secreted by one or more genera of mucus-degrading bacteria in the gut microbiome of the subject are increased 1-fold to 100000-fold compared to a control or reference sample.

74. The method of claim 72 or claim 73, wherein when the functional activity or expression levels of one or more mucus-degrading enzymes secreted by one or more genera of mucus-degrading bacteria are increased, the subject is provided an effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

75. The method of any one of claims 69-74, wherein the one or more mucus-degrading enzymes comprise proteases, sulfatases, mucinases, or glycoside hydrolases.

76. The method of claim 75, wherein the glycoside hydrolases comprise neuraminidases/sialidases, fucosidases, N-acetylglucosaminidases, galactosidases, N-acetylglucosaminidases, or N-acetylgalactosaminidases.

77. A method of predicting development of cancer therapy-induced neutropenic fever in a subject receiving a cancer therapy, the method comprising measuring levels of one or more organic acid metabolites produced following metabolism of mucin-derived carbohydrates by one or more genera of mucus-degrading bacteria in the gut microbiome of the subject, wherein:
a) the subject is likely to develop cancer therapy-induced neutropenic fever or is at risk of developing cancer therapy-induced neutropenic fever when the levels of one or more organic acid metabolites produced following metabolism of mucin-derived carbohydrates by one or more genera of mucus-degrading bacteria in the gut microbiome of the subject are decreased compared to a control or reference sample;
and/or b) the subject is not at risk or is at reduced risk of developing cancer therapy-induced neutropenic fever when the levels of one or more organic acid metabolites produced following metabolism of mucin-derived carbohydrates by one or more genera of mucus-degrading bacteria in the gut microbiome of the subject are similar to or increased compared to a control or reference sample; and
wherein the one or more genera of mucus-degrading bacteria comprise Akkermansia or Bacteroides.

78. The method of claim 77, wherein the subject is likely to develop cancer therapy-induced neutropenic fever or is at risk of developing cancer therapy-induced neutropenic fever when the levels of one or more organic acid metabolites produced following metabolism of mucin-derived carbohydrates by one or more genera of mucus-degrading bacteria in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample.

79. The method of claim 77 or claim 78, wherein when the levels of one or more organic acid metabolites produced following metabolism of mucin-derived carbohydrates by one or more genera of mucus-degrading bacteria are decreased, the subject is provided an effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

80. A method of predicting a therapy outcome for a subject in need of cancer therapy, the method comprising measuring levels of one or more organic acid metabolites produced following metabolism of mucin-derived carbohydrates by one or more genera of mucus-degrading bacteria in the gut microbiome of the subject,
wherein when the levels of one or more organic acid metabolites produced following metabolism of mucin-derived carbohydrates by one or more genera of mucus-degrading bacteria are decreased compared to a control or reference sample, the subject has an increased likelihood of developing cancer therapy-induced neutropenic fever, and
wherein the one or more genera of mucus-degrading bacteria comprise Akkermansia or Bacteroides.

81. The method of claim 80, wherein the subject has an increased likelihood of developing cancer therapy-induced neutropenic fever when the levels of one or more organic acid metabolites produced following metabolism of mucin-derived carbohydrates by one or more genera of mucus-degrading bacteria in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample.

82. The method of claim 80 or claim 81, wherein when the levels of one or more organic acid metabolites produced following metabolism of mucin-derived carbohydrates by one or more genera of mucus-degrading bacteria are decreased, the subject is provided an effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

83. The method of any one of claims 77-82, wherein the organic acid metabolites comprise propionate, acetate, butyrate, isovalerate, or valerate.

84. A method of predicting development of cancer therapy-induced neutropenic fever in a subject receiving a cancer therapy, the method comprising measuring levels of one or more ruminal metabolites in the gut microbiome of the subject, wherein:
a) the subject is likely to develop cancer therapy-induced neutropenic fever or is at risk of developing cancer therapy-induced neutropenic fever when the levels of one or more ruminal metabolites that target the growth or expansion of one or more genera of mucus-degrading bacteria in the gut microbiome of the subject are decreased compared to a control or reference sample; and/or
b) the subject is not at risk or is at reduced risk of developing cancer therapy-induced neutropenic fever when the levels of one or more ruminal metabolites that target the growth or expansion of one or more genera of mucus-degrading bacteria in the gut microbiome of the subject are similar to or increased compared to a control or reference sample; and
wherein the one or more genera of mucus-degrading bacteria comprise Akkermansia or Bacteroides.

85. The method of claim 84, wherein the subject is likely to develop cancer therapy-induced neutropenic fever or is at risk of developing cancer therapy-induced neutropenic fever when the levels of one or more ruminal metabolites in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample.

86. The method of claim 77 or claim 78, wherein when the levels of one or more ruminal metabolites that target the growth or expansion of one or more genera of mucus-degrading bacteria in the gut microbiome of the subject are decreased, the subject is provided an effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

87. A method of predicting a therapy outcome for a subject in need of cancer therapy, the method comprising measuring levels of one or more ruminal metabolites that target the growth or expansion of one or more genera of mucus-degrading bacteria in the gut microbiome of the subject,
wherein when the levels of one or more ruminal metabolites that target the growth or expansion of one or more genera of mucus-degrading bacteria in the gut microbiome of the subject are decreased compared to a control or reference sample, the subject has an increased likelihood of developing cancer therapy-induced neutropenic fever, and
wherein the one or more genera of mucus-degrading bacteria comprise Akkermansia or Bacteroides.

88. The method of claim 87, wherein the subject has an increased likelihood of developing cancer therapy-induced neutropenic fever when the levels of one or more ruminal metabolites that target the growth or expansion of one or more genera of mucus-degrading bacteria in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample.

89. The method of claim 87 or claim 88, wherein when the levels of one or more ruminal metabolites that target the growth or expansion of one or more genera of mucus-degrading bacteria in the gut microbiome of the subject are decreased, the subject is provided an effective amount of a composition comprising one or more agents targeting growth or expansion of one or more genera of mucus-degrading bacteria in the gut microbiome of the subject, one or more mucus-degrading enzyme inhibitors to inhibit mucus degradation by one or more genera of mucus-degrading bacteria in the gut microbiome of the subject, and/or one or more mediators of organic acid metabolite levels produced following metabolism of mucin-derived carbohydrates by one or more genera of mucus-degrading bacteria in the gut microbiome of the subject.

90. The method of any one of claims 84-89, wherein the one or more ruminal metabolites comprise malic acid, 3-indole acetic acid, hydrocinnamic acid, methylmalonic acid, gluconic acid, galacturonic acid, or bis-hydroxy methyl propionic acid.

91. The method of claims 63-90, wherein the control or reference sample is a sample from a subject who is diagnosed with neutropenia but who does not become febrile or develop neutropenic fever.

92. The method of any one of claims 63-83, wherein the control or reference sample is a sample from a subject who is diagnosed with neutropenia after administration of the cancer therapy but who does not become febrile or develop neutropenic fever.

93. The method of any one of claims 63-83, wherein the control or reference sample is a sample from a subject who is diagnosed with neutropenia who becomes febrile or develops neutropenic fever.

94. The method of any one of claims 63-83, wherein the control or reference sample is a sample from a subject who is diagnosed with neutropenia after administration of the cancer therapy who becomes febrile or develops neutropenic fever.

95. The method of any one of claims 63-94, wherein the subject has been diagnosed with neutropenia.

96. The method of claim 95, wherein the subject is neutropenic due to the cancer therapy received by the subject.

97. The method of claim 96, wherein the cancer therapy received by the subject comprises one or more chemotherapies, radiotherapies, and/or immunotherapies.

98. The method of claim 97, wherein the one or more chemotherapies comprise alkylating agents, marrow-suppressive agents, reduced intensity conditioning, myeloablative conditioning, non-myeloablative conditioning, or immunosuppressive drugs.

99. The method of claim 97, wherein the one or more radiotherapies comprise external radiotherapy, internal radiotherapy, radioimmunotherapy, or intraoperative radiation therapy (IORT).

100. The method of claim 97, wherein the one or more immunotherapies comprise checkpoint inhibitors, inhibitors of co-stimulatory molecules, dendritic cell therapy, CAR-T cell therapy, cytokine therapy, or adoptive T cell therapy.

101. The method of any one of claims 95-100, wherein measuring the abundance of one or more genera of mucus-degrading bacteria, the functional activity and/or expression levels of one or more mucus-degrading enzymes secreted by one or more genera of mucus-degrading bacteria, the levels of one or more organic acid metabolites produced following metabolism of mucin-derived carbohydrates by one or more genera of mucus-degrading bacteria, and/or one or more ruminal metabolites that target the growth or expansion of one or more genera of mucus-degrading bacteria in the gut microbiome of the subject occurs after the subject has been diagnosed with neutropenia.

102. The method of any one of claim 65, 68, 71, 74, or 79, wherein the composition is orally administered.

103. The method of claim 102, wherein the composition is encapsulated.

104. A method of preventing or reducing the severity of graft-versus-host disease (GVHD), the method comprising prophylactically administering to a subject receiving a hematopoietic cell transplantation (HCT) therapy and/or a neutropenic fever therapy after administration of a cancer therapy a therapeutically effective amount of a composition comprising:
a) one or more agents targeting growth or expansion of one or more genera of mucus-degrading bacteria in the gut microbiome of the subject; and/or
b) one or more carbohydrate substrates metabolized by the one or more genera of mucus-degrading bacteria in the gut microbiome of the subject;
wherein the one or more genera of mucus-degrading bacteria comprise Bacteroides, Akkermansia, Ruminococcus, and Bifidobacterium.

105. The method of claim 104, wherein subject is at a higher risk than an average person in the general population receiving the HCT therapy and/or the neutropenic fever therapy of developing GVHD.

106. The method of claim 104 or claim 105, wherein the GVHD poses a greater risk to the health or life of the subject than such a condition would pose to an average person in the general population receiving the HCT therapy and/or the neutropenic fever therapy.

107. The method of any one of claims 104-106, wherein the subject was determined to have an increased abundance of mucus-degrading bacteria in the gut microbiome compared to a control or reference sample.

108. The method of claim 107, wherein the increased abundance of mucus-degrading bacteria in the gut microbiome was determined from a fecal sample from the subject.

109. The method of any one of claims 104-108, wherein the subject was determined to have a decreased abundance of one or more commensal bacteria in the gut microbiome compared to a control or reference sample.

110. The method of claim 109, wherein the one or more commensal bacteria in the gut microbiome comprise Clostridia.

111. The method of claim 109 or claim 110, wherein the decreased abundance of commensal bacteria in the gut microbiome was determined from a fecal sample from the subject.

112. The method of any one of claims 104-111, wherein the subject was determined to have decreased levels of one or more carbohydrate substrates metabolized by the one or more genera of mucus-degrading bacteria in the gut microbiome compared to a control or reference sample.

113. The method of claim 112, wherein the decreased levels of one or more carbohydrate substrates metabolized by the one or more genera of mucus-degrading bacteria in the gut microbiome was determined from a fecal sample from the subject.

114. A method of treating GVHD in a subject receiving a HCT therapy and/or a neutropenic fever therapy after administration of a cancer therapy and having an increased abundance of one or more genera of mucus-degrading bacteria in the gut microbiome of the subject compared to a control or reference sample, the method comprising administering to the subject a therapeutically effective amount of a composition comprising one or more of the following:
a) one or more agents targeting growth or expansion of the one or more genera of mucus-degrading bacteria in the gut microbiome of the subject; and/or
b) one or more carbohydrate substrates metabolized by the one or more genera of mucus-degrading bacteria in the gut microbiome of the subject;
wherein the one or more genera of mucus-degrading bacteria comprise Bacteroides, Akkermansia, Ruminococcus, and Bifidobacterium.

115. A method of treating GVHD in a subject receiving a HCT therapy and/or a neutropenic fever therapy after administration of a cancer therapy and having and having decreased levels of one or more carbohydrate substrates metabolized by one or more genera of mucus-degrading bacteria in the gut microbiome of the subject, the method comprising administering to the subject a therapeutically effective amount of a composition comprising one or more of the following:
a) one or more agents targeting growth or expansion of one or more genera of mucus-degrading bacteria in the gut microbiome of the subject; and/or
b) one or more carbohydrate substrates metabolized by the one or more genera of mucus-degrading bacteria in the gut microbiome of the subject;
wherein the one or more genera of mucus-degrading bacteria comprise Bacteroides, Akkermansia, Ruminococcus, and Bifidobacterium.

116. A method of treating GVHD in a subject receiving a HCT therapy and/or a neutropenic fever therapy after administration of a cancer therapy and having a decreased abundance of one or more commensal bacteria in the gut microbiome of the subject compared to a control or reference sample, the method comprising administering to the subject a therapeutically effective amount of a composition comprising one or more of the following:
a) one or more agents targeting growth or expansion of one or more genera of mucus-degrading bacteria in the gut microbiome of the subject; and/or
b) one or more carbohydrate substrates metabolized by the one or more genera of mucus-degrading bacteria in the gut microbiome of the subject;
wherein the one or more genera of mucus-degrading bacteria comprise Bacteroides, Akkermansia, Ruminococcus, and Bifidobacterium.

117. The method of any one of claims 107-116, wherein the control or reference sample is a sample from a healthy subject.

118. The method of any one of claims 107-116, wherein the control or reference sample is a sample from a subject to whom the HCT therapy and/or neutropenic fever therapy is not administered.

119. The method of any one of claims 104-118, wherein the composition is administered multiple times per day.

120. The method of any one of claims 104-119, wherein the composition is administered 2, 3, 4, 5, or 6 times per day.

121. The method of any one of claims 104-120, wherein the composition is orally administered.

122. The method of claim 121, wherein the composition is encapsulated.

123. The method of any one of claims 104-122, wherein the subject does not exhibit symptoms of GVHD when the composition is administered.

124. The method of any one of claims 104-123, wherein the subject has been diagnosed with GVHD.

125. The method of claim 124, wherein the composition is administered after the subject has been diagnosed with GVHD.

126. The method of claim 124 or claim 125, wherein the composition is administered to the subject every day until the subject no longer exhibits symptoms of GVHD and/or is determined to be cured of GVHD.

127. The method of any one of claims 124-126, wherein the subject is diagnosed with GVHD due to the HCT therapy and/or the neutropenic fever therapy received by the subject.

128. The method of any one of claims 104-127, wherein the cancer therapy administered to the subject comprises one or more chemotherapies, radiotherapies, and/or immunotherapies.

129. The method of claim 128, wherein the one or more chemotherapies comprise alkylating agents, marrow-suppressive agents, reduced intensity conditioning, myeloablative conditioning, non-myeloablative conditioning, or immunosuppressive drugs.

130. The method of claim 128, wherein the one or more radiotherapies comprise external radiotherapy, internal radiotherapy, radioimmunotherapy, or intraoperative radiation therapy (IORT).

131. The method of claim 128, wherein the one or more immunotherapies comprise checkpoint inhibitors, inhibitors of co-stimulatory molecules, dendritic cell therapy, CAR-T cell therapy, cytokine therapy, or adoptive T cell therapy.

132. The method of any one of claims 104-131, wherein the neutropenic fever therapy comprises one or more broad-spectrum antibiotics.

133. The method of claim 132, wherein the one or more broad-spectrum antibiotics comprise cefepime and/or carbapenems.

134. The method of claim 133, wherein the carbapenems comprise meropenem, imipenem/cilastatin, panipenem/betamipron, biapenem, ertapenem, and/or doripenem.

135. The method of any one of claims 104-134, wherein the HCT therapy comprises autologous, allogeneic, and/or syngeneic HCT therapy.

136. The method of claim 135, wherein the HCT therapy comprises allogeneic HCT therapy.

137. The method of any one of claims 104-136, wherein the one or more agents targeting growth or expansion of one or more genera of mucus-degrading bacteria comprise antibiotics or antimicrobial proteins or peptides.

138. The method of claim 137, wherein the antibiotics comprise azithromycin.

139. The method of any one of claims 104-136, wherein the one or more agents targeting growth or expansion of one or more genera of mucus-degrading bacteria comprise bucine, methyl-β-D-galactopyranoside, resacetophenone, or serotonin.

140. The method of any one of claims 104-136, wherein the one or more agents targeting growth or expansion of one or more genera of mucus-degrading bacteria comprise one or more ruminal metabolites.

141. The method of claim 140, wherein the one or more ruminal metabolites comprise malic acid, 3-indole acetic acid, hydrocinnamic acid, methylmalonic acid, gluconic acid, galacturonic acid, or bis-hydroxy methyl propionic acid.

142. The method of any one of claims 104-141, wherein the one or more carbohydrate substrates metabolized by the one or more genera of mucus-degrading bacteria in the gut microbiome of the subject comprise arabinose, fructose, fucose, galactose, galacturonic acid, glucuronic acid, glucosamine, glucose, mannose, N-acetylglucosamine, N-acetylgalactosamine, rhamnose, ribose, xylose, pullulan, glycogen, amylopectin, inulin, levan, heparin, hyaluronan, chondroitin sulfate, polygalacturonate, rhamnogalacturonan, pectic galactan, arabinogalactan, arabinan, xylan, arabinoxylan, galactomannan, glucomannan, xyloglucan, β-glucan, cellobiose, laminarin, lichenin, dextran, and/or α-mannan.

143. The method of claim 142, wherein the one or more carbohydrate substrates metabolized by the one or more genera of mucus-degrading bacteria in the gut microbiome comprise mannose, glucose, and/or xylose.

144. The method of claim 142 or claim 143, wherein the one or more carbohydrate substrates metabolized by the one or more genera of mucus-degrading bacteria in the gut microbiome comprise xylose.

145. A method of predicting development of GVHD in a subject receiving a HCT therapy and/or a neutropenic fever therapy after administration of a cancer therapy, the method comprising measuring an abundance of one or more genera of mucus-degrading bacteria in the gut microbiome of the subject, wherein:
a) the subject is likely to develop GVHD or is at risk of developing GVHD when the abundance of the one or more genera of mucus-degrading bacteria in the gut microbiome of the subject is increased compared to a control or reference sample; and/or
b) the subject is not at risk or is at reduced risk of developing GVHD when the abundance of the one or more genera of mucus-degrading bacteria in the gut microbiome of the subject is similar to or decreased compared to a control or reference sample; and
wherein the one or more genera of mucus-degrading bacteria comprise Bacteroides, Akkermansia, Ruminococcus, and Bifidobacterium.

146. The method of claim 145, wherein the subject is likely to develop GVHD or is at risk of developing GVHD when the abundance of the one or more genera of mucus-degrading bacteria in the gut microbiome of the subject comprises more than 5%, more than 6%, more than 7%, more than 8%, more than 9%, more than 10%, more than 11%, more than 12%, more than 13%, more than 14%, or more than 15% of the total gut microbiome bacterial population compared to a control or reference sample.

147. The method of claim 145 or claim 146, wherein when the abundance of the one or more genera of mucus-degrading bacteria in the gut microbiome of the subject is increased, the subject is provided a therapeutically effective amount of a composition comprising one or more agents targeting growth or expansion of one or more genera of mucus-degrading bacteria in the gut microbiome of the subject and/or one or more carbohydrate substrates metabolized by the one or more genera of mucus-degrading bacteria in the gut microbiome of the subject.

148. A method of predicting development of GVHD in a subject receiving a HCT therapy and/or a neutropenic fever therapy after administration of a cancer therapy, the method comprising measuring the levels of one or more carbohydrate substrates metabolized by the one or more genera of mucus-degrading bacteria in the gut microbiome of the subject, wherein:
a) the subject is likely to develop GVHD or is at risk of developing GVHD when the levels of one or more carbohydrate substrates metabolized by the one or more genera of mucus-degrading bacteria in the gut microbiome of the subject are decreased compared to a control or reference sample; and/or
b) the subject is not at risk or is at reduced risk of developing GVHD when the levels of one or more carbohydrate substrates metabolized by the one or more genera of mucus-degrading bacteria in the gut microbiome of the subject are similar to or increased compared to a control or reference sample; and
wherein the one or more genera of mucus-degrading bacteria comprise Bacteroides, Akkermansia, Ruminococcus, and Bifidobacterium.

149. The method of claim 148, wherein when the levels of one or more carbohydrate substrates metabolized by the one or more genera of mucus-degrading bacteria in the gut microbiome of the subject are decreased, the subject is provided a therapeutically effective amount of a composition comprising one or more agents targeting growth or expansion of one or more genera of mucus-degrading bacteria in the gut microbiome of the subject and/or one or more carbohydrate substrates metabolized by the one or more genera of mucus-degrading bacteria in the gut microbiome of the subject.

150. A method of predicting development of GVHD in a subject receiving a HCT therapy and/or a neutropenic fever therapy after administration of a cancer therapy, the method comprising measuring the abundance of one or more commensal bacteria in the gut microbiome of the subject, wherein:
a) the subject is likely to develop GVHD or is at risk of developing GVHD when the abundance of one or more commensal bacteria in the gut microbiome of the subject is decreased compared to a control or reference sample; and/or
b) the subject is not at risk or is at reduced risk of developing GVHD when the abundance of one or more commensal bacteria in the gut microbiome of the subject is similar to or increased compared to a control or reference sample.

151. The method of claim 150, wherein the subject is likely to develop GVHD or is at risk of developing GVHD when the abundance of one or more commensal bacteria in the gut microbiome of the subject is decreased to less than 0.5%, less than 1%, less than 2%, less than 3%, less than 4%, less than 5%, less than 6%, less than 7%, less than 8%, less than 9%, or less than 10% of the total gut microbiome bacterial population compared to a control or reference sample.

152. The method of claim 150 or claim 151, wherein when the abundance of one or more commensal bacteria in the gut microbiome of the subject is decreased, the subject is provided a therapeutically effective amount of a composition comprising one or more agents targeting growth or expansion of one or more genera of mucus-degrading bacteria in the gut microbiome of the subject and/or one or more carbohydrate substrates metabolized by the one or more genera of mucus-degrading bacteria in the gut microbiome of the subject.

153. A method of predicting development of GVHD in a subject receiving a HCT therapy and/or a neutropenic fever therapy after administration of a cancer therapy, the method comprising measuring the levels of one or more ruminal metabolites that target the growth or expansion of one or more genera of mucus-degrading bacteria in the gut microbiome of the subject, wherein:
a) the subject is likely to develop GVHD or is at risk of developing GVHD when the levels of the one or more ruminal metabolites are increased compared to a control or reference sample; and/or
b) the subject is not at risk or is at reduced risk of developing GVHD when the levels of the one or more ruminal metabolites are similar to or decreased compared to a control or reference sample; and
wherein the one or more genera of mucus-degrading bacteria comprise Bacteroides, Akkermansia, Ruminococcus, and Bifidobacterium.

154. The method of claim 153, wherein the subject is likely to develop GVHD or is at risk of developing GVHD when the levels of the one or more ruminal metabolites that target the growth or expansion of one or more genera of mucus-degrading bacteria in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample.

155. The method of claim 153 or claim 154, wherein when the levels of the one or more ruminal metabolites that target the growth or expansion of one or more genera of mucus-degrading bacteria in the gut microbiome of the subject are decreased, the subject is provided a therapeutically effective amount of a composition comprising one or more agents targeting growth or expansion of one or more genera of mucus-degrading bacteria in the gut microbiome of the subject and/or one or more carbohydrate substrates metabolized by the one or more genera of mucus-degrading bacteria in the gut microbiome of the subject.

156. A method of predicting development of GVHD in a subject receiving a HCT therapy and/or a neutropenic fever therapy after administration of a cancer therapy, the method comprising measuring the levels of the one or more ruminal metabolites that target the growth or expansion the levels of one or more genera of mucus-degrading bacteria in the gut microbiome of the subject, wherein:
a) the subject is likely to develop GVHD or is at risk of developing GVHD when the levels of the ruminal metabolites are decreased compared to a control or reference sample; and/or
b) the subject is not at risk or is at reduced risk of developing GVHD when the levels of the ruminal metabolites are similar to or increased compared to a control or reference sample.

157. The method of claim 156, wherein the subject is likely to develop GVHD or is at risk of developing GVHD when the levels of the one or more ruminal metabolites that target the growth or expansion of one or more genera of mucus-degrading bacteria in the gut microbiome of the subject are decreased to less than 10 mM, less than 9 mM, less than 8 mM, less than 7 mM, less than 6 mM, less than 5 mM, less than 4 mM, less than 3 mM, less than 2 mM or less than 1 mM compared to a control or reference sample.

158. The method of claim 156 or claim 157, wherein when levels of the one or more ruminal metabolites that target the growth or expansion of one or more genera of mucus-degrading bacteria in the gut microbiome of the subject are decreased, the subject is provided a therapeutically effective amount of a composition comprising one or more agents targeting growth or expansion of one or more genera of mucus-degrading bacteria in the gut microbiome of the subject and/or one or more carbohydrate substrates metabolized by the one or more genera of mucus-degrading bacteria in the gut microbiome of the subject.

159. The method of claims 145-158, wherein the control or reference sample is a sample from a healthy subject.

160. The method of any one of claims 145-152, wherein the control or reference sample is a sample from a subject to whom the HCT therapy and/or neutropenic fever therapy is not administered.

161. The method of any one of claim 147, 149, or 152, wherein the composition is orally administered.

162. The method of claim 161, wherein the composition is encapsulated.

163. The method of any one of claims 145-162, wherein the subject has been diagnosed with GVHD.

164. The method of claim 163, wherein the subject is diagnosed with GVHD due to the HCT therapy and/or the neutropenic fever therapy received by the subject.

165. The method of any one of claims 145-164, wherein the cancer therapy administered to the subject comprises one or more chemotherapies, radiotherapies, and/or immunotherapies.

166. The method of claim 165, wherein the one or more chemotherapies comprise alkylating agents, marrow-suppressive agents, reduced intensity conditioning, myeloablative conditioning, non-myeloablative conditioning, or immunosuppressive drugs.

167. The method of claim 165, wherein the one or more radiotherapies comprise external radiotherapy, internal radiotherapy, radioimmunotherapy, or intraoperative radiation therapy (IORT).

168. The method of claim 165, wherein the one or more immunotherapies comprise checkpoint inhibitors, inhibitors of co-stimulatory molecules, dendritic cell therapy, CAR-T cell therapy, cytokine therapy, or adoptive T cell therapy.

169. The method of any one of claims 145-168, wherein the neutropenic fever therapy comprises one or more broad-spectrum antibiotics.

170. The method of claim 169, wherein the one or more broad-spectrum antibiotics comprise cefepime and/or carbapenems.

171. The method of claim 170, wherein the carbapenems comprise meropenem, imipenem/cilastatin, panipenem/betamipron, biapenem, ertapenem, and/or doripenem.

172. The method of any one of claims 145-171, wherein the HCT therapy comprises autologous, allogeneic, and/or syngeneic HCT therapy.

173. The method of claim 172, wherein the HCT therapy comprises allogeneic HCT therapy.

174. The method of any one of claims 145-173, wherein measuring the abundance of one or more genera of mucus-degrading bacteria, the levels of one or more carbohydrate substrates metabolized by the one or more genera of mucus-degrading bacteria, the abundance of one or more commensal bacteria in the gut microbiome of the subject, and/or the levels of the one or more ruminal metabolites that target the growth or expansion of one or more genera of mucus-degrading bacteria in the gut microbiome of the subject occurs after the subject has been diagnosed with GVHD.

175. The method of any one of claims 1-174, wherein the subject has been diagnosed with cancer.

176. The method of claim 175, wherein the cancer comprises a solid tumor or is a hematological malignancy.

177. The method of any one of claims 1-176, wherein the subject is in need of a transplant therapy.

178. The method of claim 177, wherein the subject has a leukemia, myeloma, or lymphoma and is in need of a hematopoietic stem cell transplant therapy.

179. The method of any one of claims 1-178, wherein the identity or abundance of the one or more genera of bacteria in the gut microbiome is determined by shotgun sequencing of the genome of the one or more genera of bacteria.

180. The method of any one of claims 1-178, wherein the identity or abundance of the one or more genera of bacteria in the gut microbiome is determined by directed sequencing of the genome of the one or more genera of bacteria.

181. The method of claim 180, wherein the directed sequencing is of 16S rRNA of the one or more genera of bacteria.


SEQUENCE LISTING (11 sequences)

SEQ ID NO: 1 - DNA, 18 bp, Artificial Sequence
  Synthetic Polynucleotide
gctgcctcccgtaggagt

SEQ ID NO: 2 - DNA, 20 bp, Artificial Sequence
  Synthetic Polynucleotide
aaactcaaakgaattgacgg

SEQ ID NO: 3 - DNA, 18 bp, Artificial Sequence
  Synthetic Polynucleotide
ctcacrrcacgagctgac

SEQ ID NO: 4 - DNA, 20 bp, Artificial Sequence
  Synthetic Polynucleotide
cgcactcttcttgcatctgc

SEQ ID NO: 5 - DNA, 20 bp, Artificial Sequence
  Synthetic Polynucleotide
taccaacggctcacattggg

SEQ ID NO: 6 - DNA, 20 bp, Artificial Sequence
  Synthetic Polynucleotide
gatgctggaaaaggcaacgg

SEQ ID NO: 7 - DNA, 20 bp, Artificial Sequence
  Synthetic Polynucleotide
agcgtgccttttccttctga

SEQ ID NO: 8 - DNA, 25 bp, Artificial Sequence
  Synthetic Polynucleotide
ggtcaccgaaagacattattcatcg

SEQ ID NO: 9 - DNA, 23 bp, Artificial Sequence
  Synthetic Polynucleotide
gccgtttgatacagatccattcc

SEQ ID NO: 10 - DNA, 21 bp, Artificial Sequence
  Synthetic Polynucleotide
cacaacagccatagcgttcca

SEQ ID NO: 11 - DNA, 23 bp, Artificial Sequence
  Synthetic Polynucleotide
atcgcaaaaataagatgggcaaa